SEQUENCE LISTING (8 sequences)

SEQ ID NO: 1 - protein, 214 aa, synthetic construct
  REGION 1..214: note = hum13B8-b light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, synthetic construct
  REGION 1..446: note = hum13B8-b heavy chain
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, synthetic construct
  REGION 1..10: note = HC CDR1
GYIFITYWMT

SEQ ID NO: 4 - protein, 16 aa, synthetic construct
  REGION 1..16: note = HC CDR2
QIFPASGSADYNEKFE

SEQ ID NO: 5 - protein, 7 aa, synthetic construct
  REGION 1..7: note = HC CDR3
GGGGFAY

SEQ ID NO: 6 - protein, 11 aa, synthetic construct
  REGION 1..11: note = LC CDR1
RTSENIYSYLA

SEQ ID NO: 7 - protein, 7 aa, synthetic construct
  REGION 1..7: note = LC CDR2
NAKTLAE

SEQ ID NO: 8 - protein, 9 aa, synthetic construct
  REGION 1..9: note = LC CDR3
QHHYGIPFT

(19) United States
(12) Patent Application Publication
(10) Pub. No.: US 2024/0391993 A1
(43) Pub. Date: Nov. 28, 2024

(54) METHODS AND COMPOSITIONS FOR TREATING PLAQUE PSORIASIS

(71) Applicant: SUN PHARMACEUTICAL INDUSTRIES LIMITED, Mumbai (IN)

(72) Inventors: Siu-Long YAO, Washington Crossing, PA (US); Mudgal KOTHEKAR, Mumbai (IN); Shantanu MEHTA, Mumbai (IN); Tushar NISHANDAR, Mumbai (IN)

(73) Assignee: SUN PHARMACEUTICAL INDUSTRIES LIMITED, Mumbai (IN)

(21) Appl. No.: 18/674,352

(22) Filed: May 24, 2024

(30) Foreign Application Priority Data
May 25, 2023 (IN) 202321036274

(51) Int. Cl. C07K 16/24; A61K 9/00; A61K 39/00; A61P 17/06

(52) U.S. Cl. CPC C07K 16/244; A61K 9/0019; A61P 17/06; A61K 2039/505; A61K 2039/54; A61K 2039/545; C07K 2317/24; C07K 2317/56

(57) ABSTRACT

This disclosure relates to methods of treating plaque psoriasis comprising administering an anti-IL-23p19 antibody hum13B8-b to a patient in need thereof. This disclosure also relates to pharmaceutical compositions of an anti-IL-23p19 antibody hum13B8-b or antigen binding fragment thereof for the treatment plaque psoriasis in a patient. This disclosure further relates to the use of an anti-IL-23p19 antibody hum13B8-b or antigen binding fragment thereof for the manufacture of a medicament for treating plaque psoriasis in a patient. In some embodiments, the disclosure relates to methods, pharmaceutical compositions, and medicaments for treating plaque psoriasis wherein treatment results in the patient maintaining a Physician's Global Assessment (PGA) score of “clear” or “almost clear” with at least a 2-point reduction from Baseline, or results in the patient maintaining at least a 50% reduction in the Psoriasis Area and Severity Index (PASI 50), or results in the patient maintaining at least a 75% reduction in the Psoriasis Area and Severity Index (PASI 75), or results in the patient maintaining at least a 90% reduction in the Psoriasis Area and Severity Index (PASI 90), or results in the patient maintaining a 100% reduction in the Psoriasis Area and Severity Index (PASI 100) for at least up to about 60 weeks to at least up to about 432 weeks. In some embodiments, the disclosure relates to methods, pharmaceutical compositions, and medicaments for treating plaque psoriasis wherein treatment results in the patient experiencing no increase in adverse events (AEs), drug-related AEs, severe adverse events (SAEs), Tier 1 AEs, or Tier 2 AEs for at least up to about 60 weeks to at least up to about 432 weeks.

### CROSS REFERENCE TO RELATED APPLICATIONS

This application claims the benefit of Indian Application No. 202321036274, filed on May 25, 2023, the disclosure of which is incorporated by reference herein in its entirety.

### REFERENCE TO AN ELECTRONIC SEQUENCE LISTING

This application is being filed electronically and includes an electronically submitted sequence listing. The sequence listing is entitled “23-0711-US_Sequence-Listing.xml” and was created on May 24, 2024, and has a size of 11,332 bytes. The sequence listing contained in this .xml file is part of the specification and is herein incorporated by reference in its entirety.

### FIELD OF THE DISCLOSURE

This disclosure relates to methods of treating plaque psoriasis comprising administering an anti-IL-23p19 antibody hum13B8-b to a patient in need thereof. This disclosure also relates to pharmaceutical compositions of an anti-IL-23p19 antibody hum13B8-b or antigen binding fragment thereof for the treatment plaque psoriasis in a patient. This disclosure further relates to the use of an anti-IL-23p19 antibody hum13B8-b or antigen binding fragment thereof for the manufacture of a medicament for treating plaque psoriasis in a patient. In some embodiments, the disclosure relates to methods, pharmaceutical compositions, and medicaments for treating plaque psoriasis wherein treatment results in the patient maintaining a Physician's Global Assessment (PGA) score of “clear” or “almost clear” with at least a 2-point reduction from Baseline, or results in the patient maintaining at least a 50% reduction in the Psoriasis Area and Severity Index (PASI 50), or results in the patient maintaining at least a 75% reduction in the Psoriasis Area and Severity Index (PASI 75), or results in the patient maintaining at least a 90% reduction in the Psoriasis Area and Severity Index (PASI 90), or results in the patient maintaining a 100% reduction in the Psoriasis Area and Severity Index (PASI 100) for at least up to about 60 weeks to at least up to about 432 weeks. In some embodiments, the disclosure relates to methods, pharmaceutical compositions, and medicaments for treating plaque psoriasis wherein treatment results in the patient experiencing no increase in adverse events (AEs), drug-related AEs, severe adverse events (SAEs), Tier 1 AEs, or Tier 2 AEs for at least up to about 60 weeks to at least up to about 432 weeks.

### BACKGROUND

Psoriasis is a chronic inflammatory skin disease that affects 2% to 3% of the global population. Plaque psoriasis is the most common form, affecting 80% to 90% of patients. This is characterized by recurrent episodes of sharply demarcated, erythematous, scaly plaques of variable size and confluence. Psoriasis manifests in a wide range of severities, with approximately 25% of patients suffering from moderate-to-severe chronic plaque psoriasis who may be treated with topical agents, phototherapy, and/or systemic agents (conventional agents and biological treatments). Biological therapies are indicated for the treatment of patients with moderate-to-severe chronic plaque psoriasis who are also candidates for phototherapy or systemic therapy. Currently approved biological treatments include tumor necrosis factor antagonist agents such as etanercept, infliximab, and adalimumab, and p40 (IL-12 and IL-23) antagonists such as ustekinumab, guselkumab, and risankizumab, and IL-17 antagonists such as secukinumab, ixekizumab, and brodalumab (Sbidian et al., “Systemic pharmacological treatments for chronic plaque psoriasis: a network meta-analysis,” Cochrane Database Syst. Rev. 12(12): CD011535 (2017); Ellis et al., Br. J. Dermatol. 180(2): 282-88 (2019)).

IL-23 is a heterodimeric cytokine consisting of a unique p19 sub-unit and a common p40 sub-unit shared with IL-12. It is mainly produced by activated myeloid cells, and signals through a heterodimeric IL-23 receptor complex consisting of a unique IL-23 receptor (IL23R) paired with IL-12Rβ1. Soon after its discovery, IL-23 was recognized as a key driver of autoimmunity in mouse models and human diseases. This has been commonly attributed to the ability of IL-23 to polarize and activate Th17 cells, a subset of T cells that has been identified as having a central role in autoimmunity. In recent years, accumulating data has implicated the IL-23/Th17 pathway in psoriasis pathogenesis. Recent genome-wide association studies have identified psoriasis risk alleles around gene regions that encode IL-23 (TL23A, IL12B) and the IL-23 receptor (IL-23R). Indeed, both p19 and p40 sub-units of IL-23 are over-expressed in psoriatic skin lesions, while the unique p35 sub-unit of IL-12 is not.

Tildrakizumab (SCH 900222/MK-3222), hereafter referred to as tildrakizumab (MK-3222), is a high-affinity (297 pM), humanized IgG1/κ antibody that specifically binds to IL-23p19 (SN 08197) but does not bind human IL-12 (IL-12p40 and p35 heterodimer) or human p40. Efficacy and safety of tildrakizumab in the treatment of patients with moderate-to-severe plaque psoriasis was demonstrated in 2 pivotal Phase 3 studies (P010 and P011) (Beck et al., Psoriasis (Auckl.) 8: 49-58 (2018); Reich et al., Lancet 390(10091): 276-88 (2017)). Taken together, the resulting analyses determined that treatment with tildrakizumab demonstrated positive change in proportions of subjects with a PASI 75, PASI 90, or PASI 100 response and the proportion of subjects with a PGA score of “clear” or “minimal” with at least a 2-grade reduction. The combined results from the two studies also showed that tildrakizumab was generally well tolerated with a low incidence of drug-related AEs and AEs leading to discontinuation of the study medication.

However, there remains a need for therapeutic options for plaque psoriasis that are effective and can be safely administered over the long-term.

### SUMMARY

Provided herein is a method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Also provided herein is a method for maintaining a Physician's Global Assessment (PGA) score of “clear” or “almost clear” with at least a 2-point reduction from Baseline in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Further provided herein is a method for maintaining at least a 50% reduction in the Psoriasis Area and Severity Index (PASI 50) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Further provided herein is a method for maintaining at least a 75% reduction in the Psoriasis Area and Severity Index (PASI 75) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Further provided herein is a method for maintaining at least a 90% reduction in the Psoriasis Area and Severity Index (PASI 90) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Further provided herein is a method for maintaining a 100% reduction in the Psoriasis Area and Severity Index (PASI 100) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

Further provided herein is a method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of hum13B8-b results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

Further provided herein is a pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

Further provided herein is a pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

Further provided herein is the use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

Further provided herein is the use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTIONS OF THE DRAWINGS

The following detailed description of the embodiments of the present disclosure can be best understood when read in conjunction with the following drawings.

FIG. 1 is a schematic showing the study design of the base and extension study. Abbreviations: PASI=Psoriasis Area and Severity Index; NR=non responders; PR=partial responders; R=responders; D/C=discontinuation.

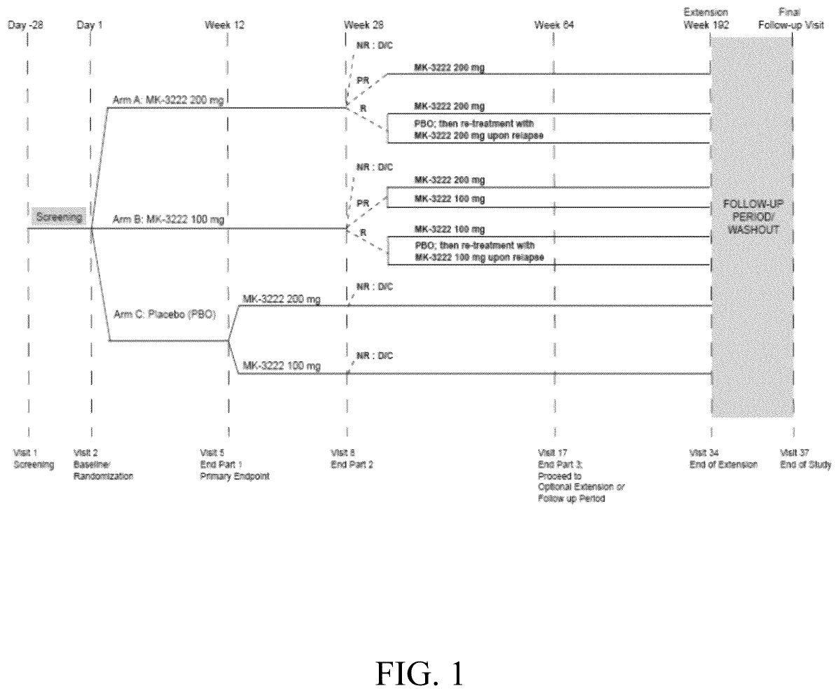

FIG. 2 is a schematic showing the study design for extensions 2 and 3.

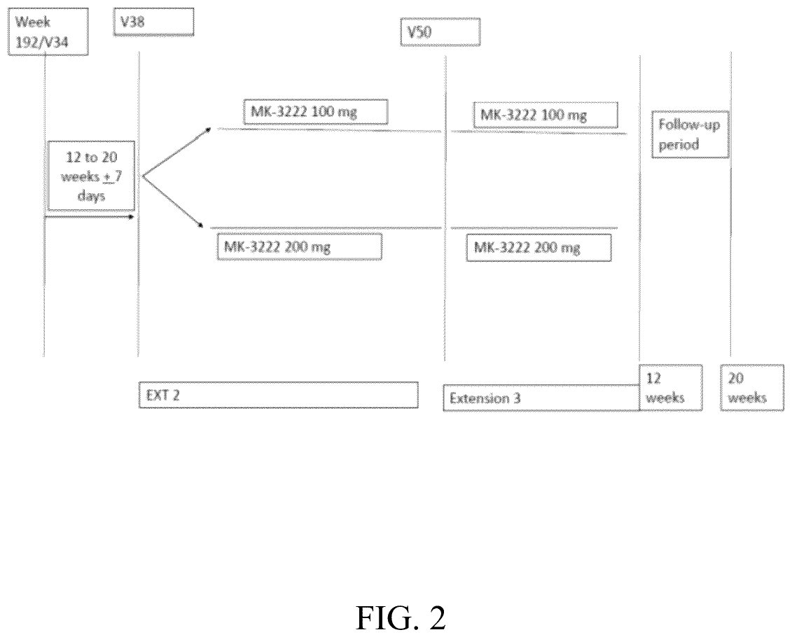

### DETAILED DESCRIPTION

The present disclosure relates to methods of treating plaque psoriasis comprising administering an anti-IL-23p19 antibody hum13B8-b to a patient in need thereof. The present disclosure also relates to pharmaceutical compositions of an anti-IL-23p19 antibody hum13B8-b or antigen binding fragment thereof for the treatment plaque psoriasis in a patient. The present disclosure further relates to the use of an anti-IL-23p19 antibody hum13B8-b or antigen binding fragment thereof for the manufacture of a medicament for treating plaque psoriasis in a patient. In some embodiments, the disclosure relates to methods, pharmaceutical compositions, and medicaments for treating plaque psoriasis wherein treatment results in the patient maintaining a Physician's Global Assessment (PGA) score of “clear” or “almost clear” with at least a 2-point reduction from Baseline, or results in the patient maintaining at least a 50% reduction in the Psoriasis Area and Severity Index (PASI 50), or results in the patient maintaining at least a 75% reduction in the Psoriasis Area and Severity Index (PASI 75), or results in the patient maintaining at least a 90% reduction in the Psoriasis Area and Severity Index (PASI 90), or results in the patient maintaining a 100% reduction in the Psoriasis Area and Severity Index (PASI 100) for at least up to about 60 weeks to at least up to about 432 weeks. In some embodiments, the disclosure relates to methods, pharmaceutical compositions, and medicaments for treating plaque psoriasis wherein treatment results in the patient experiencing no increase in adverse events (AEs), drug-related AEs, severe adverse events (SAEs), Tier 1 AEs, or Tier 2 AEs for at least up to about 60 weeks to at least up to about 432 weeks.

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. With regard to the administration of the anti-IL-23p19 antibody hum13B8-b or an antigen-binding fragment thereof as described herein, the term “about” when used with respect to a number of weeks means the number of weeks+/−7 days.

It is noted that terms like “preferably,” “commonly,” and “typically” are not used herein to limit the scope of the claimed subject matter or to imply that certain features are critical, essential, or even important to the structure or function of the claimed subject matter. Rather, these terms are merely intended to highlight alternative or additional features that can or cannot be used in a particular embodiment of the present disclosure.

For the purposes of describing and defining the present disclosure it is noted that the term “substantially” is used herein to represent the inherent degree of uncertainty that can be attributed to any quantitative comparison, value, measurement, or other representation. The term “substantially” is also used herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present.

As used in accordance with the present disclosure, unless otherwise indicated, all technical and scientific terms shall be understood to have the same meaning as commonly understood by one of ordinary skill in the art.

The present disclosure relates to relates to methods of treating plaque psoriasis comprising administering an anti-IL-23p19 antibody hum13B8-b to a patient in need thereof. This disclosure also relates to pharmaceutical compositions of an anti-IL-23p19 antibody hum13B8-b or antigen binding fragment thereof for the treatment plaque psoriasis in a patient. In one embodiment, provided herein is a method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method for maintaining a Physician's Global Assessment (PGA) score of “clear” or “almost clear” with at least a 2-point reduction from Baseline in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method for maintaining at least a 50% reduction in the Psoriasis Area and Severity Index (PASI 50) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method for maintaining at least a 75% reduction in the Psoriasis Area and Severity Index (PASI 75) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method for maintaining at least a 90% reduction in the Psoriasis Area and Severity Index (PASI 90) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method for maintaining a 100% reduction in the Psoriasis Area and Severity Index (PASI 100) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks to at least up to about 432 weeks.

In another embodiment, provided herein is a method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of hum13B8-b results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

The present disclosure also relates to pharmaceutical compositions of an anti-IL-23p19 antibody hum13B8-b or antigen binding fragment thereof for the treatment plaque psoriasis in a patient. In one embodiment, the present disclosure provides a pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks to at least up to about 432 weeks.

In another embodiment, provided herein is a pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the pharmaceutical composition results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

The present disclosure also relates to the use of an anti-IL-23p19 antibody hum13B8-b or antigen binding fragment thereof for the manufacture of a medicament for treating plaque psoriasis in a patient. In one embodiment, the present disclosure provides the use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks to at least up to about 432 weeks.

In another embodiment, provided herein is the use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein administration of the medicament results in the patient experiencing no increase in adverse events (AEs), drug-related AEs, severe adverse events (SAEs), Tier 1 AEs, or Tier 2 AEs for at least up to about 60 weeks to at least up to about 432 weeks as compared to treatment for up to about 52 weeks.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen binding fragment” as used herein refers to a portion of an intact antibody and/or refers to the antigenic determining variable domains of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In particular embodiments, the anti-IL-23p19 antibody hum13B8-b is tildrakizumab. The term “tildrakizumab” as used herein refers to a humanized anti-IL-23p19 monoclonal antibody, also known as SCH 900222 or MK-3222. Tildrakizumab is a high-affinity (297 picomolar [pM]) humanized immunoglobulin G1/kappa (IgG1/κ) antibody that specifically binds to the p19 protein of the IL-23 heterodimer but does not bind human IL-12 (IL-12/23p40 and IL12p35 heterodimer) or human IL-12/23p40. Pharmacokinetics: Tildrakizumab pharmacokinetics increases proportionally over a dose range from 50 mg to 200 mg (0.5 to 2 times the approved recommended dosage) following subcutaneous administration in subjects with plaque psoriasis. Steady-state concentrations were achieved by Week 16 following subcutaneous administration of tildrakizumab at Weeks 0, 4, and every 12 weeks thereafter. At the 100 mg dose at Week 16, the mean (±SD) steady-state trough concentrations ranged from 1.22±0.94 mcg/mL to 1.47±1.12 mcg/mL. The geometric mean (CV %) steady-state Cmax was 8.1 mcg/mL (34%). The absolute bioavailability of tildrakizumab was estimated to be 73-80% following subcutaneous injection. The peak concentration (Cmax) was reached by approximately 6 days.

In some embodiments, the anti-IL-23p19 antibody tildrakizumab can refer to ILUMYA®. ILUMYA® is administered by subcutaneous injection at a recommended dosage of 100 mg at weeks 0, 4, and every 12 weeks thereafter. In some embodiments, tildrakizumab is formulated in a 1 mL single-dose prefilled syringe containing 100 mg of tildrakizumab (i.e., 100 mg/mL). In some embodiments, ILUMYA® (tildrakizumab-asmn) injection, for subcutaneous use, is a sterile, clear to slightly opalescent, colorless to slightly yellow solution. ILUMYA® is supplied in a single-dose prefilled syringe with a glass barrel and 29-gauge fixed, ½-inch needle. In some embodiments, tildrakizumab can be formulated in: L-histidine, L-histidine hydrochloride monohydrate, polysorbate 80, and/or sucrose, in Water for Injection, with a pH of 5.7-6.3. In some embodiments, tildrakizumab is formulated in a 1 mL single-dose prefilled syringe containing 100 mg of tildrakizumab-asmn formulated in: L-histidine (0.495 mg), L-histidine hydrochloride monohydrate (1.42 mg), polysorbate 80 (0.5 mg), sucrose (70.0 mg), and Water for Injection, USP with a pH of 5.7-6.3.

In particular embodiments, the anti-IL-23p19 antibody hum13B8-b (tildrakizumab) comprises a light chain polypeptide comprising the amino acid sequence of SEQ ID NO: 1 and a heavy chain polypeptide comprising the amino acid sequence of SEQ ID NO: 2, and which is disclosed in U.S. Pat. Nos. 8,404,813 and 8,293,883, the disclosures of each of which are hereby incorporated by reference in their entireties. In other embodiments, the anti-IL-23p19 antibody hum13B8-b or an antigen-binding fragment thereof comprises a heavy chain variable domain and a light chain variable domain, wherein the heavy chain variable domain comprises CDR1, CDR2, and CDR3 sequences of the amino acid sequences of SEQ ID NOs: 3-5, and wherein the light chain variable domain comprises CDR1, CDR2, and CDR3 sequences of the amino acid sequences of SEQ ID NOs: 6-8.

Hum 13B8-b Light Chain
(SEQ ID NO: 1)
DIQMTQSPSSLSASVGDRVTITCRTSENIYSYLAWYQQKPGKAPKLLIY
NAKTLAEGVPSRFSGSGSGTDFTLTISSLQPEDFATYYCQHHYGIPFTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Hum 13B8-b Heavy Chain
(SEQ ID NO: 2)
QVQLVQSGAEVKKPGASVKVSCKASGYIFITYWMTWVRQAPGQGLEWMG
QIFPASGSADYNEKFEGRVTMTTDTSTSTAYMELRSLRSDDTAVYYCAR
GGGGFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Hum 13B8-b Heavy Chain CDR1
(SEQ ID NO: 3)
GYIFITYWMT
Hum 13B8-b Heavy Chain CDR2
(SEQ ID NO: 4)
QIFPASGSADYNEKFE
Hum 13B8-b Heavy Chain CDR3
(SEQ ID NO: 5)
GGGGFAY
Hum 13B8-b Light Chain CDR1
(SEQ ID NO: 6)
RTSENIYSYLA
Hum 13B8-b Light Chain CDR2
(SEQ ID NO: 7)
NAKTLAE
Hum 13B8-b Light Chain CDR3
(SEQ ID NO: 8)
QHHYGIPFT

As used herein, the term “subject” and “patient” are interchangeable. In some embodiments, subjects and/or patients are mammals.

A “disorder” is any condition that would benefit from treatment using the antibodies of the disclosure. “Disorder” and “condition” are used interchangeably herein and include chronic and acute disorders or diseases, including those pathological conditions that predispose a patient to the disorder in question.

The terms “treatment” or “treat” as used herein refer to both therapeutic treatment and prophylactic or preventative measures. Those in need of treatment include patients having plaque psoriasis as well as those prone to have plaque psoriasis or those in which plaque psoriasis is to be prevented. In some embodiments, the plaque psoriasis is moderate to severe plaque psoriasis.

The terms “administration” or “administering” as used herein refer to providing, contacting, and/or delivering an antibody or fragment thereof by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants. In one embodiment, administration is subcutaneous via a pre-filled syringe (PFS).

In some embodiments, the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered to the patient subcutaneously. In some embodiments, the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered to the patient by subcutaneous injection. In some embodiments, the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered to the patient using an auto-injector or prefilled syringe.

In some embodiments, the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered about every two weeks, about every four weeks, about every six weeks, about every eight weeks, about every ten weeks, or about every twelve weeks.

As used herein, the term “Week 0” refers to the first day the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered.

In some embodiments, the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

The therapy dose or therapeutically effective amount of the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament will vary depending, in part, upon the size (body weight, body surface, or organ size) and condition (the age and general health) of the patient. In some embodiments, the patient is administered one or more doses of the anti-IL-23p19 antibody hum13B8-b or an antigen-binding fragment thereof, wherein the dose is about 20 mg, 40 mg, 60 mg, 80 mg, 100 mg, 120 mg, 140 mg, 160 mg, 180 mg, or 200 mg.

The term “therapeutically effective amount” as applied to dose or amount refers to the quantity of a compound or pharmaceutical composition that is sufficient to result in a desired effect upon administration to a patient in need thereof. As used herein with respect to the pharmaceutical compositions comprising the anti-IL-23p19 antibody hum13B8-b (i.e., tildrakizumab), the term “therapeutically effective amount” also refers to the dose of a compound or pharmaceutical composition that is sufficient to produce an effective response upon administration to a patient. In some embodiments, a therapeutically effective amount of the anti-IL-23p19 antibody hum13B8-b (i.e., tildrakizumab) refers to a dose of 20 mg, 40 mg, 60 mg, 80 mg, 100 mg, 120 mg, 140 mg, 160 mg, 180 mg, or 200 mg. In some embodiments, a therapeutically effective amount of the anti-IL-23p19 antibody hum13B8-b (i.e., tildrakizumab) is a dose of 100 mg. In some embodiments, a therapeutically effective amount of the anti-IL-23p19 antibody hum13B8-b (i.e., tildrakizumab) is a dose of 100 mg at week 0 and every 12 weeks thereafter. In some embodiments, a therapeutically effective amount of the anti-IL-23p19 antibody hum13B8-b (i.e., tildrakizumab) is a dose of 100 mg at weeks 0, 4, and every 12 weeks thereafter. In some embodiments, a therapeutically effective amount of the anti-IL-23p19 antibody hum13B8-b (i.e., tildrakizumab) is a dose of 200 mg at week 0 and every 12 weeks thereafter. In some embodiments, a therapeutically effective amount of the anti-IL-23p19 antibody hum13B8-b (i.e., tildrakizumab) is a dose of 200 mg at weeks 0, 4, and every 12 weeks thereafter.

In some embodiments, the first dose and the subsequent dose of the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament are the same. In some embodiments, the first dose and the subsequent dose of the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament are different. In some embodiments, the first dose is 100 mg. In some embodiments, the first dose is 200 mg. In some embodiments, the subsequent dose is 100 mg. In some embodiments, the subsequent dose is 200 mg. In some embodiments, the first dose and the subsequent dose are 100 mg. In some embodiments, the first dose and the subsequent dose are 200 mg. In some embodiments, the first dose and the subsequent dose contain 100 mg hum13B8-b. In some embodiments, the first dose and the subsequent dose contain 200 mg hum13B8-b.

In some embodiments, the first dose, the second dose, and the subsequent dose of the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament are the same. In some embodiments, the first dose, the second dose, and the subsequent dose of the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament are different. In some embodiments, the first dose is 100 mg. In some embodiments, the first dose is 200 mg. In some embodiments, the second dose is 100 mg. In some embodiments, the second dose is 200 mg. In some embodiments, the subsequent dose is 100 mg. In some embodiments, the subsequent dose is 200 mg. In some embodiments, the first dose, the second dose, and the subsequent dose are 100 mg. In some embodiments, the first dose, the second dose, and the subsequent dose are 200 mg. In some embodiments, the first dose, the second dose, and the subsequent dose contain 100 mg hum13B8-b. In some embodiments, the first dose, the second dose, and the subsequent dose contain 200 mg hum13B8-b.

Physician Global Assessment (PGA) of Skin (Whole body) refers to a 5-point, 0-4 measure that is a useful clinician assessment of psoriasis lesions on the skin based on degree of erythema, thickness, and scale averaged over the entire body. Each of the clinical signs are assessed on a 0-5 scale (0=clear, 1=minimal, 2=mild, 3=moderate, and 4=severe). In some embodiments, a significant improvement of plaque psoriasis as assessed by Physician Global Assessment of Skin (Whole body) can refer to subjects with a PGA of skin (whole body) score of “clear” or “almost clear” with at least a 2-point reduction from Baseline. In some embodiments, a significant improvement of plaque psoriasis as assessed by Physician Global Assessment of Skin (Whole body) can refer to subjects with a PGA of skin (whole body) score of “clear” with at least a 2-point reduction from Baseline. In some embodiments, a significant improvement of plaque psoriasis as assessed by Physician Global Assessment of Skin (Whole body) can refer to subjects with a PGA of skin (whole body) score of “clear” or “almost clear” with at least a 2-point reduction from Baseline for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks. In some embodiments, a significant improvement of plaque psoriasis as assessed by Physician Global Assessment of Skin (Whole body) can refer to subjects with a PGA of skin (whole body) score of “clear” with at least a 2-point reduction from Baseline for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Psoriasis Area and Severity Index (PASI) is used to determine the treatment response (PASI 50, PASI 75, PASI 90, and PASI 100) in subjects with plaque psoriasis. The PASI includes scores on erythema, thickness, scaling, and percentage of body surface area (BSA) affected. In some embodiments, a significant improvement of plaque psoriasis as assessed by PASI can refer to a subject achieving at least a 50% improvement in the PASI score from Baseline for at least up to about 60 weeks. In some embodiments, a significant improvement of plaque psoriasis as assessed by PASI can refer to a subject achieving at least a 75% improvement in the PASI score from Baseline for at least up to about 60 weeks. In some embodiments, a significant improvement of plaque psoriasis as assessed by PASI can refer to a subject achieving at least a 90% improvement in the PASI score from Baseline for at least up to about 60 weeks. In some embodiments, a significant improvement of plaque psoriasis as assessed by PASI can refer to a subject achieving at least a 100% improvement in the PASI score from Baseline for at least up to about 60 weeks. In some embodiments, a significant improvement of plaque psoriasis as assessed by PASI can refer to a subject achieving at least a 50%, 75%, 90%, or 100% improvement in the PASI score from Baseline for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

As used herein, the term “significant improvement” refers to significant positive effect in a response in patients taking the anti-IL-23p19 antibody hum13B8-b or an antigen-binding fragment thereof, pharmaceutical composition of an anti-IL-23p19 antibody hum13B8-b, or anti-IL-23p19 antibody hum13B8-b medicament relative to patients taking a placebo. In some embodiments, a significant improvement of plaque psoriasis is assessed by Physician Global Assessment (PGA) of Skin (Whole body) or Psoriasis Area and Severity Index (PASI). In certain embodiments, the significant improvement refers to a statistically significant improvement. In certain embodiments, the term “statistically significant” means having a probability of less than 10% under the relevant null hypothesis (i.e., p<0.1). In some embodiments, a p-value less than 0.05 is considered to be statistically significant. In some embodiments, a p-value less than 0.01 is considered to be statistically significant. In some embodiments, a p-value less than 0.005 is considered to be statistically significant. In some embodiments, a p-value less than 0.0025 is considered to be statistically significant. In some embodiments, a p-value less than 0.001 is considered to be statistically significant. In certain embodiments, statistical tests will be 2-sided at the 5% significance level, and point estimates are accompanied with 2-sided 95% confidence intervals (CIs), where applicable.

In some embodiments, a significant improvement can refer to an improvement of plaque psoriasis of at least 10%, 15%, 20%, 25%, 30%, 35%, 40%, 45%, 50%, 55%, 60%, 65%, 70%, 75%, 80%, 85%, 90%, 95%, 100%, 150%, or 200% or more as assessed by Physician Global Assessment (PGA) of Skin (Whole body) or Psoriasis Area and Severity Index (PASI).

In some embodiments, a significant improvement can refer to an at least 2-fold, 3-fold, 4-fold, 5-fold, or 10-fold, or more than 10-fold improvement of plaque psoriasis as assessed by Physician Global Assessment (PGA) of Skin (Whole body) or Psoriasis Area and Severity Index (PASI).

The terms “pharmaceutical composition” or “therapeutic composition” as used herein refer to a compound or composition capable of inducing a desired therapeutic effect when properly administered to a patient. One embodiment of the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier” as used herein refer to one or more formulation materials suitable for accomplishing or enhancing the delivery of one or more antibodies of the disclosure.

Pharmaceutical compositions comprising tildrakizumab, either alone or in combination with prophylactic agents, therapeutic agents, and/or pharmaceutically acceptable carriers are provided. The pharmaceutical compositions comprising tildrakizumab provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art.

### Embodiments

Embodiment 1: A method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 2: The method according to embodiment 1, wherein the plaque psoriasis is moderate to severe plaque psoriasis.

Embodiment 3: The method according to embodiment 1, wherein hum13B8-b is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 4: The method according to embodiment 1, wherein hum13B8-b is administered to the patient about every 12 weeks.

Embodiment 5: The method according to embodiment 1, wherein hum13B8-b is administered to the patient subcutaneously.

Embodiment 6: The method according to embodiment 5, wherein hum13B8-b is administered to the patient by subcutaneous injection.

Embodiment 7: The method according to embodiment 6, wherein hum13B8-b is administered to the patient using an auto-injector or prefilled syringe.

Embodiment 8: The method according to embodiment 1, wherein a therapeutically effective amount of hum13B8-b is administered to the patient.

Embodiment 9: The method according to embodiment 1, wherein 100 mg or 200 mg of hum13B8-b is administered to the patient.

Embodiment 10: The method according to embodiment 9, wherein 100 mg of hum13B8-b is administered to the patient.

Embodiment 11: The method according to embodiment 9, wherein 200 mg of hum13B8-b is administered to the patient.

Embodiment 12: The method according to embodiment 1, wherein a first dose of hum13B8-b is administered to the patient on week 0 and a subsequent dose of hum13B8-b is administered to the patient about every 12 weeks thereafter.

Embodiment 13: The method according to embodiment 12, wherein the first dose and the subsequent dose are the same.

Embodiment 14: The method according to embodiment 12, wherein the first dose and the subsequent dose are different.

Embodiment 15: The method according to embodiment 12, wherein the first dose is 100 mg.

Embodiment 16: The method according to embodiment 12, wherein the first dose is 200 mg.

Embodiment 17: The method according to embodiment 12, wherein the subsequent dose is 100 mg.

Embodiment 18: The method according to embodiment 12, wherein the subsequent dose is 200 mg.

Embodiment 19: The method according to embodiment 13, wherein the first dose and the subsequent dose are 100 mg.

Embodiment 20: The method according to embodiment 13, wherein the first dose and the subsequent dose are 200 mg.

Embodiment 21: The method according to embodiment 14, wherein the first dose is 100 mg and the subsequent dose is 200 mg.

Embodiment 22: The method according to embodiment 14, wherein the first dose is 200 mg and the subsequent dose is 100 mg.

Embodiment 23: The method according to embodiment 12, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 24: The method according to embodiment 1, wherein a first dose of hum13B8-b is administered to the patient on week 0, a second dose of hum13B8-b is administered to the patient at about 4 weeks, and a subsequent dose of hum13B8-b is administered to the patient about every 4 to 12 weeks thereafter.

Embodiment 25: The method according to embodiment 24, wherein the first dose, the second dose, and the subsequent dose are the same.

Embodiment 26: The method according to embodiment 25, wherein the first dose, the second dose, and the subsequent dose are 100 mg.

Embodiment 27: The method according to embodiment 25, wherein the first dose, the second dose, and the subsequent dose are 200 mg.

Embodiment 28: The method according to embodiment 24, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 29: The method according to embodiment 1, wherein administration of hum13B8-b results in the patient maintaining a Physician's Global Assessment (PGA) score of “clear” or “almost clear” with at least a 2-point reduction from Baseline for at least up to about 60 weeks.

Embodiment 30: The method according to embodiment 29, wherein administration of hum13B8-b results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 31: The method according to embodiment 1, wherein administration of hum13B8-b results in the patient maintaining at least a 50% reduction in the Psoriasis Area and Severity Index (PASI 50) for at least up to about 60 weeks.

Embodiment 32: The method according to embodiment 31, wherein administration of hum13B8-b results in the patient maintaining at least a 50% reduction in the Psoriasis Area and Severity Index (PASI 50) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 33: The method according to embodiment 1, wherein administration of hum13B8-b results in the patient maintaining at least a 75% reduction in the Psoriasis Area and Severity Index (PASI 75) for at least up to about 60 weeks.

Embodiment 34: The method according to embodiment 33, wherein administration of hum13B8-b results in the patient maintaining at least a 75% reduction in the Psoriasis Area and Severity Index (PASI 75) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 35: The method according to embodiment 1, wherein administration of hum13B8-b results in the patient maintaining at least a 90% reduction in the Psoriasis Area and Severity Index (PASI 90) for at least up to about 60 weeks.

Embodiment 36: The method according to embodiment 35, wherein administration of hum13B8-b results in the patient maintaining at least a 90% reduction in the Psoriasis Area and Severity Index (PASI 90) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 37: The method according to embodiment 1, wherein administration of hum13B8-b results in the patient maintaining a 100% reduction in the Psoriasis Area and Severity Index (PASI 100) for at least up to about 60 weeks.

Embodiment 38: The method according to embodiment 37, wherein administration of hum13B8-b results in the patient maintaining a 100% reduction in the Psoriasis Area and Severity Index (PASI 100) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 39: The method according to embodiment 1, wherein administration of hum13B8-b results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 40: The method according to embodiment 39, wherein administration of hum13B8-b results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 41: A method for maintaining a Physician's Global Assessment (PGA) score of “clear” or “almost clear” with at least a 2-point reduction from Baseline in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 42: A method for maintaining at least a 50% reduction in the Psoriasis Area and Severity Index (PASI 50) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 43: A method for maintaining at least a 75% reduction in the Psoriasis Area and Severity Index (PASI 75) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 44: A method for maintaining at least a 90% reduction in the Psoriasis Area and Severity Index (PASI 90) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 45: A method for maintaining a 100% reduction in the Psoriasis Area and Severity Index (PASI 100) in a patient with plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein hum13B8-b is administered to the patient for at least up to about 60 weeks.

Embodiment 46: The method according to any one of embodiments 41-45, wherein the plaque psoriasis is moderate to severe plaque psoriasis.

Embodiment 47: The method according to any one of embodiments 41-45, wherein hum13B8-b is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 48: The method according to any one of embodiments 41-45, wherein hum13B8-b is administered to the patient about every 12 weeks.

Embodiment 49: The method according to any one of embodiments 41-45, wherein hum13B8-b is administered to the patient subcutaneously.

Embodiment 50: The method according to embodiment 49, wherein hum13B8-b is administered to the patient by subcutaneous injection.

Embodiment 51: The method according to embodiment 50, wherein hum13B8-b is administered to the patient using an auto-injector or prefilled syringe.

Embodiment 52: The method according to any one of embodiments 41-45, wherein a therapeutically effective amount of hum13B8-b is administered to the patient.

Embodiment 53: The method according to any one of embodiments 41-45, wherein 100 mg or 200 mg of hum13B8-b is administered to the patient.

Embodiment 54: The method according to embodiment 53, wherein 100 mg of hum13B8-b is administered to the patient.

Embodiment 55: The method according to embodiment 53, wherein 200 mg of hum13B8-b is administered to the patient.

Embodiment 56: The method according to any one of embodiments 41-45, wherein a first dose of hum13B8-b is administered to the patient on week 0 and a subsequent dose of hum13B8-b is administered to the patient about every 12 weeks thereafter.

Embodiment 57: The method according to embodiment 56, wherein the first dose and the subsequent dose are the same.

Embodiment 58: The method according to embodiment 56, wherein the first dose and the subsequent dose are different.

Embodiment 59: The method according to embodiment 56, wherein the first dose is 100 mg.

Embodiment 60: The method according to embodiment 56, wherein the first dose is 200 mg.

Embodiment 61: The method according to embodiment 56, wherein the subsequent dose is 100 mg.

Embodiment 62: The method according to embodiment 56, wherein the subsequent dose is 200 mg.

Embodiment 63: The method according to embodiment 57, wherein the first dose and the subsequent dose are 100 mg.

Embodiment 64: The method according to embodiment 57, wherein the first dose and the subsequent dose are 200 mg.

Embodiment 65: The method according to embodiment 58, wherein the first dose is 100 mg and the subsequent dose is 200 mg.

Embodiment 66: The method according to embodiment 58, wherein the first dose is 200 mg and the subsequent dose is 100 mg.

Embodiment 67: The method according to embodiment 56, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 68: The method according to any one of embodiments 41-45, wherein a first dose of hum13B8-b is administered to the patient on week 0, a second dose of hum13B8-b is administered to the patient at about 4 weeks, and a subsequent dose of hum13B8-b is administered to the patient about every 4 to 12 weeks thereafter.

Embodiment 69: The method according to embodiment 68, wherein the first dose, the second dose, and the subsequent dose are the same.

Embodiment 70: The method according to embodiment 69, wherein the first dose, the second dose, and the subsequent dose are 100 mg.

Embodiment 71: The method according to embodiment 69, wherein the first dose, the second dose, and the subsequent dose are 200 mg.

Embodiment 72: The method according to embodiment 68, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 73: A pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 74: The pharmaceutical composition according to embodiment 73, wherein the plaque psoriasis is moderate to severe plaque psoriasis.

Embodiment 75: The pharmaceutical composition according to either embodiment 73 or embodiment 74, wherein the pharmaceutical composition is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 76: The pharmaceutical composition according to any one of embodiments 73-75, wherein the pharmaceutical composition is administered to the patient about every 12 weeks.

Embodiment 77: The pharmaceutical composition according to any one of embodiments 73-76, wherein the pharmaceutical composition is administered to the patient subcutaneously.

Embodiment 78: The pharmaceutical composition according to any one of embodiments 73-77, wherein the pharmaceutical composition is administered to the patient by subcutaneous injection.

Embodiment 79: The pharmaceutical composition according to any one of embodiments 73-78, wherein the pharmaceutical composition is administered to the patient using an auto-injector or prefilled syringe.

Embodiment 80: The pharmaceutical composition according to any one of embodiments 73-79, wherein a therapeutically effective amount of the pharmaceutical composition is administered to the patient.

Embodiment 81: The pharmaceutical composition according to any one of embodiments 73-80, wherein a dose of the pharmaceutical composition comprises 100 mg or 200 mg hum13B8-b.

Embodiment 82: The pharmaceutical composition according to embodiment 81, wherein a dose of the pharmaceutical composition comprises 100 mg hum13B8-b.

Embodiment 83: The pharmaceutical composition according to embodiment 81, wherein a dose of the pharmaceutical composition comprises 200 mg hum13B8-b.

Embodiment 84: The pharmaceutical composition according to any one of embodiments 73-83, wherein a first dose of the pharmaceutical composition is administered to the patient on week 0 and a subsequent dose of the pharmaceutical composition is administered to the patient about every 12 weeks thereafter.

Embodiment 85: The pharmaceutical composition according to embodiment 84, wherein the first dose of the pharmaceutical composition and the subsequent dose of the pharmaceutical composition are the same.

Embodiment 86: The pharmaceutical composition according to embodiment 84, wherein the first dose of the pharmaceutical composition and the subsequent dose of the pharmaceutical composition are different.

Embodiment 87: The pharmaceutical composition according to embodiment 84, wherein the first dose of the pharmaceutical composition comprises 100 mg hum13B8-b.

Embodiment 88: The pharmaceutical composition according to embodiment 84, wherein the first dose of the pharmaceutical composition comprises 200 mg hum13B8-b.

Embodiment 89: The pharmaceutical composition according to embodiment 84, wherein the subsequent dose of the pharmaceutical composition comprises 100 mg hum13B8-b.

Embodiment 90: The pharmaceutical composition according to embodiment 84, wherein the subsequent dose of the pharmaceutical composition comprises 200 mg hum13B8-b.

Embodiment 91: The pharmaceutical composition according to embodiment 85, wherein the first dose of the pharmaceutical composition and the subsequent dose of the pharmaceutical composition comprise 100 mg hum13B8-b.

Embodiment 92: The pharmaceutical composition according to embodiment 85, wherein the first dose of the pharmaceutical composition and the subsequent dose of the pharmaceutical composition comprise 200 mg hum13B8-b.

Embodiment 93: The pharmaceutical composition according to embodiment 86, wherein the first dose of the pharmaceutical composition comprises 100 mg hum13B8-b and the subsequent dose of the pharmaceutical composition comprises 200 mg hum13B8-b.

Embodiment 94: The pharmaceutical composition according to embodiment 86, wherein the first dose of the pharmaceutical composition comprises 200 mg hum13B8-b and the subsequent dose of the pharmaceutical composition comprises 100 mg hum13B8-b.

Embodiment 95: The pharmaceutical composition according to embodiment 84, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 96: The pharmaceutical composition according to any one of embodiments 73-83, wherein a first dose of the pharmaceutical composition is administered to the patient on week 0, a second dose of the pharmaceutical composition is administered to the patient at about 4 weeks, and a subsequent dose of the pharmaceutical composition is administered to the patient about every 4 to 12 weeks thereafter.

Embodiment 97: The pharmaceutical composition according to embodiment 96, wherein the first dose of the pharmaceutical composition, the second dose of the pharmaceutical composition, and the subsequent dose of the pharmaceutical composition are the same.

Embodiment 98: The pharmaceutical composition according to embodiment 97, wherein the first dose of the pharmaceutical composition, the second dose of the pharmaceutical composition, and the subsequent dose of the pharmaceutical composition comprise 100 mg humB138-b.

Embodiment 99: The pharmaceutical composition according to embodiment 97, wherein the first dose of the pharmaceutical composition, the second dose of the pharmaceutical composition, and the subsequent dose of the pharmaceutical composition comprise 200 mg hum13B8-b.

Embodiment 100: The pharmaceutical composition according to embodiment 96, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 101: The pharmaceutical composition according to any one of embodiments 73-100, wherein administration of the pharmaceutical composition results in the patient maintaining a Physician's Global Assessment (PGA) score of “clear” or “almost clear” with at least a 2-point reduction from Baseline for at least up to about 60 weeks.

Embodiment 102: The pharmaceutical composition according to any one of embodiments 73-101, wherein administration of the pharmaceutical composition results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 103: The pharmaceutical composition according to any one of embodiments 73-102, wherein administration of the pharmaceutical composition results in the patient maintaining at least a 50% reduction in the Psoriasis Area and Severity Index (PASI 50) for at least up to about 60 weeks.

Embodiment 104: The pharmaceutical composition according to any one of embodiments 73-103, wherein administration of the pharmaceutical composition results in the patient maintaining at least a 50% reduction in the Psoriasis Area and Severity Index (PASI 50) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 105: The pharmaceutical composition according to any one of embodiments 73-102, wherein administration of the pharmaceutical composition results in the patient maintaining at least a 75% reduction in the Psoriasis Area and Severity Index (PASI 75) for at least up to about 60 weeks.

Embodiment 106: The pharmaceutical composition according to any one of embodiments 73-102 or 105, wherein administration of the pharmaceutical composition results in the patient maintaining at least a 75% reduction in the Psoriasis Area and Severity Index (PASI 75) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 107: The pharmaceutical composition according to any one of embodiments 73-102, wherein administration of the pharmaceutical composition results in the patient maintaining at least a 90% reduction in the Psoriasis Area and Severity Index (PASI 90) for at least up to about 60 weeks.

Embodiment 108: The pharmaceutical composition according to any one of embodiments 73-102 or 107, wherein administration of the pharmaceutical composition results in the patient maintaining at least a 90% reduction in the Psoriasis Area and Severity Index (PASI 90) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 109: The pharmaceutical composition according to any one of embodiments 73-102, wherein administration of the pharmaceutical composition results in the patient maintaining a 100% reduction in the Psoriasis Area and Severity Index (PASI 100) for at least up to about 60 weeks.

Embodiment 110: The pharmaceutical composition according to any one of embodiments 73-102 or 109, wherein administration of the pharmaceutical composition results in the patient maintaining a 100% reduction in the Psoriasis Area and Severity Index (PASI 100) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 111: The pharmaceutical composition according to any one of embodiments 73-110, wherein administration of the pharmaceutical composition results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 112: The pharmaceutical composition according to any one of embodiments 73-111, wherein administration of the pharmaceutical composition results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 113: A pharmaceutical composition of an anti-IL-23p19 antibody hum13B8-b for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 114: A pharmaceutical composition of an anti-IL-23p19 antibody hum13B8-b for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 115: A pharmaceutical composition of an anti-IL-23p19 antibody hum13B8-b for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 116: A pharmaceutical composition of an anti-IL-23p19 antibody hum13B8-b for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 117: A pharmaceutical composition of an anti-IL-23p19 antibody hum13B8-b for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the pharmaceutical composition is administered to the patient for at least up to about 60 weeks.

Embodiment 118: The pharmaceutical composition according to any one of embodiments 113-117, wherein the plaque psoriasis is moderate to severe plaque psoriasis.

Embodiment 119: The pharmaceutical composition according to any one of embodiments 113-118, wherein the pharmaceutical composition is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 120: The pharmaceutical composition according to any one of embodiments 113-119, wherein the pharmaceutical composition is administered to the patient about every 12 weeks.

Embodiment 121: The pharmaceutical composition according to any one of embodiments 113-120, wherein the pharmaceutical composition is administered to the patient subcutaneously.

Embodiment 122: The pharmaceutical composition according to any one of embodiments 113-121, wherein the pharmaceutical composition is administered to the patient by subcutaneous injection.

Embodiment 123: The pharmaceutical composition according to any one of embodiments 113-122, wherein the pharmaceutical composition is administered to the patient using an auto-injector or prefilled syringe.

Embodiment 124: The pharmaceutical composition according to any one of embodiments 113-123, wherein a therapeutically effective amount of the pharmaceutical composition is administered to the patient.

Embodiment 125: The pharmaceutical composition according to any one of embodiments 113-124, wherein a dose of the pharmaceutical composition comprises 100 mg or 200 mg hum13B8-b.

Embodiment 126: The pharmaceutical composition according to embodiment 125, wherein a dose of the pharmaceutical composition comprises 100 mg hum13B8-b.

Embodiment 127: The pharmaceutical composition according to embodiment 125, wherein a dose of the pharmaceutical composition comprises 200 mg hum13B8-b.

Embodiment 128: The pharmaceutical composition according to any one of embodiments 113-127, wherein a first dose of the pharmaceutical composition is administered to the patient on week 0 and a subsequent dose of the pharmaceutical composition is administered to the patient about every 12 weeks thereafter.

Embodiment 129: The pharmaceutical composition according to embodiment 128, wherein the first dose of the pharmaceutical composition and the subsequent dose of the pharmaceutical composition are the same.

Embodiment 130: The pharmaceutical composition according to embodiment 128, wherein the first dose of the pharmaceutical composition and the subsequent dose of the pharmaceutical composition are different.

Embodiment 131: The pharmaceutical composition according to embodiment 128, wherein the first dose of the pharmaceutical composition comprises 100 mg hum13B8-b.

Embodiment 132: The pharmaceutical composition according to embodiment 128, wherein the first dose of the pharmaceutical composition comprises 200 mg hum13B8-b.

Embodiment 133: The pharmaceutical composition according to embodiment 128, wherein the subsequent dose of the pharmaceutical composition comprises 100 mg hum13B8-b.

Embodiment 134: The pharmaceutical composition according to embodiment 128, wherein the subsequent dose of the pharmaceutical composition comprises 200 mg hum13B8-b.

Embodiment 135: The pharmaceutical composition according to embodiment 129, wherein the first dose of the pharmaceutical composition and the subsequent dose of the pharmaceutical composition comprises 100 mg hum13B8-b.

Embodiment 136: The pharmaceutical composition according to embodiment 129, wherein the first dose of the pharmaceutical composition and the subsequent dose of the pharmaceutical composition comprise 200 mg hum13B8-b.

Embodiment 137: The pharmaceutical composition according to embodiment 130, wherein the first dose of the pharmaceutical composition comprises 100 mg hum13B8-b and the subsequent dose of the pharmaceutical composition comprises 200 mg hum13B8-b.

Embodiment 138: The pharmaceutical composition according to embodiment 130, wherein the first dose of the pharmaceutical composition comprises 200 mg hum13B8-b and the subsequent dose of the pharmaceutical composition comprises 100 mg hum13B8-b.

Embodiment 139: The pharmaceutical composition according to embodiment 128, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 140: The pharmaceutical composition according to any one of embodiments 113-127, wherein a first dose of the pharmaceutical composition is administered to the patient on week 0, a second dose of the pharmaceutical composition is administered to the patient at about 4 weeks, and a subsequent dose of the pharmaceutical composition is administered to the patient about every 4 to 12 weeks thereafter.

Embodiment 141: The pharmaceutical composition according to embodiment 140, wherein the first dose of the pharmaceutical composition, the second dose of the pharmaceutical composition, and the subsequent dose of the pharmaceutical composition are the same.

Embodiment 142: The pharmaceutical composition according to embodiment 141, wherein the first dose of the pharmaceutical composition, the second dose of the pharmaceutical composition, and the subsequent dose of the pharmaceutical composition comprise 100 mg hum13B8-b.

Embodiment 143: The pharmaceutical composition according to embodiment 141, wherein the first dose of the pharmaceutical composition, the second dose of the pharmaceutical composition, and the subsequent dose of the pharmaceutical composition comprise 200 mg hum13B8-b.

Embodiment 144: The pharmaceutical composition according to embodiment 140, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 145: Use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 146: The use according to embodiment 145, wherein the plaque psoriasis is moderate to severe plaque psoriasis.

Embodiment 147: The use according to embodiment 145, wherein the medicament is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 148: The use according to embodiment 145, wherein the medicament is administered to the patient about every 12 weeks.

Embodiment 149: The use according to embodiment 145, wherein the medicament is administered to the patient subcutaneously.

Embodiment 150: The use according to embodiment 149, wherein the medicament is administered to the patient by subcutaneous injection.

Embodiment 151: The use according to embodiment 150, wherein the medicament is administered to the patient using an auto-injector or prefilled syringe.

Embodiment 152: The use according to embodiment 145, wherein a therapeutically effective amount of the medicament is administered to the patient.

Embodiment 153: The use according to embodiment 145, wherein a dose of the medicament comprises 100 mg or 200 mg hum13B8-b.

Embodiment 154: The use according to embodiment 153, wherein a dose of the medicament comprises 100 mg hum13B8-b.

Embodiment 155: The use according to embodiment 153, wherein a dose of the medicament comprises 200 mg hum13B8-b.

Embodiment 156: The use according to embodiment 145, wherein a first dose of the medicament is administered to the patient on week 0 and a subsequent dose of the medicament is administered to the patient about every 12 weeks thereafter.

Embodiment 157: The use according to embodiment 156, wherein the first dose of the medicament and the subsequent dose of the medicament are the same.

Embodiment 158: The use according to embodiment 156, wherein the first dose of the medicament and the subsequent dose of the medicament are different.

Embodiment 159: The use according to embodiment 156, wherein the first dose of the medicament comprises 100 mg hum13B8-b.

Embodiment 160: The use according to embodiment 156, wherein the first dose of the medicament comprises 200 mg hum13B8-b.

Embodiment 161: The use according to embodiment 156, wherein the subsequent dose of the medicament comprises 100 mg hum13B8-b.

Embodiment 162: The use according to embodiment 156, wherein the subsequent dose of the medicament comprises 200 mg hum13B8-b.

Embodiment 163: The use according to embodiment 157, wherein the first dose of the medicament and the subsequent dose of the medicament comprise 100 mg hum13B8-b.

Embodiment 164: The use according to embodiment 157, wherein the first dose of the medicament and the subsequent dose of the medicament comprise 200 mg hum13B8-b.

Embodiment 165: The use according to embodiment 158, wherein the first dose of the medicament comprises 100 mg hum13B8-b and the subsequent dose of the medicament comprises 200 mg hum13B8-b.

Embodiment 166: The use according to embodiment 158, wherein the first dose of the medicament comprises 200 mg hum13B8-b and the subsequent dose of the medicament comprises 100 mg hum13B8-b.

Embodiment 167: The use according to embodiment 156, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 168: The use according to embodiment 145, wherein a first dose of the medicament is administered to the patient on week 0, a second dose of the medicament is administered to the patient at about 4 weeks, and a subsequent dose of the medicament is administered to the patient about every 4 to 12 weeks thereafter.

Embodiment 169: The use according to embodiment 168, wherein the first dose of the medicament, the second dose of the medicament, and the subsequent dose of the medicament are the same.

Embodiment 170: The use according to embodiment 169, wherein the first dose of the medicament, the second dose of the medicament, and the subsequent dose of the medicament comprise 100 mg humB138-b.

Embodiment 171: The use according to embodiment 169, wherein the first dose of the medicament, the second dose of the medicament, and the subsequent dose of the medicament comprise 200 mg hum13B8-b.

Embodiment 172: The use according to embodiment 168, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 173: The use according to embodiment 145, wherein administration of the medicament results in the patient maintaining a Physician's Global Assessment (PGA) score of “clear” or “almost clear” with at least a 2-point reduction from Baseline for at least up to about 60 weeks.

Embodiment 174: The use according to embodiment 173, wherein administration of the medicament results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 175: The use according to embodiment 145, wherein administration of the medicament results in the patient maintaining at least a 50% reduction in the Psoriasis Area and Severity Index (PASI 50) for at least up to about 60 weeks.

Embodiment 176: The use according to embodiment 175, wherein administration of the medicament results in the patient maintaining at least a 50% reduction in the Psoriasis Area and Severity Index (PASI 50) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 177: The use according to embodiment 145, wherein administration of the medicament results in the patient maintaining at least a 75% reduction in the Psoriasis Area and Severity Index (PASI 75) for at least up to about 60 weeks.

Embodiment 178: The use according to embodiment 177, wherein administration of the medicament results in the patient maintaining at least a 75% reduction in the Psoriasis Area and Severity Index (PASI 75) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 179: The use according to embodiment 145, wherein administration of the medicament results in the patient maintaining at least a 90% reduction in the Psoriasis Area and Severity Index (PASI 90) for at least up to about 60 weeks.

Embodiment 180: The use according to embodiment 179, wherein administration of the medicament results in the patient maintaining at least a 90% reduction in the Psoriasis Area and Severity Index (PASI 90) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 181: The use according to embodiment 145, wherein administration of the medicament results in the patient maintaining a 100% reduction in the Psoriasis Area and Severity Index (PASI 100) for at least up to about 60 weeks.

Embodiment 182: The use according to embodiment 181, wherein administration of the medicament results in the patient maintaining a 100% reduction in the Psoriasis Area and Severity Index (PASI 100) for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 183: The use according to embodiment 145, wherein administration of the medicament results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 184: The use according to embodiment 183, wherein administration of the medicament results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 185: Use of an anti-IL-23p19 antibody hum13B8-b for the manufacture of a medicament for maintaining a Physician's Global Assessment (PGA) score of “clear” or “almost clear” with at least a 2-point reduction from Baseline in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 186: Use of an anti-IL-23p19 antibody hum13B8-b for the manufacture of a medicament for maintaining at least a 50% reduction in the Psoriasis Area and Severity Index (PASI 50) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 187: Use of an anti-IL-23p19 antibody hum13B8-b for the manufacture of a medicament for maintaining at least a 75% reduction in the Psoriasis Area and Severity Index (PASI 75) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 188: Use of an anti-IL-23p19 antibody hum13B8-b for the manufacture of a medicament for maintaining at least a 90% reduction in the Psoriasis Area and Severity Index (PASI 90) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 189: Use of an anti-IL-23p19 antibody hum13B8-b for the manufacture of a medicament for maintaining a 100% reduction in the Psoriasis Area and Severity Index (PASI 100) in a patient with plaque psoriasis, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; and wherein the medicament is administered to the patient for at least up to about 60 weeks.

Embodiment 190: The use according to any one of embodiments 185-189, wherein the plaque psoriasis is moderate to severe plaque psoriasis.

Embodiment 191: The use according to any one of embodiments 185-189, wherein the medicament is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 192: The use according to any one of embodiments 185-189, wherein the medicament is administered to the patient about every 12 weeks.

Embodiment 193: The use according to any one of embodiments 185-189, wherein the medicament is administered to the patient subcutaneously.

Embodiment 194: The use according to embodiment 193, wherein the medicament is administered to the patient by subcutaneous injection.

Embodiment 195: The use according to embodiment 194, wherein the medicament is administered to the patient using an auto-injector or prefilled syringe.

Embodiment 196: The use according to any one of embodiments 185-189, wherein a therapeutically effective amount of the medicament is administered to the patient.

Embodiment 197: The use according to any one of embodiments 185-189, wherein a dose of the medicament comprises 100 mg or 200 mg hum13B8-b.

Embodiment 198: The use according to embodiment 197, wherein a dose of the medicament comprises 100 mg hum13B8-b.

Embodiment 199: The use according to embodiment 197, wherein a dose of the medicament comprises 200 mg hum13B8-b.

Embodiment 200: The use according to any one of embodiments 185-189, wherein a first dose of the medicament is administered to the patient on week 0 and a subsequent dose of the medicament is administered to the patient about every 12 weeks thereafter.

Embodiment 201: The use according to embodiment 200, wherein the first dose of the medicament and the subsequent dose of the medicament are the same.

Embodiment 202: The use according to embodiment 200, wherein the first dose of the medicament and the subsequent dose of the medicament are different.

Embodiment 203: The use according to embodiment 200, wherein the first dose of the medicament comprises 100 mg hum13B8-b.

Embodiment 204: The use according to embodiment 200, wherein the first dose of the medicament comprises 200 mg hum13B8-b.

Embodiment 205: The use according to embodiment 200, wherein the subsequent dose of the medicament comprises 100 mg hum13B8-b.

Embodiment 206: The use according to embodiment 200, wherein the subsequent dose of the medicament comprises 200 mg hum13B8-b.

Embodiment 207: The use according to embodiment 201, wherein the first dose of the medicament and the subsequent dose of the medicament comprises 100 mg hum13B8-b.

Embodiment 208: The use according to embodiment 201, wherein the first dose of the medicament and the subsequent dose of the medicament comprise 200 mg hum13B8-b.

Embodiment 209: The use according to embodiment 202, wherein the first dose of the medicament comprises 100 mg hum13B8-b and the subsequent dose of the medicament comprises 200 mg hum13B8-b.

Embodiment 210: The use according to embodiment 202, wherein the first dose of the medicament comprises 200 mg hum13B8-b and the subsequent dose of the medicament comprises 100 mg hum13B8-b.

Embodiment 211: The use according to embodiment 200, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 212: The use according to any one of embodiments 185-189, wherein a first dose of the medicament is administered to the patient on week 0, a second dose of the medicament is administered to the patient at about 4 weeks, and a subsequent dose of the medicament is administered to the patient about every 4 to 12 weeks thereafter.

Embodiment 213: The use according to embodiment 212, wherein the first dose of the medicament, the second dose of the medicament, and the subsequent dose of the medicament are the same.

Embodiment 214: The use according to embodiment 213, wherein the first dose of the medicament, the second dose of the medicament, and the subsequent dose of the medicament comprise 100 mg hum13B8-b.

Embodiment 215: The use according to embodiment 213, wherein the first dose of the medicament, the second dose of the medicament, and the subsequent dose of the medicament comprise 200 mg hum13B8-b.

Embodiment 216: The use according to embodiment 212, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 217: A method of treating plaque psoriasis comprising administering an anti-IL-23p19 antibody hum13B8-b to a patient in need thereof, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein hum13B8-b is administered to the patient for at least up to about 60 weeks; and wherein the patient has a reduced risk of a cardiac adverse event as compared to the risk of a cardiac adverse event for treatment with (a) an anti-IL-23p19 antibody other than hum13B8-b, or (b) an anti-IL-17 antibody for at least up to about 60 weeks.

Embodiment 218: The method according to embodiment 217, wherein the cardiac adverse event is pericarditis, atrial fibrillation, or coronary artery disease.

Embodiment 219: The method according to embodiment 217, wherein the plaque psoriasis is moderate to severe plaque psoriasis.

Embodiment 220: The method according to embodiment 217, wherein administration of hum13B8-b results in a reduced risk of a cardiac adverse event as compared to the risk of a cardiac adverse event for treatment with (a) an anti-IL-23p19 antibody other than hum13B8-b, or (b) an anti-IL-17 antibody for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 221: The method according to embodiment 217, wherein hum13B8-b is administered to the patient about every 12 weeks.

Embodiment 222: The method according to embodiment 217, wherein hum13B8-b is administered to the patient subcutaneously.

Embodiment 223: The method according to embodiment 222, wherein hum13B8-b is administered to the patient by subcutaneous injection.

Embodiment 224: The method according to embodiment 223, wherein hum13B8-b is administered to the patient using an auto-injector or prefilled syringe.

Embodiment 225: The method according to embodiment 217, wherein a therapeutically effective amount of hum13B8-b is administered to the patient.

Embodiment 226: The method according to embodiment 217, wherein 100 mg or 200 mg of hum13B8-b is administered to the patient.

Embodiment 227: The method according to embodiment 226, wherein 100 mg of hum13B8-b is administered to the patient.

Embodiment 228: The method according to embodiment 226, wherein 200 mg of hum13B8-b is administered to the patient.

Embodiment 229: The method according to embodiment 217, wherein a first dose of hum13B8-b is administered to the patient on week 0 and a subsequent dose of hum13B8-b is administered to the patient about every 12 weeks thereafter.

Embodiment 230: The method according to embodiment 229, wherein the first dose and the subsequent dose are the same.

Embodiment 231: The method according to embodiment 229, wherein the first dose and the subsequent dose are different.

Embodiment 232: The method according to embodiment 229, wherein the first dose is 100 mg.

Embodiment 233: The method according to embodiment 229, wherein the first dose is 200 mg.

Embodiment 234: The method according to embodiment 229, wherein the subsequent dose is 100 mg.

Embodiment 235: The method according to embodiment 229, wherein the subsequent dose is 200 mg.

Embodiment 236: The method according to embodiment 230, wherein the first dose and the subsequent dose are 100 mg.

Embodiment 237: The method according to embodiment 230, wherein the first dose and the subsequent dose are 200 mg.

Embodiment 238: The method according to embodiment 231, wherein the first dose is 100 mg and the subsequent dose is 200 mg.

Embodiment 239: The method according to embodiment 231, wherein the first dose is 200 mg and the subsequent dose is 100 mg.

Embodiment 240: The method according to embodiment 229, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 241: The method according to embodiment 217, wherein a first dose of hum13B8-b is administered to the patient on week 0, a second dose of hum13B8-b is administered to the patient at about 4 weeks, and a subsequent dose of hum13B8-b is administered to the patient about every 4 to 12 weeks thereafter.

Embodiment 242: The method according to embodiment 241, wherein the first dose, the second dose, and the subsequent dose are the same.

Embodiment 243: The method according to embodiment 242, wherein the first dose, the second dose, and the subsequent dose are 100 mg.

Embodiment 244: The method according to embodiment 242, wherein the first dose, the second dose, and the subsequent dose are 200 mg.

Embodiment 245: The method according to embodiment 241, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 246: A pharmaceutical composition of an anti-IL-23p19 antibody hum13B8-b for the treatment of plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein the pharmaceutical composition is administered to the patient for at least up to about 60 weeks; and wherein the patient has a reduced risk of a cardiac adverse event as compared to the risk of a cardiac adverse event for treatment with (a) a pharmaceutical composition of an anti-IL-23p19 antibody other than hum13B8-b, or (b) a pharmaceutical composition of an anti-IL-17 antibody for at least up to about 60 weeks.

Embodiment 247: The pharmaceutical composition according to embodiment 246, wherein the cardiac adverse event is pericarditis, atrial fibrillation, or coronary artery disease.

Embodiment 248: The pharmaceutical composition according to either embodiment 246 or embodiment 247, wherein the plaque psoriasis is moderate to severe plaque psoriasis.

Embodiment 249: The pharmaceutical composition according to any one of embodiments 246-248, wherein administration of the pharmaceutical composition results in a reduced risk of a cardiac adverse event as compared to the risk of a cardiac adverse event for treatment with (a) a pharmaceutical composition of an anti-IL-23p19 antibody other than hum13B8-b, or (b) a pharmaceutical composition of an anti-IL-17 antibody for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 250: The pharmaceutical composition according to any one of embodiments 246-249, wherein the pharmaceutical composition is administered to the patient about every 12 weeks.

Embodiment 251: The pharmaceutical composition according to any one of embodiments 246-250, wherein the pharmaceutical composition is administered to the patient subcutaneously.

Embodiment 252: The pharmaceutical composition according to any one of embodiments 246-251, wherein the pharmaceutical composition is administered to the patient by subcutaneous injection.

Embodiment 253: The pharmaceutical composition according to any one of embodiments 246-252, wherein the pharmaceutical composition is administered to the patient using an auto-injector or prefilled syringe.

Embodiment 254: The pharmaceutical composition according to any one of embodiments 246-253, wherein a therapeutically effective amount of the pharmaceutical composition is administered to the patient.

Embodiment 255: The pharmaceutical composition according to any one of embodiments 246-254, wherein a dose of the pharmaceutical composition comprises 100 mg or 200 mg hum13B8-b.

Embodiment 256: The pharmaceutical composition according to embodiment 255, wherein a dose of the pharmaceutical composition comprises 100 mg hum13B8-b.

Embodiment 257: The pharmaceutical composition according to embodiment 255, wherein a dose of the pharmaceutical composition comprises 200 mg hum13B8-b.

Embodiment 258: The pharmaceutical composition according to any one of embodiments 246-257, wherein a first dose of the pharmaceutical composition is administered to the patient on week 0 and a subsequent dose of the pharmaceutical composition is administered to the patient about every 12 weeks thereafter.

Embodiment 259: The pharmaceutical composition according to embodiment 258, wherein the first dose of the pharmaceutical composition and the subsequent dose of the pharmaceutical composition are the same.

Embodiment 260: The pharmaceutical composition according to embodiment 258, wherein the first dose of the pharmaceutical composition and the subsequent dose of the pharmaceutical composition are different.

Embodiment 261: The pharmaceutical composition according to embodiment 258, wherein the first dose of the pharmaceutical composition comprises 100 mg hum13B8-b.

Embodiment 262: The pharmaceutical composition according to embodiment 258, wherein the first dose of the pharmaceutical composition comprises 200 mg hum13B8-b.

Embodiment 263: The pharmaceutical composition according to embodiment 258, wherein the subsequent dose of the pharmaceutical composition comprises 100 mg hum13B8-b.

Embodiment 264: The pharmaceutical composition according to embodiment 258, wherein the subsequent dose of the pharmaceutical composition comprises 200 mg hum13B8-b.

Embodiment 265: The pharmaceutical composition according to embodiment 259, wherein the first dose of the pharmaceutical composition and the subsequent dose of the pharmaceutical composition comprise 100 mg hum13B8-b.

Embodiment 266: The pharmaceutical composition according to embodiment 259, wherein the first dose of the pharmaceutical composition and the subsequent dose of the pharmaceutical composition comprise 200 mg hum13B8-b.

Embodiment 267: The pharmaceutical composition according to embodiment 260, wherein the first dose of the pharmaceutical composition comprises 100 mg hum13B8-b and the subsequent dose of the pharmaceutical composition comprises 200 mg hum13B8-b g.

Embodiment 268: The pharmaceutical composition according to embodiment 260, wherein the first dose of the pharmaceutical composition comprises 200 mg hum13B8-b and the subsequent dose of the pharmaceutical composition comprises 100 mg hum13B8-b.

Embodiment 269: The pharmaceutical composition according to embodiment 258, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 270: The pharmaceutical composition according to any one of embodiments 246-257, wherein a first dose of the pharmaceutical composition is administered to the patient on week 0, a second dose of the pharmaceutical composition is administered to the patient at about 4 weeks, and a subsequent dose of the pharmaceutical composition is administered to the patient about every 4 to 12 weeks thereafter.

Embodiment 271: The pharmaceutical composition according to embodiment 270, wherein the first dose of the pharmaceutical composition, the second dose of the pharmaceutical composition, and the subsequent dose of the pharmaceutical composition are the same.

Embodiment 272: The pharmaceutical composition according to embodiment 271, wherein the first dose of the pharmaceutical composition, the second dose of the pharmaceutical composition, and the subsequent dose of the pharmaceutical composition comprise 100 mg humB138-b.

Embodiment 273: The pharmaceutical composition according to embodiment 271, wherein the first dose of the pharmaceutical composition, the second dose of the pharmaceutical composition, and the subsequent dose of the pharmaceutical composition comprise 200 mg hum13B8-b.

Embodiment 274: The pharmaceutical composition according to embodiment 270, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 275: Use of an anti-IL-23p19 antibody hum13B8-b for the manufacture of a medicament for treating plaque psoriasis in a patient, wherein hum13B8-b comprises: (i) a light chain polypeptide comprising the amino acid sequence of SEQ ID NO: 1, and (ii) a heavy chain polypeptide comprising the amino acid sequence of SEQ ID NO: 2; wherein the medicament is administered to the patient for at least up to about 60 weeks; and wherein the patient has a reduced risk of a cardiac adverse event as compared to the risk of a cardiac adverse event for treatment with (a) a medicament of an anti-IL-23p19 antibody other than hum13B8-b, or (b) a medicament of an anti-IL-17 antibody for at least up to about 60 weeks.

Embodiment 276: The use according to embodiment 275, wherein the cardiac adverse event is pericarditis, atrial fibrillation, or coronary artery disease.

Embodiment 277: The use according to embodiment 275, wherein the plaque psoriasis is moderate to severe plaque psoriasis.

Embodiment 278: The use according to embodiment 275, wherein administration of the medicament results in a reduced risk of a cardiac adverse event as compared to the risk of a cardiac adverse event for treatment with (a) a medicament of an anti-IL-23p19 antibody other than hum13B8-b, or (b) a medicament of an anti-IL-17 antibody for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 279: The use according to embodiment 275, wherein the medicament is administered to the patient about every 12 weeks.

Embodiment 280: The use according to embodiment 275, wherein the medicament is administered to the patient subcutaneously.

Embodiment 281: The use according to embodiment 280, wherein the medicament is administered to the patient by subcutaneous injection.

Embodiment 282: The use according to embodiment 281, wherein the medicament is administered to the patient using an auto-injector or prefilled syringe.

Embodiment 283: The use according to embodiment 275, wherein a therapeutically effective amount of the medicament is administered to the patient.

Embodiment 284: The use according to embodiment 275, wherein a dose of the medicament comprises 100 mg or 200 mg hum13B8-b.

Embodiment 285: The use according to embodiment 284, wherein a dose of the medicament comprises 100 mg hum13B8-b.

Embodiment 286: The use according to embodiment 284, wherein a dose of the medicament comprises 200 mg hum13B8-b.

Embodiment 287: The use according to embodiment 275, wherein a first dose of the medicament is administered to the patient on week 0 and a subsequent dose of the medicament is administered to the patient about every 12 weeks thereafter.

Embodiment 288: The use according to embodiment 287, wherein the first dose of the medicament and the subsequent dose of the medicament are the same.

Embodiment 289: The use according to embodiment 287, wherein the first dose of the medicament and the subsequent dose of the medicament are different.

Embodiment 290: The use according to embodiment 287, wherein the first dose of the medicament comprises 100 mg hum13B8-b.

Embodiment 291: The use according to embodiment 287, wherein the first dose of the medicament comprises 200 mg hum13B8-b.

Embodiment 292: The use according to embodiment 287, wherein the subsequent dose of the medicament comprises 100 mg hum13B8-b.

Embodiment 293: The use according to embodiment 287, wherein the subsequent dose of the medicament comprises 200 mg hum13B8-b.

Embodiment 294: The use according to embodiment 288, wherein the first dose of the medicament and the subsequent dose of the medicament comprise 100 mg hum13B8-b.

Embodiment 295: The use according to embodiment 288, wherein the first dose of the medicament and the subsequent dose of the medicament comprise 200 mg hum13B8-b.

Embodiment 296: The use according to embodiment 289, wherein the first dose of the medicament comprises 100 mg hum13B8-b and the subsequent dose of the medicament comprises 200 mg hum13B8-b g.

Embodiment 297: The use according to embodiment 289, wherein the first dose of the medicament comprises 200 mg hum13B8-b and the subsequent dose of the medicament comprises 100 mg hum13B8-b.

Embodiment 298: The use according to embodiment 287, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 299: The use according to embodiment 275, wherein a first dose of the medicament is administered to the patient on week 0, a second dose of the medicament is administered to the patient at about 4 weeks, and a subsequent dose of the medicament is administered to the patient about every 4 to 12 weeks thereafter.

Embodiment 300: The use according to embodiment 299, wherein the first dose of the medicament, the second dose of the medicament, and the subsequent dose of the medicament are the same.

Embodiment 301: The use according to embodiment 300, wherein the first dose of the medicament, the second dose of the medicament, and the subsequent dose of the medicament comprise 100 mg humB138-b.

Embodiment 302: The use according to embodiment 300, wherein the first dose of the medicament, the second dose of the medicament, and the subsequent dose of the medicament comprise 200 mg hum13B8-b.

Embodiment 303: The use according to embodiment 299, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 304: A method of treating plaque psoriasis comprising administering an anti-IL-23p19 antibody to a patient in need thereof, wherein the anti-IL-23p19 antibody is administered to the patient for at least up to about 60 weeks; wherein administration of the anti-IL-23p19 antibody results in the patient maintaining a Physician's Global Assessment (PGA) score of “clear” or “almost clear” with at least a 2-point reduction from Baseline for at least up to about 60 weeks; wherein administration of the anti-IL-23p19 antibody results in the patient maintaining at least a 50% reduction in the Psoriasis Area and Severity Index (PASI 50) for at least up to about 60 weeks; and wherein administration of the anti-IL-23p19 antibody results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 305: The method according to embodiment 304, wherein the AE, drug-related AE, SAE, Tier 1 AE, or Tier 2 AE is a cardiac adverse event.

Embodiment 306: The method according to embodiment 304, wherein the patient has a reduced risk of a cardiac adverse event as compared to the risk of a cardiac adverse event for treatment with (a) an anti-IL-23p19 antibody other than hum13B8-b, or (b) an anti-IL-17 antibody for at least up to about 60 weeks.

Embodiment 307: The method according to either embodiment 305 or embodiment 306, wherein the cardiac adverse event is pericarditis, atrial fibrillation, or coronary artery disease.

Embodiment 308: The method according to embodiment 304, wherein the plaque psoriasis is moderate to severe plaque psoriasis.

Embodiment 309: The method according to embodiment 304, wherein the anti-IL-23p19 antibody is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 310: The method according to embodiment 304, wherein administration of the anti-IL-23p19 antibody results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 311: The method according to embodiment 304, wherein administration of the anti-IL-23p19 antibody results in the patient maintaining at least a 50% reduction in the Psoriasis Area and Severity Index (PASI 50) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 312: The method according to embodiment 304, wherein administration of the anti-IL-23p19 antibody results in the patient maintaining at least a 75% reduction in the Psoriasis Area and Severity Index (PASI 75) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 313: The method according to embodiment 304, wherein administration of the anti-IL-23p19 antibody results in the patient maintaining at least a 90% reduction in the Psoriasis Area and Severity Index (PASI 90) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 314: The method according to embodiment 304, wherein administration of the anti-IL-23p19 antibody results in the patient maintaining a 100% reduction in the Psoriasis Area and Severity Index (PASI 100) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 315: The method according to embodiment 304, wherein administration of the anti-IL-23p19 antibody results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 316: The method according to embodiment 304, wherein the anti-IL-23p19 antibody is administered to the patient about every 12 weeks.

Embodiment 317: The method according to embodiment 304, wherein the anti-IL-23p19 antibody is administered to the patient subcutaneously.

Embodiment 318: The method according to embodiment 317, wherein the anti-IL-23p19 antibody is administered to the patient by subcutaneous injection.

Embodiment 319: The method according to embodiment 318, wherein the anti-IL-23p19 antibody is administered to the patient using an auto-injector or prefilled syringe.

Embodiment 320: The method according to embodiment 304, wherein a therapeutically effective amount of the anti-IL-23p19 antibody is administered to the patient.

Embodiment 321: The method according to embodiment 304, wherein 100 mg or 200 mg of the anti-IL-23p19 antibody is administered to the patient.

Embodiment 322: The method according to embodiment 304, wherein a first dose of the anti-IL-23p19 antibody is administered to the patient on week 0 and a subsequent dose of the anti-IL-23p19 antibody is administered to the patient about every 12 weeks thereafter.

Embodiment 323: The method according to embodiment 322, wherein: (a) the first dose and the subsequent dose are 100 mg; or (b) the first dose and the subsequent dose are 200 mg.

Embodiment 324: The method according to embodiment 322, wherein: (a) the first dose is 100 mg and the subsequent dose is 200 mg; or (b) the first dose is 200 mg and the subsequent dose is 100 mg.

Embodiment 325: The method according to embodiment 322, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 326: The method according to embodiment 304, wherein a first dose of the anti-IL-23p19 antibody is administered to the patient on week 0, a second dose of the anti-IL-23p19 antibody is administered to the patient at about 4 weeks, and a subsequent dose of the anti-IL-23p19 antibody is administered to the patient about every 4 to 12 weeks thereafter.

Embodiment 327: The method according to embodiment 326, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 328: The method according to any one of embodiments 304-327, wherein the anti-IL-23p19 antibody is hum13B8-b and wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 329: A pharmaceutical composition of an anti-IL-23p19 antibody for the treatment of plaque psoriasis in a patient; wherein the pharmaceutical composition is administered to the patient for at least up to about 60 weeks; wherein administration of the pharmaceutical composition results in the patient maintaining a Physician's Global Assessment (PGA) score of “clear” or “almost clear” with at least a 2-point reduction from Baseline for at least up to about 60 weeks; wherein administration of the pharmaceutical composition results in the patient maintaining at least a 50% reduction in the Psoriasis Area and Severity Index (PASI 50) for at least up to about 60 weeks; and wherein administration of the pharmaceutical composition results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 330: The pharmaceutical composition according to embodiment 329, wherein the AE, drug-related AE, SAE, Tier 1 AE, or Tier 2 AE is a cardiac adverse event.

Embodiment 331: The pharmaceutical composition according to either embodiment 329 or embodiment 330, wherein the patient has a reduced risk of a cardiac adverse event as compared to the risk of a cardiac adverse event for treatment with (a) a pharmaceutical composition of an anti-IL-23p19 antibody other than hum13B8-b, or (b) a pharmaceutical composition of an anti-IL-17 antibody for at least up to about 60 weeks.

Embodiment 332: The pharmaceutical composition according to any one of embodiments 329-331, wherein the cardiac adverse event is pericarditis, atrial fibrillation, or coronary artery disease.

Embodiment 333: The pharmaceutical composition according to any one of embodiments 329-332, wherein the plaque psoriasis is moderate to severe plaque psoriasis.

Embodiment 334: The pharmaceutical composition according to any one of embodiments 329-333, wherein the pharmaceutical composition is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 335: The pharmaceutical composition according to any one of embodiments 329-334, wherein administration of the pharmaceutical composition results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 336: The pharmaceutical composition according to any one of embodiments 329-335, wherein administration of the pharmaceutical composition results in the patient maintaining at least a 50% reduction in the Psoriasis Area and Severity Index (PASI 50) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 337: The pharmaceutical composition according to any one of embodiments 329-335, wherein administration of the pharmaceutical composition results in the patient maintaining at least a 75% reduction in the Psoriasis Area and Severity Index (PASI 75) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 338: The pharmaceutical composition according to embodiment 329-335, wherein administration of the pharmaceutical composition results in the patient maintaining at least a 90% reduction in the Psoriasis Area and Severity Index (PASI 90) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 339: The pharmaceutical composition according to embodiment 329-335, wherein administration of the pharmaceutical composition results in the patient maintaining a 100% reduction in the Psoriasis Area and Severity Index (PASI 100) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 340: The pharmaceutical composition according to any one of embodiments 329-339, wherein administration of the pharmaceutical composition results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 341: The pharmaceutical composition according to any one of embodiments 329-340, wherein the pharmaceutical composition is administered to the patient about every 12 weeks.

Embodiment 342: The pharmaceutical composition according to any one of embodiments 329-341, wherein the pharmaceutical composition is administered to the patient subcutaneously.

Embodiment 343: The pharmaceutical composition according to any one of embodiments 329-342, wherein the pharmaceutical composition is administered to the patient by subcutaneous injection.

Embodiment 344: The pharmaceutical composition according to any one of embodiments 329-343, wherein the pharmaceutical composition is administered to the patient using an auto-injector or prefilled syringe.

Embodiment 345: The pharmaceutical composition according to any one of embodiments 329-344, wherein a therapeutically effective amount of the pharmaceutical composition is administered to the patient.

Embodiment 346: The pharmaceutical composition according to any one of embodiments 329-345, a dose of the pharmaceutical composition comprises 100 mg or 200 mg of the anti-IL-23p19 antibody.

Embodiment 347: The pharmaceutical composition according to any one of embodiments 329-346, wherein a first dose of the pharmaceutical composition is administered to the patient on week 0 and a subsequent dose of the pharmaceutical composition is administered to the patient about every 12 weeks thereafter.

Embodiment 348: The pharmaceutical composition according to embodiment 347, wherein: (a) the first dose and the subsequent dose of the pharmaceutical composition comprise 100 mg of the anti-IL-23p19 antibody; or (b) the first dose and the subsequent dose of the pharmaceutical composition comprise 200 mg of the anti-IL-23p19 antibody.

Embodiment 349: The pharmaceutical composition according to embodiment 347, wherein: (a) the first dose of the pharmaceutical composition comprises 100 mg of the anti-IL-23p19 antibody and the subsequent dose of the pharmaceutical composition comprises 200 mg of the anti-IL-23p19 antibody; or (b) the first dose of the pharmaceutical composition comprises 200 mg of the anti-IL-23p19 antibody and the subsequent dose of the pharmaceutical composition comprises 100 mg of the anti-IL-23p19 antibody.

Embodiment 350: The pharmaceutical composition according to embodiment 347, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 351: The pharmaceutical composition according to any one of embodiments 329-346, wherein a first dose of the pharmaceutical composition is administered to the patient on week 0, a second dose of the pharmaceutical composition is administered to the patient at about 4 weeks, and a subsequent dose of the pharmaceutical composition is administered to the patient about every 4 to 12 weeks thereafter.

Embodiment 352: The pharmaceutical composition according to embodiment 331, wherein the subsequent dose of the pharmaceutical composition is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 353: The pharmaceutical composition according to any one of embodiments 329-352, wherein the anti-IL-23p19 antibody is hum13B8-b and wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

Embodiment 354: Use of an anti-IL-23p19 antibody for the manufacture of a medicament for treating plaque psoriasis in a patient; wherein the medicament is administered to the patient for at least up to about 60 weeks; wherein administration of the medicament results in the patient maintaining a Physician's Global Assessment (PGA) score of “clear” or “almost clear” with at least a 2-point reduction from Baseline for at least up to about 60 weeks; wherein administration of the medicament results in the patient maintaining at least a 50% reduction in the Psoriasis Area and Severity Index (PASI 50) for at least up to about 60 weeks; and wherein administration of the medicament results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

Embodiment 355: The use according to embodiment 354, wherein the AE, drug-related AE, SAE, Tier 1 AE, or Tier 2 AE is a cardiac adverse event.

Embodiment 356: The use according to embodiment 354, wherein the patient has a reduced risk of a cardiac adverse event as compared to the risk of a cardiac adverse event for treatment with (a) a medicament of an anti-IL-23p19 antibody other than hum13B8-b, or (b) a medicament of an anti-IL-17 antibody for at least up to about 60 weeks.

Embodiment 357: The use according to either embodiment 355 or embodiment 356, wherein the cardiac adverse event is pericarditis, atrial fibrillation, or coronary artery disease.

Embodiment 358: The use according to embodiment 354, wherein the plaque psoriasis is moderate to severe plaque psoriasis.

Embodiment 359: The use according to embodiment 354, wherein the medicament is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 360: The use according to embodiment 354, wherein administration of the medicament results in the patient maintaining a Physician's Global Assessment (PGA) score of “clear” or “almost clear” with at least a 2-point reduction from Baseline for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 361: The use according to embodiment 354, wherein administration of the medicament results in the patient maintaining at least a 50% reduction in the Psoriasis Area and Severity Index (PASI 50) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 362: The use according to embodiment 354, wherein administration of the medicament results in the patient maintaining at least a 75% reduction in the Psoriasis Area and Severity Index (PASI 75) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 363: The use according to embodiment 354, wherein administration of the medicament results in the patient maintaining at least a 90% reduction in the Psoriasis Area and Severity Index (PASI 90) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 364: The use according to embodiment 354, wherein administration of the medicament results in the patient maintaining a 100% reduction in the Psoriasis Area and Severity Index (PASI 100) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 365: The use according to embodiment 354, wherein administration of the medicament results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

Embodiment 366: The use according to embodiment 354, wherein the medicament is administered to the patient about every 12 weeks.

Embodiment 367: The use according to embodiment 354, wherein the medicament is administered to the patient subcutaneously.

Embodiment 368: The use according to embodiment 367, wherein the medicament is administered to the patient by subcutaneous injection.

Embodiment 369: The use according to embodiment 368, wherein the medicament is administered to the patient using an auto-injector or prefilled syringe.

Embodiment 370: The use according to embodiment 354, wherein a therapeutically effective amount of the medicament is administered to the patient.

Embodiment 371: The use according to embodiment 354, a dose of the medicament comprises 100 mg or 200 mg of the anti-IL-23p19 antibody.

Embodiment 372: The use according to embodiment 354, wherein a first dose of the medicament is administered to the patient on week 0 and a subsequent dose of the medicament is administered to the patient about every 12 weeks thereafter.

Embodiment 373: The use according to embodiment 372, wherein: (a) the first dose and the subsequent dose of the medicament comprise 100 mg of the anti-IL-23p19 antibody; or (b) the first dose and the subsequent dose of the medicament comprise 200 mg of the anti-IL-23p19 antibody.

Embodiment 374: The use according to embodiment 372, wherein: (a) the first dose of the medicament comprises 100 mg of the anti-IL-23p19 antibody and the subsequent dose of the medicament comprises 200 mg of the anti-IL-23p19 antibody; or (b) the first dose of the medicament comprises 200 mg of the anti-IL-23p19 antibody and the subsequent dose of the medicament comprises 100 mg of the anti-IL-23p19 antibody.

Embodiment 375: The use according to embodiment 374, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 376: The use according to embodiment 354, wherein a first dose of the medicament is administered to the patient on week 0, a second dose of the medicament is administered to the patient at about 4 weeks, and a subsequent dose of the medicament is administered to the patient about every 4 to 12 weeks thereafter.

Embodiment 377: The use according to embodiment 376, wherein the subsequent dose of the medicament is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

Embodiment 378: The use according to any one of embodiments 354-377, wherein the anti-IL-23p19 antibody is hum13B8-b and wherein hum13B8-b comprises: (i) a light chain polypeptide comprising the amino acid sequence of SEQ ID NO: 1, and (ii) a heavy chain polypeptide comprising the amino acid sequence of SEQ ID NO: 2.

### Examples

The Examples that follow are illustrative of specific embodiments of the disclosure, and various uses thereof. They are set forth for explanatory purposes only and should not be construed as limiting the scope of the disclosure in any way.

Disclosed herein is a multicenter, randomized, double-blind, placebo-controlled study to assess the long-term (i.e., >52 weeks) safety and tolerability of tildrakizumab (MK-3222) in subjects with moderate-to-severe chronic plaque psoriasis.

### Study Design

### Selection of Doses

Subjects who completed a 52-week base study were eligible for inclusion in the extension studies. Eligible subjects received one of two active doses of tildrakizumab (MK-3222), 100 mg or 200 mg, for a minimum of 4 years. Dose assignment (100 mg or 200 mg) was the same dose the subject received at the end of the base study. During the open-label long-term extension phase of the study, subjects received tildrakizumab (MK-3222) in either the pre-filled syringe (PFS) or auto-injector (AI) format.

### Study Duration

Given the chronic nature of psoriasis, the efficacy and safety profiles of psoriasis therapies, which are usually established in randomized controlled trials of relatively short duration, need to be determined over extended treatment periods. Long-term extension studies provide an opportunity to assess long-term safety and maintenance of efficacy.

This long-term extension study was designed to provide an opportunity to the subjects enrolled in the base study to continue their treatment for a minimum of additional 4 years. During this period, they were regularly monitored for maintenance of treatment response and long-term safety and tolerability of subcutaneous tildrakizumab.

### Study Population

Subjects (≥18 years of age) with a diagnosis of moderate-to-severe chronic plaque psoriasis (defined by ≥10% body surface area involvement, PGA score ≥3, and PASI score ≥12 at baseline) were enrolled in the study. Randomized subjects with prior exposure to biologics therapy for psoriasis, and randomized subjects with a diagnosis of psoriatic arthritis at baseline were enrolled in the base study. Subjects on topical psoriasis treatment (excluding Class VI or VII low-potency topical corticosteroids), conventional systemic psoriasis therapy (e.g., cyclosporine, methotrexate, acitretin, fumaric acid esters) or phototherapy (e.g., ultraviolet [UV]-B light phototherapy, psoralen-UVA (PUVA) therapy, tanning salon or home-administered UVB), under treatment with an injectable or oral corticosteroids, under treatment with a biological agent (including monoclonal antibodies, alefacept), under treatment with injectable or oral corticosteroids, treatment with a biological agent (including monoclonal antibodies, alefacept), under treatment with a non-biological investigational agent, and subjects taking a biological investigational agent were excluded from the study.

### Treatments

Subjects in the extension study received 100 mg (n=381) or 200 mg (n=349) of tildrakizumab (MK-3222) or placebo every 12 weeks for a minimum of 212 weeks, following their last dose of treatment in the 52-week base study. Subjects received tildrakizumab (MK-3222) delivered via a pre-filled syringe (PFS), of which some doses were wholly contained within an auto-injector (AI). There were no randomization or stratification in the extension study. Subjects received the same dose of tildrakizumab via PFS or AI that was received at the end of the base study. The product names are provided in Table 1.

TABLE 1
Product Descriptions

| Product Name and Potency | Dosage Form |
|---|---|
| Tildrakizumab (MK-3222), 100 mg/mL | Pre-filled syringe |
| Tildrakizumab (MK-3222) | Pre-filled syringe |
| matching placebo, 0 mg/mL | |
| Tildrakizumab (MK-3222), 100 mg/mL | Auto-injector |

The study periods were referred to as “Extension 1” from Week 60 (Visit 15) to Week 244 (Visit 31); “Extension 2” referred to the study period from week 256 to Week 264 (Visit 35) until 108 weeks after Visit 35 (i.e., Week 364 to Week 372) (Visit 44). “Extension 3” referred to the study period comprising the additional treatment visits after Visit 44. Assessments during the extension studies are provided in Tables 2-7.

TABLE 2
Schedule of Assessments (Extension 1)
Timing of Evaluation and Procedures (Relative to Initiation of Product)

| | Visit Number | | | | | | | | | | | | | | | | | |
|---|---|---|---|---|---|---|---|---|---|---|---|---|---|---|---|---|---|---|
| | Visits | | | | | | | | | | | | | | | | | |
| | 15 | 16 | 17 | 18 | 19 | 20 | 21 | 22 | 23 | 24 | 25 | 26 | 27 | 28 | 29 | 30 | 31 | ET |
| | Scheduled Day/Week | | | | | | | | | | | | | | | | | |
| | Week | | | | | | | | | | | | | | | | | |
| | 60 | 64 | 76 | 88 | 100 | 112 | 124 | 136 | 148 | 160 | 172 | 184 | 196 | 208 | 220 | 232 | 244¹² | |
| | Scheduling Window¹ | | | | | | | | | | | | | | | | | |
| | ±7 days | | | | | | | | | | | | | | | | | |
| Review Entry Criteria | X | | | | | | | | | | | | | | | | | |
| Concomitant Medications | X | X | X | X | X | X | X | X | X | X | X | X | X | X | X | X | X | X |
| Obtain Blood and Urine | X | | X | | X | | X | | X | | X | | X | | X | | X | X |
| Samples for Laboratory | | | | | | | | | | | | | | | | | | |
| Safety Assessments⁸ | | | | | | | | | | | | | | | | | | |
| Urine Pregnancy Test⁵ | X | X | X | X | X | X | X | X | X | X | X | X | X | X | X | X | X | X |
| Obtain PK Sample⁷ | | | X | | X | | X | | X | | X | | X | | X | | X | X |
| Obtain ADA Sample⁷ | | | X | | X | | X | | X | | X | | X | | X | | X | X |
| Perform ECG test | X | | X | | X | | X | | X | | X | | X | | X | | X | X |
| Perform Complete | | | | | | | | | | | | | | | | | | X |
| Physical Exam | | | | | | | | | | | | | | | | | | |
| Assess Vital Signs | X | X | X | X | X | X | X | X | X | X | X | X | X | X | X | X | X | X |
| (temp, HR, RR, BP) | | | | | | | | | | | | | | | | | | |
| PASI | X | X | X | X | X | X | X | X | X | | X | | X | | X | | X | X |
| PGA | X | X | X | X | X | X | X | X | X | | X | | X | | X | | X | X |
| Subject Self- | X | X | X | X | X | X | X | X | X | X | X | X | X | X | X | X | X | |
| Administration Training | | | | | | | | | | | | | | | | | | |
| Self-Administration | X¹³ | X | X | X | X | X | X | X | X | X | X | X | X | X | X | X | X | |
| of Study Medication | | | | | | | | | | | | | | | | | | |
| by PFS or AI | | | | | | | | | | | | | | | | | | |
| Subject Diary | X | X | X | X | X | X | X | X | X | X | X | X | X | X | X | X | X | |
| Completion | | | | | | | | | | | | | | | | | | |
| Dispense Study | X | X | X | X | X | X | X | X | X | X | X | X | X | X | X | X | X | |
| Medication | | | | | | | | | | | | | | | | | | |
| Investigational | X | X | X | X | X | X | X | X | X | X | X | X | X | X | X | X | X | X |
| Product Accountability | | | | | | | | | | | | | | | | | | |
| Monitor for | X | X | X | X | X | X | X | X | X | X | X | X | X | X | X | X | X | X |
| Adverse Events | | | | | | | | | | | | | | | | | | |
| Assess for Severe | X | X | X | X | X | X | X | X | X | X | X | X | X | X | X | X | X | X |
| Infections⁹ | | | | | | | | | | | | | | | | | | |

TABLE 3
Schedule of Assessments (Extension Study 2)

| | EXT 2 Visit number | | | | | | | | | | | |
|---|---|---|---|---|---|---|---|---|---|---|---|---|
| | 1 | 2 | 3 | 4 | 5 | 6 | 7 | 8 | 9 | 10 | | |
| | Visit number | | | | | | | | | | | |
| | 35 | 36 | 37 | 38 | 39 | 40 | 41 | 42 | 43 | 44 | ET¹⁸ | Final |
| | Timing (+/−7 day window) | | | | | | | | | | | |
| | week | | | | | | | | | | | Last |
| | 244/V31 + | V35 + | V35 + | V35 + | V35 + | V35 + | V35 + | V35 + | V35 + | V35 + | | Dose + |
| | 12-20 | 12 | 24 | 36 | 48 | 60 | 72 | 84 | 96 | 108 | | 12 |
| | weeks¹⁷ | weeks | weeks | weeks | weeks | weeks | weeks | weeks | weeks | weeks | | Weeks |
| Sign Informed Consent | X | | | | | | | | | | | |
| Concomitant Medications | X | X | X | X | X | X | X | X | X | X | X | X |
| Obtain Blood and Urine | | X | | X | | X | | X | | X | X | |
| Samples for Laboratory | | | | | | | | | | | | |
| Safety Assessments | | | | | | | | | | | | |
| Urine Pregnancy Test | X | X | X | X | X | X | X | X | X | X | X | |
| Obtain PK sample | | X | | X | | X | | X | | X | X | |
| Obtain ADA sample | | X | | X | | X | | X | | X | X | |
| Perform ECG test | | X | | X | | X | | X | | X | X | |
| Perform Complete | | | | | | | | | | | X | |
| Physical Exam | | | | | | | | | | | | |
| Assess Vital Signs | X | X | X | X | X | X | X | X | X | X | X | |
| (temp, HR, RR, BP) | | | | | | | | | | | | |
| PASI | X | X | | X | | X | | X | | X | X | |
| | (optional) | | | | | | | | | | | |
| PGA | X | X | | X | | X | | X | | X | X | |
| | (optional) | | | | | | | | | | | |
| Subject Self- | X | X | X | X | X | X | X | X | X | X | | |
| Administration Training | | | | | | | | | | | | |
| Self-Administration | X | X | X | X | X | X | X | X | X | X | | |
| of Study Medication | | | | | | | | | | | | |
| by PFS or AI | | | | | | | | | | | | |
| Subject Diary | X | X | X | X | X | X | X | X | X | X | | |
| Completion | | | | | | | | | | | | |
| Dispense Study | X | X | X | X | X | X | X | X | X | X | | |
| Medication | | | | | | | | | | | | |
| Investigational | X | X | X | X | X | X | X | X | X | X | X | |
| Product Accountability | | | | | | | | | | | | |
| Monitor for | X | X | X | X | X | X | X | X | X | X | X | X |
| Adverse Events | | | | | | | | | | | | |
| Assess for Severe | X | X | X | X | X | X | X | X | X | X | X | X |
| Infections | | | | | | | | | | | | |
| Date Commercial | | | | | | | | | | | | X |
| Drug Started | | | | | | | | | | | | |

TABLE 4
Schedule of Assessments (Extension Study 3)

| | EXT 3 Visit number | | | | | | |
|---|---|---|---|---|---|---|---|
| | 1 | 2 | 3 | 4 | 5 | | |
| | Visit number | | | | | | |
| | 60 | 61 | 62 | 63 | 64 | ET²³ | Final |
| | Timing (+/−7-day window) | | | | | | |
| | | V60 + | V60 + | V60 + | V60 + | | |
| | | 12 | 24 | 36 | 48 | | Last dose + |
| | V60/Baseline¹⁹ | weeks | weeks | weeks | weeks²² | | 12 weeks |
| Sign Informed Consent | X²⁰ | | | | | | |
| Concomitant Medications | X | X | X | X | X | X | X |
| Obtain Blood and Urine | | X | | X | | X | |
| Samples for Laboratory | | | | | | | |
| Safety Assessments | | | | | | | |
| Urine Pregnancy Test | X | X | X | X | X | X | |
| Obtain PK Sample | | X | | X | | X | |
| Obtain ADA Sample | | X | | X | | X | |
| Perform ECG test | | X | | X | | X | |
| Perform Complete | | | | | | X | |
| Physical Exam | | | | | | | |
| Assess Vital Signs | X | X | X | X | X | X | |
| (temp, HR, RR, BP) | | | | | | | |
| PASI | X | X | | X | | X | |
| PGA | X | X | | X | | X | |
| Subject Self- | X | X | X | X | X | | |
| Administration Training | | | | | | | |
| Self-Administration | X | X | X | X | X | | |
| of Study Medication | | | | | | | |
| by PFS or AI | | | | | | | |
| Subject Diary Completion | X | X | X | X | X | | |
| Dispense Study Medication | X | X | X | X | X | | |
| Investigational Product | X | X | X | X | X | X | |
| Accountability | | | | | | | |
| Monitor for | X | X | X | X | X | X | X |
| Adverse Events | | | | | | | |
| Assess for Severe | X | X | X | X | X | X | X |
| Infections | | | | | | | |
| Date Commercial | | | | | | | X |
| Drug Started | | | | | | | |

TABLE 5
Schedule of Assessments - Follow-up Period
(Applied to Base and Extension Studies)

| Visit Number | | | |
|---|---|---|---|
| 32¹⁵ | 33¹⁶ | 34 | |
| Weeks from Last Visit | | | |
| 6 | 12 | 20 | ET |
| Visit Window (+/− Calendar days) ¹ | | | |
| 7 | 7 | 7 | |

| | | | |
|---|---|---|---|
| Obtain Blood and Urine Samples for Laboratory | | X | X |
| Safety Assessments⁸ | | | |
| Obtain ADA Sample | | X | X |
| Obtain PK Sample | X | X | X |
| Perform ECG Test | | X | X |
| Perform Complete Physical Exam | | X | X |
| Assess Vital Signs (Temp, RR, HR, BP) | X | X | X |
| Measure Weight (in kg) | | X | X |
| Perform Urine Pregnancy Test⁵ | X | X | X |
| Monitor Adverse Events | X | X | X |
| Assess for Severe Infections⁹ | X | X | X |
| Review Concomitant Medications | X | X | X |
| Investigational Product Accountability | | | X |

TABLE 6
Schedule of Assessments - Follow-up Period (Extension 2 Study)

| | EXT 2 Visit number | | |
|---|---|---|---|
| | 11 | 12 | |
| | Visit Number | | |
| | 45 | 46 | ET |
| | Weeks from Last Visit treatment visit in EXT 2 | | |
| | 12 | 20 | |
| | Visit Window (+/− Calendar days) | | |
| | 7 | 7 | |
| Obtain blood and urine samples for laboratory safety assessments | | X | X |
| Obtain ADA sample | | X | X |
| Obtain PK sample | X | X | X |
| Perform ECG test | | X | X |
| Perform Complete Physical Exam | | X | X |
| Assess vital signs (Temp, RR, HR, BP) | X | X | X |
| Measure weight (in kg) | | X | X |
| Perform Urine Pregnancy Test | X | X | X |
| Monitor Adverse Events | X | X | X |
| Assess for severe infections | X | X | X |
| Review Concomitant Medications | X | X | X |
| Investigational Product Accountability | | | X |

Abbreviations: ADA = anti-drug antibody; AI = auto-injector; BP = blood pressure; BSA = Body Surface Area; cm = centimeters; DNA = deoxyribonucleic acid; ECG = electrocardiogram; HBV = Hepatitis B Virus; HCV = Hepatitis C Virus; HIV = Human Immunodeficiency Virus; HR = Heart Rate; hsCRP = High-sensitivity C-Reactive Protein; kg = kilograms; ICF = Informed Consent Form; PASI = Psoriasis Area and Severity Index; PFS = Pre-filled Syringe; PGA = Physician's Global Assessment; PK = pharmacokinetic; RR = Respiratory Rate; TB = Tuberculosis; Temp = Body temperature

TABLE 7
Schedule of Assessments - Follow-up Period (Extension 3 Study)

| | EXT 3 Visit number | | |
|---|---|---|---|
| | 11 | 12 | |
| | Visit Number | | |
| | 67 | 68 | ET |
| | Weeks from Last Visit treatment visit in EXT 3 | | |
| | 12 | 20 | |
| | Visit Window (+/− Calendar days) | | |
| | 7 | 7 | |
| Obtain blood and urine samples for laboratory safety assessments | | X | X |
| Obtain ADA sample | | X | X |
| Obtain PK sample | X | X | X |
| Perform ECG test | | X | X |
| Perform Complete Physical Exam | | X | X |
| Assess vital signs (Temp, RR, HR, BP) | X | X | X |
| Measure weight (in kg) | | X | X |
| Perform Urine Pregnancy Test | X | X | X |
| Monitor Adverse Events | X | X | X |
| Assess for severe infections | X | X | X |
| Review Concomitant Medications | X | X | X |
| Investigational Product Accountability | | | X |

Abbreviations: ADA = anti-drug antibody; AI = auto-injector; BP = blood pressure; BSA = Body Surface Area; cm = centimeters; DNA = deoxyribonucleic acid; ECG = electrocardiogram; HBV = Hepatitis B Virus; HCV = Hepatitis C Virus; HIV = Human Immunodeficiency Virus; HR = Heart Rate; hsCRP = High-sensitivity C-Reactive Protein; kg = kilograms; ICF = Informed Consent Form; PASI = Psoriasis Area and Severity Index; PFS = Pre-filled Syringe; PGA = Physician's Global Assessment; PK = pharmacokinetic; RR = Respiratory Rate; TB = Tuberculosis; Temp = Body temperature

Early termination visit (ET): For subjects terminating the study prior to the end of Parts 1, 2, or 3, procedures required to be completed within 7 days of early termination. Subjects who terminated early were required to participate in the follow-up period as scheduled.

Note: Subjects were required to continue on study-approved concomitant medications only during the follow-up period but could be placed on appropriate therapies for safety concerns or significant worsening of psoriasis based on the judgment of the investigator.
1. In the event that a subject was not able to visit the investigational site within the visit window as stipulated in the study flowchart, the visit could be rescheduled at another time with prior agreement of the Sponsor. Every attempt required to be made to reschedule as close as possible to the original visit day.
2. Informed consent required to be obtained before any study procedures were performed.
3. Informed consent for pharmacogenetic samples required to obtained before the DNA sample. DNA sample for analysis required to be obtained pre-dose, on Day 1 (or with the next scheduled blood draw), as the last sample drawn, on randomized subjects only, or at a later date as soon as the informed consent was obtained. A subject unwilling to consent to these procedures could still be included in the study; however, pharmacogenetic samples were not allowed to be obtained.
4. Any subject who was started on prophylactic treatment for latent TB during the Screening Period could be randomized 4 weeks after initiation of treatment without need for rescreening.
5. Women of childbearing potential only. If more frequent pregnancy testing was required by local law, the testing was performed as required. Follicle-stimulating hormone test was only required for postmenopausal females <45 years of age, or females with cessation of menses >3 months and <1 year.
6. Optional for subjects, at sites with capability of taking whole body photographs only. Whole body photographs were being used for visual evaluation only and were not included in any analyses. A subject unwilling to consent to these procedures could still be included in the study; however, whole body photography was not allowed to be obtained.
7. Blood samples to be taken pre-dose at tildrakizumab (MK-3222) dosing visits for measurement of tildrakizumab (MK-3222) PK and/or ADA.
8. Safety laboratory tests consisted of:

Hematology: red blood cell (RBC), hematocrit, hemoglobin, platelets, white blood cell (WBC), eosinophils, neutrophils, lymphocytes, monocytes, and basophils

Chemistry: potassium, sodium, chloride, bicarbonate, creatinine, blood urea nitrogen (BUN), total bilirubin, alkaline phosphatase, aspartate aminotransferase (AST; SGOT), alanine aminotransferase (ALT; SGPT), gamma-glutamyl transferase (GGT), lactate dehydrogenase (LDH), total protein, albumin, calcium, inorganic phosphorus, glucose, complete fasting lipid panel (lipids at baseline, Week 12 and Week 52 only), and hsCRP

Urinalysis dipstick: pH, specific gravity, protein, glucose, ketones, blood to be performed at the site. If the dipstick is positive (i.e., trace or above) a sample should be sent to the central lab for a microscopic exam.
9. To be performed only for randomized subjects who reported a severe infection during the study.
10. Subjects were required to receive the first dose of study medication within 24 hours of randomization. All assessments and procedures were required to be done prior to study medication administration. Subjects underwent twice-weekly self-injections of etanercept or etanercept placebo at home after Visit 3 and up to Visit 6 (Week 12). Following the Visit 6 dose and up to Visit 9 (Week 28), subjects continued self-administration of etanercept once-weekly at home. Subjects received doses of tildrakizumab (MK-3222) or tildrakizumab (MK-3222) placebo at Visits 2 and 4, and could self-administer subsequent doses of tildrakizumab (MK-3222) or tildrakizumab (MK-3222) placebo at home following Visit 4.

Study medication administered at the study site, through Week 28, was performed by a third-party administrator who remained blinded to subject treatment allocation. This was a precautionary measure in the event subtle differences in the drug product or primary container were observed.
11. Any subject who discontinued at Week 28 (including subjects discontinued due to response status) was required to have an ET visit performed in place of the Week 28 visit.
12. Subjects could continue to receive treatment beyond 244 weeks if the product was not expected to be available in the subject's local market or until the Sponsor withdraws its marketing authorization application (for Canada only), whichever came first. Procedures that were performed during every visit until 244 weeks continued to be performed at every visit and those that were shifted to be performed at every other visit continued to be performed at every other visit until initiation of the follow-up period or until the subject was transitioned to commercial drug.
13. Following database lock of the base study, subjects received study medication in an open-label manner for the remainder of the long-term extension study. During this open-label phase of the extension study, tildrakizumab (MK-3222) was delivered either via the same type of PFS or with the same type of PFS that had been used during the base study, but wholly contained within an AI.
14. All subjects underwent a 20-week follow-up/wash-out period to monitor safety/tolerability, PK and ADA response. Subjects returned for 2 visits in the follow-up period occurring at 12 and 20 weeks after the subject's last visit in the treatment period (base or extension). Follow-up period was not applicable for subjects who were switched from study medication to commercial drug at the discretion of the treating investigator. Subjects, who had already entered the follow-up period before this version of the protocol was implemented, resumed the study medication after completion of minimum 12 weeks of the follow-up period.
15. There were only 2 visits required as part of the follow-up period (Visits 33 and 34). The Week 6 visit in the follow-up period (Visit 32) was no longer required and had been removed (grayed out) for all subjects entering the follow-up period following implementation of this amendment. Subjects were scheduled for their first visit of the follow-up period (Visit 33) 12 weeks after their last visit in the treatment period (base or extension).
16. Visit numbers could change for subjects who received treatment beyond 244 weeks.
17. For all ongoing subjects, informed consent for the protocol amendment was required to be obtained at the subject's next scheduled/unscheduled visit and, at the latest, at V35. If a subject was currently in the follow-up period and tildrakizumab was commercially available by the time that V35 could be scheduled, the subject was required to return for the next scheduled follow-up visit and sign the ICF documentation. The date that subjects had been switched to commercial drug and the number of weeks completed in the follow-up period were required to be noted in the source documentation.
18. An ET visit was only required if a subject discontinued treatment early (after V35/during the Extension 2) and before the drug was commercially available or before the marketing authorization application was withdrawn by the Sponsor (for Canada only). For these subjects, the follow-up period was applicable.
19. To enter Extension 3, there was required to be a treatment free period of at least 12 weeks between the last dose administered in Extension 2 and the first dose at an Extension 3 visit. Subjects who had already entered the follow-up period before this version of the protocol was implemented resumed study medication after completion of a minimum of 12 weeks of the follow-up period. The maximum timeframe for performing Visit 60 (the start of Extension 3) was 20 weeks [+7 days] after last dose in Extension 2 (this can be an individual visit that depends on the subject's schedule at the time of the end of the Extension 2 study).
20. For all ongoing subjects in Canada, informed consent for this protocol amendment were required to be obtained at the subject's next scheduled/unscheduled visit but no later than visit 60 (first visit of Extension 3). If a subject was in the follow-up period by the time that first visit in Extension 3 could be scheduled, the subject was required to return for the next scheduled follow-up visit and sign the ICF.
21. All subjects underwent a 20-week follow-up/wash-out period to monitor safety/tolerability, PK, and ADA response. Subjects returned for two visits in the follow-up period occurring at 12 and 20 weeks after the subject's last visit in the Extension 3 treatment period. The follow-up period was not applicable for subjects who were switched to commercial drug. The subjects could be switched to commercial drug at the discretion of the treating investigator. If the subject was switched to the commercially available drug, the subjects were required to return for a final visit 12 weeks after the last dose of study medication to be switched to the commercial drug. The date the subject was switched to commercial drug or a new therapy were required to be documented in the source documentation and any appropriate safety documentation (only for AE and concomitant medication) follow-up was required to be completed.
22. Procedures that had been performed during every visit until V60+48 weeks continued to be performed at every visit and those that had been shifted to every other visit continued to be performed at every other visit until initiation of the follow-up period or until the subject was transitioned to commercial drug.
23. An ET visit was only required if a subject discontinued treatment early, i.e., after V60/during the Extension 3 and before the drug was commercially available or at the time the marketing authorization application was withdrawn by the Sponsor. For these subjects, the follow-up period was applicable.

### Study Design

The study design was a long-term safety extension study in subjects with moderate-to-severe chronic plaque psoriasis following a 52-week, Phase 3, randomized, active comparator and placebo-controlled, parallel-design study to evaluate the efficacy and safety/tolerability of subcutaneous tildrakizumab.

There was no randomization or stratification in the extension study. Subjects received tildrakizumab 100 mg or 200 mg every 12 weeks via prefilled syringe or auto injector, which was the same treatment received at the completion of the base study. The long-term extension study was initially conducted in a double-blind manner and then conducted in an open-label manner following database lock of the base study. Eligible subjects participated for a minimum of 212 weeks from the time when the subjects entered the extension study (Extension 1), including 192 weeks of treatment, followed by 20 weeks of follow-up period to monitor safety/tolerability, PK, and ADA response. Subjects continued to receive tildrakizumab treatment beyond 244 weeks (Extension 2 [from Visit 31] and Extension 3 [from Visit 44]).

### Variables Measured

Efficacy variables measured included Psoriasis Area and Severity Index (PASI) 50, PASI 75, PASI 90, and PASI 100, and PGA responses. Pharmacokinetic variables measured included exposure of tildrakizumab (MK-3222), covariates which impact PK behavior of tildrakizumab and the correlation between PK patterns with efficacy parameters. Immunogenicity variables measured include anti-drug antibody (ADA) status (positive or negative/inconclusive, treatment-emergent or non-treatment-emergent, neutralizing antibody positive or negative) and its correlation with PK, efficacy, and safety.

### Analysis Populations

Four data sets were analyzed: (1) Full Analysis Set (FAS) including all subjects who entered extension study and received at least one dose of extension study treatment, based on the treatment assigned; (2) All Subjects as Treated (ASaT) including all subjects who entered the extension study and received at least one dose of extension study treatment, based on the treatment received; (3) PK evaluable including all subjects who entered extension and had at least 1 PK data point after dosing tildrakizumab; and (4) ADA evaluable including all subjects who entered the extension phase and had at least one ADA data point after dosing tildrakizumab. The FAS was used for the efficacy analysis.

### Psoriasis Area and Severity Index

PASI (a quantitative rating score for measuring the severity of psoriatic lesions based on area coverage and plaque appearance) was used to measure erythema (redness), induration (thickness), scaling, and coverage of plaques (i.e., body surface area covered with lesions) of subjects. Measurements were taken at the time point as indicated in Tables 2-4. PASI score ranges from 0 (no psoriasis on the body) to 72 (the most severe case of psoriasis). PASI 50, PASI 75, PASI 90, and PASI 100 represent the status of achieving ≥50%, ≥75%, ≥90%, and 100% reduction from baseline in PASI score, respectively.

### Physician Global Assessment

The overall severity of psoriasis lesions using the PGA at the time points was determined at the time points as indicated in Tables 2-4. Lesions were graded for thickness, erythema, and scaling on a 0 to 5-point scale. The sum of the 3 scales was divided by 3 to obtain the final PGA score.

### Adverse Events

Per the ICH, an AE is defined as any untoward medical occurrence in a patient or clinical investigation subject administered a pharmaceutical product and which does not necessarily have to have a causal relationship with this treatment. An AE could therefore be any unfavorable and unintended sign (including an abnormal laboratory finding, for example), symptom, or disease temporally associated with the use of a medicinal product, whether or not considered related to the medicinal product.

A list of the Tier 1, Tier 2, and Tier 3 safety endpoints is provided in Table 9. For adverse experiences (specific terms as well as system organ class terms) that were not pre-specified as Tier 1 or Tier 2 endpoints, membership in Tier 2 requires that at least 4 subjects in any treatment group exhibited the event; all other adverse experiences belonged to Tier 3. Continuous measures, such as changes from baseline in laboratory, vital signs, and ECG parameters, that were not pre-specified as Tier 1 endpoints were considered Tier 3 safety parameters.

### Serious Adverse Events

An SAE is any untoward medical occurrence or effect that at any dose:
1. Resulted in death;
2. Was life-threatening;
3. Requires hospitalization or prolongation of existing inpatients' hospitalization;
4. Results in a persistent or significant disability or incapacity; and/or
5. Was a congenital anomaly or birth defect;
6. Was a cancer;
7. Was associated with an overdose;
8. Was an Other Important Medical Event.

Life-threatening in the definition of an SAE refers to an event in which the subject was at risk of death at the time of the event; it does not refer to an event which hypothetically might have caused death if it were more severe.

Medical judgment required to be exercised in deciding whether an AE/reaction is serious in other situations. Important AEs/reactions that were not immediately life-threatening or did not result in death or hospitalization but might jeopardize the subject or might require intervention to prevent one of the other outcomes listed in the definition above, had to be considered serious. These were considered “Other Important Medical Events”.

### Adverse Events of Special Interest

Adverse events of special interest that were identified a priori constituted “Tier 1” safety/tolerability endpoints:
severe infections
malignancies (excluding carcinoma in situ of the cervix)
non-melanoma skin cancer
confirmed Extended MACE
drug-related hypersensitivity reactions (e.g., anaphylaxis, urticaria, angioedema).

### Clinical Laboratory Evaluation—Safety

Laboratory tests (Table 8) for hematology, high-sensitivity C-reactive protein (hsCRP), blood chemistry, and urinalysis were performed at the time points as specified in Section Tables 2-4. Blood samples for laboratory tests were taken prior to investigational product(s) administration and analysis performed using standard laboratory procedures. Test results outside the reference range and considered clinically significant by the investigator, were repeated at appropriate time intervals until the result returned to baseline or was determined to be not clinically significant.

TABLE 8
Laboratory Tests

| Hematology | Chemistry | Urinalysis |
|---|---|---|
| Basophils | Albumin | Blood |
| Eosinophils | Alkaline phosphatase | Glucose |
| Hematocrit | Alanine transaminase (ALT; SGPT) | Ketones |
| Hemoglobin | Aspartate aminotransferase (AST; SGOT) | Microscopic exam (if dipstick |
| | | was positive) |
| Lymphocytes | Bicarbonate | pH |
| Monocytes | Blood urea nitrogen (BUN) | Protein |
| Neutrophils | Calcium | Specific gravity |
| Platelets | Chloride | |
| Red blood cells | Creatinine | |
| White blood cells | Gamma-glutamyl transpeptidase (GGT) | |
| | Glucose | |
| | High-sensitive C-reactive protein | |
| | (hs-CRP) | |
| | Inorganic phosphorus | |
| | Lactate dehydrogenase | |
| | Potassium | |
| | Sodium | |
| | Total Bilirubin | |
| | Total protein | |

### Vital Sign Measurements

Vital signs were obtained at the time points specified in Tables 2-4. Systolic and diastolic blood pressure (mm Hg), respiratory rate, heart rate (bpm) rate (bpm), and temperature were recorded. Clinically significant changes in vital sign measurements from baseline were recorded as adverse events.

### 12-Lead ECG

A 12-lead electrocardiogram (ECG) was performed at the time points specified in Tables 2-4. Clinically significant changes in the ECG from baseline for subsequent ECGs were recorded as adverse events.

### Physical Examination

A complete physical examination was performed on all subjects at the time points specified in Tables 2-4. Any medical condition found during the Screening and Baseline physical examination in the base study was recorded and clinically significant changes from the Screening and/or Baseline physical examination were recorded as adverse events.

### Pharmacokinetic Measurements

A minimum of 2 mL sample of blood was collected into the appropriate tubes for determinations of serum concentrations of tildrakizumab prior to study medication administration at the time points specified in Tables 2-4. Sample collection times were recorded.

Serum tildrakizumab assays were performed using a validated electrochemiluminescence immunoassay (ECL). During the study period, three different validated ECL methods were employed, all of which had a conventional bridging assay design, that used biotinylated hIL-23 as the capture reagent and Meso Scale Discovery® SULFO-TAG™ labelled IL-23 or anti-tildrakizumab antibody as detection reagent. Responses were read on a MesoScale Discovery® platform (lower limit of quantitation of 3.12 ng/mL or 20 ng/mL when using SULFO-TAG™ labelled IL-23 and 25 ng/mL when using SULFO-TAG™ Anti-Tildrakizumab).

### Immunogenicity Measurements

A minimum of 6 mL sample of blood was collected into the appropriate tubes for determinations of anti-tildrakizumab antibodies prior to study medication administration at the time points specified in Tables 2-4. A validated bridging electrochemiluminescence immunoassay was used for the detection of ADA in human serum. Biotin and SULFO-TAG™ labelled tildrakizumab were used for capture and detection of anti-tildrakizumab antibodies respectively. Assay responses were read on a MesoScale Discovery® platform. The drug tolerance level for the anti-tildrakizumab ADA assay was 6 μg/mL for 500 ng/mL of ADA in serum. Bioanalysis of ADA was carried out using the using the standard 3-tiered assay approach that consisted of screening (Tier 1), confirmation (Tier 2), and antibody characterization (titer and neutralizing capacity) (Tier 3). For confirmed ADA positive samples, the immune response was further characterized for antibody titer and neutralizing capacity, which is the ability of the ADA to neutralize the binding of tildrakizumab to its biologic target (hIL-23).

### Data Quality Assurance

Clinical studies were subject to Quality Control (QC) and Quality Assurance oversight, including independent audits. Quality Control and Quality Assurance activities were intrinsic to all clinical study-related activities. Such activities included, but were not limited to, on-site monitoring inclusive of source data verification, medical monitoring of clinical study data and resultant databases and quality reviews of regulatory submission documents.

### Statistical Analysis

The primary efficacy endpoint for this study was the proportion of subjects with Psoriasis Area and Severity Index (PASI) 50 response over time among PASI 50 responders at week 52 of the baseline study. A second endpoint was the proportion of subjects with PASI 75 response over time among PASI 75 responders at week 52. A third endpoint was the proportion of subjects with PASI 90 response over time among PASI 90 responders at week 52. A fourth endpoint was the proportion of subjects with PASI 100 response over time among PASI 100 responders at week 52. A fifth endpoint was the proportion of subjects with a physician's global assessment (PGA) score of “clear” or “minimal” with at least a 2-grade reduction from baseline over time. A sixth endpoint was the change and percent change in PASI score from the base study baseline over time.

### General Approaches

Continuous data were assumed to be normally distributed and summarized in terms of mean, standard deviation (SD), median, minimum, maximum and number of observations. Categorical data were summarized in terms of the number of subjects providing data at the relevant time point (n), frequency counts and percentages. Percentages were presented to one decimal place. Percentages were not presented for zero counts. Percentages were calculated using (n) as the denominator.

Outputs were produced using SAS® version 9.2 or a later version. The REPORT procedure was used to produce all tables and listings whenever possible. The GPLOT procedure was used to produce all figures whenever possible. All statistical appendices (supportive SAS output) were output directly from the appropriate SAS procedure.

### Analysis Populations

The analysis sets analyzed in the extension study were: (1) Full Analysis Set (FAS): All subjects who entered extension study and received at least one dose of extension study treatment, based on the treatment assigned; (2) All Subjects as Treated (ASaT): All subjects who entered extension and received at least one dose of extension study treatment, based on the treatment received; (3) PK evaluable: All subjects who entered extension and had at least one PK data point after dosing tildrakizumab; (4) ADA evaluable: All subjects who entered extension and had at least one ADA data point after dosing tildrakizumab.

### Baseline Characteristics

Baseline characteristics including baseline PASI and PGA score and medical conditions were summarized by treatment group and overall, for all subjects entered the extension study. No statistical hypothesis tests were performed on these characteristics.

### Treatment Compliance

Drug accountability data was collected for tildrakizumab (MK-3222) during the extension study. Compliance was measured for subjects: (1) received unscheduled study medication injections, (2) missed an injection, and (3) received incorrect study medication dose. Numbers and percentages of subject and injection visits with any deviation in these measures were reported. Compliance was calculated using the following formula: Percent Compliance=Number of Injections Received/Number of Injections Patient Should Have Received×100. Summary statistics were provided on percent compliance by treatment group and overall for all randomized subjects.

### Extent of Exposure

The range of duration (in weeks) on study medication and the mean number of weeks on study medication were calculated by treatment group that subjects were originally randomized to, during the extension study only and during the base study and the extension study.

### Efficacy Endpoint Methodology

For all efficacy results, baseline was defined as the last measurement taken prior to the first study medication in the base study. Week 52 was the last scheduled assessment recorded prior to the last dose in the base study. Descriptive summary statistics were provided by treatment group for all efficacy endpoints over time during the extension study. No formal statistical analysis was performed to compare between two tildrakizumab (MK-3222) doses. Descriptive summary statistics were provided by treatment group (tildrakizumab 100 mg and 200 mg groups) for all safety parameters.

### Safety Endpoint Methodology

Safety and tolerability were assessed by clinical review of all relevant parameters including AEs, laboratory tests, 12-Lead ECG, and vital signs measurements. No between-treatment comparison was performed in the extension study. Descriptive summary statistics were provided by treatment group (tildrakizumab 100 mg and 200 mg groups) for all safety parameters. Table 9 provides an overview of safety endpoints by tier.

TABLE 9
List of Safety Endpoints by Tier

| Safety Tier | Safety Endpoint^{†} |
|---|---|
| Tier 1 | Percent of subjects with severe infections^{††} |
| | Percent of subjects with malignancies (excluding carcinoma in situ of |
| | the cervix) |
| | Percent of subjects with non-melanoma skin cancer |
| | Percent of subjects with melanoma skin cancer |
| | Percent of subjects with confirmed Extended MACE |
| | Percent of subjects with drug-related hypersensitivity reactions |
| | (anaphylaxis, urticaria, angioedema, etc) |
| Tier 2 | Percent of subjects with any AE |
| | Percent of subjects with any drug-related AE |
| | Percent of subjects with any SAE |
| | Percent of subjects with discontinuation due to AE |
| | Percent of subjects with discontinuation due to drug-related AE |
| | Percent of subjects with specific AEs, SOCs, or PDLCs |
| | (incidence ≥ 4 of subjects in one of the treatment groups) |
| | Percent of subjects with death |
| | Percent of subjects with any AE associated with anti-drug antibodies |
| | Percent of subjects with confirmed MACE |
| | Percent of subjects with confirmed thrombotic/embolic/ischemic |
| | cardiovascular events (including MACE and Extended MACE) |
| Tier 3 | Percent of subjects with specific AEs, SOCs or PDLCs^{‡} |
| | (incidence < 4 of subjects in all of the treatment groups) |
| | Change from Baseline Results (Labs, ECGs, Vital Signs) |

^{†}Adverse Experience references refer to both Clinical and Laboratory AEs.
^{††}Defined as any infection meeting the regulatory definition of a serious adverse event, or any infection requiring IV antibiotics whether or not reported as a serious event, as per the regulatory definition.
^{‡}AE = Adverse Experience; ECGs—Electrocardiogram; IV = Intravenous; MACE = Major Adverse Cardiovascular Event; SAE = Serious Adverse Event; SOC = System Organ Class; PDLC = Pre-Defined Limit of Change

### Pharmacokinetic Endpoint Methodology

Descriptive statistics of tildrakizumab (MK-3222) concentrations were presented by time point and treatment group. Serum concentrations of tildrakizumab (MK-3222) at each nominal sample time were calculated and reported, including: arithmetic mean, SD, arithmetic coefficient of variation, median, standard error (SE), range, geometric mean, geometric coefficient of variation, and the number of observations. Values below the limit of quantitation were imputed as ½ lower level of quantification to enable reporting of the geometric mean. The data were reported to three significant digits and coefficient of variation values were rounded to one decimal. Further, the impact of ADA status on tildrakizumab (MK-3222) serum concentrations was explored. PK concentrations were simulated for all subjects in this study.

### Immunogenicity Endpoint Methodology

Post-dose ADA assay results were evaluated for immunogenicity assessment. “Post-dose samples” as used herein are samples collected after administration of tildrakizumab. Subjects were grouped based on the actual treatment received, rather than the treatment group at randomization and a baseline sample taken prior to dosing to assess for pre-existing antibodies that may be detected by the ADA assays. Subjects were considered positive if at least one post-dose sample was positive in the ADA confirmatory assay. Positive subjects were categorized as treatment-emergent positive if the positive sample occurred following treatment with tildrakizumab or non-treatment emergent positive if the subject had an immune response present at baseline and the response was not boosted following treatment.

Samples with a negative test result in the screening or confirmatory anti-tildrakizumab assay were confirmed to be negative if the tildrakizumab concentration was below 6 μg/mL. Tildrakizumab levels above 6 μg/mL are above the drug tolerance level (DTL) and could interfere with the ADA assay. The immunogenicity status of a subject was confirmed to be negative if all pre-treatment and post-dose samples were negative in the confirmatory assays and if the concentration of tildrakizumab in the last post-dose sample was below the DTL.

Subjects were categorized into immunogenicity categories as described in Table 10. The overall immunogenicity incidence was defined as the proportion of treatment-emergent positive subjects to the number of evaluable subjects. The proportion of non-treatment-emergent positive subjects was similarly reported. For ADA positive subjects (based on the confirmatory assay), the immune response was further characterized for antibody titer and neutralizing capacity.

TABLE 10
Immunogenicity Subject Status Definitions

| Immunogenicity Subject Status | |
|---|---|
| Definitions Subject Status | Definition |
| Negative | All pre-treatment and post-dose samples were negative in |
| | the ADA confirmatory assay and the drug concentration |
| | in the last post-dose sample was below the respective |
| | DTL for the ADA assay. |
| Inconclusive | All pre-treatment and post-dose samples were negative in |
| | the ADA confirmatory assay AND the drug concentration |
| | in the last post-dose sample was equal or above the |
| | respective DTL for the ADA assay. |
| Treatment-emergent Positive | Pre-treatment sample was negative and at least 1 post- |
| | dose sample was positive in the ADA confirmatory assay |
| | (treatment-induced positive). |
| | Pre-treatment and post-dose samples were both positive |
| | in the ADA confirmatory assay and the titer increased |
| | post-dose by ≥ 2-fold (treatment boosted positive). |
| Non-treatment- emergent Positive | Pre-treatment sample was positive and post-dose samples |
| | were negative in the ADA confirmatory assay. |
| | Pre-treatment and post-dose samples were positive in the |
| | ADA confirmatory assay with a < 2-fold increase in titer |
| | post-dose |

ADA = anti-drug antibodies; DTL = drug tolerance level.

### Study Subjects

### Disposition of Subjects

The disposition of all subjects by treatment groups for the extension study (Extension 1, 2, and 3) is provided in Table 11.

The tildrakizumab 100 mg group 1 contained 381 subjects and the 200 mg Group 2 contained 349 subjects. The percentage of subjects who completed Extension 1 was similar between the two treatment groups (290 [76.1%] subjects and 272 [77.9%] subjects in the tildrakizumab 100 mg and 200 mg groups, respectively). Of the 730 subjects who entered Extension 1, 166 (22.7%) subjects discontinued the study (23.6% and 21.8% in the tildrakizumab 100 mg group and 200 mg group, respectively). The most common reason for discontinuation in each treatment group in Extension 1 was withdrawal by subjects (8.1% overall, and 9.2% and 6.9% in the tildrakizumab 100 mg group and 200 mg group, respectively). A total of 455 (62.3%) subjects entered the follow-up period of Extension 1, and 273 (37.4%) subjects did not enter the follow-up period of Extension 1.

After completion of the Extension 1 follow-up, 249 subjects entered Extension 2. A total of 95 subjects entered Extension 2 and 51 subjects did not enter the follow-up period of Extension 2.

A total of 31 subjects entered Extension 3 (20 subjects and 11 subjects in the tildrakizumab 100 mg group and 200 mg group, respectively). All subjects completed Extension 3.

TABLE 11
Disposition of Subjects (All Subjects Entered Extension Study)

| | MK-3222 100 mg | | MK-3222 200 mg | | Total | |
|---|---|---|---|---|---|---|
| | n | (%) | n | (%) | n | (%) |
| Subjects in Population^{†} | 381 | | 349 | | 730 | |

Study Disposition

| | | | | | | |
|---|---|---|---|---|---|---|
| Extension 1 | | | | | | |
| Number of Subjects who | 290 | (76.1) | 272 | (77.9) | 562 | (77.0) |
| completed extension 1 | | | | | | |
| Number of subjects who | 90 | (23.6) | 76 | (21.8) | 166 | (22.7) |
| discontinued from study | | | | | | |
| Reason for discontinuation | | | | | | |
| Adverse Events | 14 | (3.7) | 11 | (3.2) | 25 | (3.4) |
| Death | 3 | (0.8) | 1 | (0.3) | 4 | (0.5) |
| Lack of Efficacy | 10 | (2.6) | 11 | (3.2) | 21 | (2.9) |
| Lost to Follow-up | 15 | (3.9) | 13 | (3.7) | 28 | (3.8) |
| Non-Compliance with | 2 | (0.5) | 2 | (0.6) | 4 | (0.5) |
| Study Drug | | | | | | |
| Physician Decision | 8 | (2.1) | 11 | (3.2) | 19 | (2.6) |
| Pregnancy | 2 | (0.5) | 1 | (0.3) | 3 | (0.4) |
| Progressive Disease | 0 | | 2 | (0.6) | 2 | (0.3) |
| Protocol Violation | 0 | | 0 | | 0 | |
| Study Terminated by | 0 | | 0 | | 0 | |
| Sponsor | | | | | | |
| Withdrawal by Subjects | 35 | (9.2) | 24 | (6.9) | 59 | (8.1) |
| Other Protocol Specified | 1 | (0.3) | 0 | | 1 | (0.1) |
| Criteria | | | | | | |
| Follow-Up | | | | | | |
| Entered Follow-up | 224 | (58.8) | 231 | (66.2) | 455 | (62.3) |
| Did Not Enter Follow-up | 156 | (40.9) | 117 | (33.5) | 273 | (37.4) |
| Extension 2^{‡} | | | | | | |
| Number of Subjects who | 31 | (8.1) | 21 | (6.0) | 52 | (7.1) |
| completed extension 2 | | | | | | |
| Number of subjects who | 46 | (12.1) | 48 | (13.8) | 94 | (12.9) |
| discontinued from study | | | | | | |
| Reason for discontinuation | | | | | | |
| Adverse Events | 2 | (0.5) | 0 | | 2 | (0.3) |
| Death | 0 | | 0 | | 0 | |
| Lack of Efficacy | 4 | (1.0) | 1 | (0.3) | 5 | (0.7) |
| Lost to Follow-up | 0 | | 0 | | 0 | |
| Non-Compliance with | 0 | | 0 | | 0 | |
| Study Drug | | | | | | |
| Physician Decision | 1 | (0.3) | 2 | (0.6) | 3 | (0.4) |
| Pregnancy | 0 | | 0 | | 0 | |
| Progressive Disease | 0 | | 0 | | 0 | |
| Protocol Violation | 0 | | 0 | | 0 | |
| Study Terminated by | 12 | (3.1) | 27 | (7.7) | 39 | (5.3) |
| Sponsor | | | | | | |
| Withdrawal by Subjects | 10 | (2.6) | 8 | (2.3) | 18 | (2.5) |
| Other Protocol Specified | 17 | (4.5) | 10 | (2.9) | 27 | (3.7) |
| Criteria | | | | | | |
| Follow-Up | | | | | | |
| Entered Follow-up | 45 | (11.8) | 50 | (14.3) | 95 | (13.0) |
| Did Not Enter Follow-up | 32 | (8.4) | 19 | (5.4) | 51 | (7.0) |
| Extension 3 | | | | | | |
| Number of Subjects who | 20 | (5.2) | 11 | (3.2) | 31 | (4.2) |
| completed extension 3 | | | | | | |
| Number of subjects who | 0 | | 0 | | 0 | |
| discontinued from study | | | | | | |
| Reason for discontinuation | | | | | | |
| Adverse Events | 0 | | 0 | | 0 | |
| Death | 0 | | 0 | | 0 | |
| Lack of Efficacy | 0 | | 0 | | 0 | |
| Lost to Follow-up | 0 | | 0 | | 0 | |
| Non-Compliance with | 0 | | 0 | | 0 | |
| Study Drug | | | | | | |
| Physician Decision | 0 | | 0 | | 0 | |
| Pregnancy | 0 | | 0 | | 0 | |
| Progressive Disease | 0 | | 0 | | 0 | |
| Protocol Violation | 0 | | 0 | | 0 | |
| Study Terminated by | 0 | | 0 | | 0 | |
| Sponsor | | | | | | |
| Withdrawal by Subjects | 0 | | 0 | | 0 | |
| Other Protocol Specified | 0 | | 0 | | 0 | |
| Criteria | | | | | | |
| Follow-Up | | | | | | |
| Entered Follow-up | 1 | (0.3) | 0 | | 1 | (0.1) |
| Did Not Enter Follow-up | 19 | (5.0) | 11 | (3.2) | 30 | (4.1) |

^{†}Number of subjects who completed base and received at least 1 dose of study medication in extension.
^{‡}There are 249 subjects in total entering Extension 2 Study. Out of these 249 subjects, 22 subjects have unknown status due to early site closure and 81 subjects switched to commercial drug without completion or discontinuation status. Therefore, only 146 subjects have their completion/discontinuation information captured in the EDC database. Out of these 146 subjects, 52 subjects completed Extension 2 Study and 94 subjects discontinued.
Note:
Extension 1 subjects USUBJID 3222-011_330700010 and 3222-011_480700012 were not included as they were missing completion/discontinuation information.

### Protocol Deviations

An overall summary of major protocol deviations for all subjects who entered the extension study is provided in Table 12. The most common major protocol deviations were related to informed consent (310 [42.5%] subjects) and investigational product administration or study treatment (143 [19.6%] subjects), and procedures or tests (110 [15.1%] subjects).

TABLE 12
Summary of Major Protocol Deviations: Overall
(All Subjects Entered Extension Study)

| | Number of Subjects |
|---|---|
| Deviation Category | (n/N) (%) |
| AE SAE | 89/730 (12.2) |
| Disallowed Medications | 101/730 (13.8) |
| Inc/Excl Criteria | 6/730 (0.8) |
| Informed Consent | 310/730 (42.5) |
| Investigator Obligations | 3/730 (0.4) |
| IP admin/study treat | 143/730 (19.6) |
| Other | 1/730 (0.1) |
| Procedures/Tests | 110/730 (15.1) |
| Subject Data | 6/730 (0.8) |
| Test Article | 2/730 (0.3) |
| Visit Schedule | 89/730 (12.2) |

AE = Adverse event; n = Number of subjects with major protocol deviations reported; N = Total number of subjects entered extension study; SAE = Serious adverse event.

### Demographic and Baseline Characteristics

Subject characteristics by treatment group and overall, in all subjects entered the extension study is summarized in Table 13.

TABLE 13
Subject Baseline Characteristics (All
Subjects Entered Extension Study)

| | MK-3222 | MK-3222 | |
|---|---|---|---|
| | 100 mg | 200 mg | Total |
| | n (%) | n (%) | n (%) |
| Subjects in population | 381 | 349 | 730 |

Gender

| | | | |
|---|---|---|---|
| Male | 291 (76.4) | 242 (69.3) | 533 (73.0) |
| Female | 90 (23.6) | 107 (30.7) | 197 (27.0) |

Age (years)

| | | | |
|---|---|---|---|
| <65 | 354 (92.9) | 323 (92.6) | 677 (92.7) |
| ≥65 | 27 (7.1) | 26 (7.4) | 53 (7.3) |
| Mean | 44.2 | 45.6 | 44.9 |
| SD | 13.26 | 12.77 | 13.03 |
| Median | 44.0 | 46.0 | 45.0 |
| Range | 19 to 80 | 19 to 76 | 19 to 80 |

Race

| | | | |
|---|---|---|---|
| American Indian or | 1 (0.3) | 0 | 1 (0.1) |
| AlaskaNative | | | |
| Black | 8 (2.1) | 3 (0.9) | 11 (1.5) |
| Native Hawaiian or | 0 | 0 | 0 |
| Other Pacific Island | | | |
| White | 351 (92.1) | 329 (94.3) | 680 (93.2) |
| Asian | 7 (1.8) | 8 (2.3) | 15 (2.1) |
| Multi-Racial | 7 (1.8) | 3 (0.9) | 10 (1.4) |
| Missing | 7 (1.8) | 6 (1.7) | 13 (1.8) |

Ethnicity^{†}

| | | | |
|---|---|---|---|
| Hispanic or Latino | 22 (5.8) | 26 (7.4) | 48 (6.6) |
| Not Hispanic or Latino | 347 (91.1) | 313 (89.7) | 660 (90.4) |
| Not Reported | 4 (1.0) | 4 (1.1) | 8 (1.1) |
| Unknown | 4 (1.0) | 2 (0.6) | 6 (0.8) |

Weight (kg)

| | | | |
|---|---|---|---|
| Subjects with data | 381 | 349 | 730 |
| Mean | 88.43 | 88.96 | 88.68 |
| SD | 21.384 | 21.488 | 21.421 |
| Median | 86.10 | 86.70 | 86.50 |
| Range | 49.5 to 166.0 | 44.0 to 158.4 | 44.0 to 166.0 |

Height (cm)

| | | | |
|---|---|---|---|
| Subjects with data | 380 | 349 | 729 |
| Mean | 174.36 | 173.23 | 173.82 |
| SD | 9.365 | 10.247 | 9.806 |
| Median | 175.00 | 173.00 | 174.00 |
| Range | 150.0 to 196.0 | 117.5 to 198.0 | 117.5 to 198.0 |

Psoriatic Arthritis

| | | | |
|---|---|---|---|
| Yes | 57 (15.0) | 48 (13.8) | 105 (14.4) |
| No | 324 (85.0) | 301 (86.2) | 625 (85.6) |

Body Surface Area (%)

| | | | |
|---|---|---|---|
| Subjects with data | 378 | 345 | 723 |
| Mean | 32.6 | 30.1 | 31.4 |
| SD | 18.01 | 15.75 | 17.00 |
| Median | 28.0 | 26.0 | 27.0 |
| Range | 10 to 96 | 8 to 89 | 8 to 96 |

^{†}Not reported: if ethnicity is not provided or available, Unknown: if ethnicity is not known, not observed, not recorded or refused.
Baseline is defined as the last measurement taken prior to the first study medication in the base study.
SD = Standard Deviation.

### Baseline Disease Characteristics

A summary of baseline efficacy endpoints is provided by treatment group for all subjects entered the extension study in Table 14.

TABLE 14
Summary of Baseline Efficacy Endpoints (All Subjects
Entered Extension Study) Extension Study

| | MK-3222 | MK-3222 | |
|---|---|---|---|
| | 100 mg | 200 mg | Total |
| | n (%) | n (%) | n (%) |
| Subjects in | 381 | 349 | 730 |
| population | | | |

PASI Score

| | | | |
|---|---|---|---|
| Subjects | 380 | 348 | 728 |
| with data | | | |
| Mean | 19.8 | 19.3 | 19.6 |
| SD | 7.65 | 6.89 | 7.29 |
| Median | 17.5 | 17.2 | 17.4 |
| Range | 5.3 to 54.8 | 8.1 to 50.4 | 5.3 to 54.8 |

PGA Score

| | | | |
|---|---|---|---|
| Subjects | 378 | 345 | 723 |
| with data | | | |
| <3 | 17 (4.5) | 7 (2.0) | 24 (3.3) |
| 3 | 238 (62.5) | 215 (61.6) | 453 (62.1) |
| 4 | 117 (30.7) | 115 (33.0) | 232 (31.8) |
| 5 | 6 (1.6) | 8 (2.3) | 14 (1.9) |

PASI = Psoriasis Area Severity Index; PGA = Physician's Global Assessment; SD = Standard deviation.
Baseline was defined as the last measurement taken prior to the first study medication

Baseline efficacy parameters were similar between treatment groups. Baseline mean PASI scores were 19.8 and 19.3 in the tildrakizumab 100 mg group and 200 mg group, respectively. Subjects with PGA score of 3 were 62.1% overall, with 62.5% and 61.6% in the tildrakizumab 100 mg group and 200 mg group.

### Treatment Compliance

Treatment compliance for the overall extension study (Extension 1, 2, and 3 combined) is summarized by treatment group for all subjects randomized in the extension study in Table 15.

TABLE 15
Summary of Treatment Compliance to MK-3222 (All
Subjects Randomized - Extension) Extension Study

| | MK-3222 100 mg | | MK-3222 200 mg | | Total | |
|---|---|---|---|---|---|---|
| | N = 381 | | N = 349 | | N = 730 | |
| | n | (%) | n | (%) | n | (%) |
| Extension Study | | | | | | |
| Received unscheduled | 5 | (1.3) | 5 | (1.4) | 10 | (1.4) |
| injection | | | | | | |
| Missed an injection | 37 | (9.7) | 42 | (12.0) | 79 | (10.8) |
| Received incorrect dose | 12 | (3.1) | 29 | (8.3) | 41 | (5.6) |
| Reason for incorrect dose | | | | | | |
| Adverse Event | 4 | (1.0) | 6 | (1.7) | 10 | (1.4) |
| Device Failure | 1 | (0.3) | 13 | (3.7) | 14 | (1.9) |
| General Compliance Problems | 1 | (0.3) | 2 | (0.6) | 3 | (0.4) |
| Medication Error | 1 | (0.3) | 3 | (0.9) | 4 | (0.5) |
| Other | 7 | (1.8) | 13 | (3.7) | 20 | (2.7) |
| Treatment Compliance | | | | | | |
| ≤20% | 21 | (5.5) | 10 | (2.9) | 31 | (4.2) |
| >20% to ≤40% | 22 | (5.8) | 29 | (8.3) | 51 | (7.0) |
| >40% to ≤60% | 23 | (6.0) | 17 | (4.9) | 40 | (5.5) |
| >60% to ≤80% | 76 | (19.9) | 79 | (22.6) | 155 | (21.2) |
| >80% | 239 | (62.7) | 214 | (61.3) | 453 | (62.1) |
| Summary Statistics for | | | | | | |
| Treatment Compliance (%) | | | | | | |

| | | | |
|---|---|---|---|
| Mean | 82.84 | 83.03 | 82.95 |
| SD | 21.705 | 20.794 | 21.190 |
| Median | 88.89 | 88.89 | 88.89 |
| Range | 5.88 to 100.00 | 5.88 to 100.00 | 5.88 to 100.00 |

Compliance was calculated as (number of injections received/number of injection should have received) × 100%.
SD = Standard deviation.

The mean overall compliance during the extension study was 82.95%. No meaningful differences were observed in treatment compliance between the treatment groups (82.84% and 83.03% in the tildrakizumab 100 mg group and 200 mg group, respectively.

### Extent of Exposure

A summary of the extent of exposure to tildrakizumab 100 mg and 200 mg is provided by treatment group for all randomized subjects in the extension study in Table 16.

The median number of weeks on tildrakizumab was 192.0 weeks in the tildrakizumab 100 mg group and 192.0 weeks in the tildrakizumab 200 mg group.

TABLE 16
Extent of Exposure to MK-3222 (All
Subjects Randomized - Extension)

| | | MK-3222 | MK-3222 |
|---|---|---|---|
| | | 100 mg | 200 mg |
| ≤24 | Weeks | 16 | 8 |
| 25-48 | Weeks | 14 | 20 |
| 49-72 | Weeks | 17 | 12 |
| 73-96 | Weeks | 7 | 11 |
| 97-120 | Weeks | 15 | 7 |
| 121-144 | Weeks | 7 | 8 |
| 145-168 | Weeks | 10 | 10 |
| 169-192 | Weeks | 161 | 123 |
| >192 | Weeks | 129 | 148 |

| | | |
|---|---|---|
| Total Subjects | 376 | 347 |
| Duration Range | 12 to 344 | 12 to 344 |
| Mean Duration | 188.7 | 188.6 |
| Median Duration | 192.0 | 192.0 |

Each subject was counted once on each applicable dosage category row.
This table reflects transient cross-treatments.
Only subjects treated during extension are included in the table.

### Efficacy and Other Evaluations

### Example 1. PAST50 Response Over Time Among PAST50 Responders at Week 52

The PASI0 response, among those who were responders at Week 52, was maintained in subjects who remained in the study at specified time points throughout the extension study (≥97.9% and ≥95.7% the subjects in the tildrakizumab 100 mg group and the tildrakizumab 200 mg group, respectively). See Table 17.

TABLE 17
Proportion of Subjects with PASI50 Response Over Time - PASI50
Responders at Week 52 (Full Analysis Set) Extension Study

| | | |
|---|---|---|
| | MK-3222 | MK-3222 |
| | 100 mg | 200 mg |
| Statistics | N = 381 | N = 349 |

PASI50 (Week 60)

| | | |
|---|---|---|
| Subjects with data | 373 | 341 |
| Responders (%)^{†} | 367 (98.4) | 336 (98.5) |

PASI50 (Week 64)

| | | |
|---|---|---|
| Subjects with data | 368 | 338 |
| Responders (%)^{†} | 364 (98.9) | 332 (98.2) |

PASI50 (Week 76)

| | | |
|---|---|---|
| Subjects with data | 367 | 333 |
| Responders (%)^{†} | 364 (99.2) | 327 (98.2) |

PASI50 (Week 88)

| | | |
|---|---|---|
| Subjects with data | 360 | 333 |
| Responders (%)^{†} | 355 (98.6) | 326 (97.9) |

PASI50 (Week 100)

| | | |
|---|---|---|
| Subjects with data | 352 | 329 |
| Responders (%)^{†} | 347 (98.6) | 318 (96.7) |

PASI50 (Week 112)

| | | |
|---|---|---|
| Subjects with data | 346 | 320 |
| Responders (%)^{†} | 340 (98.3) | 310 (96.9) |

PASI50 (Week 124)

| | | |
|---|---|---|
| Subjects with data | 340 | 309 |
| Responders (%)^{†} | 334 (98.2) | 296 (95.8) |

PASI50 (Week 136)

| | | |
|---|---|---|
| Subjects with data | 328 | 303 |
| Responders (%)^{†} | 321 (97.9) | 291 (96.0) |

PASI50 (Week 148)

| | | |
|---|---|---|
| Subjects with data | 320 | 296 |
| Responders (%)^{†} | 315 (98.4) | 290 (98.0) |

PASI50 (Week 172)

| | | |
|---|---|---|
| Subjects with data | 317 | 293 |
| Responders (%)^{†} | 314 (99.1) | 287 (98.0) |

PASI50 (Week 196)

| | | |
|---|---|---|
| Subjects with data | 305 | 284 |
| Responders (%)^{†} | 302 (99.0) | 277 (97.5) |

PASI50 (Week 220)

| | | |
|---|---|---|
| Subjects with data | 294 | 277 |
| Responders (%)^{†} | 292 (99.3) | 275 (99.3) |

PASI50 (Week 244)

| | | |
|---|---|---|
| Subjects with data | 285 | 270 |
| Responders (%)^{†} | 283 (99.3) | 267 (98.9) |

PASI50 (Week 268)

| | | |
|---|---|---|
| Subjects with data | 102 | 84 |
| Responders (%)^{†} | 101 (99.0) | 84 (100.0) |

PASI50 (Week 292)

| | | |
|---|---|---|
| Subjects with data | 81 | 69 |
| Responders (%)^{†} | 80 (98.8) | 66 (95.7) |

PASI50 (Week 316)

| | | |
|---|---|---|
| Subjects with data | 47 | 34 |
| Responders (%)^{†} | 47 (100.0) | 34 (100.0) |

PASI50 (Week 340)

| | | |
|---|---|---|
| Subjects with data | 48 | 27 |
| Responders (%)^{†} | 48 (100.0) | 26 (96.3) |

PASI50 (Week 364)

| | | |
|---|---|---|
| Subjects with data | 11 | 9 |
| Responders (%)^{†} | 11 (100.0) | 9 (100.0) |

PASI50 (Week 384)

| | | |
|---|---|---|
| Subjects with data | 12 | 4 |
| Responders (%)^{†} | 12 (100.0) | 4 (100.0) |

PASI50 (Week 396)

| | | |
|---|---|---|
| Subjects with data | 1 | 0 |
| Responders (%)^{†} | 1 (100.0) | 0 |

^{†}Percentages are based on subjects with data.
N = Number of randomized subjects who received at least one dose of study medication in extension study.
PASI = Psoriasis Area and Severity Index.

### Example 2. PASI75 Response Over Time Among PASI75 Responders at Week 52

The PASI75 response, among those who were PAS75 responders at Week 52, was maintained in the subjects who remained in the study at specified time points throughout the extension study and was enhanced in the tildrakizumab 100 mg group than in the tildrakizumab 200 mg group at almost all timepoints (≥91.3% and ≥87.1% of the subjects in the tildrakizumab 100 mg group and 200 mg group, respectively). See Table 18.

TABLE 18
Proportion of Subjects with PASI75 Response Over Time - PASI75
Responders at Week 52 (Full Analysis Set) Extension Study

| | | |
|---|---|---|
| | MK-3222 | MK-3222 |
| | 100 mg | 200 mg |
| Statistics | N = 381 | N = 349 |

PASI75 (Week 60)

| | | |
|---|---|---|
| Subjects with data | 344 | 303 |
| Responders (%)^{†} | 332 (96.5) | 284 (93.7) |

PASI75 (Week 64)

| | | |
|---|---|---|
| Subjects with data | 340 | 301 |
| Responders (%)^{†} | 325 (95.6) | 275 (91.4) |

PASI75 (Week 76)

| | | |
|---|---|---|
| Subjects with data | 340 | 296 |
| Responders (%)^{†} | 326 (95.9) | 270 (91.2) |

PASI75 (Week 88)

| | | |
|---|---|---|
| Subjects with data | 333 | 296 |
| Responders (%)^{†} | 308 (92.5) | 265 (89.5) |

PASI75 (Week 100)

| | | |
|---|---|---|
| Subjects with data | 327 | 293 |
| Responders (%)^{†} | 301 (92.0) | 257 (87.7) |

PASI75 (Week 112)

| | | |
|---|---|---|
| Subjects with data | 321 | 286 |
| Responders (%)^{†} | 295 (91.9) | 251 (87.8) |

PASI75 (Week 124)

| | | |
|---|---|---|
| Subjects with data | 316 | 279 |
| Responders (%)^{†} | 294 (93.0) | 251 (90.0) |

PASI75 (Week 136)

| | | |
|---|---|---|
| Subjects with data | 307 | 275 |
| Responders (%)^{†} | 291 (94.8) | 251 (91.3) |

PASI75 (Week 148)

| | | |
|---|---|---|
| Subjects with data | 301 | 269 |
| Responders (%)^{†} | 278 (92.4) | 249 (92.6) |

PASI75 (Week 172)

| | | |
|---|---|---|
| Subjects with data | 299 | 267 |
| Responders (%)^{†} | 280 (93.6) | 241 (90.3) |

PASI75 (Week 196)

| | | |
|---|---|---|
| Subjects with data | 289 | 260 |
| Responders (%)^{†} | 264 (91.3) | 236 (90.8) |

PASI75 (Week 220)

| | | |
|---|---|---|
| Subjects with data | 278 | 256 |
| Responders (%)^{†} | 259 (93.2) | 230 (89.8) |

PASI75 (Week 244)

| | | |
|---|---|---|
| Subjects with data | 269 | 251 |
| Responders (%)^{†} | 250 (92.9) | 234 (93.2) |

PASI75 (Week 268)

| | | |
|---|---|---|
| Subjects with data | 93 | 76 |
| Responders (%)^{†} | 88 (94.6) | 70 (92.1) |

PASI75 (Week 292)

| | | |
|---|---|---|
| Subjects with data | 74 | 62 |
| Responders (%)^{†} | 70 (94.6) | 54 (87.1) |

PASI75 (Week 316)

| | | |
|---|---|---|
| Subjects with data | 45 | 31 |
| Responders (%)^{†} | 43 (95.6) | 31 (100.0) |

PASI75 (Week 340)

| | | |
|---|---|---|
| Subjects with data | 47 | 25 |
| Responders (%)^{†} | 47 (100.0) | 24 (96.0) |

PASI75 (Week 364)

| | | |
|---|---|---|
| Subjects with data | 11 | 8 |
| Responders (%)^{†} | 11 (100.0) | 8 (100.0) |

PASI75 (Week 384)

| | | |
|---|---|---|
| Subjects with data | 12 | 4 |
| Responders (%)^{†} | 12 (100.0) | 4 (100.0) |

PASI75 (Week 396)

| | | |
|---|---|---|
| Subjects with data | 1 | 0 |
| Responders (%)^{†} | 1 (100.0) | 0 |

^{†}Percentages are based on subjects with data.
N = Number of randomized subjects who received at least one dose of study medication in extension study.
PASI = Psoriasis Area and Severity Index.

### Example 3. PAST90 Response Over Time Among PAST90 Responders at Week 52

The PASI90 response, among those who were PASI90 responders at Week 52, was maintained in subjects who remained in the study at the specified time points throughout the extension study and was enhanced at most of timepoints in the tildrakizumab 100 mg group than in the tildrakizumab 200 mg group (≥80.2% and ≥73.6% of the subjects in the tildrakizumab 100 mg group and the tildrakizumab 200 mg group, respectively). See Table 19.

TABLE 19
Proportion of Subjects with PASI90 Response Over Time - PASI90
Responders at Week 52 (Full Analysis Set) Extension Study

| | | |
|---|---|---|
| | MK-3222 | MK-3222 |
| | 100 mg | 200 mg |
| Statistics | N = 381 | N = 349 |

PASI90 (Week 60)

| | | |
|---|---|---|
| Subjects with data | 261 | 194 |
| Responders (%)^{†} | 243 (93.1) | 171 (88.1) |

PASI90 (Week 64)

| | | |
|---|---|---|
| Subjects with data | 258 | 192 |
| Responders (%)^{†} | 237 (91.9) | 165 (85.9) |

PASI90 (Week 76)

| | | |
|---|---|---|
| Subjects with data | 258 | 192 |
| Responders (%)^{†} | 230 (89.1) | 164 (85.4) |

PASI90 (Week 88)

| | | |
|---|---|---|
| Subjects with data | 251 | 192 |
| Responders (%)^{†} | 212 (84.5) | 168 (87.5) |

PASI90 (Week 100)

| | | |
|---|---|---|
| Subjects with data | 249 | 191 |
| Responders (%)^{†} | 208 (83.5) | 161 (84.3) |

PASI90 (Week 112)

| | | |
|---|---|---|
| Subjects with data | 245 | 188 |
| Responders (%)^{†} | 208 (84.9) | 157 (83.5) |

PASI90 (Week 124)

| | | |
|---|---|---|
| Subjects with data | 244 | 184 |
| Responders (%)^{†} | 202 (82.8) | 149 (81.0) |

PASI90 (Week 136)

| | | |
|---|---|---|
| Subjects with data | 239 | 184 |
| Responders (%)^{†} | 201 (84.1) | 149 (81.0) |

PASI90 (Week 148)

| | | |
|---|---|---|
| Subjects with data | 234 | 181 |
| Responders (%)^{†} | 189 (80.8) | 144 (79.6) |

PASI90 (Week 172)

| | | |
|---|---|---|
| Subjects with data | 234 | 178 |
| Responders (%)^{†} | 190 (81.2) | 145 (81.5) |

PASI90 (Week 196)

| | | |
|---|---|---|
| Subjects with data | 227 | 173 |
| Responders (%)^{†} | 182 (80.2) | 140 (80.9) |

PASI90 (Week 220)

| | | |
|---|---|---|
| Subjects with data | 218 | 171 |
| Responders (%)^{†} | 176 (80.7) | 131 (76.6) |

PASI90 (Week 244)

| | | |
|---|---|---|
| Subjects with data | 211 | 169 |
| Responders (%)^{†} | 171 (81.0) | 129 (76.3) |

PASI90 (Week 268)

| | | |
|---|---|---|
| Subjects with data | 75 | 53 |
| Responders (%)^{†} | 64 (85.3) | 39 (73.6) |

PASI90 (Week 292)

| | | |
|---|---|---|
| Subjects with data | 60 | 47 |
| Responders (%)^{†} | 50 (83.3) | 36 (76.6) |

PASI90 (Week 316)

| | | |
|---|---|---|
| Subjects with data | 38 | 20 |
| Responders (%)^{†} | 31 (81.6) | 16 (80.0) |

PASI90 (Week 340)

| | | |
|---|---|---|
| Subjects with data | 39 | 17 |
| Responders (%)^{†} | 32 (82.1) | 16 (94.1) |

PASI90 (Week 364)

| | | |
|---|---|---|
| Subjects with data | 10 | 6 |
| Responders (%)^{†} | 9 (90.0) | 6 (100.0) |

PASI90 (Week 384)

| | | |
|---|---|---|
| Subjects with data | 9 | 3 |
| Responders (%)^{†} | 9 (100.0) | 3 (100.0) |

PASI90 (Week 396)

| | | |
|---|---|---|
| Subjects with data | 1 | 0 |
| Responders (%)^{†} | 1 (100.0) | 0 |

^{†}Percentages are based on subjects with data.
N = Number of randomized subjects who received at least one dose of study medication in extension study.
PASI = Psoriasis Area and Severity Index.

### Example 4. PASI100 Response Over Time Among PASI100 Responders at Week 52

The proportion of subjects with a PASI 100 response over time among those who were PASI100 responders at Week 52. The proportion of subjects with PASI100 response decreased approximately 20 percentage points from Week 60 to Week 244 in both treatment groups (from 84.0% to 65.7% in the tildrakizumab 100 mg group and from 79.8% to 62.4% in the tildrakizumab 200 mg group) during the extension study. From Week 268 to Week 384 (during Extension 2 and 3), the proportion of subjects with PASI 100 response increased (from 68.400 to 10000 in the tildrakizumab 100 mg group and from 58.600 to 10000 in the tildrakizumab 200 mg group). See Table 20.

TABLE 20
Proportion of Subjects with PASI100 Response Over Time - PASI100
Responders at Week 52 (Full Analysis Set) Extension Study

| | | |
|---|---|---|
| | MK-3222 | MK-3222 |
| | 100 mg | 200 mg |
| Statistics | N = 381 | N = 349 |

PASI100 (Week 60)

| | | |
|---|---|---|
| Subjects with data | 131 | 99 |
| Responders (%)^{†} | 110 (84.0) | 79 (79.8) |

PASI100 (Week 64)

| | | |
|---|---|---|
| Subjects with data | 128 | 99 |
| Responders (%)^{†} | 104 (81.3) | 67 (67.7) |

PASI100 (Week 76)

| | | |
|---|---|---|
| Subjects with data | 128 | 97 |
| Responders (%)^{†} | 96 (75.0) | 68 (70.1) |

PASI100 (Week 88)

| | | |
|---|---|---|
| Subjects with data | 125 | 98 |
| Responders (%)^{†} | 91 (72.8) | 67 (68.4) |

PASI100 (Week 100)

| | | |
|---|---|---|
| Subjects with data | 125 | 97 |
| Responders (%)^{†} | 82 (65.6) | 68 (70.1) |

PASI100 (Week 112)

| | | |
|---|---|---|
| Subjects with data | 123 | 95 |
| Responders (%)^{†} | 86 (69.9) | 62 (65.3) |

PASI100 (Week 124)

| | | |
|---|---|---|
| Subjects with data | 123 | 94 |
| Responders (%)^{†} | 83 (67.5) | 57 (60.6) |

PASI100 (Week 136)

| | | |
|---|---|---|
| Subjects with data | 122 | 93 |
| Responders (%)^{†} | 85 (69.7) | 61 (65.6) |

PASI100 (Week 148)

| | | |
|---|---|---|
| Subjects with data | 119 | 91 |
| Responders (%)^{†} | 77 (64.7) | 55 (60.4) |

PASI100 (Week 172)

| | | |
|---|---|---|
| Subjects with data | 120 | 89 |
| Responders (%)^{†} | 76 (63.3) | 55 (61.8) |

PASI100 (Week 196)

| | | |
|---|---|---|
| Subjects with data | 117 | 85 |
| Responders (%)^{†} | 82 (70.1) | 49 (57.6) |

PASI100 (Week 220)

| | | |
|---|---|---|
| Subjects with data | 112 | 86 |
| Responders (%)^{†} | 75 (67.0) | 48 (55.8) |

PASI100 (Week 244)

| | | |
|---|---|---|
| Subjects with data | 108 | 85 |
| Responders (%)^{†} | 71 (65.7) | 53 (62.4) |

PASI100 (Week 268)

| | | |
|---|---|---|
| Subjects with data | 38 | 29 |
| Responders (%)^{†} | 26 (68.4) | 17 (58.6) |

PASI100 (Week 292)

| | | |
|---|---|---|
| Subjects with data | 31 | 24 |
| Responders (%)^{†} | 23 (74.2) | 15 (62.5) |

PASI100 (Week 316)

| | | |
|---|---|---|
| Subjects with data | 19 | 9 |
| Responders (%)^{†} | 14 (73.7) | 6 (66.7) |

PASI100 (Week 340)

| | | |
|---|---|---|
| Subjects with data | 20 | 10 |
| Responders (%)^{†} | 15 (75.0) | 7 (70.0) |

PASI100 (Week 364)

| | | |
|---|---|---|
| Subjects with data | 4 | 3 |
| Responders (%)^{†} | 3 (75.0) | 3 (100.0) |

PASI100 (Week 384)

| | | |
|---|---|---|
| Subjects with data | 3 | 2 |
| Responders (%)^{†} | 3 (100.0) | 2 (100.0) |

^{†}Percentages are based on subjects with data.
N = Number of randomized subjects who received at least one dose of study medication in extension study.
PASI = Psoriasis Area and Severity Index.

### Example 5. PASI 75, PASI 90, and PASI 100 Responses Over Time

The proportion of subjects with PASI 75, PASI 90, and PASI 100 responses over time is summarized for the treatment groups in Table 21. Within each PASI response (i.e., PASI 75, PASI 90, and PASI 100), the proportion of subjects with PASI response was maintained throughout the extension study among subjects who remained in the extension study at the specified time points. The proportion of subjects with PASI 75, PASI 90, and PASI 100 responses over time was not different between the subjects who were treated with PFS and those who were treated with AI. The proportion of subjects with PASI 75 responses was higher in the subjects who self-injected than in subjects who did not self-inject at most of timepoints in the tildrakizumab 100 mg group. The proportion of subjects with PASI 90 or PASI 100 responses was higher in the subjects who self-injected than in the subjects who did not self-inject in both treatment groups at most of timepoints during the extension study.

TABLE 21
Proportion of Subjects with a PASI75, PASI90, and PASI100
Response Over Time (Full Analysis Set) Extension Study

| | | PASI75 | | PASI90 | | PASI100 | |
|---|---|---|---|---|---|---|---|
| Treatment | N | n | (%) | n | (%) | n | (%) |

Week 52

| | | | | | | | |
|---|---|---|---|---|---|---|---|
| MK-3222 100 mg | 376 | 346 | (92.0) | 262 | (69.7) | 131 | (34.8) |
| MK-3222 200 mg | 344 | 305 | (88.7) | 195 | (56.7) | 99 | (28.8) |

Week 60

| | | | | | | | |
|---|---|---|---|---|---|---|---|
| MK-3222 100 mg | 378 | 345 | (91.3) | 265 | (70.1) | 145 | (38.4) |
| MK-3222 200 mg | 345 | 299 | (86.7) | 203 | (58.8) | 100 | (29.0) |

Week 64

| | | | | | | | |
|---|---|---|---|---|---|---|---|
| MK-3222 100 mg | 372 | 334 | (89.8) | 262 | (70.4) | 139 | (37.4) |
| MK-3222 200 mg | 342 | 294 | (86.0) | 204 | (59.6) | 98 | (28.7) |

Week 76

| | | | | | | | |
|---|---|---|---|---|---|---|---|
| MK-3222 100 mg | 371 | 340 | (91.6) | 261 | (70.4) | 128 | (34.5) |
| MK-3222 200 mg | 337 | 286 | (84.9) | 211 | (62.6) | 100 | (29.7) |

Week 88

| | | | | | | | |
|---|---|---|---|---|---|---|---|
| MK-3222 100 mg | 364 | 320 | (87.9) | 240 | (65.9) | 125 | (34.3) |
| MK-3222 200 mg | 336 | 285 | (84.8) | 207 | (61.6) | 106 | (31.5) |

Week 100

| | | | | | | | |
|---|---|---|---|---|---|---|---|
| MK-3222 100 mg | 355 | 314 | (88.5) | 229 | (64.5) | 112 | (31.5) |
| MK-3222 200 mg | 332 | 278 | (83.7) | 202 | (60.8) | 104 | (31.3) |

Week 112

| | | | | | | | |
|---|---|---|---|---|---|---|---|
| MK-3222 100 mg | 349 | 303 | (86.8) | 229 | (65.6) | 118 | (33.8) |
| MK-3222 200 mg | 323 | 269 | (83.3) | 195 | (60.4) | 102 | (31.6) |

Week 124

| | | | | | | | |
|---|---|---|---|---|---|---|---|
| MK-3222 100 mg | 343 | 304 | (88.6) | 238 | (69.4) | 118 | (34.4) |
| MK-3222 200 mg | 312 | 267 | (85.6) | 198 | (63.5) | 106 | (34.0) |

Week 136

| | | | | | | | |
|---|---|---|---|---|---|---|---|
| MK-3222 100 mg | 331 | 298 | (90.0) | 229 | (69.2) | 124 | (37.5) |
| MK-3222 200 mg | 306 | 268 | (87.6) | 186 | (60.8) | 103 | (33.7) |

Week 148

| | | | | | | | |
|---|---|---|---|---|---|---|---|
| MK-3222 100 mg | 322 | 286 | (88.8) | 207 | (64.3) | 113 | (35.1) |
| MK-3222 200 mg | 299 | 265 | (88.6) | 184 | (61.5) | 90 | (30.1) |

Week 172

| | | | | | | | |
|---|---|---|---|---|---|---|---|
| MK-3222 100 mg | 320 | 292 | (91.3) | 220 | (68.8) | 117 | (36.6) |
| MK-3222 200 mg | 295 | 254 | (86.1) | 186 | (63.1) | 102 | (34.6) |

Week 196

| | | | | | | | |
|---|---|---|---|---|---|---|---|
| MK-3222 100 mg | 307 | 273 | (88.9) | 210 | (68.4) | 119 | (38.8) |
| MK-3222 200 mg | 286 | 249 | (87.1) | 181 | (63.3) | 94 | (32.9) |

Week 220

| | | | | | | | |
|---|---|---|---|---|---|---|---|
| MK-3222 100 mg | 296 | 267 | (90.2) | 207 | (69.9) | 115 | (38.9) |
| MK-3222 200 mg | 279 | 242 | (86.7) | 172 | (61.6) | 91 | (32.6) |

Week 244

| | | | | | | | |
|---|---|---|---|---|---|---|---|
| MK-3222 100 mg | 286 | 262 | (91.6) | 204 | (71.3) | 111 | (38.8) |
| MK-3222 200 mg | 272 | 245 | (90.1) | 167 | (61.4) | 104 | (38.2) |

Week 268

| | | | | | | | |
|---|---|---|---|---|---|---|---|
| MK-3222 100 mg | 103 | 96 | (93.2) | 75 | (72.8) | 40 | (38.8) |
| MK-3222 200 mg | 85 | 78 | (91.8) | 55 | (64.7) | 32 | (37.6) |

Week 292

| | | | | | | | |
|---|---|---|---|---|---|---|---|
| MK-3222 100 mg | 82 | 77 | (93.9) | 59 | (72.0) | 32 | (39.0) |
| MK-3222 200 mg | 71 | 62 | (87.3) | 49 | (69.0) | 32 | (45.1) |

Week 316

| | | | | | | | |
|---|---|---|---|---|---|---|---|
| MK-3222 100 mg | 48 | 46 | (95.8) | 35 | (72.9) | 23 | (47.9) |
| MK-3222 200 mg | 35 | 35 | (100.0) | 26 | (74.3) | 17 | (48.6) |

Week 340

| | | | | | | | |
|---|---|---|---|---|---|---|---|
| MK-3222 100 mg | 49 | 49 | (100.0) | 36 | (73.5) | 27 | (55.1) |
| MK-3222 200 mg | 29 | 28 | (96.6) | 24 | (82.8) | 15 | (51.7) |

Week 364

| | | | | | | | |
|---|---|---|---|---|---|---|---|
| MK-3222 100 mg | 11 | 11 | (100.0) | 9 | (81.8) | 6 | (54.5) |
| MK-3222 200 mg | 9 | 9 | (100.0) | 8 | (88.9) | 4 | (44.4) |

Week 384

| | | | | | | | |
|---|---|---|---|---|---|---|---|
| MK-3222 100 mg | 12 | 12 | (100.0) | 9 | (75.0) | 5 | (41.7) |
| MK-3222 200 mg | 4 | 4 | (100.0) | 3 | (75.0) | 3 | (75.0) |

Week 396

| | | | | | | | |
|---|---|---|---|---|---|---|---|
| MK-3222 100 mg | 1 | 1 | (100.0) | 1 | (100.0) | 1 | (100.0) |
| MK-3222 200 mg | 0 | 0 | | 0 | | 0 | |

N = Number of randomized subjects who received at least one dose of study medication in study part and with valid value at baseline and at the time point for endpoint.
n = the number of responders at the visit.
No imputation of missing data.
PASI = Psoriasis Area and Severity Index.
Week 52: Last scheduled assessment recorded prior to the last dose in the base study.

### Example 6. Change and Percent Change in PAST Score Over Time

Summaries of change (Table 22) and percent change (Table 23) from baseline in PASI scores over time (FAS). Mean changes in PASI scores were maintained in both treatment groups during the extension study. Similarly, mean percent changes in PASI scores were maintained over time in the two treatment groups. No meaningful differences in changes from baseline in PASI scores over time were observed between subjects who were treated with PFS and those who were treated with AI.

TABLE 22
Summary of Change From Baseline in Psoriasis Area and
Severity Index Score Over Time (Full Analysis Set)

| | | | Time | | | | | |
|---|---|---|---|---|---|---|---|---|
| | | Baseline | Point | Change from Baseline | | | | |
| Treatment | N | Mean (SD) | Mean (SD) | Mean (SD) | Q1 | Median | Q3 | 95% CI |

Week 52

| | | | | | | | | |
|---|---|---|---|---|---|---|---|---|
| MK-3222 100 mg | 376 | 19.8 (7.64) | 1.6 (2.30) | −18.2 (7.23) | −21.0 | −16.2 | −13.6 | (−18.9, −17.5) |
| MK-3222 200 mg | 344 | 19.3 (6.88) | 2.0 (2.14) | −17.3 (6.81) | −20.6 | −16.0 | −12.3 | (−18.1, −16.6) |

Week 60

| | | | | | | | | |
|---|---|---|---|---|---|---|---|---|
| MK-3222 100 mg | 378 | 19.8 (7.66) | 1.6 (2.29) | −18.2 (7.53) | −21.0 | −16.3 | −13.7 | (−19.0, −17.4) |
| MK-3222 200 mg | 345 | 19.4 (6.90) | 2.1 (2.58) | −17.3 (6.86) | −20.2 | −15.9 | −12.7 | (−18.0, −16.6) |

Week 64

| | | | | | | | | |
|---|---|---|---|---|---|---|---|---|
| MK-3222 100 mg | 372 | 19.8 (7.66) | 1.6 (2.46) | −18.1 (7.33) | −20.7 | −16.3 | −13.6 | (−18.9, −17.4) |
| MK-3222 200 mg | 342 | 19.4 (6.89) | 2.1 (2.70) | −17.3 (6.98) | −20.2 | −15.9 | −12.7 | (−18.0, −16.5) |

Week 76

| | | | | | | | | |
|---|---|---|---|---|---|---|---|---|
| MK-3222 100 mg | 371 | 19.8 (7.66) | 1.6 (2.30) | −18.2 (7.50) | −21.0 | −16.2 | −13.6 | (−19.0, −17.5) |
| MK-3222 200 mg | 337 | 19.3 (6.95) | 2.0 (2.52) | −17.3 (6.99) | −20.5 | −15.7 | −12.8 | (−18.1, −16.6) |

Week 88

| | | | | | | | | |
|---|---|---|---|---|---|---|---|---|
| MK-3222 100 mg | 364 | 19.8 (7.71) | 1.9 (2.71) | −17.9 (7.31) | −20.5 | −16.3 | −13.6 | (−18.7, −17.2) |
| MK-3222 200 mg | 336 | 19.4 (6.96) | 2.1 (2.73) | −17.2 (6.91) | −19.9 | −15.8 | −12.8 | (−18.0, −16.5) |

Week 100

| | | | | | | | | |
|---|---|---|---|---|---|---|---|---|
| MK-3222 100 mg | 355 | 19.8 (7.63) | 2.0 (2.65) | −17.8 (7.22) | −20.2 | −16.3 | −13.4 | (−18.6, −17.1) |
| MK-3222 200 mg | 332 | 19.3 (6.97) | 2.2 (3.06) | −17.1 (7.00) | −19.9 | −15.6 | −12.7 | (−17.9, −16.4) |

Week 112

| | | | | | | | | |
|---|---|---|---|---|---|---|---|---|
| MK-3222 100 mg | 349 | 19.8 (7.67) | 2.0 (2.66) | −17.8 (7.38) | −20.4 | −16.3 | −13.1 | (−18.6, −17.0) |
| MK-3222 200 mg | 323 | 19.3 (6.87) | 2.3 (2.95) | −17.1 (6.79) | −19.8 | −15.5 | −12.8 | (−17.8, −16.3) |

Week 124

| | | | | | | | | |
|---|---|---|---|---|---|---|---|---|
| MK-3222 100 mg | 343 | 19.7 (7.45) | 1.8 (2.65) | −17.9 (7.40) | −20.7 | −16.2 | −13.4 | (−18.7, −17.1) |
| MK-3222 200 mg | 312 | 19.2 (6.89) | 2.1 (2.91) | −17.0 (7.01) | −19.9 | −15.5 | −12.8 | (−17.8, −16.2) |

Week 136

| | | | | | | | | |
|---|---|---|---|---|---|---|---|---|
| MK-3222 100 mg | 331 | 19.7 (7.57) | 1.8 (2.86) | −17.9 (7.15) | −20.8 | −16.2 | −13.3 | (−18.7, −17.1) |
| MK-3222 200 mg | 306 | 19.2 (7.00) | 1.9 (2.56) | −17.3 (7.09) | −20.0 | −15.6 | −12.8 | (−18.1, −16.5) |

Week 148

| | | | | | | | | |
|---|---|---|---|---|---|---|---|---|
| MK-3222 100 mg | 322 | 19.7 (7.52) | 1.9 (2.81) | −17.8 (7.23) | −20.7 | −16.2 | −13.2 | (−18.6, −17.0) |
| MK-3222 200 mg | 299 | 19.3 (7.04) | 1.9 (2.57) | −17.4 (7.13) | −20.6 | −15.5 | −12.8 | (−18.2, −16.6) |

Week 172

| | | | | | | | | |
|---|---|---|---|---|---|---|---|---|
| MK-3222 100 mg | 320 | 19.7 (7.54) | 1.7 (2.46) | −18.0 (7.16) | −20.5 | −16.3 | −13.8 | (−18.8, −17.2) |
| MK-3222 200 mg | 295 | 19.3 (7.02) | 2.1 (3.24) | −17.2 (6.97) | −20.4 | −15.2 | −12.8 | (−18.0, −16.4) |

Week 196

| | | | | | | | | |
|---|---|---|---|---|---|---|---|---|
| MK-3222 100 mg | 307 | 19.6 (7.46) | 1.8 (2.70) | −17.9 (7.29) | −20.6 | −16.2 | −13.3 | (−18.7, −17.0) |
| MK-3222 200 mg | 286 | 19.2 (7.01) | 1.9 (2.79) | −17.3 (7.13) | −20.4 | −15.6 | −13.0 | (−18.2, −16.5) |

Week 220

| | | | | | | | | |
|---|---|---|---|---|---|---|---|---|
| MK-3222 100 mg | 296 | 19.6 (7.36) | 1.6 (2.27) | −18.0 (7.06) | −20.8 | −16.1 | −13.7 | (−18.8, −17.2) |
| MK-3222 200 mg | 279 | 19.0 (6.93) | 2.0 (2.63) | −17.1 (6.73) | −20.0 | −15.2 | −12.9 | (−17.9, −16.3) |

Week 244

| | | | | | | | | |
|---|---|---|---|---|---|---|---|---|
| MK-3222 100 mg | 286 | 19.6 (7.35) | 1.6 (2.32) | −18.0 (7.10) | −20.7 | −16.3 | −13.7 | (−18.8, −17.2) |
| MK-3222 200 mg | 272 | 19.1 (6.98) | 1.8 (2.37) | −17.3 (6.91) | −20.4 | −15.5 | −13.0 | (−18.2, −16.5) |

Week 268

| | | | | | | | | |
|---|---|---|---|---|---|---|---|---|
| MK-3222 100 mg | 103 | 20.1 (7.68) | 1.6 (2.45) | −18.5 (6.87) | −21.2 | −16.6 | −13.6 | (−19.8, −17.1) |
| MK-3222 200 mg | 85 | 19.7 (6.80) | 1.6 (2.13) | −18.0 (6.80) | −20.4 | −15.5 | −13.3 | (−19.5, −16.6) |

Week 292

| | | | | | | | | |
|---|---|---|---|---|---|---|---|---|
| MK-3222 100 mg | 82 | 19.8 (7.32) | 1.6 (2.08) | −18.2 (6.74) | −21.1 | −16.6 | −13.6 | (−19.7, −16.7) |
| MK-3222 200 mg | 71 | 18.8 (6.00) | 1.9 (2.94) | −16.9 (6.14) | −18.9 | −15.5 | −13.2 | (−18.4, −15.5) |

Week 316

| | | | | | | | | |
|---|---|---|---|---|---|---|---|---|
| MK-3222 100 mg | 48 | 19.4 (7.32) | 1.5 (2.34) | −18.0 (6.33) | −21.0 | −16.3 | −14.5 | (−19.8, −16.1) |
| MK-3222 200 mg | 35 | 17.8 (4.55) | 1.0 (1.40) | −16.8 (4.30) | −18.3 | −15.7 | −13.8 | (−18.3, −15.3) |

Week 340

| | | | | | | | | |
|---|---|---|---|---|---|---|---|---|
| MK-3222 100 mg | 49 | 20.3 (8.07) | 1.1 (1.83) | −19.2 (6.93) | −21.6 | −17.4 | −14.9 | (−21.2, −17.2) |
| MK-3222 200 mg | 29 | 20.3 (7.88) | 1.5 (4.38) | −18.8 (8.34) | −25.1 | −16.2 | −14.0 | (−22.0, −15.6) |

Week 364

| | | | | | | | | |
|---|---|---|---|---|---|---|---|---|
| MK-3222 100 mg | 11 | 17.5 (4.49) | 0.8 (1.36) | −16.6 (3.80) | −21.6 | −15.2 | −14.1 | (−19.2, −14.1) |
| MK-3222 200 mg | 9 | 20.2 (5.41) | 0.7 (1.59) | −19.5 (5.50) | −25.9 | −17.5 | −16.5 | (−23.7, −15.3) |

Week 384

| | | | | | | | | |
|---|---|---|---|---|---|---|---|---|
| MK-3222 100 mg | 12 | 21.6 (11.66) | 1.6 (2.25) | −20.0 (9.78) | −20.7 | −17.0 | −14.5 | (−26.2, −13.7) |
| MK-3222 200 mg | 4 | 18.4 (5.62) | 1.1 (2.20) | −17.3 (5.93) | −21.1 | −15.3 | −13.5 | (−26.7, −7.8) |

Week 396

| | | | | | | | | |
|---|---|---|---|---|---|---|---|---|
| MK-3222 100 mg | 1 | 14.4 (N/A) | 0.0 (N/A) | −14.4 (N/A) | −14.4 | −14.4 | −14.4 | N/A |
| MK-3222 200 mg | 0 | | | | | | | |

CI = Confidence Interval; Q1 = 25th percentile; Q3 = 75th percentile; SD = Standard Deviation.
N = Number of randomized subjects who received at least one dose of study medication in study part and with valid value at baseline and at the time point for endpoint.
Baseline was defined as the last measurement taken prior to the first study medication in the base study.
Week 52: Last scheduled assessment recorded prior to the last dose in the base study.

TABLE 23
Summary of Percent Change From Baseline in Psoriasis Area and Severity Index Score Over Time (Full Analysis Set)

| | | | Time | | | | | |
|---|---|---|---|---|---|---|---|---|
| | | Baseline | Point | Percent Change from Baseline | | | | |
| Treatment | N | Mean (SD) | Mean (SD) | Mean (SD) | Q1 | Median | Q3 | 95% CI |

Week 52

| | | | | | | | | | |
|---|---|---|---|---|---|---|---|---|---|
| MK-3222 100 mg | 376 | 19.8 (7.64) | 1.6 (2.30) | −91.9 | (10.68) | −100.0 | −96.1 | −87.6 | (−93.0, −90.9) |
| MK-3222 200 mg | 344 | 19.3 (6.88) | 2.0 (2.14) | −89.3 | (11.14) | −100.0 | −92.5 | −81.9 | (−90.5, −88.1) |

Week 60

| | | | | | | | | | |
|---|---|---|---|---|---|---|---|---|---|
| MK-3222 100 mg | 378 | 19.8 (7.66) | 1.6 (2.29) | −91.4 | (12.97) | −100.0 | −96.8 | −87.0 | (−92.7, −90.1) |
| MK-3222 200 mg | 345 | 19.4 (6.90) | 2.1 (2.58) | −89.0 | (13.11) | −100.0 | −93.1 | −84.2 | (−90.4, −87.7) |

Week 64

| | | | | | | | | | |
|---|---|---|---|---|---|---|---|---|---|
| MK-3222 100 mg | 372 | 19.8 (7.66) | 1.6 (2.46) | −91.7 | (11.87) | −100.0 | −96.9 | −87.9 | (−92.9, −90.5) |
| MK-3222 200 mg | 342 | 19.4 (6.89) | 2.1 (2.70) | −88.9 | (13.83) | −100.0 | −93.8 | −83.3 | (−90.4, −87.5) |

Week 76

| | | | | | | | | | |
|---|---|---|---|---|---|---|---|---|---|
| MK-3222 100 mg | 371 | 19.8 (7.66) | 1.6 (2.30) | −91.7 | (11.92) | −100.0 | −96.2 | −87.9 | (−92.9, −90.5) |
| MK-3222 200 mg | 337 | 19.3 (6.95) | 2.0 (2.52) | −89.3 | (13.42) | −100.0 | −94.3 | −83.6 | (−90.7, −87.8) |

Week 88

| | | | | | | | | | |
|---|---|---|---|---|---|---|---|---|---|
| MK-3222 100 mg | 364 | 19.8 (7.71) | 1.9 (2.71) | −90.3 | (13.32) | −100.0 | −95.2 | −86.1 | (−91.6, −88.9) |
| MK-3222 200 mg | 336 | 19.4 (6.96) | 2.1 (2.73) | −88.9 | (13.58) | −100.0 | −93.3 | −82.6 | (−90.3, −87.4) |

Week 100

| | | | | | | | | | |
|---|---|---|---|---|---|---|---|---|---|
| MK-3222 100 mg | 355 | 19.8 (7.63) | 2.0 (2.65) | −89.8 | (13.38) | −100.0 | −94.3 | −85.3 | (−91.2, −88.4) |
| MK-3222 200 mg | 332 | 19.3 (6.97) | 2.2 (3.06) | −88.5 | (14.69) | −100.0 | −94.0 | −82.7 | (−90.1, −86.9) |

Week 112

| | | | | | | | | | |
|---|---|---|---|---|---|---|---|---|---|
| MK-3222 100 mg | 349 | 19.8 (7.67) | 2.0 (2.66) | −89.5 | (14.67) | −100.0 | −95.0 | −84.6 | (−91.1, −88.0) |
| MK-3222 200 mg | 323 | 19.3 (6.87) | 2.3 (2.95) | −88.3 | (14.92) | −100.0 | −94.4 | −82.7 | (−89.9, −86.7) |

Week 124

| | | | | | | | | | |
|---|---|---|---|---|---|---|---|---|---|
| MK-3222 100 mg | 343 | 19.7 (7.45) | 1.8 (2.65) | −90.1 | (17.90) | −100.0 | −95.1 | −87.2 | (−92.0, −88.2) |
| MK-3222 200 mg | 312 | 19.2 (6.89) | 2.1 (2.91) | −88.5 | (15.95) | −100.0 | −94.6 | −82.0 | (−90.3, −86.7) |

Week 136

| | | | | | | | | | |
|---|---|---|---|---|---|---|---|---|---|
| MK-3222 100 mg | 331 | 19.7 (7.57) | 1.8 (2.86) | −90.4 | (16.07) | −100.0 | −95.5 | −86.9 | (−92.2, −88.7) |
| MK-3222 200 mg | 306 | 19.2 (7.00) | 1.9 (2.56) | −89.4 | (14.01) | −100.0 | −94.5 | −84.7 | (−91.0, −87.8) |

Week 148

| | | | | | | | | | |
|---|---|---|---|---|---|---|---|---|---|
| MK-3222 100 mg | 322 | 19.7 (7.52) | 1.9 (2.81) | −90.0 | (14.94) | −100.0 | −95.2 | −84.3 | (−91.6, −88.4) |
| MK-3222 200 mg | 299 | 19.3 (7.04) | 1.9 (2.57) | −89.8 | (13.25) | −100.0 | −93.6 | −85.0 | (−91.3, −88.3) |

Week 172

| | | | | | | | | | |
|---|---|---|---|---|---|---|---|---|---|
| MK-3222 100 mg | 320 | 19.7 (7.54) | 1.7 (2.46) | −90.9 | (15.05) | −100.0 | −95.5 | −86.7 | (−92.5, −89.2) |
| MK-3222 200 mg | 295 | 19.3 (7.02) | 2.1 (3.24) | −89.2 | (14.61) | −100.0 | −93.8 | −84.1 | (−90.9, −87.5) |

Week 196

| | | | | | | | | | |
|---|---|---|---|---|---|---|---|---|---|
| MK-3222 100 mg | 307 | 19.6 (7.46) | 1.8 (2.70) | −90.6 | (14.35) | −100.0 | −95.6 | −86.4 | (−92.2, −89.0) |
| MK-3222 200 mg | 286 | 19.2 (7.01) | 1.9 (2.79) | −89.9 | (13.97) | −100.0 | −95.2 | −85.5 | (−91.5, −88.3) |

Week 220

| | | | | | | | | | |
|---|---|---|---|---|---|---|---|---|---|
| MK-3222 100 mg | 296 | 19.6 (7.36) | 1.6 (2.27) | −91.8 | (11.49) | −100.0 | −96.5 | −87.6 | (−93.1, −90.5) |
| MK-3222 200 mg | 279 | 19.0 (6.93) | 2.0 (2.63) | −89.7 | (12.23) | −100.0 | −94.3 | −83.7 | (−91.2, −88.3) |

Week 244

| | | | | | | | | | |
|---|---|---|---|---|---|---|---|---|---|
| MK-3222 100 mg | 286 | 19.6 (7.35) | 1.6 (2.32) | −91.8 | (12.78) | −100.0 | −95.6 | −89.2 | (−93.3, −90.3) |
| MK-3222 200 mg | 272 | 19.1 (6.98) | 1.8 (2.37) | −90.7 | (11.94) | −100.0 | −95.5 | −85.6 | (−92.1, −89.2) |

Week 268

| | | | | | | | | | |
|---|---|---|---|---|---|---|---|---|---|
| MK-3222 100 mg | 103 | 20.1 (7.68) | 1.6 (2.45) | −92.3 | (10.83) | −100.0 | −96.2 | −88.3 | (−94.4, −90.2) |
| MK-3222 200 mg | 85 | 19.7 (6.80) | 1.6 (2.13) | −91.3 | (10.72) | −100.0 | −95.2 | −86.4 | (−93.6, −89.0) |

Week 292

| | | | | | | | | | |
|---|---|---|---|---|---|---|---|---|---|
| MK-3222 100 mg | 82 | 19.8 (7.32) | 1.6 (2.08) | −92.2 | (11.00) | −100.0 | −95.9 | −88.4 | (−94.6, −89.8) |
| MK-3222 200 mg | 71 | 18.8 (6.00) | 1.9 (2.94) | −90.2 | (15.12) | −100.0 | −97.5 | −85.7 | (−93.8, −86.6) |

Week 316

| | | | | | | | | | |
|---|---|---|---|---|---|---|---|---|---|
| MK-3222 100 mg | 48 | 19.4 (7.32) | 1.5 (2.34) | −93.1 | (10.34) | −100.0 | −98.1 | −88.8 | (−96.1, −90.1) |
| MK-3222 200 mg | 35 | 17.8 (4.55) | 1.0 (1.40) | −94.8 | (6.76) | −100.0 | −99.3 | −89.3 | (−97.1, −92.5) |

Week 340

| | | | | | | | | | |
|---|---|---|---|---|---|---|---|---|---|
| MK-3222 100 mg | 49 | 20.3 (8.07) | 1.1 (1.83) | −95.4 | (6.51) | −100.0 | −100.0 | −89.6 | (−97.3, −93.5) |
| MK-3222 200 mg | 29 | 20.3 (7.88) | 1.5 (4.38) | −93.5 | (16.81) | −100.0 | −100.0 | −95.5 | (−99.9, −87.1) |

Week 364

| | | | | | | | | | |
|---|---|---|---|---|---|---|---|---|---|
| MK-3222 100 mg | 11 | 17.5 (4.49) | 0.8 (1.36) | −95.9 | (5.85) | −100.0 | −100.0 | −92.7 | (−99.8, −91.9) |
| MK-3222 200 mg | 9 | 20.2 (5.41) | 0.7 (1.59) | −96.7 | (7.32) | −100.0 | −98.6 | −98.2 | (−102.3, −91.1) |

Week 384

| | | | | | | | | | |
|---|---|---|---|---|---|---|---|---|---|
| MK-3222 100 mg | 12 | 21.6 (11.66) | 1.6 (2.25) | −93.9 | (6.93) | −100.0 | −95.0 | −89.1 | (−98.3, −89.5) |
| MK-3222 200 mg | 4 | 18.4 (5.62) | 1.1 (2.20) | −94.1 | (11.70) | −100.0 | −100.0 | −88.3 | (−112.8, −75.5) |

Week 396

| | | | | | | | | | |
|---|---|---|---|---|---|---|---|---|---|
| MK-3222 100 mg | 1 | 14.4 (N/A) | 0.0 (N/A) | −100.0 | (N/A) | −100.0 | −100.0 | −100.0 | N/A |

| | |
|---|---|
| MK-3222 200 mg | 0 |

CI = Confidence Interval; Q1 = 25th percentile; Q3 = 75th percentile; SD = Standard Deviation.
N = Number of randomized subjects who received at least one dose of study medication in study part and with valid value at baseline and at the time point for endpoint.
Baseline was defined as the last measurement taken prior to the first study medication in the base study.
Week 52: Last scheduled assessment recorded prior to the last dose in the base study.

### Example 7. PGA Score of “Clear” or Minimal” with at Least a 2 Grade Reduction from Baseline Over Time

The proportions of subjects with a PGA score of “clear” or “minimal,” with at least a two-grade point reduction from baseline over time during the extension study is provided in Table 24. The proportion of subjects with a PGA score of “clear” or “minimal,” with at least a two-grade point reduction from baseline were similar at most timepoints in the two treatment groups and were maintained during the extension study. The proportions of subjects with PGA score of “clear” or “minimal” with at least a 2-grade point reductions from baseline was not different between subjects treated with PFS and those treated with AI during the extension study. The proportion of subjects with PGA score of “clear” or “minimal” with at least two-grade point reductions from baseline was higher in subjects who started self-injecting tildrakizumab than in subjects who did not self-inject in both treatment groups, particularly in the tildrakizumab 200 mg group, at most timepoints.

TABLE 24
Proportion of subjects with PGA Score of clear or minimal, with at least
two-grade reduction from baseline over time (Full Analysis Set)

| | | PGA Score of | | PGA Score of | | PGA Score of | |
|---|---|---|---|---|---|---|---|
| | | Clear or Minimal | | Minimal | | Clear | |
| Treatment | N | n | (%) | n | (%) | n | (%) |

Week 52

| | | | | | | | |
|---|---|---|---|---|---|---|---|
| MK-3222 100 mg | 375 | 281 | (74.9) | 127 | (33.9) | 154 | (41.1) |
| MK-3222 200 mg | 341 | 250 | (73.3) | 141 | (41.3) | 109 | (32.0) |

Week 60

| | | | | | | | |
|---|---|---|---|---|---|---|---|
| MK-3222 100 mg | 374 | 277 | (74.1) | 108 | (28.9) | 169 | (45.2) |
| MK-3222 200 mg | 342 | 242 | (70.8) | 124 | (36.3) | 118 | (34.5) |

Week 64

| | | | | | | | |
|---|---|---|---|---|---|---|---|
| MK-3222 100 mg | 370 | 276 | (74.6) | 119 | (32.2) | 157 | (42.4) |
| MK-3222 200 mg | 339 | 233 | (68.7) | 118 | (34.8) | 115 | (33.9) |

Week 76

| | | | | | | | |
|---|---|---|---|---|---|---|---|
| MK-3222 100 mg | 369 | 272 | (73.7) | 124 | (33.6) | 148 | (40.1) |
| MK-3222 200 mg | 334 | 221 | (66.2) | 103 | (30.8) | 118 | (35.3) |

Week 88

| | | | | | | | |
|---|---|---|---|---|---|---|---|
| MK-3222 100 mg | 362 | 234 | (64.6) | 99 | (27.3) | 135 | (37.3) |
| MK-3222 200 mg | 333 | 223 | (67.0) | 100 | (30.0) | 123 | (36.9) |

Week 100

| | | | | | | | |
|---|---|---|---|---|---|---|---|
| MK-3222 100 mg | 351 | 231 | (65.8) | 106 | (30.2) | 125 | (35.6) |
| MK-3222 200 mg | 329 | 219 | (66.6) | 103 | (31.3) | 116 | (35.3) |

Week 112

| | | | | | | | |
|---|---|---|---|---|---|---|---|
| MK-3222 100 mg | 346 | 221 | (63.9) | 91 | (26.3) | 130 | (37.6) |
| MK-3222 200 mg | 320 | 209 | (65.3) | 96 | (30.0) | 113 | (35.3) |

Week 124

| | | | | | | | |
|---|---|---|---|---|---|---|---|
| MK-3222 100 mg | 341 | 237 | (69.5) | 107 | (31.4) | 130 | (38.1) |
| MK-3222 200 mg | 309 | 200 | (64.7) | 84 | (27.2) | 116 | (37.5) |

Week 136

| | | | | | | | |
|---|---|---|---|---|---|---|---|
| MK-3222 100 mg | 329 | 229 | (69.6) | 99 | (30.1) | 130 | (39.5) |
| MK-3222 200 mg | 303 | 203 | (67.0) | 89 | (29.4) | 114 | (37.6) |

Week 148

| | | | | | | | |
|---|---|---|---|---|---|---|---|
| MK-3222 100 mg | 321 | 210 | (65.4) | 87 | (27.1) | 123 | (38.3) |
| MK-3222 200 mg | 296 | 196 | (66.2) | 94 | (31.8) | 102 | (34.5) |

Week 172

| | | | | | | | |
|---|---|---|---|---|---|---|---|
| MK-3222 100 mg | 316 | 222 | (70.3) | 91 | (28.8) | 131 | (41.5) |
| MK-3222 200 mg | 292 | 182 | (62.3) | 74 | (25.3) | 108 | (37.0) |

Week 196

| | | | | | | | |
|---|---|---|---|---|---|---|---|
| MK-3222 100 mg | 305 | 206 | (67.5) | 82 | (26.9) | 124 | (40.7) |
| MK-3222 200 mg | 283 | 183 | (64.7) | 82 | (29.0) | 101 | (35.7) |

Week 220

| | | | | | | | |
|---|---|---|---|---|---|---|---|
| MK-3222 100 mg | 292 | 214 | (73.3) | 94 | (32.2) | 120 | (41.1) |
| MK-3222 200 mg | 275 | 177 | (64.4) | 80 | (29.1) | 97 | (35.3) |

Week 244

| | | | | | | | |
|---|---|---|---|---|---|---|---|
| MK-3222 100 mg | 284 | 206 | (72.5) | 87 | (30.6) | 119 | (41.9) |
| MK-3222 200 mg | 269 | 183 | (68.0) | 75 | (27.9) | 108 | (40.1) |

Week 268

| | | | | | | | |
|---|---|---|---|---|---|---|---|
| MK-3222 100 mg | 101 | 76 | (75.2) | 35 | (34.7) | 41 | (40.6) |
| MK-3222 200 mg | 84 | 58 | (69.0) | 25 | (29.8) | 33 | (39.3) |

Week 292

| | | | | | | | |
|---|---|---|---|---|---|---|---|
| MK-3222 100 mg | 80 | 57 | (71.3) | 23 | (28.8) | 34 | (42.5) |
| MK-3222 200 mg | 70 | 49 | (70.0) | 15 | (21.4) | 34 | (48.6) |

Week 316

| | | | | | | | |
|---|---|---|---|---|---|---|---|
| MK-3222 100 mg | 46 | 31 | (67.4) | 8 | (17.4) | 23 | (50.0) |
| MK-3222 200 mg | 34 | 27 | (79.4) | 9 | (26.5) | 18 | (52.9) |

Week 340

| | | | | | | | |
|---|---|---|---|---|---|---|---|
| MK-3222 100 mg | 47 | 36 | (76.6) | 8 | (17.0) | 28 | (59.6) |
| MK-3222 200 mg | 29 | 25 | (86.2) | 8 | (27.6) | 17 | (58.6) |

Week 364

| | | | | | | | |
|---|---|---|---|---|---|---|---|
| MK-3222 100 mg | 11 | 8 | (72.7) | 2 | (18.2) | 6 | (54.5) |
| MK-3222 200 mg | 9 | 8 | (88.9) | 4 | (44.4) | 4 | (44.4) |

Week 384

| | | | | | | | |
|---|---|---|---|---|---|---|---|
| MK-3222 100 mg | 12 | 7 | (58.3) | 2 | (16.7) | 5 | (41.7) |
| MK-3222 200 mg | 4 | 3 | (75.0) | 0 | | 3 | (75.0) |

Week 396

| | | | | | | |
|---|---|---|---|---|---|---|
| MK-3222 100 mg | 1 | 1 | (100.0) | 0 | 1 | (100.0) |
| MK-3222 200 mg | 0 | 0 | | 0 | 0 | |

N = Number of randomized subjects who received at least one dose of study medication in study part and with valid value at baseline and at the time point for endpoint.
n = the number of responders at the visit.
No imputation of missing data.
PGA = Physician's Global Assessment.
Week 52: Last scheduled assessment recorded prior to the last dose in the base study.

### Example 8. Proportion of Subjects Who Self-Injected with Pre-Filled Syringe

The proportion of subjects who self-injected with the PFS for the full analysis set is shown in Table 25. The proportion of subjects who self-injected with the PFS ranged between 65.9% and 76.9% until Week 124 and was similar across the two treatment groups.

TABLE 25
Proportion of subjects who self-inject with
pre-filled syringe (Full Analysis Set)

Subject Who Self-Inject

| | | | |
|---|---|---|---|
| Treatment | N | n | (%) |

Week 60

| | | | |
|---|---|---|---|
| MK-3222 100 mg | 380 | 259 | 68.2 |
| MK-3222 200 mg | 349 | 230 | 65.9 |

Week 64

| | | | |
|---|---|---|---|
| MK-3222 100 mg | 375 | 260 | 69.3 |
| MK-3222 200 mg | 346 | 229 | 66.2 |

Week 76

| | | | |
|---|---|---|---|
| MK-3222 100 mg | 370 | 261 | 70.5 |
| MK-3222 200 mg | 341 | 230 | 67.4 |

Week 88

| | | | |
|---|---|---|---|
| MK-3222 100 mg | 359 | 260 | 72.4 |
| MK-3222 200 mg | 339 | 230 | 67.8 |

Week 100

| | | | |
|---|---|---|---|
| MK-3222 100 mg | 355 | 259 | 73.0 |
| MK-3222 200 mg | 329 | 229 | 69.6 |

Week 112

| | | | |
|---|---|---|---|
| MK-3222 100 mg | 347 | 259 | 74.6 |
| MK-3222 200 mg | 320 | 229 | 71.6 |

Week 124

| | | | |
|---|---|---|---|
| MK-3222 100 mg | 337 | 259 | 76.9 |
| MK-3222 200 mg | 312 | 227 | 72.8 |

Week 136

| | | | |
|---|---|---|---|
| MK-3222 100 mg | 330 | 159 | 48.2 |
| MK-3222 200 mg | 306 | 139 | 45.4 |

Week 148

| | | | |
|---|---|---|---|
| MK-3222 100 mg | 322 | 77 | 23.9 |
| MK-3222 200 mg | 300 | 83 | 27.7 |

Week 160

| | | | |
|---|---|---|---|
| MK-3222 100 mg | 321 | 32 | 10.0 |
| MK-3222 200 mg | 293 | 24 | 8.2 |

Week 172

| | | | |
|---|---|---|---|
| MK-3222 100 mg | 316 | 19 | 6.0 |
| MK-3222 200 mg | 294 | 8 | 2.7 |

Week 184

| | | | |
|---|---|---|---|
| MK-3222 100 mg | 308 | 12 | 3.9 |
| MK-3222 200 mg | 289 | 2 | 0.7 |

Week 196

| | | | |
|---|---|---|---|
| MK-3222 100 mg | 303 | 2 | 0.7 |
| MK-3222 200 mg | 282 | 0 | |

Week 208

| | | | |
|---|---|---|---|
| MK-3222 100 mg | 300 | 2 | 0.7 |
| MK-3222 200 mg | 279 | 0 | |

N = Number of randomized subjects who received at least one dose of study medication in study part at the visit.
n = Number of subjects who self-Injected with Pre-Filled Syringe at the visit.

### Example 9. Proportion of Subjects Who Self-Injected with Auto Injector (AI)

The proportion of subjects who self-injected with the AI is provided for full analysis set by time point and treatment group is shown in Table 26. The proportion of subjects who self-injected with the AI increased as they shifted from PFS to AI and was slightly higher in the tildrakizumab 100 mg group compared to the tildrakizumab 200 mg group.

TABLE 26
Proportion of subjects who self-injected with auto injector

Subject Who Self-Inject

| | | | |
|---|---|---|---|
| Treatment | N | n | (%) |

Week 124

| | | | |
|---|---|---|---|
| MK-3222 100 mg | 337 | 1 | 0.3 |
| MK-3222 200 mg | 312 | 2 | 0.6 |

Week 136

| | | | |
|---|---|---|---|
| MK-3222 100 mg | 330 | 98 | 29.7 |
| MK-3222 200 mg | 306 | 91 | 29.7 |

Week 148

| | | | |
|---|---|---|---|
| MK-3222 100 mg | 322 | 177 | 55.0 |
| MK-3222 200 mg | 300 | 143 | 47.7 |

Week 160

| | | | |
|---|---|---|---|
| MK-3222 100 mg | 321 | 221 | 68.8 |
| MK-3222 200 mg | 293 | 198 | 67.6 |

Week 172

| | | | |
|---|---|---|---|
| MK-3222 100 mg | 316 | 233 | 73.7 |
| MK-3222 200 mg | 294 | 215 | 73.1 |

Week 184

| | | | |
|---|---|---|---|
| MK-3222 100 mg | 308 | 233 | 75.6 |
| MK-3222 200 mg | 289 | 218 | 75.4 |

Week 196

| | | | |
|---|---|---|---|
| MK-3222 100 mg | 303 | 242 | 79.9 |
| MK-3222 200 mg | 282 | 215 | 76.2 |

Week 208

| | | | |
|---|---|---|---|
| MK-3222 100 mg | 300 | 237 | 79.0 |
| MK-3222 200 mg | 279 | 211 | 75.6 |

Week 220

| | | | |
|---|---|---|---|
| MK-3222 100 mg | 295 | 236 | 80.0 |
| MK-3222 200 mg | 277 | 209 | 75.5 |

Week 232

| | | | |
|---|---|---|---|
| MK-3222 100 mg | 292 | 235 | 80.5 |
| MK-3222 200 mg | 276 | 208 | 75.4 |

Week 244

| | | | |
|---|---|---|---|
| MK-3222 100 mg | 290 | 233 | 80.3 |
| MK-3222 200 mg | 269 | 202 | 75.1 |

Week 256

| | | | |
|---|---|---|---|
| MK-3222 100 mg | 132 | 102 | 77.3 |
| MK-3222 200 mg | 113 | 84 | 74.3 |

Week 268

| | | | |
|---|---|---|---|
| MK-3222 100 mg | 104 | 80 | 76.9 |
| MK-3222 200 mg | 90 | 63 | 70.0 |

Week 280

| | | | |
|---|---|---|---|
| MK-3222 100 mg | 100 | 77 | 77.0 |
| MK-3222 200 mg | 91 | 63 | 69.2 |

Week 292

| | | | |
|---|---|---|---|
| MK-3222 100 mg | 92 | 72 | 78.3 |
| MK-3222 200 mg | 73 | 48 | 65.8 |

Week 304

| | | | |
|---|---|---|---|
| MK-3222 100 mg | 72 | 53 | 73.6 |
| MK-3222 200 mg | 59 | 36 | 61.0 |

Week 316

| | | | |
|---|---|---|---|
| MK-3222 100 mg | 51 | 34 | 66.7 |
| MK-3222 200 mg | 38 | 21 | 55.3 |

Week 328

| | | | |
|---|---|---|---|
| MK-3222 100 mg | 51 | 38 | 74.5 |
| MK-3222 200 mg | 31 | 21 | 67.7 |

Week 340

| | | | |
|---|---|---|---|
| MK-3222 100 mg | 49 | 37 | 75.5 |
| MK-3222 200 mg | 26 | 18 | 69.2 |

Week 352

| | | | |
|---|---|---|---|
| MK-3222 100 mg | 23 | 19 | 82.6 |
| MK-3222 200 mg | 10 | 7 | 70.0 |

Week 364

| | | | |
|---|---|---|---|
| MK-3222 100 mg | 1 | 1 | 100.0 |
| MK-3222 200 mg | 3 | 0 | |

Week 384

| | | | |
|---|---|---|---|
| MK-3222 100 mg | 20 | 13 | 65.0 |
| MK-3222 200 mg | 11 | 5 | 45.5 |

Week 396

| | | | |
|---|---|---|---|
| MK-3222 100 mg | 20 | 13 | 65.0 |
| MK-3222 200 mg | 11 | 5 | 45.5 |

N = Number of randomized subjects who received at least one dose of study medication in study part at the visit.
n = Number of subjects who self-Injected with Auto-Injector at the visit.

### Example 10. Proportion of Subjects and Clinical Site Experiences and Device Failures with Use of the Auto Injector (AI)

The proportion of subjects with any adverse experience or with any device failure among subjects who were treated with AI is provided by time point and treatment group in Table 27. The proportion of subjects with any adverse experience or with any device failure was ≤0.5% and ≤1.6%, respectively.

TABLE 27
Proportion of subjects with adverse Experience and device failures
over time in subjects treated auto-injector (Full Analysis Set)

| | |
|---|---|
| Adverse | Device |
| Experience | Failure |

| | | | | | |
|---|---|---|---|---|---|
| Treatment | N | n | (%) | n | (%) |

Week 124

| | | | |
|---|---|---|---|
| MK-3222 100 mg | 4 | 0 | 0 |
| MK-3222 200 mg | 2 | 0 | 0 |

Week 136

| | | | |
|---|---|---|---|
| MK-3222 100 mg | 124 | 0 | 0 |
| MK-3222 200 mg | 121 | 0 | 0 |

Week 148

| | | | | | |
|---|---|---|---|---|---|
| MK-3222 100 mg | 214 | 1 | 0.5 | 0 | |
| MK-3222 200 mg | 192 | 0 | | 3 | 1.6 |

Week 160

| | | | | |
|---|---|---|---|---|
| MK-3222 100 mg | 278 | 0 | 1 | 0.4 |
| MK-3222 200 mg | 257 | 0 | 2 | 0.8 |

Week 172

| | | | | |
|---|---|---|---|---|
| MK-3222 100 mg | 290 | 0 | 0 | |
| MK-3222 200 mg | 278 | 0 | 2 | 0.7 |

Week 184

| | | | | | |
|---|---|---|---|---|---|
| MK-3222 100 mg | 292 | 0 | | 1 | 0.3 |
| MK-3222 200 mg | 281 | 1 | 0.4 | 3 | 1.1 |

Week 196

| | | | | | |
|---|---|---|---|---|---|
| MK-3222 100 mg | 297 | 1 | 0.3 | 2 | 0.7 |
| MK-3222 200 mg | 278 | 0 | | 1 | 0.4 |

Week 208

| | | | | |
|---|---|---|---|---|
| MK-3222 100 mg | 293 | 0 | 1 | 0.3 |
| MK-3222 200 mg | 275 | 0 | 0 | |

Week 220

| | | | | | |
|---|---|---|---|---|---|
| MK-3222 100 mg | 290 | 1 | 0.3 | 1 | 0.3 |
| MK-3222 200 mg | 273 | 0 | | 1 | 0.4 |

Week 232

| | | | | | |
|---|---|---|---|---|---|
| MK-3222 100 mg | 287 | 1 | 0.3 | 0 | |
| MK-3222 200 mg | 271 | 0 | | 1 | 0.4 |

Week 244

| | | | | |
|---|---|---|---|---|
| MK-3222 100 mg | 286 | 0 | 0 | |
| MK-3222 200 mg | 264 | 0 | 3 | 1.1 |

Week 256

| | | | |
|---|---|---|---|
| MK-3222 100 mg | 130 | 0 | 0 |
| MK-3222 200 mg | 112 | 0 | 0 |

Week 268

| | | | |
|---|---|---|---|
| MK-3222 100 mg | 103 | 0 | 0 |
| MK-3222 200 mg | 89 | 0 | 0 |

Week 280

| | | | |
|---|---|---|---|
| MK-3222 100 mg | 97 | 0 | 0 |
| MK-3222 200 mg | 90 | 0 | 0 |

Week 292

| | | | |
|---|---|---|---|
| MK-3222 100 mg | 91 | 0 | 0 |
| MK-3222 200 mg | 70 | 0 | 0 |

Week 304

| | | | |
|---|---|---|---|
| MK-3222 100 mg | 71 | 0 | 0 |
| MK-3222 200 mg | 58 | 0 | 0 |

Week 316

| | | | |
|---|---|---|---|
| MK-3222 100 mg | 50 | 0 | 0 |
| MK-3222 200 mg | 37 | 0 | 0 |

Week 328

| | | | |
|---|---|---|---|
| MK-3222 100 mg | 50 | 0 | 0 |
| MK-3222 200 mg | 30 | 0 | 0 |

Week 340

| | | | |
|---|---|---|---|
| MK-3222 100 mg | 48 | 0 | 0 |
| MK-3222 200 mg | 26 | 0 | 0 |

Week 352

| | | | |
|---|---|---|---|
| MK-3222 100 mg | 22 | 0 | 0 |
| MK-3222 200 mg | 10 | 0 | 0 |

Week 364

| | | | |
|---|---|---|---|
| MK-3222 100 mg | 1 | 0 | 0 |
| MK-3222 200 mg | 3 | 0 | 0 |

Week 384

| | | | |
|---|---|---|---|
| MK-3222 100 mg | 20 | 0 | 0 |
| MK-3222 200 mg | 11 | 0 | 0 |

Week 396

| | | | |
|---|---|---|---|
| MK-3222 100 mg | 20 | 0 | 0 |
| MK-3222 200 mg | 11 | 0 | 0 |

N = Number of subjects treated with auto-injector at the visit.
n = Number of subjects with adverse experience/device failure at the visit.

### Pharmacokinetics

Pharmacokinetic samples were analyzed over a period of 4 years from collection, using three different validated methods. The majority of samples (i.e., 11,658 out of 12,868) were analyzed within 1 year of collection. Approximately 914 samples among 12,868 were analyzed within 2 years from collection. PK Tables, Figures and Listings, PK concentration data were determined and overall the mean concentrations of tildrakizumab from Week 76 to Week 244 in extension 1 are comparable for each dose group. The mean tildrakizumab concentrations in 200 mg dose group are higher than at 100 mg dose group indicating a dose dependent increase in PK.

### Immunogenicity Analysis

ADA samples were analyzed for a period of 4.5 years from collection. Among 12,441 samples analyzed, 12,394 samples were analyzed within a period of 3.6 years which is the longest stability period reported in literature for ADA (Pihl et al., Bioanalysis 6(10): 1409-13 (2014)). The ADA evaluable population included subjects with at least one ADA sample after dosing with tildrakizumab.

### Example 11. Incidence of Anti-Drug Antibody

Anti-tildrakizumab immunogenicity status at the end of the base study and at the end of the extension study are provided by ADA evaluable subjects in the extension study in Table 28.

TABLE 28
Summary of Subject Anti-MK-3222 Immunogenicity
Status By Dose Level

| | MK-3222 Treatment in Extension | | |
|---|---|---|---|
| | MK-3222 | MK-3222 | |
| | 100 mg | 200 mg | Total |
| Evaluable subjects^{†} | 379 | 346 | 725 |
| Conclusive subjects^{‡} | 316 (83.4%) | 270 (78.0%) | 586 (80.8%) |
| Inconclusive^{‡} | 63 (16.6%) | 76 (22.0%) | 139 (19.2%) |

Immunogenicity status^{‡}

| | | | |
|---|---|---|---|
| Negative | 263 (69.4%) | 228 (65.9%) | 491 (67.7%) |
| Non-treatment emergent | 18 (4.7%) | 21 (6.1%) | 39 (5.4%) |
| positive | | | |
| Treatment emergent | 35 (9.2%) | 21 (6.1%) | 56 (7.7%) |
| positive | | | |

All ADA positive subjects^{‡}

| | | | |
|---|---|---|---|
| Total positive subjects | 53 (14.0%) | 42 (12.1%) | 95 (13.1%) |

Maximum Post-dose Titer^{§}

| | | | |
|---|---|---|---|
| 0.5 (<1) | 3 | 5 | 8 |
| 1 | 11 | 8 | 19 |
| 2 | 4 | 5 | 9 |
| 4 | 5 | 1 | 6 |
| 8 | 1 | 1 | 2 |
| 16 | 1 | 0 | 1 |
| 32 | 1 | 0 | 1 |
| 64 | 0 | 2 | 2 |
| 128 | 2 | 0 | 2 |
| 256 | 0 | 1 | 1 |
| 1024 | 1 | 0 | 1 |
| 2048 | 1 | 0 | 1 |

Neutralizing antibody results
Non-treatment emergent subjects

| | | | |
|---|---|---|---|
| Neutralizing negative^{‡, ∥} | 17 (4.5%) | 17 (4.9%) | 34 (4.7%) |
| Neutralizing positive^{‡, ∥} | 1 (0.3%) | 3 (0.9%) | 4 (0.6%) |

Treatment emergent subjects

| | | | |
|---|---|---|---|
| Neutralizing negative^{‡, ∥} | 13 (3.4%) | 13 (3.8%) | 26 (3.6%) |
| Neutralizing positive^{‡, ∥} | 10 (2.6%) | 3 (0.9%) | 13 (1.8%) |

^{†}Included were subjects with at least one ADA sample available after treatment with MK-3222.
^{‡}Denominator was total number of evaluable subjects.
^{§}Represents the number of subjects who showed a post-dose maximum titer in each titer category. Includes subjects who had titers <1, these subjects were counted in the 0.5 category.
^{∥}Only samples which were positive in the anti-MK-3222 antibody assay were tested in the neutralizing anti-MK-3222 assay. Subjects considered positive for neutralizing antibodies had at least one sample that tested positive in the neutralizing antibody assay.

Out of 725 ADA evaluable subjects, 586 (80.8%) subjects had conclusive results, 491 (67.7%) subjects were ADA negative, and 95 (13.1%) subjects were ADA positive, of which 39 (5.4%) subjects were non-treatment-emergent ADA positive, and 56 (7.7%) subjects were treatment-emergent ADA positive.

The proportion of ADA positive subjects was similar between the two treatment groups (53 [14.0%] subjects in the tildrakizumab 100 mg and 42 [12.1%] subjects in the tildrakizumab 200 mg). The proportion of treatment-emergent ADA positive subjects was higher in the tildrakizumab 100 mg group (35 [9.2%] subjects) compared with the tildrakizumab 200 mg group (21[6.1%] subjects). The maximum post-dose titer of the subjects ranged from <1 (represented as 0.5) to 2048. Across treatment groups, 13 (1.8%) treatment-emergent ADA positive subjects and 4 (0.6%) non-treatment-emergent ADA positive subjects had at least one sample test positive for neutralizing antibodies (NAbs). The proportion of subjects with treatment-emergent positive NAb positive and non-treatment-emergent positive NAb positive was low (<2%) in the two treatment groups.

### Example 12. Correlation of Anti-Drug Antibody with Efficacy

A summary of anti-tildrakizumab immunogenicity status and proportion of subjects with PASI 75 response in the extension study is provided by subject immunogenicity category over time in Table 29. The numbers of subjects with treatment-emergent positive NAb positive with PASI 75 response ≤10 subjects in the tildrakizumab 100 mg group and ≤3 subjects in the tildrakizumab 200 mg group at each timepoint during the extension study. Of these, the proportion of subjects who achieved PASI 75 response ranged from 75% to 86% in the tildrakizumab 100 mg group and from 33% to 100% in the tildrakizumab 200 mg group at each timepoint until Week 244.

TABLE 29
Subject immunogenicity status and proportion with PASI75 response
by subject immunogenicity category over time (Full Analysis Set)

| | MK-3222 100 mg PASI (N = 372) | | MK-3222 200 mg PASI (N = 339) | |
|---|---|---|---|---|
| | Subjects in | Subjects | Subjects in | Subjects |
| | ADA category | who achieved | ADA category | who achieved |
| | with evaluable | endpoint/Subjects | with evaluable | endpoint/Subjects |
| | samples^{†} and | in ADA category | samples^{†} and | in ADA category |
| | PASI scores | Ratio (%) | PASI scores | Ratio (%) |
| Week 76 | | | | |
| Total Evaluable for ADA^{†} | 366 | 336/366 (92%) | 331 | 281/331 (85%) |
| Treatment emergent positive NAb-POS^{‡} | 10 | 8/10 (80%) | 3 | 1/3 (33%) |
| Treatment emergent positive NAb-NEG^{‡} | 12 | 12/12 (100%) | 13 | 13/13 (100%) |
| Treatment emergent ADA positive^{‡} | 34 | 32/34 (94%) | 21 | 18/21 (86%) |
| Non-treatment emergent positive NAb-POS^{§} | 1 | 1/1 (100%) | 3 | 3/3 (100%) |
| Non-treatment emergent positive NAb-NEG^{§} | 16 | 14/16 (88%) | 15 | 13/15 (87%) |
| Non-treatment emergent ADA positive^{§} | 17 | 15/17 (88%) | 19 | 17/19 (89%) |
| Inconclusive^{∥} | 60 | 55/60 (92%) | 74 | 61/74 (82%) |
| ADA Negative^{#} | 255 | 234/255 (92%) | 217 | 185/217 (85%) |
| Week 100 | | | | |
| Total Evaluable for ADA^{†} | 352 | 311/352 (88%) | 328 | 274/328 (84%) |
| Treatment emergent positive NAb-POS^{‡} | 8 | 6/8 (75%) | 2 | 1/2 (50%) |
| Treatment emergent positive NAb-NEG^{‡} | 11 | 11/11 (100%) | 12 | 11/12 (92%) |
| Treatment emergent ADA positive^{‡} | 29 | 26/29 (90%) | 19 | 16/19 (84%) |
| Non-treatment emergent positive NAb-POS^{§} | 1 | 1/1 (100%) | 3 | 3/3 (100%) |
| Non-treatment emergent positive NAb-NEG^{§} | 15 | 13/15 (87%) | 15 | 13/15 (87%) |
| Non-treatment emergent ADA positive^{§} | 16 | 14/16 (88%) | 19 | 17/19 (89%) |
| Inconclusive^{∥} | 58 | 49/58 (84%) | 73 | 61/73 (84%) |
| ADA Negative^{#} | 249 | 222/249 (89%) | 217 | 180/217 (83%) |
| Week 124 | | | | |
| Total Evaluable for ADA^{†} | 340 | 302/340 (89%) | 309 | 265/309 (86%) |
| Treatment emergent positive NAb-POS^{‡} | 8 | 6/8 (75%) | 2 | 1/2 (50%) |
| Treatment emergent positive NAb-NEG^{‡} | 11 | 11/11 (100%) | 12 | 11/12 (92%) |
| Treatment emergent ADA positive^{‡} | 30 | 26/30 (87%) | 19 | 16/19 (84%) |
| Non-treatment emergent positive NAb-POS^{§} | 1 | 1/1 (100%) | 3 | 3/3 (100%) |
| Non-treatment emergent positive NAb-NEG^{§} | 14 | 13/14 (93%) | 15 | 12/15 (80%) |
| Non-treatment emergent ADA positive^{§} | 15 | 14/15 (93%) | 19 | 16/19 (84%) |
| Inconclusive^{∥} | 58 | 51/58 (88%) | 72 | 59/72 (82%) |
| ADA Negative^{#} | 237 | 211/237 (89%) | 199 | 174/199 (87%) |
| Week 148 | | | | |
| Total Evaluable for ADA^{†} | 320 | 284/320 (89%) | 296 | 262/296 (89%) |
| Treatment emergent positive NAb-POS^{‡} | 7 | 6/7 (86%) | 2 | 1/2 (50%) |
| Treatment emergent positive NAb-NEG^{‡} | 10 | 10/10 (100%) | 12 | 11/12 (92%) |
| Treatment emergent ADA positive^{‡} | 28 | 26/28 (93%) | 19 | 16/19 (84%) |
| Non-treatment emergent positive NAb-POS^{§} | 1 | 1/1 (100%) | 3 | 3/3 (100%) |
| Non-treatment emergent positive NAb-NEG^{§} | 13 | 13/13 (100%) | 14 | 11/14 (79%) |
| Non-treatment emergent ADA positive^{§} | 14 | 14/14 (100%) | 18 | 15/18 (83%) |
| Inconclusive^{∥} | 57 | 48/57 (84%) | 70 | 60/70 (86%) |
| ADA Negative^{#} | 221 | 196/221 (89%) | 189 | 171/189 (90%) |
| Week 172 | | | | |
| Total Evaluable for ADA^{†} | 317 | 290/317 (91%) | 293 | 252/293 (86%) |
| Treatment emergent positive NAb-POS^{‡} | 7 | 6/7 (86%) | 2 | 1/2 (50%) |
| Treatment emergent positive NAb-NEG^{‡} | 9 | 9/9 (100%) | 12 | 10/12 (83%) |
| Treatment emergent ADA positive^{‡} | 27 | 25/27 (93%) | 18 | 15/18 (83%) |
| Non-treatment emergent positive NAb-POS^{§} | 1 | 1/1 (100%) | 3 | 3/3 (100%) |
| Non-treatment emergent positive NAb-NEG^{§} | 13 | 13/13 (100%) | 14 | 11/14 (79%) |
| Non-treatment emergent ADA positive^{§} | 14 | 14/14 (100%) | 18 | 15/18 (83%) |
| Inconclusive^{∥} | 58 | 53/58 (91%) | 69 | 59/69 (86%) |
| ADA Negative^{#} | 218 | 198/218 (91%) | 188 | 163/188 (87%) |
| Week 196 | | | | |
| Total Evaluable for ADA^{†} | 304 | 271/304 (89%) | 285 | 248/285 (87%) |
| Treatment emergent positive NAb-POS^{‡} | 7 | 6/7 (86%) | 2 | 2/2 (100%) |
| Treatment emergent positive NAb-NEG^{‡} | 9 | 9/9 (100%) | 11 | 10/11 (91%) |
| Treatment emergent ADA positive^{‡} | 26 | 25/26 (96%) | 17 | 16/17 (94%) |
| Non-treatment emergent positive NAb-POS^{§} | 1 | 1/1 (100%) | 3 | 3/3 (100%) |
| Non-treatment emergent positive NAb-NEG^{§} | 12 | 11/12 (92%) | 13 | 11/13 (85%) |
| Non-treatment emergent ADA positive^{§} | 13 | 12/13 (92%) | 17 | 15/17 (88%) |
| Inconclusive^{∥} | 56 | 49/56 (88%) | 68 | 56/68 (82%) |
| ADA Negative^{#} | 209 | 185/209 (89%) | 183 | 161/183 (88%) |
| Week 220 | | | | |
| Total Evaluable for ADA^{†} | 293 | 265/293 (90%) | 278 | 241/278 (87%) |
| Treatment emergent positive NAb-POS^{‡} | 7 | 6/7 (86%) | 2 | 1/2 (50%) |
| Treatment emergent positive NAb-NEG^{‡} | 9 | 9/9 (100%) | 12 | 11/12 (92%) |
| Treatment emergent ADA positive^{‡} | 26 | 25/26 (96%) | 18 | 16/18 (89%) |
| Non-treatment emergent positive NAb-POS^{§} | 1 | 1/1 (100%) | 3 | 3/3 (100%) |
| Non-treatment emergent positive NAb-NEG^{§} | 11 | 11/11 (100%) | 13 | 11/13 (85%) |
| Non-treatment emergent ADA positive^{§} | 12 | 12/12 (100%) | 17 | 15/17 (88%) |
| Inconclusive^{∥} | 55 | 48/55 (87%) | 67 | 59/67 (88%) |
| ADA Negative^{#} | 200 | 180/200 (90%) | 176 | 151/176 (86%) |
| Week 244 | | | | |
| Total Evaluable for ADA^{†} | 283 | 260/283 (92%) | 271 | 244/271 (90%) |
| Treatment emergent positive NAb-POS^{‡} | 7 | 6/7 (86%) | 2 | 2/2 (100%) |
| Treatment emergent positive NAb-NEG^{‡} | 9 | 9/9 (100%) | 12 | 9/12 (75%) |
| Treatment emergent ADA positive^{‡} | 25 | 24/25 (96%) | 18 | 15/18 (83%) |
| Non-treatment emergent positive NAb-POS^{§} | 1 | 1/1 (100%) | 3 | 3/3 (100%) |
| Non-treatment emergent positive NAb-NEG^{§} | 10 | 10/10 (100%) | 13 | 12/13 (92%) |
| Non-treatment emergent ADA positive^{§} | 11 | 11/11 (100%) | 17 | 16/17 (94%) |
| Inconclusive^{∥} | 52 | 45/52 (87%) | 65 | 56/65 (86%) |
| ADA Negative^{#} | 195 | 180/195 (92%) | 171 | 157/171 (92%) |
| Week 268 | | | | |
| Total Evaluable for ADA^{†} | 101 | 94/101 (93%) | 84 | 77/84 (92%) |
| Treatment emergent positive NAb-POS^{‡} | 1 | 0/1 (0%) | 1 | 1/1 (100%) |
| Treatment emergent positive NAb-NEG^{‡} | 3 | 3/3 (100%) | 4 | 4/4 (100%) |
| Treatment emergent ADA positive^{‡} | 8 | 7/8 (88%) | 8 | 8/8 (100%) |
| Non-treatment emergent positive NAb-POS^{§} | 1 | 1/1 (100%) | 0 | 0/0 |
| Non-treatment emergent positive NAb-NEG^{§} | 3 | 3/3 (100%) | 3 | 3/3 (100%) |
| Non-treatment emergent ADA positive^{§} | 4 | 4/4 (100%) | 3 | 3/3 (100%) |
| Inconclusive^{∥} | 15 | 15/15 (100%) | 25 | 22/25 (88%) |
| ADA Negative^{#} | 74 | 68/74 (92%) | 48 | 44/48 (92%) |
| Week 292 | | | | |
| Total Evaluable for ADA^{†} | 80 | 75/80 (94%) | 70 | 61/70 (87%) |
| Treatment emergent positive NAb-POS^{‡} | 1 | 0/1 (0%) | 1 | 1/1 (100%) |
| Treatment emergent positive NAb-NEG^{‡} | 2 | 2/2 (100%) | 3 | 3/3 (100%) |
| Treatment emergent ADA positive^{‡} | 6 | 5/6 (83%) | 6 | 6/6 (100%) |
| Non-treatment emergent positive NAb-POS^{§} | 0 | 0/0 | 0 | 0/0 |
| Non-treatment emergent positive NAb-NEG^{§} | 2 | 2/2 (100%) | 3 | 3/3 (100%) |
| Non-treatment emergent ADA positive^{§} | 2 | 2/2 (100%) | 4 | 4/4 (100%) |
| Inconclusive^{∥} | 16 | 15/16 (94%) | 20 | 16/20 (80%) |
| ADA Negative^{#} | 56 | 53/56 (95%) | 40 | 35/40 (88%) |
| Week 316 | 46 | 44/46 (96%) | 34 | 34/34 (100%) |
| Total Evaluable for ADA^{†} | | | | |
| Treatment emergent positive NAb-POS^{‡} | 1 | 1/1 (100%) | 1 | 1/1 (100%) |
| Treatment emergent positive NAb-NEG^{‡} | 2 | 2/2 (100%) | 1 | 1/1 (100%) |
| Treatment emergent ADA positive^{‡} | 3 | 3/3 (100%) | 3 | 3/3 (100%) |
| Non-treatment emergent positive NAb-POS^{§} | 0 | 0/0 | 0 | 0/0 |
| Non-treatment emergent positive NAb-NEG^{§} | 1 | 1/1 (100%) | 0 | 0/0 |
| Non-treatment emergent ADA positive^{§} | 1 | 1/1 (100%) | 1 | 1/1 (100%) |
| Inconclusive^{∥} | 4 | 4/4 (100%) | 5 | 5/5 (100%) |
| ADA Negative^{#} | 38 | 36/38 (95%) | 25 | 25/25 (100%) |
| Week 340 | | | | |
| Total Evaluable for ADA^{†} | 47 | 47/47 (100%) | 28 | 27/28 (96%) |
| Treatment emergent positive NAb-POS^{‡} | 0 | 0/0 | 1 | 1/1 (100%) |
| Treatment emergent positive NAb-NEG^{‡} | 2 | 2/2 (100%) | 0 | 0/0 |
| Treatment emergent ADA positive^{‡} | 3 | 3/3 (100%) | 2 | 2/2 (100%) |
| Non-treatment emergent positive NAb-POS^{§} | 0 | 0/0 | 0 | 0/0 |
| Non-treatment emergent positive NAb-NEG^{§} | 1 | 1/1 (100%) | 1 | 0/1 (0%) |
| Non-treatment emergent ADA positive^{§} | 1 | 1/1 (100%) | 2 | 1/2 (50%) |
| Inconclusive^{∥} | 5 | 5/5 (100%) | 5 | 5/5 (100%) |
| ADA Negative^{#} | 38 | 38/38 (100%) | 19 | 19/19 (100%) |
| Week 364 | | | | |
| Total Evaluable for ADA^{†} | 11 | 11/11 (100%) | 9 | 9/9 (100%) |
| Treatment emergent positive NAb-POS^{‡} | 0 | 0/0 | 0 | 0/0 |
| Treatment emergent positive NAb-NEG^{‡} | 0 | 0/0 | 0 | 0/0 |
| Treatment emergent ADA positive^{‡} | 0 | 0/0 | 0 | 0/0 |
| Non-treatment emergent positive NAb-POS^{§} | 0 | 0/0 | 0 | 0/0 |
| Non-treatment emergent positive NAb-NEG^{§} | 0 | 0/0 | 0 | 0/0 |
| Non-treatment emergent ADA positive^{§} | 0 | 0/0 | 0 | 0/0 |
| Inconclusive^{∥} | 0 | 0/0 | 0 | 0/0 |
| ADA Negative^{#} | 11 | 11/11 (100%) | 9 | 9/9 (100%) |
| Week 396 | | | | |
| Total Evaluable for ADA^{†} | 1 | 1/1 (100%) | 0 | 0/0 |
| Treatment emergent positive NAb-POS^{‡} | 0 | 0/0 | 0 | 0/0 |
| Treatment emergent positive NAb-NEG^{‡} | 0 | 0/0 | 0 | 0/0 |
| Treatment emergent ADA positive^{‡} | 0 | 0/0 | 0 | 0/0 |
| Non-treatment emergent positive NAb-POS^{§} | 0 | 0/0 | 0 | 0/0 |
| Non-treatment emergent positive NAb-NEG^{§} | 0 | 0/0 | 0 | 0/0 |
| Non-treatment emergent ADA positive^{§} | 0 | 0/0 | 0 | 0/0 |
| Inconclusive^{∥} | 0 | 0/0 | 0 | 0/0 |
| ADA Negative^{#} | 1 | 1/1 (100%) | 0 | 0/0 |
| Week 420 | | | | |
| Total Evaluable for ADA^{†} | 0 | 0/0 | 0 | 0/0 |
| Treatment emergent positive NAb-POS^{‡} | 0 | 0/0 | 0 | 0/0 |
| Treatment emergent positive NAb-NEG^{‡} | 0 | 0/0 | 0 | 0/0 |
| Treatment emergent ADA positive^{‡} | 0 | 0/0 | 0 | 0/0 |
| Non-treatment emergent positive NAb-POS^{§} | 0 | 0/0 | 0 | 0/0 |
| Non-treatment emergent positive NAb-NEG^{§} | 0 | 0/0 | 0 | 0/0 |
| Non-treatment emergent ADA positive^{§} | 0 | 0/0 | 0 | 0/0 |
| Inconclusive^{∥} | 0 | 0/0 | 0 | 0/0 |
| ADA Negative^{#} | 0 | 0/0 | 0 | 0/0 |

N = Number of randomized subjects who received at least one dose of study medication in study part and with valid value at baseline and the time point for endpoint; PASI = Psoriasis Area and Severity Index.
^{†}Subjects evaluable for immunogenicity (ADA = anti-drug antibody) analysis were those with 1 or more samples obtained after MK-3222 administration.
^{‡}Treatment emergent positive includes subjects who had a negative pre-treatment (baseline) sample and at least 1 positive post-dose sample (treatment-induced positive) and also subjects with positive pre-treatment and post-dose samples and the titer increased post-dose by >=2-fold (treatment boosted positive). NAb-POS = neutralizing antibody positive; NAb-NEG = neutralizing antibody negative.
^{§}Non-treatment emergent positive includes subjects who had a positive pre-treatment (baseline) sample and also subjects with positive pre-treatment and post-dose samples in which the titer changed post-dose by <2-fold. NAb-POS = neutralizing antibody positive; NAb-NEG = neutralizing antibody negative.
^{∥}Inconclusive subjects had negative pre-treatment and post-dose samples and the MK-3222 concentration in the last post-dose sample was equal or above the drug tolerance limit for the ADA assay.
^{#}Negative subjects had negative pre-treatment and post-dose samples and the MK-3222 concentration in the last post-dose sample was below the drug tolerance limit for the ADA assay.

The proportion of subjects with PASI 75 response was smaller in subjects with treatment-emergent positive NAb positive than those with treatment-emergent positive NAb negative at most of timepoints. The numbers of subjects in the ADA inconclusive and ADA negative categories were elevated as compared to those with treatment-emergent positive NAb positive and NAb negative categories. The proportion of ADA inconclusive or negative subjects with PASI 75 response was high (ranged from 84% to 100% and from 89% to 100%, respectively, in the tildrakizumab 100 mg group and ranged from 80% to 100% and from 83% to 100%, respectively, in the tildrakizumab 200 mg group) at each timepoint during the extension study.

The proportion of subjects with PASI 75 response was elevated in the ADA inconclusive, ADA negative, and total ADA evaluable subjects than in subjects with treatment-emergent positive NAb positive.

The numbers of subjects with treatment-emergent positive NAb positive with PASI 90, and 100 response were small in both treatment groups (≤10 subjects in the tildrakizumab 100 mg group and ≤3 subjects in the tildrakizumab 200 mg group) at each timepoint during the extension study.

The proportion of subjects with treatment-emergent positive NAb positive and with PASI 90 response ranged from 0% to 86% in the tildrakizumab 100 mg group (until week 316) and from 33% to 100% in the tildrakizumab 200 mg group (until week 340) at each timepoint. The proportion of subjects with treatment-emergent positive Nab negative and with PASI 90 response ranged from 78% to 100% in the tildrakizumab 100 mg group (until Week 340) and from 50% to 100% in the tildrakizumab 200 mg group (until Week 316) at each timepoint. After the above-stated time points, there were no subjects in each of the two ADA categories in the two treatment groups.

The proportion of subjects with treatment-emergent positive NAb positive and with PASI100 response ranged from 0% to 57% in the tildrakizumab 100 mg group (until week 316) and from 0% to 50% in the tildrakizumab 200 mg group (until Week 340) at each timepoint. The proportion of subjects with treatment-emergent positive Nab negative and with PASI 100 response ranged from 33% to 100% in the tildrakizumab 100 mg group (until week 340) and from 9% to 100% in the tildrakizumab 200 mg group (until Week 316) at each timepoint. After the above stated time points, there were no subjects in each of the two ADA categories in the two treatment groups.

No notable differences in the proportion of subjects with PASI 90 response were observed in the ADA inconclusive, ADA negative, and total ADA evaluable subjects than in subjects with treatment-emergent positive NAb positive. The proportion of subjects with PASI 100 response was slightly higher in the ADA inconclusive, ADA negative, and ADA evaluable subjects than in subjects with treatment-emergent positive NAb positive in the tildrakizumab 100 mg group.

### Example 13. Correlation of Anti-Drug Immunogenicity Status with PGA Score of “Clear” or “Minimal” Over Time

A summary of anti-tildrakizumab immunogenicity status and proportion of subjects with a PGA score of “minimal” or “clear,” with at least a 2-grade reduction from baseline over time during the extension study is provided by subject immunogenicity category in Table 30.

TABLE 30
Subject Immunogenicity Status and Proportion with PGA Minimal or Clear, with at Least 2 Grade Reduction
from Baseline by Subject Immunogenicity Category Over Time (Full Analysis Set - Extension)

| | MK-3222 100 mg | | | MK-3222 200 mg | | |
|---|---|---|---|---|---|---|
| | PGA (N = 370) | | | PGA (N = 336) | | |
| | Subjects in | Subjects who | | Subjects in | Subjects who | |
| | ADA | achieved | | ADA | achieved | |
| | category with | endpoint/ | | category with | endpoint/ | |
| | evaluable | Subjects in | | evaluable | Subjects in | |
| | samples^{†} and | ADA category | | samples^{†} and | ADA category | |
| | PGA scores | Ratio (%) | | PGA scores | Ratio (%) | |
| Week 76 | | | | | | |
| Total Evaluable for ADA^{†} | 364 | 269/364 | (74%) | 328 | 217/328 | (66%) |
| Treatment emergent positive NAb-POS^{‡} | 10 | 7/10 | (70%) | 3 | 1/3 | (33%) |
| Treatment emergent positive NAb-NEG^{‡} | 12 | 12/12 | (100%) | 13 | 11/13 | (85%) |
| Treatment emergent ADA positive^{‡} | 34 | 29/34 | (85%) | 21 | 16/21 | (76%) |
| Non-treatment emergent positive NAb- | 1 | 1/1 | (100%) | 3 | 2/3 | (67%) |
| POS^{§} | | | | | | |
| Non-treatment emergent positive NAb- | 16 | 15/16 | (94%) | 15 | 10/15 | (67%) |
| NEG^{§} | | | | | | |
| Non-treatment emergent ADA positive^{§} | 17 | 16/17 | (94%) | 19 | 13/19 | (68%) |
| Inconclusive^{∥} | 58 | 42/58 | (72%) | 74 | 50/74 | (68%) |
| ADA Negative^{#} | 255 | 182/255 | (71%) | 214 | 138/214 | (64%) |
| Week 100 | | | | | | |
| Total Evaluable for ADA^{†} | 348 | 229/348 | (66%) | 325 | 216/325 | (66%) |
| Treatment emergent positive NAb-POS^{‡} | 8 | 5/8 | (63%) | 2 | 1/2 | (50%) |
| Treatment emergent positive NAb-NEG^{‡} | 11 | 11/11 | (100%) | 12 | 9/12 | (75%) |
| Treatment emergent ADA positive^{‡} | 29 | 23/29 | (79%) | 19 | 14/19 | (74%) |
| Non-treatment emergent positive NAb- | 1 | 1/1 | (100%) | 3 | 1/3 | (33%) |
| POS^{§} | | | | | | |
| Non-treatment emergent positive NAb- | 15 | 14/15 | (93%) | 15 | 9/15 | (60%) |
| NEG^{§} | | | | | | |
| Non-treatment emergent ADA positive^{§} | 16 | 15/16 | (94%) | 19 | 11/19 | (58%) |
| Inconclusive^{∥} | 55 | 34/55 | (62%) | 73 | 45/73 | (62%) |
| ADA Negative^{#} | 248 | 157/248 | (63%) | 214 | 146/214 | (68%) |
| Week 124 | | | | | | |
| Total Evaluable for ADA^{†} | 338 | 235/338 | (70%) | 306 | 199/306 | (65%) |
| Treatment emergent positive NAb-POS^{‡} | 8 | 5/8 | (63%) | 2 | 1/2 | (50%) |
| Treatment emergent positive NAb-NEG^{‡} | 11 | 11/11 | (100%) | 12 | 8/12 | (67%) |
| Treatment emergent ADA positive^{‡} | 30 | 25/30 | (83%) | 19 | 13/19 | (68%) |
| Non-treatment emergent positive NAb- | 1 | 0/1 | (0%) | 3 | 3/3 | (100%) |
| POS^{§} | | | | | | |
| Non-treatment emergent positive NAb- | 14 | 11/14 | (79%) | 15 | 10/15 | (67%) |
| NEG^{§} | | | | | | |
| Non-treatment emergent ADA positive^{§} | 15 | 11/15 | (73%) | 19 | 14/19 | (74%) |
| Inconclusive^{∥} | 56 | 36/56 | (64%) | 72 | 45/72 | (63%) |
| ADA Negative^{#} | 237 | 163/237 | (69%) | 196 | 127/196 | (65%) |
| Week 148 | | | | | | |
| Total Evaluable for ADA^{†} | 319 | 209/319 | (66%) | 293 | 195/293 | (67%) |
| Treatment emergent positive NAb-POS^{‡} | 7 | 5/7 | (71%) | 2 | 1/2 | (50%) |
| Treatment emergent positive NAb-NEG^{‡} | 10 | 8/10 | (80%) | 12 | 8/12 | (67%) |
| Treatment emergent ADA positive^{‡} | 28 | 20/28 | (71%) | 19 | 13/19 | (68%) |
| Non-treatment emergent positive NAb- | 1 | 1/1 | (100%) | 3 | 3/3 | (100%) |
| POS^{§} | | | | | | |
| Non-treatment emergent positive NAb- | 13 | 11/13 | (85%) | 14 | 10/14 | (71%) |
| NEG^{§} | | | | | | |
| Non-treatment emergent ADA positive^{§} | 14 | 12/14 | (86%) | 18 | 14/18 | (78%) |
| Inconclusive^{∥} | 56 | 37/56 | (66%) | 70 | 44/70 | (63%) |
| ADA Negative^{#} | 221 | 140/221 | (63%) | 186 | 124/186 | (67%) |
| Week 172 | | | | | | |
| Total Evaluable for ADA^{†} | 313 | 220/313 | (70%) | 290 | 181/290 | (62%) |
| Treatment emergent positive NAb-POS^{‡} | 7 | 5/7 | (71%) | 2 | 1/2 | (50%) |
| Treatment emergent positive NAb-NEG^{‡} | 9 | 8/9 | (89%) | 12 | 7/12 | (58%) |
| Treatment emergent ADA positive^{‡} | 27 | 22/27 | (81%) | 18 | 11/18 | (61%) |
| Non-treatment emergent positive NAb- | 1 | 1/1 | (100%) | 3 | 3/3 | (100%) |
| POS^{§} | | | | | | |
| Non-treatment emergent positive NAb- | 13 | 11/13 | (85%) | 14 | 7/14 | (50%) |
| NEG^{§} | | | | | | |
| Non-treatment emergent ADA positive^{§} | 14 | 12/14 | (86%) | 18 | 11/18 | (61%) |
| Inconclusive^{∥} | 56 | 38/56 | (68%) | 69 | 44/69 | (64%) |
| ADA Negative^{#} | 216 | 148/216 | (69%) | 185 | 115/185 | (62%) |
| Week 196 | | | | | | |
| Total Evaluable for ADA^{†} | 302 | 204/302 | (68%) | 282 | 183/282 | (65%) |
| Treatment emergent positive NAb-POS^{‡} | 7 | 5/7 | (71%) | 2 | 2/2 | (100%) |
| Treatment emergent positive NAb-NEG^{‡} | 9 | 9/9 | (100%) | 11 | 7/11 | (64%) |
| Treatment emergent ADA positive^{‡} | 26 | 23/26 | (88%) | 17 | 13/17 | (76%) |
| Non-treatment emergent positive NAb- | 1 | 1/1 | (100%) | 3 | 3/3 | (100%) |
| POS^{§} | | | | | | |
| Non-treatment emergent positive NAb- | 12 | 9/12 | (75%) | 13 | 11/13 | (85%) |
| NEG^{§} | | | | | | |
| Non-treatment emergent ADA positive^{§} | 13 | 10/13 | (77%) | 17 | 15/17 | (88%) |
| Inconclusive^{∥} | 54 | 33/54 | (61%) | 68 | 44/68 | (65%) |
| ADA Negative^{#} | 209 | 138/209 | (66%) | 180 | 111/180 | (62%) |
| Week 220 | | | | | | |
| Total Evaluable for ADA^{†} | 289 | 212/289 | (73%) | 274 | 177/274 | (65%) |
| Treatment emergent positive NAb-POS^{‡} | 7 | 6/7 | (86%) | 1 | 0/1 | (0%) |
| Treatment emergent positive NAb-NEG^{‡} | 9 | 8/9 | (89%) | 12 | 7/12 | (58%) |
| Treatment emergent ADA positive^{‡} | 26 | 21/26 | (81%) | 17 | 11/17 | (65%) |
| Non-treatment emergent positive NAb- | 1 | 1/1 | (100%) | 3 | 2/3 | (67%) |
| POS^{§} | | | | | | |
| Non-treatment emergent positive NAb- | 11 | 7/11 | (64%) | 13 | 8/13 | (62%) |
| NEG^{§} | | | | | | |
| Non-treatment emergent ADA positive^{§} | 12 | 8/12 | (67%) | 17 | 11/17 | (65%) |
| Inconclusive^{∥} | 53 | 37/53 | (70%) | 67 | 44/67 | (66%) |
| ADA Negative^{#} | 198 | 146/198 | (74%) | 173 | 111/173 | (64%) |
| Week 244 | | | | | | |
| Total Evaluable for ADA^{†} | 281 | 204/281 | (73%) | 268 | 183/268 | (68%) |
| Treatment emergent positive NAb-POS^{‡} | 7 | 6/7 | (86%) | 2 | 1/2 | (50%) |
| Treatment emergent positive NAb-NEG^{‡} | 9 | 9/9 | (100%) | 12 | 8/12 | (67%) |
| Treatment emergent ADA positive^{‡} | 25 | 24/25 | (96%) | 18 | 13/18 | (72%) |
| Non-treatment emergent positive NAb- | 1 | 1/1 | (100%) | 3 | 2/3 | (67%) |
| POS^{§} | | | | | | |
| Non-treatment emergent positive NAb- | 10 | 9/10 | (90%) | 13 | 8/13 | (62%) |
| NEG^{§} | | | | | | |
| Non-treatment emergent ADA positive^{§} | 11 | 10/11 | (91%) | 17 | 11/17 | (65%) |
| Inconclusive^{∥} | 50 | 32/50 | (64%) | 65 | 48/65 | (74%) |
| ADA Negative^{#} | 195 | 138/195 | (71%) | 168 | 111/168 | (66%) |
| Week 268 | | | | | | |
| Total Evaluable for ADA^{†} | 99 | 75/99 | (76%) | 83 | 58/83 | (70%) |
| Treatment emergent positive NAb-POS^{‡} | 1 | 0/1 | (0%) | 1 | 1/1 | (100%) |
| Treatment emergent positive NAb-NEG^{‡} | 3 | 3/3 | (100%) | 4 | 4/4 | (100%) |
| Treatment emergent ADA positive^{‡} | 8 | 7/8 | (88%) | 8 | 8/8 | (100%) |
| Non-treatment emergent positive NAb- | 1 | 0/1 | (0%) | 0 | 0/0 | |
| POS^{§} | | | | | | |
| Non-treatment emergent positive NAb- | 3 | 3/3 | (100%) | 3 | 2/3 | (67%) |
| NEG^{§} | | | | | | |
| Non-treatment emergent ADA positive^{§} | 4 | 3/4 | (75%) | 3 | 2/3 | (67%) |
| Inconclusive^{∥} | 13 | 9/13 | (69%) | 25 | 16/25 | (64%) |
| ADA Negative^{#} | 74 | 56/74 | (76%) | 47 | 32/47 | (68%) |
| Week 292 | | | | | | |
| Total Evaluable for ADA^{†} | 78 | 55/78 | (71%) | 69 | 49/69 | (71%) |
| Treatment emergent positive NAb-POS^{‡} | 1 | 0/1 | (0%) | 1 | 1/1 | (100%) |
| Treatment emergent positive NAb-NEG^{‡} | 2 | 2/2 | (100%) | 3 | 2/3 | (67%) |
| Treatment emergent ADA positive^{‡} | 6 | 4/6 | (67%) | 6 | 5/6 | (83%) |
| Non-treatment emergent positive NAb- | 0 | 0/0 | | 0 | 0/0 | |
| POS^{§} | | | | | | |
| Non-treatment emergent positive NAb- | 2 | 2/2 | (100%) | 3 | 2/3 | (67%) |
| NEG^{§} | | | | | | |
| Non-treatment emergent ADA positive^{§} | 2 | 2/2 | (100%) | 4 | 3/4 | (75%) |
| Inconclusive^{∥} | 14 | 9/14 | (64%) | 20 | 14/20 | (70%) |
| ADA Negative^{#} | 56 | 40/56 | (71%) | 39 | 27/39 | (69%) |
| Week 316 | | | | | | |
| Total Evaluable for ADA^{†} | 44 | 30/44 | (68%) | 33 | 27/33 | (82%) |
| Treatment emergent positive NAb-POS^{‡} | 1 | 0/1 | (0%) | 1 | 1/1 | (100%) |
| Treatment emergent positive NAb-NEG^{‡} | 2 | 2/2 | (100%) | 1 | 1/1 | (100%) |
| Treatment emergent ADA positive^{‡} | 3 | 2/3 | (67%) | 3 | 3/3 | (100%) |
| Non-treatment emergent positive NAb- | 0 | 0/0 | | 0 | 0/0 | |
| POS^{§} | | | | | | |
| Non-treatment emergent positive NAb- | 1 | 1/1 | (100%) | 0 | 0/0 | |
| NEG^{§} | | | | | | |
| Non-treatment emergent ADA positive^{§} | 1 | 1/1 | (100%) | 1 | 1/1 | (100%) |
| Inconclusive^{∥} | 2 | 2/2 | (100%) | 5 | 4/5 | (80%) |
| ADA Negative^{#} | 38 | 25/38 | (66%) | 24 | 19/24 | (79%) |
| Week 340 | | | | | | |
| Total Evaluable for ADA^{†} | 45 | 34/45 | (76%) | 28 | 25/28 | (89%) |
| Treatment emergent positive NAb-POS^{‡} | 0 | 0/0 | | 1 | 1/1 | (100%) |
| Treatment emergent positive NAb-NEG^{‡} | 2 | 2/2 | (100%) | 0 | 0/0 | |
| Treatment emergent ADA positive^{‡} | 3 | 3/3 | (100%) | 2 | 2/2 | (100%) |
| Non-treatment emergent positive NAb- | 0 | 0/0 | | 0 | 0/0 | |
| POS^{§} | | | | | | |
| Non-treatment emergent positive NAb- | 1 | 1/1 | (100%) | 1 | 0/1 | (0%) |
| NEG^{§} | | | | | | |
| Non-treatment emergent ADA positive^{§} | 1 | 1/1 | (100%) | 2 | 1/2 | (50%) |
| Inconclusive^{∥} | 3 | 3/3 | (100%) | 5 | 5/5 | (100%) |
| ADA Negative^{#} | 38 | 27/38 | (71%) | 19 | 17/19 | (89%) |
| Week 364 | | | | | | |
| Total Evaluable for ADA^{†} | 11 | 8/11 | (73%) | 9 | 8/9 | (89%) |
| Treatment emergent positive NAb-POS^{‡} | 0 | 0/0 | | 0 | 0/0 | |
| Treatment emergent positive NAb-NEG^{‡} | 0 | 0/0 | | 0 | 0/0 | |
| Treatment emergent ADA positive^{‡} | 0 | 0/0 | | 0 | 0/0 | |
| Non-treatment emergent positive NAb- | 0 | 0/0 | | 0 | 0/0 | |
| POS^{§} | | | | | | |
| Non-treatment emergent positive NAb- | 0 | 0/0 | | 0 | 0/0 | |
| NEG^{§} | | | | | | |
| Non-treatment emergent ADA positive^{§} | 0 | 0/0 | | 0 | 0/0 | |
| Inconclusive^{∥} | 0 | 0/0 | | 0 | 0/0 | |
| ADA Negative^{#} | 11 | 8/11 | (73%) | 9 | 8/9 | (89%) |

| Week 396 | | | | | |
|---|---|---|---|---|---|
| Total Evaluable for ADA^{†} | 1 | 1/1 | (100%) | 0 | 0/0 |
| Treatment emergent positive NAb-POS^{‡} | 0 | 0/0 | | 0 | 0/0 |
| Treatment emergent positive NAb-NEG^{‡} | 0 | 0/0 | | 0 | 0/0 |
| Treatment emergent ADA positive^{‡} | 0 | 0/0 | | 0 | 0/0 |
| Non-treatment emergent positive NAb- | 0 | 0/0 | | 0 | 0/0 |
| POS^{§} | | | | | |
| Non-treatment emergent positive NAb- | 0 | 0/0 | | 0 | 0/0 |
| NEG^{§} | | | | | |
| Non-treatment emergent ADA positive^{§} | 0 | 0/0 | | 0 | 0/0 |
| Inconclusive^{∥} | 0 | 0/0 | | 0 | 0/0 |
| ADA Negative^{#} | 1 | 1/1 | (100%) | 0 | 0/0 |

| | | | | |
|---|---|---|---|---|
| Week 420 | | | | |
| Total Evaluable for ADA^{†} | 0 | 0/0 | 0 | 0/0 |
| Treatment emergent positive NAb-POS^{‡} | 0 | 0/0 | 0 | 0/0 |
| Treatment emergent positive NAb-NEG^{‡} | 0 | 0/0 | 0 | 0/0 |
| Treatment emergent ADA positive^{‡} | 0 | 0/0 | 0 | 0/0 |
| Non-treatment emergent positive NAb- | 0 | 0/0 | 0 | 0/0 |
| POS^{§} | | | | |
| Non-treatment emergent positive NAb- | 0 | 0/0 | 0 | 0/0 |
| NEG^{§} | | | | |
| Non-treatment emergent ADA positive^{§} | 0 | 0/0 | 0 | 0/0 |
| Inconclusive^{∥} | 0 | 0/0 | 0 | 0/0 |
| ADA Negative^{#} | 0 | 0/0 | 0 | 0/0 |

N = Number of randomized subjects who received at least one dose of study medication in study part and with valid value at baseline and the time point for endpoint; PGA = Physician's Global Assessment.
^{†}Subjects evaluable for immunogenicity (ADA = anti-drug antibody) analysis were those with 1 or more samples obtained after MK-3222 administration.
^{‡}Treatment emergent positive includes subjects who had a negative pre-treatment (baseline) sample and at least 1 positive post-dose sample (treatment-induced positive) and also subjects with positive pre-treatment and post-dose samples and the titer increased post-dose by >= 2-fold (treatment boosted positive). NAb-POS = neutralizing antibody positive; NAb-NEG = neutralizing antibody negative.
^{§}Non-treatment emergent positive includes subjects who had a positive pre-treatment (baseline) sample and also subjects with positive pre-treatment and post-dose samples in which the titer changed post-dose by < 2-fold. NAb-POS = neutralizing antibody positive; NAb-NEG = neutralizing antibody negative.
^{∥}Inconclusive subjects had negative pre-treatment and post-dose samples and the MK-3222 concentration in the last post-dose sample was equal or above the drug tolerance limit for the ADA assay.
^{#}Negative subjects had negative pre-treatment and post-dose samples and the MK-3222 concentration in the last post-dose sample was below the drug tolerance limit for the ADA assay.

The numbers of subjects with treatment-emergent positive NAb positive and with PGA scores of “clear” or “minimal” were small (≤10 subjects in the tildrakizumab 100 mg group and ≤3 subjects in the tildrakizumab 200 mg group) at each time point during the extension study. Of these, the proportion of subjects with a PGA score of “minimal” or “clear,” ranged from 63% to 86% (except Week 268 onward when there was only 1 subject with a PGA score of“minimal” or “clear”) in the tildrakizumab 100 mg group and from 33% to 100% (except Week 220 when there was only 1 subject with a PGA score of “minimal” or “clear”) in the tildrakizumab 200 mg group at each timepoint.

The proportion of subjects with a PGA score of “minimal” or “clear” was similar between the total ADA evaluable subjects and subjects with treatment-emergent positive NAb positive in the tildrakizumab 100 mg while it was slightly higher in the total ADA evaluable subjects than the subjects with treatment-emergent positive NAb positive in the tildrakizumab 200 mg. The proportion of subjects with a PGA score of “minimal” or “clear” was higher in subjects with treatment-emergent positive NAb negative than in subjects with treatment-emergent positive NAb positive at most of timepoints. The number of subjects with treatment-emergent positive NAb positive and subjects with treatment-emergent positive NAb negative was small. In addition, the number of subjects with treatment-emergent positive NAb positive at each timepoint in both treatment groups was smaller than those with treatment-emergent positive NAb negative.

The proportion of ADA inconclusive or negative subjects who achieved a PGA score of “minimal” or “clear” ranged from 61% to 100% and from 63% to 100%, respectively, in the tildrakizumab 100 mg group and ranged from 62% to 100% and from 62% to 89%, respectively, in the tildrakizumab 200 mg group) at each timepoint during the extension study.

### Efficacy, Pharmacokinetics, and Immunogenicity Results Summary

### CONCLUSION

### Efficacy Results:

Baseline efficacy parameters were generally similar between the two treatment groups. The PASI 50, PASI 75, and PASI 90 response among those who were responders at Week 52 in both tildrakizumab treatment groups was maintained in a majority of subjects who remained in the study at specified time points throughout the extension study. The proportion of subjects with PASI 100 response among those who were responders at Week 52 decreased approximately 20 percentage points from Week 60 to Week 244 in both treatment groups. The PASI 50, PASI 75, PASI 90, and PASI 100 responses irrespective of subjects who were responders at Week 52 in both tildrakizumab treatment groups were maintained throughout the extension study. Mean changes in PASI scores were maintained in both treatment groups during the extension study. Similarly, mean percent changes in PASI scores were maintained over time in the two treatment groups. The proportions of subjects with a PGA score of “clear” or “minimal,” with at least a 2 Grade point reduction from baseline were similar at most of timepoints in the two tildrakizumab treatment groups and were maintained during the extension study. The proportion of subjects with any adverse experience or with any device failure among subjects who were treated with AI was very low over time (≤0.5% and ≤1.6%, respectively).

### Pharmacokinetic Results:

Based on available PK concentration data in extension 1 (up to week 244), the mean concentrations of tildrakizumab from week 76 to week 244 were comparable for each dose group. Mean concentrations in 200 mg dose group are higher than at 100 mg dose group suggesting a dose dependent increase in PK.

### Immunogenicity Results

Out of 725 ADA evaluable subjects, overall, 586 (80.8%) subjects had conclusive results. Of the 725 ADA evaluable subjects, 491 (67.7%) subjects were ADA negative, and 39 (5.4%) subjects were non-treatment-emergent ADA positive while 56 (7.7%) subjects were treatment-emergent ADA positive. The proportion of ADA positive subjects was similar between the two treatment groups. The proportion of treatment-emergent ADA positive subjects was numerically higher in the tildrakizumab 100 mg group (35 [9.2%] subjects) compared with the tildrakizumab 200 mg group (21[6.1%] subjects). The number of subjects with treatment-emergent positive NAb positive with PASI 75, 90, and 100 response and numbers of subjects with treatment-emergent positive NAb positive and with PGA scores of “clear” or “minimal” with at least a two-grade point reduction from baseline was ≤10 subjects in the tildrakizumab 100 mg group and ≤3 subjects in the tildrakizumab 200 mg group at each timepoint during the extension study.

### Safety Evaluation—Brief Summary of Adverse Events

An overall summary of AEs reported during the extension study is provided by treatment group in Table 31.

TABLE 31
Adverse Events Summary (All subjects treated as treated - Extension) Extension Study

| | MK-3222 | | MK-3222 | | | |
|---|---|---|---|---|---|---|
| | 100 mg | | 100 mg | | Total | |
| | n | (%) | n | (%) | n | (%) |
| Subjects in population^{†} | 381 | | 349 | | 730 | |
| with one or more adverse events | 321 | (84.3) | 307 | (88.0) | 628 | (86.0) |
| with no adverse events | 60 | (15.7) | 42 | (12.0) | 102 | (14.0) |
| with drug-related^{‡} adverse events | 91 | (23.9) | 99 | (28.4) | 190 | (26.0) |
| with serious adverse events | 87 | (22.8) | 79 | (22.6) | 166 | (22.7) |
| with serious drug-related^{‡} adverse events | 8 | (2.1) | 6 | (1.7) | 14 | (1.9) |
| who died | 4 | (1.0) | 2 | (0.6) | 6 | (0.8) |
| discontinued^{§} due to an adverse event | 16 | (4.2) | 13 | (3.7) | 29 | (4.0) |
| discontinued^{§} due to a drug-related^{‡} adverse event | 4 | (1.0) | 3 | (0.9) | 7 | (1.0) |
| discontinued^{§} due to a serious adverse event | 7 | (1.8) | 8 | (2.3) | 15 | (2.1) |
| discontinued^{§} due to a serious drug-related^{‡} | 1 | (0.3) | 1 | (0.3) | 2 | (0.3) |
| adverse event | | | | | | |

^{†}Subjects who took at least one dose of extension study medication based on the treatment actually received.
^{‡}Determined by the investigator to be related to the drug.
₌^{§}Study medication withdrawn.

No meaningful differences in the overall frequency of AEs were observed between the two treatment groups (321 [84.3%] subjects and 307 [88.0%] subjects in the tildrakizumab 100 mg group and the tildrakizumab 200 mg group, respectively).

The frequencies of drug-related AEs (91 [23.9%] subjects and 99 [28.4%] subjects), SAEs (87 [22.8%] subjects and 79 [22.6%] subjects in the tildrakizumab 100 mg group and the tildrakizumab 200 mg group, respectively), and discontinuations due to any AE (16 [4.2%] and 13 [3.7%] subjects) were similar between the two treatment groups. The frequencies of serious drug-related AEs, discontinuations due to a drug-related AE and serious drug-related AEs were also similar between the two treatment groups.

The overall frequencies of AEs and drug-related AEs were higher in subjects treated with PFS compared to those treated with AI while the overall frequency of SAEs was higher in subjects treated with AI compared to those treated with PFS.

The cumulative exposure-adjusted frequencies of overall AEs and drug-related AEs were slightly higher in the tildrakizumab 200 mg than in the tildrakizumab 100 mg while those of SAEs, serious drug-related AEs, and AEs leading to discontinuation were similar between the two tildrakizumab treatment groups in the base study.

A similar trend was observed for the 5-year cumulative exposure-adjusted frequencies in the base and the extension study. The frequencies of overall AEs and drug-related AEs gradually decreased with longer duration of treatment (overall AEs: Year 1: 64.0%; Year 2: 56.7%; Year 3: 51.4%; Year 4: 39.2%, drug-related AEs: Year 1: 16.3%; Year 2: 12.3%; Year 3: 11.9%; Year 4: 6.7%) and were similar between the two treatment groups.

### Most Frequently Reported Adverse Events

The proportion of subjects with AEs by SOC were generally similar in both treatment groups except for Ear and labyrinth disorders SOC where the proportion of AEs was higher in the tildrakizumab 100 mg group than in the tildrakizumab 200 mg group.

Overall, the most frequently reported AEs by SOC occurred in the Infections and Infestations SOC (248 [65.1%] subjects and 231 [66.2%] subjects in the tildrakizumab 100 mg group and the tildrakizumab 200 mg group, respectively), with no notable difference between the two treatment groups. The most frequently reported AE by PT was nasopharyngitis (150 [39.4%] subjects and 132 [37.8%] subjects in the tildrakizumab 100 mg group and the tildrakizumab 200 mg group, respectively).

No meaningful differences in AEs reported by SOC and PT were observed between subjects treated with PFS and those treated with AI during the extension study. The 5-year cumulative exposure-adjusted frequencies of AEs by SOC and PT were generally similar.

### Drug-Related Adverse Events

A summary of drug-related AEs reported during the extension study is provided by treatment group, SOC, and PT in Table 32.

TABLE 32
Subjects with Drug-Related adverse events (All subjects as treated - extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more drug-related adverse events | 91 | (23.9) | 99 | (28.4) | 190 | (26.0) |
| With no drug-related adverse events | 290 | (76.1) | 250 | (71.6) | 540 | (74.0) |
| Blood and lymphatic system disorders | 2 | (0.5) | 3 | (0.9) | 5 | (0.7) |
| Anaemia | 1 | (0.3) | 0 | | 1 | (0.1) |
| Leukocytosis | 0 | | 1 | (0.3) | 1 | (0.1) |
| Leukopenia | 1 | (0.3) | 0 | | 1 | (0.1) |
| Lymphopenia | 0 | | 1 | (0.3) | 1 | (0.1) |
| Thrombocytopenia | 0 | | 1 | (0.3) | 1 | (0.1) |
| Cardiac disorders | 3 | (0.8) | 2 | (0.6) | 5 | (0.7) |
| Bundle branch block left | 0 | | 1 | (0.3) | 1 | (0.1) |
| Myocarditis | 1 | (0.3) | 0 | | 1 | (0.1) |
| Nodal rhythm | 0 | | 1 | (0.3) | 1 | (0.1) |
| Tachycardia | 2 | (0.5) | 0 | | 2 | (0.3) |
| Ear and labyrinth disorders | 1 | (0.3) | 1 | (0.3) | 2 | (0.3) |
| Ear pain | 1 | (0.3) | 0 | | 1 | (0.1) |
| Vertigo | 0 | | 1 | (0.3) | 1 | (0.1) |
| Endocrine disorders | 1 | (0.3) | 0 | | 1 | (0.1) |
| Hyperthyroidism | 1 | (0.3) | 0 | | 1 | (0.1) |
| Thyrotoxic crisis | 1 | (0.3) | 0 | | 1 | (0.1) |
| Eye disorders | 0 | | 2 | (0.6) | 2 | (0.3) |
| Dry eye | 0 | | 1 | (0.3) | 1 | (0.1) |
| Xerophthalmia | 0 | | 1 | (0.3) | 1 | (0.1) |
| Gastrointestinal disorders | 10 | (2.6) | 10 | (2.9) | 20 | (2.7) |
| Abdominal discomfort | 1 | (0.3) | 0 | | 1 | (0.1) |
| Abdominal pain | 0 | | 2 | (0.6) | 2 | (0.3) |
| Abdominal pain lower | 1 | (0.3) | 0 | | 1 | (0.1) |
| Abdominal pain upper | 0 | | 2 | (0.6) | 2 | (0.3) |
| Aphthous ulcer | 1 | (0.3) | 0 | | 1 | (0.1) |
| Cheilitis | 0 | | 1 | (0.3) | 1 | (0.1) |
| Colitis | 1 | (0.3) | 0 | | 1 | (0.1) |
| Crohn's disease | 1 | (0.3) | 0 | | 1 | (0.1) |
| Diarrhoea | 1 | (0.3) | 2 | (0.6) | 3 | (0.4) |
| Dyspepsia | 1 | (0.3) | 0 | | 1 | (0.1) |
| Dysphagia | 1 | (0.3) | 0 | | 1 | (0.1) |
| Gastrointestinal inflammation | 1 | (0.3) | 0 | | 1 | (0.1) |
| Mesenteric artery thrombosis | 1 | (0.3) | 0 | | 1 | (0.1) |
| Oesophagitis | 0 | | 1 | (0.3) | 1 | (0.1) |
| Oral pain | 0 | | 2 | (0.6) | 2 | (0.3) |
| Proctitis | 0 | | 1 | (0.3) | 1 | (0.1) |
| Rectal haemorrhage | 0 | | 1 | (0.3) | 1 | (0.1) |
| Reflux gastritis | 1 | (0.3) | 0 | | 1 | (0.1) |
| Toothache | 0 | | 1 | (0.3) | 1 | (0.1) |
| General disorders and administration site | 13 | (3.4) | 16 | (4.6) | 29 | (4.0) |
| conditions | | | | | | |
| Application site swelling | 0 | | 1 | (0.3) | 1 | (0.1) |
| Asthenia | 1 | (0.3) | 0 | | 1 | (0.1) |
| Chest pain | 0 | | 1 | (0.3) | 1 | (0.1) |
| Fatigue | 2 | (0.5) | 1 | (0.3) | 3 | (0.4) |
| Illness | 0 | | 1 | (0.3) | 1 | (0.1) |
| Influenza like illness | 0 | | 3 | (0.9) | 3 | (0.4) |
| Injection site bruising | 2 | (0.5) | 0 | | 2 | (0.3) |
| Injection site discomfort | 0 | | 1 | (0.3) | 1 | (0.1) |
| Injection site erythema | 0 | | 4 | (1.1) | 4 | (0.5) |
| Injection site haematoma | 2 | (0.5) | 1 | (0.3) | 3 | (0.4) |
| Injection site haemorrhage | 2 | (0.5) | 0 | | 2 | (0.3) |
| Injection site pain | 2 | (0.5) | 3 | (0.9) | 5 | (0.7) |
| Injection site pruritus | 0 | | 3 | (0.9) | 3 | (0.4) |
| Injection site reaction | 1 | (0.3) | 0 | | 1 | (0.1) |
| Injection site swelling | 0 | | 2 | (0.6) | 2 | (0.3) |
| Pyrexia | 2 | (0.5) | 2 | (0.6) | 4 | (0.5) |
| Infections and infestations | 67 | (17.6) | 85 | (24.4) | 152 | (20.8) |
| Actinomycotic skin infection | 0 | | 1 | (0.3) | 1 | (0.1) |
| Acute sinusitis | 1 | (0.3) | 2 | (0.6) | 3 | (0.4) |
| Anal fungal infection | 0 | | 1 | (0.3) | 1 | (0.1) |
| Appendicitis | 1 | (0.3) | 1 | (0.3) | 2 | (0.3) |
| Arthritis infective | 1 | (0.3) | 1 | (0.3) | 2 | (0.3) |
| Body tinea | 0 | | 2 | (0.6) | 2 | (0.3) |
| Bronchitis | 7 | (1.8) | 8 | (2.3) | 15 | (2.1) |
| Conjunctivitis | 0 | | 3 | (0.9) | 3 | (0.4) |
| Cystitis | 0 | | 2 | (0.6) | 2 | (0.3) |
| Diverticulitis | 1 | (0.3) | 0 | | 1 | (0.1) |
| Ear infection | 0 | | 2 | (0.6) | 2 | (0.3) |
| Epididymitis | 0 | | 1 | (0.3) | 1 | (0.1) |
| Erysipelas | 2 | (0.5) | 3 | (0.9) | 5 | (0.7) |
| Folliculitis | 1 | (0.3) | 0 | | 1 | (0.1) |
| Fungal skin infection | 1 | (0.3) | 2 | (0.6) | 3 | (0.4) |
| Furuncle | 0 | (0.1) | 1 | (0.3) | 1 | (0.1) |
| Gastroenteritis | 4 | (1.0) | 4 | (1.1) | 8 | (1.1) |
| Gastrointestinal infection | 3 | (0.8) | 3 | (0.9) | 6 | (0.8) |
| Gingivitis | 1 | (0.3) | 1 | (0.3) | 2 | (0.3) |
| Helicobacter infection | 0 | | 1 | (0.3) | 1 | (0.1) |
| Herpes simplex | 0 | | 1 | (0.3) | 1 | (0.1) |
| Herpes zoster | 1 | (0.3) | 0 | | 1 | (0.1) |
| Infected cyst | 1 | (0.3) | 0 | | 1 | (0.1) |
| Influenza | 1 | (0.3) | 8 | (2.3) | 9 | (1.2) |
| Large intestine infection | 0 | | 1 | (0.3) | 1 | (0.1) |
| Laryngitis | 0 | | 1 | (0.3) | 1 | (0.1) |
| Meningitis viral | 0 | | 1 | (0.3) | 1 | (0.1) |
| Nasopharyngitis | 43 | (11.3) | 49 | (14.0) | 92 | (12.6) |
| Oesophageal candidiasis | 1 | (0.3) | 0 | | 1 | (0.1) |
| Onychomycosis | 1 | (0.3) | 2 | (0.6) | 3 | (0.4) |
| Oral candidiasis | 0 | | 1 | (0.3) | 1 | (0.1) |
| Oral herpes | 3 | (0.8) | 2 | (0.6) | 5 | (0.7) |
| Oropharyngeal candidiasis | 0 | | 1 | (0.3) | 1 | (0.1) |
| Otitis media | 1 | (0.3) | 4 | (1.1) | 5 | (0.7) |
| Paronychia | 1 | (0.3) | 0 | | 1 | (0.1) |
| Peritonitis | 0 | | 1 | (0.3) | 1 | (0.1) |
| Pertussis | 0 | | 1 | (0.3) | 1 | (0.1) |
| Pharyngitis | 4 | (1.0) | 3 | (0.9) | 7 | (1.0) |
| Pneumonia | 0 | | 2 | (0.6) | 2 | (0.3) |
| Pneumonia mycoplasmal | 0 | | 1 | (0.3) | 1 | (0.1) |
| Pulpitis dental | 1 | (0.3) | 2 | (0.6) | 3 | (0.4) |
| Pyoderma | 1 | (0.3) | 0 | | 1 | (0.1) |
| Respiratory tract infection viral | 0 | | 1 | (0.3) | 1 | (0.1) |
| Rhinitis | 10 | (2.6) | 6 | (1.7) | 16 | (2.2) |
| Sinusitis | 6 | (1.6) | 4 | (1.1) | 10 | (1.4) |
| Soft tissue infection | 0 | | 1 | (0.3) | 1 | (0.1) |
| Tinea infection | 0 | | 2 | (0.6) | 2 | (0.3) |
| Tinea pedis | 2 | (0.5) | 6 | (1.7) | 8 | (1.1) |
| Tinea versicolour | 0 | | 1 | (0.3) | 1 | (0.1) |
| Tonsillitis | 3 | (0.8) | 4 | (1.1) | 7 | (1.0) |
| Tooth abscess | 1 | (0.3) | 0 | | 1 | (0.1) |
| Upper respiratory tract infection | 11 | (2.9) | 13 | (3.7) | 24 | (3.3) |
| Urinary tract infection | 7 | (1.8) | 4 | (1.1) | 11 | (1.5) |
| Vaginal infection | 0 | | 1 | (0.3) | 1 | (0.1) |
| Vulvovaginal candidiasis | 0 | | 1 | (0.3) | 1 | (0.1) |
| Vulvovaginal mycotic infection | 1 | (0.3) | 1 | (0.3) | 2 | (0.3) |
| Wound infection | 1 | (0.3) | 0 | | 1 | (0.1) |
| Investigations | 4 | (1.0) | 5 | (1.4) | 9 | (1.2) |
| Bacterial test | 0 | | 1 | (0.3) | 1 | (0.1) |
| Blood bilirubin increased | 0 | | 1 | (0.3) | 1 | (0.1) |
| Blood lactate dehydrogenase increased | 1 | (0.3) | 0 | | 1 | (0.1) |
| C-reactive protein increased | 1 | (0.3) | 0 | | 1 | (0.1) |
| Fungal test positive | 0 | | 1 | (0.3) | 1 | (0.1) |
| Liver function test increased | 1 | (0.3) | 0 | | 1 | (0.1) |
| Nitrite urine present | 0 | | 1 | (0.3) | 1 | (0.1) |
| Platelet count decreased | 1 | (0.3) | 0 | | 1 | (0.1) |
| Total lung capacity decreased | 0 | | 1 | (0.3) | 1 | (0.1) |
| Urinary sediment present | 0 | | 1 | (0.3) | 1 | (0.1) |
| Weight decreased | 1 | (0.3) | 0 | | 1 | (0.1) |
| Metabolism and nutrition disorders | 2 | (0.5) | 0 | | 2 | (0.3) |
| Hypercholesterolaemia | 1 | (0.3) | 0 | | 1 | (0.1) |
| Hypokalaemia | 1 | (0.3) | 0 | | 1 | (0.1) |
| Musculoskeletal and connective tissue disorders | 5 | (1.3) | 7 | (2.0) | 12 | (1.6) |
| Arthralgia | 1 | (0.3) | 2 | (0.6) | 3 | (0.4) |
| Back pain | 0 | | 1 | (0.3) | 1 | (0.1) |
| Muscle spasms | 2 | (0.5) | 1 | (0.3) | 3 | (0.4) |
| Musculoskeletal pain | 1 | (0.3) | 0 | | 1 | (0.1) |
| Myalgia | 0 | | 1 | (0.3) | 1 | (0.1) |
| Myositis | 0 | | 1 | (0.3) | 1 | (0.1) |
| Osteitis | 0 | | 1 | (0.3) | 1 | (0.1) |
| Polymyalgia rheumatica | 0 | | 1 | (0.3) | 1 | (0.1) |
| Psoriatic arthropathy | 1 | (0.3) | 0 | | 1 | (0.1) |
| Neoplasms benign, malignant and unspecified (incl | 2 | (0.5) | 3 | (0.9) | 5 | (0.7) |
| cysts and polyps) | | | | | | |
| Basal cell carcinoma | 0 | | 1 | (0.3) | 1 | (0.1) |
| Dysplastic naevus | 0 | | 1 | (0.3) | 1 | (0.1) |
| Rectal adenocarcinoma | 1 | (0.3) | 0 | | 1 | (0.1) |
| Skin papilloma | 1 | (0.3) | 1 | (0.3) | 2 | (0.3) |
| Nervous system disorders | 6 | (1.6) | 5 | (1.4) | 11 | (1.5) |
| Facial paralysis | 0 | | 1 | (0.3) | 1 | (0.1) |
| Headache | 3 | (0.8) | 5 | (1.4) | 8 | (1.1) |
| Migraine | 1 | (0.3) | 0 | | 1 | (0.1) |
| Paraesthesia | 1 | (0.3) | 0 | | 1 | (0.1) |
| Syncope | 1 | (0.3) | 0 | | 1 | (0.1) |
| Psychiatric disorders | 1 | (0.3) | 0 | | 1 | (0.1) |
| Anxiety | 1 | (0.3) | 0 | | 1 | (0.1) |
| Renal and urinary disorders | 3 | (0.8) | 2 | (0.6) | 5 | (0.7) |
| Haematuria | 2 | (0.5) | 0 | | 2 | (0.3) |
| Proteinuria | 1 | (0.3) | 2 | (0.6) | 3 | (0.4) |
| Reproductive system and breast disorders | 1 | (0.3) | 3 | (0.9) | 4 | (0.5) |
| Balanoposthitis | 0 | | 1 | (0.3) | 1 | (0.1) |
| Genital rash | 0 | | 1 | (0.3) | 1 | (0.1) |
| Prostatitis | 1 | (0.3) | 1 | (0.3) | 2 | (0.3) |
| Respiratory, thoracic and mediastinal disorders | 10 | (2.6) | 12 | (3.4) | 22 | (3.0) |
| Asthma | 1 | (0.3) | 0 | | 1 | (0.1) |
| Chronic obstructive pulmonary disease | 0 | | 1 | (0.3) | 1 | (0.1) |
| Cough | 2 | (0.5) | 6 | (1.7) | 8 | (1.1) |
| Dysphonia | 1 | (0.3) | 1 | (0.3) | 2 | (0.3) |
| Oropharyngeal pain | 5 | (1.3) | 5 | (1.4) | 10 | (1.4) |
| Productive cough | 0 | | 1 | (0.3) | 1 | (0.1) |
| Throat irritation | 1 | (0.3) | 0 | | 1 | (0.1) |
| Skin and subcutaneous tissue disorders | 9 | (2.4) | 12 | (3.4) | 21 | (2.9) |
| Acne | 0 | | 1 | (0.3) | 1 | (0.1) |
| Actinic keratosis | 0 | | 1 | (0.3) | 1 | (0.1) |
| Dermatitis | 0 | | 1 | (0.3) | 1 | (0.1) |
| Drug eruption | 0 | | 1 | (0.3) | 1 | (0.1) |
| Intertrigo | 1 | (0.3) | 1 | (0.3) | 2 | (0.3) |
| Pruritus | 3 | (0.8) | 3 | (0.9) | 6 | (0.8) |
| Psoriasis | 3 | (0.8) | 2 | (0.6) | 5 | (0.7) |
| Rosacea | 0 | | 1 | (0.3) | 1 | (0.1) |
| Skin erosion | 1 | (0.3) | 0 | | 1 | (0.1) |
| Skin hyperpigmentation | 1 | (0.3) | 0 | | 1 | (0.1) |
| Urticaria | 1 | (0.3) | 1 | (0.3) | 2 | (0.3) |
| Vascular disorders | 7 | (1.8) | 5 | (1.4) | 12 | (1.6) |
| Deep vein thrombosis | 1 | (0.3) | 0 | | 1 | (0.1) |
| Hypertension | 6 | (1.6) | 4 | (1.1) | 10 | (1.4) |
| Hypertensive crisis | 1 | (0.3) | 0 | | 1 | (0.1) |
| Thrombosis | 0 | | 1 | (0.3) | 1 | (0.1) |

^{†}Subjects who took at least one dose of extension study medication based on the treatment actually received. Subjects were counted only once in the overall category. A subject may appear in different categories. MedDRA Version: Merck T2-MedDRA.0 and MedDRA24.0

No meaningful differences were observed in the overall frequencies of drug-related AEs between the two treatment groups (91 [23.9%] subjects and 99 [28.4%] subjects in tildrakizumab 100 mg group and the tildrakizumab 200 mg group, respectively). The most frequently reported drug-related AEs by SOC was the Infections and Infestations (152 [20.8%] subjects overall; 67 [17.6%] subjects in tildrakizumab 100 mg group and 85 [24.4%] subjects in the tildrakizumab 200 mg group). The most frequently reported drug-related AE was nasopharyngitis (92 [12.6%] subjects overall; 43 [11.3%] subjects in the tildrakizumab 100 mg group and 49 [14.0%] subjects in the tildrakizumab 200 mg group).

No meaningful differences in the frequencies of drug-related AEs by SOC and PT were observed between subjects treated with PFS and those treated with AI during the extension study. No notable differences were observed in the 5-year cumulative exposure-adjusted frequencies of drug-related AEs by SOC and PT in the two tildrakizumab groups.

### Analysis of Deaths, Other Serious Adverse Events, and Other Clinically Meaningful Adverse Events (Deaths, Other Serious Adverse Events, Discontinuation Due to Adverse Events and Other Adverse Events of Special Interest)

### Deaths

A by-subject listing of SAEs resulting in deaths during the extension study is provided in Table 33. Deaths were reported in 6 subjects (4 subjects and 2 subjects in the tildrakizumab 100 mg and the tildrakizumab 200 mg group, respectively) during the extension study. Of these, 2 subjects died due to complete suicide, 1 subject due to toxicity to various agents, 1 subject due to acute myocardial infarctions, 1 subject due to asphyxia, and 1 subject (Subject ID: 208347) due to an unknown cause. All deaths were considered not related to study medication.

TABLE 33
Listing of Subjects with Serious Adverse Events Resulting in Death (All subjects as treated) Extensions study

| | | | | | | | | | |
|---|---|---|---|---|---|---|---|---|---|
| | | Rel | | | | | | | |
| | | Day of | | | | | | | |
| | Treatment | Onset | | | | | | | |
| | Phase | Study | | | | | | | |
| Subject | (Treatment | Part^{‡}/Study | Adverse | | | | | Action | |
| ID | Received) | Start^{§} | Event | Duration | Intensity | Serious | Related | Taken | Outcome |

MK-3222 100 mg
Trial Number = 3222-011, Site Number = 0169, Gender = M, Race = White, Age = 45 Years, Rel Day of Study Medication
Discontinuance^{†} = 879, Rel Day of Last Study Medication Discontinuance^{††} = 879

| | | | | | | | | |
|---|---|---|---|---|---|---|---|---|
| 202410 | Extension | 886/1306 | Completed | Severe | Y | N | None | Fatal |
| | Study | | suicide | | | | | |
| | (MK-3222 | | | | | | | |
| | 100 mg) | | | | | | | |

Trial Number = 3222-011, Site Number = 1171, Gender = F, Race = White, Age = 21 Years, Rel Day of Study Medication
Discontinuance^{†} = 1213, Rel Day of Last Study Medication Discontinuance^{††} = 1213

| | | | | | | | | |
|---|---|---|---|---|---|---|---|---|
| 202453 | Extension | 1303/1723 | Completed | Severe | Y | N | Discontinued | Fatal |
| | Study | | suicide | | | | | |
| | (MK-3222 | | | | | | | |
| | 100 mg) | | | | | | | |

Trial Number = 3222-011, Site Number = 0187, Gender = M, Race = White, Age = 38 Years, Rel Day of Study Medication
Discontinuance^{†} = 506, Rel Day of Last Study Medication Discontinuance^{††} = 506

| | | | | | | | | | |
|---|---|---|---|---|---|---|---|---|---|
| 208347 | Extension | 552/978 | Death | 1.0 | Severe | Y | N | None | Fatal |
| | Study | | | Days | | | | | |
| | (MK-3222 | | | | | | | | |
| | 100 mg) | | | | | | | | |

Trial Number = 3222-011, Site Number = 0166, Gender = M, Race = White, Age = 58 Years, Rel Day of Study Medication
Discontinuance^{†} = 204, Rel Day of Last Study Medication Discontinuance^{††} = 204

| | | | | | | | | | |
|---|---|---|---|---|---|---|---|---|---|
| 208404 | Extension | 226/640 | Toxicity to | 1.0 | Severe | Y | N | None | Fatal |
| | Study | | various | Days | | | | | |
| | (MK-3222 | | agents | | | | | | |
| | 100 mg) | | | | | | | | |

MK-3222 200 mg
Trial Number = 3222-011, Site Number = 4304, Gender = M, Race = White, Age = 52 Years, Rel Day of Study Medication
Discontinuance^{†} = 711, Rel Day of Last Study Medication Discontinuance^{††} = 711

| | | | | | | | | | |
|---|---|---|---|---|---|---|---|---|---|
| 213177 | Extension | 764/1181 | Acute | 1.0 | Severe | Y | N | None | Fatal |
| | Study | | myocardial | Days | | | | | |
| | (MK-3222 | | infarction | | | | | | |
| | 200 mg) | | | | | | | | |

Trial Number = 3222-011, Site Number = 4801, Gender = M, Race = White, Age = 24 Years, Rel Day of Study Medication
Discontinuance^{†} = 284, Rel Day of Last Study Medication Discontinuance^{††} = 284

| | | | | | | | | |
|---|---|---|---|---|---|---|---|---|
| 213180 | Extension | 326/746 | Asphyxia | Severe | Y | N | Discontinued | Fatal |
| | Study | | | | | | | |
| | (MK-3222 | | | | | | | |
| | 200 mg) | | | | | | | |

^{†}Relative Day of Study Medication Discontinuance is defined as the day of the last recorded dose of study medication for the subject relative to the start of study medication.
^{††}Relative Day of Study Medication Discontinuance from Last Part is defined as the day of the last recorded dose of study medication for the subject relative to the start of study medication in the last treatment phase.
^{‡}Rel Day of Onset is from the start of study part.
^{§}Rel Day of Onset is from the start of study. Action Taken: None = DOSE NOT CHANGED, Reduced = DOSE REDUCED, Interrupted = DRUG INTERRUPTED, Discontinued = DRUG WITHDRAWN,

### Other Serious Adverse Events

A summary of SAEs reported during the extension study is provided in Table 34. One or more SAEs were reported in 87 (22.8%) subjects and 79 (22.60%) subjects in the tildrakizumab 100 mg group and the tildrakizumab 200 mg group, respectively. Overall, the frequencies of SAEs by SOC were low (<50%) and similar between the two treatment groups. The most frequently reported SAEs by SOC were: Infections and infestations SOC (14 [3.7%] subjects and 11 [3.2%] subjects in the tildrakizumab 100 mg group and the tildrakizumab 200 mg group, respectively) and Neoplasms benign, malignant and unspecified (incl cysts and polyps) SOC (12 [3.1%] subjects and 16 [4.6%] subjects in the tildrakizumab 100 mg group and the tildrakizumab 200 mg group, respectively).

TABLE 34
Subjects with Serious Adverse Events (All Subjects
as Treated - Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more serious adverse events | 87 | (22.8) | 79 | (22.6) | 166 | (22.7) |
| With no serious adverse events | 294 | (77.2) | 270 | (77.4) | 564 | (77.3) |
| Blood and lymphatic system disorders | 1 | (0.3) | 1 | (0.3) | 2 | (0.3) |
| Anaemia | 1 | (0.3) | 1 | (0.3) | 2 | (0.3) |
| Cardiac disorders | 7 | (1.8) | 9 | (2.6) | 16 | (2.2) |
| Acute myocardial infarction | 1 | (0.3) | 3 | (0.9) | 4 | (0.5) |
| Atrial fibrillation | 1 | (0.3) | 1 | (0.3) | 2 | (0.3) |
| Atrial tachycardia | 1 | (0.3) | 0 | | 1 | (0.1) |
| Atrioventricular block second degree | 1 | (0.3) | 0 | | 1 | (0.1) |
| Cardiac failure congestive | 0 | | 1 | (0.3) | 1 | (0.1) |
| Coronary artery disease | 2 | (0.5) | 2 | (0.6) | 4 | (0.5) |
| Coronary artery stenosis | 0 | | 2 | (0.6) | 2 | (0.3) |
| Myocardial infarction | 1 | (0.3) | 1 | (0.3) | 2 | (0.3) |
| Myocardial ischaemia | 0 | | 1 | (0.3) | 1 | (0.1) |
| Myocarditis | 1 | (0.3) | 0 | | 1 | (0.1) |
| Supraventricular tachycardia | 1 | (0.3) | 0 | | 1 | (0.1) |
| Congenital, familial and genetic disorders | 1 | (0.3) | 0 | | 1 | (0.1) |
| Keratosis follicular | 1 | (0.3) | 0 | | 1 | (0.1) |
| Endocrine disorders | 2 | (0.5) | 0 | | 2 | (0.3) |
| Goitre | 1 | (0.3) | 0 | | 1 | (0.1) |
| Thyrotoxic crisis | 1 | (0.3) | 0 | | 1 | (0.1) |
| Eye disorders | 0 | | 3 | (0.9) | 3 | (0.4) |
| Cataract | 0 | | 2 | (0.6) | 2 | (0.3) |
| Optic ischaemic neuropathy | 0 | | 1 | (0.3) | 1 | (0.1) |
| Gastrointestinal disorders | 8 | (2.1) | 7 | (2.0) | 15 | (2.1) |
| Abdominal pain upper | 1 | (0.3) | 0 | | 1 | (0.1) |
| Diverticulum intestinal | 0 | | 1 | (0.3) | 1 | (0.1) |
| Duodenal polyp | 0 | | 1 | (0.3) | 1 | (0.1) |
| Duodenal ulcer haemorrhage | 1 | (0.3) | 0 | | 1 | (0.1) |
| Enterocolitis | 1 | (0.3) | 0 | | 1 | (0.1) |
| Gastric perforation | 0 | | 1 | (0.3) | 1 | (0.1) |
| Gastric ulcer haemorrhage | 0 | | 1 | (0.3) | 1 | (0.1) |
| Gastritis erosive | 1 | (0.3) | 0 | | 1 | (0.1) |
| Hiatus hernia | 1 | (0.3) | 0 | | 1 | (0.1) |
| Inguinal hernia | 1 | (0.3) | 1 | (0.3) | 2 | (0.3) |
| Large intestine polyp | 0 | | 1 | (0.3) | 1 | (0.1) |
| Malocclusion | 0 | | 1 | (0.3) | 1 | (0.1) |
| Mesenteric artery thrombosis | 1 | (0.3) | 0 | | 1 | (0.1) |
| Pancreatitis acute | 1 | (0.3) | 0 | | 1 | (0.1) |
| Umbilical hernia | 1 | (0.3) | 1 | (0.3) | 2 | (0.3) |
| General disorders and administration site | 4 | (1.0) | 1 | (0.3) | 5 | (0.7) |
| conditions | | | | | | |
| Chest pain | 1 | (0.3) | 0 | | 1 | (0.1) |
| Death | 1 | (0.3) | 0 | | 1 | (0.1) |
| Impaired healing | 1 | (0.3) | 0 | | 1 | (0.1) |
| Non-cardiac chest pain | 1 | (0.3) | 1 | (0.3) | 2 | (0.3) |
| Hepatobiliary disorders | 5 | (1.3) | 0 | | 5 | (0.7) |
| Cholecystitis | 1 | (0.3) | 0 | | 1 | (0.1) |
| Cholelithiasis | 4 | (1.0) | 0 | | 4 | (0.5) |
| Infections and infestations | 14 | (3.7) | 11 | (3.2) | 25 | (3.4) |
| Abdominal wall abscess | 1 | (0.3) | 0 | | 1 | (0.1) |
| Appendicitis | 3 | (0.8) | 2 | (0.6) | 5 | (0.7) |
| Cellulitis | 1 | (0.3) | 0 | | 1 | (0.1) |
| Chronic sinusitis | 0 | | 1 | (0.3) | 1 | (0.1) |
| Device related sepsis | 0 | | 1 | (0.3) | 1 | (0.1) |
| Diverticulitis | 3 | (0.8) | 2 | (0.6) | 5 | (0.7) |
| Epididymitis | 1 | (0.3) | 0 | | 1 | (0.1) |
| Erysipelas | 1 | (0.3) | 0 | | 1 | (0.1) |
| Gastroenteritis bacterial | 0 | | 1 | (0.3) | 1 | (0.1) |
| Infected dermal cyst | 0 | | 1 | (0.3) | 1 | (0.1) |
| Large intestine infection | 0 | | 1 | (0.3) | 1 | (0.1) |
| Meningitis viral | 0 | | 1 | (0.3) | 1 | (0.1) |
| Otitis media acute | 0 | | 1 | (0.3) | 1 | (0.1) |
| Peritonitis | 0 | | 1 | (0.3) | 1 | (0.1) |
| Pneumonia | 2 | (0.5) | 1 | (0.3) | 3 | (0.4) |
| Pneumonia mycoplasmal | 0 | | 1 | (0.3) | 1 | (0.1) |
| Scrotal abscess | 1 | (0.3) | 0 | | 1 | (0.1) |
| Sinusitis | 0 | | 1 | (0.3) | 1 | (0.1) |
| Tonsillitis | 1 | (0.3) | 0 | | 1 | (0.1) |
| Viral infection | 1 | (0.3) | 0 | | 1 | (0.1) |
| Injury, poisoning and procedural | 12 | (3.1) | 10 | (2.9) | 22 | (3.0) |
| complications | | | | | | |
| Bone contusion | 0 | | 1 | (0.3) | 1 | (0.1) |
| Chest injury | 1 | (0.3) | 0 | | 1 | (0.1) |
| Facial bones fracture | 0 | | 1 | (0.3) | 1 | (0.1) |
| Foot fracture | 1 | (0.3) | 0 | | 1 | (0.1) |
| Humerus fracture | 1 | (0.3) | 0 | | 1 | (0.1) |
| Incisional hernia | 1 | (0.3) | 0 | | 1 | (0.1) |
| Ligament rupture | 0 | | 1 | (0.3) | 1 | (0.1) |
| Limb injury | 0 | | 1 | (0.3) | 1 | (0.1) |
| Lower limb fracture | 1 | (0.3) | 0 | | 1 | (0.1) |
| Meniscus injury | 0 | | 2 | (0.6) | 2 | (0.3) |
| Multiple fractures | 0 | | 1 | (0.3) | 1 | (0.1) |
| Multiple injuries | 1 | (0.3) | 0 | | 1 | (0.1) |
| Patella fracture | 0 | | 1 | (0.3) | 1 | (0.1) |
| Procedural haemorrhage | 1 | (0.3) | 0 | | 1 | (0.1) |
| Procedural pain | 1 | (0.3) | 0 | | 1 | (0.1) |
| Skin laceration | 1 | (0.3) | 0 | | 1 | (0.1) |
| Tendon rupture | 1 | (0.3) | 1 | (0.3) | 2 | (0.3) |
| Thoracic vertebral fracture | 0 | | 1 | (0.3) | 1 | (0.1) |
| Toxicity to various agents | 1 | (0.3) | 0 | | 1 | (0.1) |
| Traumatic intracranial haematoma | 1 | (0.3) | 0 | | 1 | (0.1) |
| Wrist fracture | 1 | (0.3) | 0 | | 1 | (0.1) |
| Investigations | 0 | | 1 | (0.3) | 1 | (0.1) |
| Transaminases increased | 0 | | 1 | (0.3) | 1 | (0.1) |
| Metabolism and nutrition disorders | 2 | (0.5) | 4 | (1.1) | 6 | (0.8) |
| Diabetes mellitus | 0 | | 1 | (0.3) | 1 | (0.1) |
| Obesity | 1 | (0.3) | 1 | (0.3) | 2 | (0.3) |
| Overweight | 0 | | 1 | (0.3) | 1 | (0.1) |
| Tetany | 0 | | 1 | (0.3) | 1 | (0.1) |
| Type 2 diabetes mellitus | 1 | (0.3) | 0 | | 1 | (0.1) |
| Musculoskeletal and connective tissue disorders | 10 | (2.6) | 9 | (2.6) | 19 | (2.6) |
| Bursitis | 0 | | 1 | (0.3) | 1 | (0.1) |
| Intervertebral disc degeneration | 1 | (0.3) | 0 | | 1 | (0.1) |
| Intervertebral disc protrusion | 3 | (0.8) | 2 | (0.6) | 5 | (0.7) |
| Jaw cyst | 0 | | 1 | (0.3) | 1 | (0.1) |
| Osteoarthritis | 1 | (0.3) | 1 | (0.3) | 2 | (0.3) |
| Patellofemoral pain syndrome | 0 | | 1 | (0.3) | 1 | (0.1) |
| Psoriatic arthropathy | 3 | (0.8) | 0 | | 3 | (0.4) |
| Rotator cuff syndrome | 1 | (0.3) | 1 | (0.3) | 2 | (0.3) |
| Spinal pain | 1 | (0.3) | 0 | | 1 | (0.1) |
| Spinal stenosis | 0 | | 1 | (0.3) | 1 | (0.1) |
| Spondylolisthesis | 1 | (0.3) | 1 | (0.3) | 2 | (0.3) |
| Neoplasms benign, malignant and unspecified | 12 | (3.1) | 16 | (4.6) | 28 | (3.8) |
| (incl cysts and polyps) | | | | | | |
| Basal cell carcinoma | 3 | (0.8) | 4 | (1.1) | 7 | (1.0) |
| Bladder cancer | 1 | (0.3) | 0 | | 1 | (0.1) |
| Bladder transitional cell carcinoma | 0 | | 1 | (0.3) | 1 | (0.1) |
| Bowen's disease | 1 | (0.3) | 0 | | 1 | (0.1) |
| Breast cancer metastatic | 0 | | 1 | (0.3) | 1 | (0.1) |
| Colon cancer | 0 | | 1 | (0.3) | 1 | (0.1) |
| Enchondromatosis | 0 | | 1 | (0.3) | 1 | (0.1) |
| Hepatic adenoma | 1 | (0.3) | 0 | | 1 | (0.1) |
| Lipoma | 1 | (0.3) | 1 | (0.3) | 2 | (0.3) |
| Malignant melanoma | 0 | | 1 | (0.3) | 1 | (0.1) |
| Malignant melanoma in situ | 0 | | 1 | (0.3) | 1 | (0.1) |
| Ovarian cancer | 1 | (0.3) | 0 | | 1 | (0.1) |
| Prostate cancer | 0 | | 1 | (0.3) | 1 | (0.1) |
| Rectal adenocarcinoma | 1 | (0.3) | 2 | (0.6) | 3 | (0.4) |
| Renal cell carcinoma | 1 | (0.3) | 0 | | 1 | (0.1) |
| Salivary gland adenoma | 0 | | 1 | (0.3) | 1 | (0.1) |
| Testicular neoplasm | 0 | | 1 | (0.3) | 1 | (0.1) |
| Uterine leiomyoma | 2 | (0.5) | 0 | | 2 | (0.3) |
| Nervous system disorders | 9 | (2.4) | 8 | (2.3) | 17 | (2.3) |
| Carotid artery stenosis | 1 | (0.3) | 0 | | 1 | (0.1) |
| Carpal tunnel syndrome | 0 | | 1 | (0.3) | 1 | (0.1) |
| | 00 | | | | | |
| Cerebral ischaemia | 0 | | 1 | (0.3) | 1 | (0.1) |
| Cerebrovascular accident | 2 | (0.5) | 1 | (0.3) | 3 | (0.4) |
| Dizziness | 0 | | 1 | (0.3) | 1 | (0.1) |
| Epilepsy | 1 | (0.3) | 0 | | 1 | (0.1) |
| Facial paresis | 0 | | 1 | (0.3) | 1 | (0.1) |
| Headache | 0 | | 1 | (0.3) | 1 | (0.1) |
| Intensive care unit acquired weakness | 0 | | 1 | (0.3) | 1 | (0.1) |
| Intracranial aneurysm | 1 | (0.3) | 0 | | 1 | (0.1) |
| Ischaemic cerebral infarction | 0 | | 1 | (0.3) | 1 | (0.1) |
| Ischaemic stroke | 2 | (0.5) | 0 | | 2 | (0.3) |
| Lumbar radiculopathy | 1 | (0.3) | 0 | | 1 | (0.1) |
| Radiculopathy | 1 | (0.3) | 0 | | 1 | (0.1) |
| Syncope | 1 | (0.3) | 1 | (0.3) | 2 | (0.3) |
| Pregnancy, puerperium and perinatal | 1 | (0.3) | 0 | | 1 | (0.1) |
| conditions | | | | | | |
| Gestational diabetes | 1 | (0.3) | 0 | | 1 | (0.1) |
| Product issues | 0 | | 1 | (0.3) | 1 | (0.1) |
| Device dislocation | 0 | | 1 | (0.3) | 1 | (0.1) |
| Psychiatric disorders | 7 | (1.8) | 1 | (0.3) | 8 | (1.1) |
| Alcohol use disorder | 1 | (0.3) | 0 | | 1 | (0.1) |
| Alcoholism | 1 | (0.3) | 0 | | 1 | (0.1) |
| Completed suicide | 2 | (0.5) | 0 | | 2 | (0.3) |
| Depression | 2 | (0.5) | 1 | (0.3) | 3 | (0.4) |
| Drug dependence | 1 | (0.3) | 0 | | 1 | (0.1) |
| Insomnia | 1 | (0.3) | 0 | | 1 | (0.1) |
| Renal and urinary disorders | 2 | (0.5) | 6 | (1.7) | 8 | (1.1) |
| Acute kidney injury | 1 | (0.3) | 0 | | 1 | (0.1) |
| Calculus urinary | 2 | (0.5) | 0 | | 2 | (0.3) |
| Cystitis noninfective | 0 | | 1 | (0.3) | 1 | (0.1) |
| Nephrolithiasis | 1 | (0.3) | 3 | (0.9) | 4 | (0.5) |
| Renal cyst | 0 | | 1 | (0.3) | 1 | (0.1) |
| Renal infarct | 0 | | 1 | (0.3) | 1 | (0.1) |
| Reproductive system and breast disorders | 2 | (0.5) | 2 | (0.6) | 4 | (0.5) |
| Adenomyosis | 1 | (0.3) | 0 | | 1 | (0.1) |
| Benign prostatic hyperplasia | 1 | (0.3) | 0 | | 1 | (0.1) |
| Uterine haemorrhage | 0 | | 1 | (0.3) | 1 | (0.1) |
| Uterovaginal prolapse | 0 | | 1 | (0.3) | 1 | (0.1) |
| Respiratory, thoracic and mediastinal | 5 | (1.3) | 5 | (1.4) | 10 | (1.4) |
| disorders | | | | | | |
| Asphyxia | 0 | | 1 | (0.3) | 1 | (0.1) |
| Asthma | 0 | | 1 | (0.3) | 1 | (0.1) |
| Chronic obstructive pulmonary disease | 0 | | 1 | (0.3) | 1 | (0.1) |
| Haemothorax | 1 | (0.3) | 0 | | 1 | (0.1) |
| Laryngeal granuloma | 1 | (0.3) | 0 | | 1 | (0.1 |
| Nasal septum deviation | 1 | (0.3) | 1 | (0.3) | 2 | (0.3) |
| Nasal turbinate hypertrophy | 0 | | 1 | (0.3) | 1 | (0.1) |
| Pneumothorax spontaneous | 1 | (0.3) | 0 | | 1 | (0.1) |
| Pulmonary embolism | 1 | (0.3) | 0 | | 1 | (0.1) |
| Respiratory tract irritation | 0 | | 1 | (0.3) | 1 | (0.1) |
| Skin and subcutaneous tissue disorders | 3 | (0.8) | 0 | | 3 | (0.4) |
| Drug eruption | 1 | (0.3) | 0 | | 1 | (0.1) |
| Psoriasis | 2 | (0.5) | 0 | | 2 | (0.3) |
| Surgical and medical procedures | 0 | | 1 | (0.3) | 1 | (0.1) |
| High frequency ablation | 0 | | 1 | (0.3) | 1 | (0.1) |
| Vascular disorders | 6 | (1.6) | 5 | (1.4) | 11 | (1.5) |
| Deep vein thrombosis | 1 | (0.3) | 1 | (0.3) | 2 | (0.3) |
| Haematoma | 1 | (0.3) | 0 | | 1 | (0.1) |
| Hypertension | 0 | | 1 | (0.3) | 1 | (0.1) |
| Hypertensive crisis | 1 | (0.3) | 1 | (0.3) | 2 | (0.3) |
| Peripheral arterial occlusive disease | 2 | (0.5) | 1 | (0.3) | 3 | (0.4) |
| Varicose vein | 2 | (0.5) | 1 | (0.3) | 3 | (0.4) |

^{†}Subjects who took at least one dose of extension study medication based on the treatment actually received. Subjects were counted only once in the overall category. A subject may appear in different categories. MedDRA Version: Merck T2-MedDRA.0 and MedDRA24.0

No notable differences in the frequencies of SAEs were observed between subjects treated with PFS and those treated with AI during extension study. Overall, the frequencies of SAEs by SOC were low (<3%), and the most frequently reported SAE by SOC was Infections and infestations SOC in subjects treated with PFS (12 [1.6%]) and Neoplasms benign, malignant and unspecified (incl cysts and polyps) in subjects treated with AI (20 [2.7%]). No notable differences in the 5-year exposure-adjusted frequencies of exposure-adjusted summary of subjects with SAEs were reported during base and extension study. SAEs were observed between the two tildrakizumab treatment groups.

A summary of serious drug-related AEs reported during extension study is provided in Table 35. The overall frequency of serious drug-related AEs during the extension study was low (1.9%) and similar between the treatment groups (8 [2.1%] subjects and 6 [1.7%] subjects in the tildrakizumab 100 mg group and the tildrakizumab 200 mg group, respectively).

TABLE 35
Subjects with Serious Drug-Related Adverse Events (All
Subjects as Treated -Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more serious drug-related | 8 | (2.1) | 6 | (1.7) | 14 | (1.9) |
| adverse | | | | | | |
| events | | | | | | |
| With no serious drug-related adverse events | 373 | (97.9) | 343 | (98.3) | 716 | (98.1) |
| Cardiac disorders | 1 | (0.3) | 0 | | 1 | (0.1) |
| Myocarditis | 1 | (0.3) | 0 | | 1 | (0.1) |
| Endocrine disorders | 1 | (0.3) | 0 | | 1 | (0.1) |
| Thyrotoxic crisis | 1 | (0.3) | 0 | | 1 | (0.1) |
| Gastrointestinal disorders | 1 | (0.3) | 0 | | 1 | (0.1) |
| Mesenteric artery thrombosis | 1 | (0.3) | 0 | | 1 | (0.1) |
| Infections and infestations | 3 | (0.8) | 4 | (1.1) | 7 | (1.0) |
| Appendicitis | 1 | (0.3) | 1 | (0.3) | 2 | (0.3) |
| Diverticulitis | 1 | (0.3) | 0 | | 1 | (0.1) |
| Large intestine infection | 0 | | 1 | (0.3) | 1 | (0.1) |
| Meningitis viral | 0 | | 1 | (0.3) | 1 | (0.1) |
| Peritonitis | 0 | | 1 | (0.3) | 1 | (0.1) |
| Pneumonia mycoplasmal | 0 | | 1 | (0.3) | 1 | (0.1) |
| Tonsillitis | 1 | (0.3) | 0 | | 1 | (0.1) |
| Neoplasms benign, malignant and | 1 | (0.3) | 1 | (0.3) | 2 | (0.3) |
| unspecified | | | | | | |
| (incl cysts and polyps) | | | | | | |
| Basal cell carcinoma | 0 | | 1 | (0.3) | 1 | (0.1) |
| Rectal adenocarcinoma | 1 | (0.3) | 0 | | 1 | (0.1) |
| Respiratory, thoracic and mediastinal | 0 | | 1 | (0.3) | 1 | (0.1) |
| disorders | | | | | | |
| Chronic obstructive pulmonary disease | 0 | | 1 | (0.3) | 1 | (0.1) |
| Vascular disorders | 1 | (0.3) | 0 | | 1 | (0.1) |
| Hypertensive crisis | 1 | (0.3) | 0 | | 1 | (0.1) |

^{†}Subjects who took at least one dose of extension study medication based on the treatment actually received. Subjects were counted only once in the overall category. A subject may appear in different categories. MedDRA Version: Merck T2-MedDRA.0 and MedDRA24.0

No notable differences were observed in the 5-year cumulative exposure-adjusted frequencies of serious drug-related AEs between the two tildrakizumab treatment groups.

### Discontinuations Due to Adverse Events

### Adverse Events Resulting in Discontinuation of Study Medication

A summary of AEs that led to discontinuation from study medication reported during the extension study is provided by treatment group, SOC, and PT in Table 36. The frequencies of AEs that led to discontinuation of study medication were similar in the two treatment groups (29 [4.0%] overall, 16 [4.2%] subjects and 13 [3.7%] subjects in the tildrakizumab 100 mg group and the tildrakizumab 200 mg group, respectively).

TABLE 36
Subjects with Adverse Events Resulting in Discontinuation of Study
Medication (All Subjects as Treated - Extension) Extension Study

| | MK-3222 100 mg | | MK-3222 200 mg | | Total | |
|---|---|---|---|---|---|---|
| | n | (%) | n | (%) | n | (%) |
| Subjects in population^{†} | 381 | | 349 | | 730 | |
| With one or more adverse events | 16 | (4.2) | 13 | (3.7) | 29 | (4.0) |
| resulting in discontinuation of | | | | | | |
| study medication | | | | | | |
| With no adverse events resulting | 365 | (95.8) | 336 | (96.3) | 701 | (96.0) |
| in discontinuation of study | | | | | | |
| medication | | | | | | |
| Endocrine disorders | 1 | (0.3) | 0 | | 1 | (0.1) |
| Hyperthyroidism | 1 | (0.3) | 0 | | 1 | (0.1) |
| Thyrotoxic crisis | 1 | (0.3) | 0 | | 1 | (0.1) |
| Eye disorders | 0 | | 1 | (0.3) | 1 | (0.1) |
| Optic ischaemic neuropathy | 0 | | 1 | (0.3) | 1 | (0.1) |
| Gastrointestinal disorders | 1 | (0.3) | 0 | | 1 | (0.1) |
| Dysphagia | 1 | (0.3) | 0 | | 1 | (0.1) |
| Infections and infestations | 1 | (0.3) | 2 | (0.6) | 3 | (0.4) |
| Bronchitis | 0 | | 1 | (0.3) | 1 | (0.1) |
| Diverticulitis | 1 | (0.3) | 0 | | 1 | (0.1) |
| Nasopharyngitis | 0 | | 1 | (0.3) | 1 | (0.1) |
| Investigations | 1 | (0.3) | 0 | | 1 | (0.1) |
| Aspartate aminotransferase | 1 | (0.3) | 0 | | 1 | (0.1) |
| increased | | | | | | |
| Musculoskeletal and connective | 6 | (1.6) | 1 | (0.3) | 7 | (1.0) |
| tissue disorders | | | | | | |
| Arthritis | 1 | (0.3) | 1 | (0.3) | 2 | (0.3) |
| Psoriatic arthropathy | 5 | (1.3) | 0 | | 5 | (0.7) |
| Neoplasms benign, malignant | 2 | (0.5) | 5 | (1.4) | 7 | (1.0) |
| and unspecified (incl cysts and | | | | | | |
| polyps) | | | | | | |
| Breast cancer metastatic | 0 | | 1 | (0.3) | 1 | (0.1) |
| Colon cancer | 0 | | 1 | (0.3) | 1 | (0.1) |
| Malignant melanoma | 0 | | 1 | (0.3) | 1 | (0.1) |
| Ovarian cancer | 1 | (0.3) | 0 | | 1 | (0.1) |
| Prostate cancer | 0 | | 1 | (0.3) | 1 | (0.1) |
| Rectal adenocarcinoma | 0 | | 1 | (0.3) | 1 | (0.1) |
| Renal cell carcinoma | 1 | (0.3) | 0 | | 1 | (0.1) |
| Psychiatric disorders | 1 | (0.3) | 0 | | 1 | (0.1) |
| Completed suicide | 1 | (0.3) | 0 | | 1 | (0.1) |
| Respiratory, thoracic and | 0 | | 2 | (0.6) | 2 | (0.3) |
| mediastinal disorders | | | | | | |
| Asphyxia | 0 | | 1 | (0.3) | 1 | (0.1) |
| Chronic obstructive pulmonary | 0 | | 1 | (0.3) | 1 | (0.1) |
| disease | | | | | | |
| Skin and subcutaneous tissue | 3 | (0.8) | 3 | (0.9) | 6 | (0.8) |
| disorders | | | | | | |
| Eczema | 1 | (0.3) | 0 | | 1 | (0.1) |
| Psoriasis | 2 | (0.5) | 3 | (0.9) | 5 | (0.7) |

^{†}Subjects who took at least one dose of extension study medication based on the treatment actually received. Subjects were counted only once in the overall category. A subject may appear in different categories. A system organ class or specific adverse event appears on this report only if its incidence in one or more of the columns is greater than or equal to the percent incidence specified in the report title, after rounding. MedDRA Version: Merck T2-MedDRA.0 and MedDRA24.0

No notable difference in the frequencies of AEs resulting in discontinuation of study medication by SOC, and PT were observed between subjects treated with PFS and those treated with AI during the extension study. A 5-year cumulative exposure-adjusted summary of subjects with AEs resulting in discontinuation of study medication.

### Drug-Related Adverse Events Resulting in Discontinuation of Study Medication

The frequencies of drug related AEs that led to discontinuation of study medication were similar in the two treatment groups (7 [1.0%] subjects overall, 4 [1.0%] subjects and 3 [0.9%] subjects in the tildrakizumab 100 mg group and the tildrakizumab 200 mg group, respectively). A 5-year cumulative exposure-adjusted summary of subjects with drug-related AEs resulting in discontinuation of study medication. No notable differences were observed in the 5-year cumulative exposure adjusted-frequencies of drug-related AEs resulting in discontinuation of study medication between the two tildrakizumab treatment groups.

### Other Adverse Events of Special Interest

### Tier 1 Adverse Events

An overall summary of Tier 1 AEs reported during the extension study was provided by treatment group in Table 37.

TABLE 37
Subjects with Tier 1 Adverse Events (All Subjects
as Treated-Extension) Extension Study

| | MK-3222 100 mg | | MK-3222 200 mg | | Total | |
|---|---|---|---|---|---|---|
| | n | (%) | n | (%) | n | (%) |
| Subjects in population^{†} | 381 | | 349 | | 730 | |
| With Severe Infections^{‡} | 15 | (3.9) | 11 | (3.2) | 26 | (3.6) |
| With Malignancies^{§} | 9 | (2.4) | 13 | (3.7) | 22 | (3.0) |
| With Non-Melanoma Skin Cancer | 5 | (1.3) | 4 | (1.1) | 9 | (1.2) |
| With Melanoma Skin Cancer | 0 | | 2 | (0.6) | 2 | (0.3) |
| With Confirmed Extended MACE^{\|\|} | 7 | (1.8) | 9 | (2.6) | 16 | (2.2) |
| With Drug Related Hypersensitivity | 1 | (0.3) | 2 | (0.6) | 3 | (0.4) |
| Reactions | | | | | | |

^{†}Subjects who took at least one dose of extension study medication based on the treatment actually received.
^{‡}Defined as infection meeting the regulatory definition of a serious adverse event, or any infection requiring IV antibiotics whether or not reported as a serious adverse event, as per the regulatory definition.
^{§}Excluding carcinoma in situ of the cervix.
^{||}Includes non-fatal myocardial infarction, non-fatal stroke, unstable angina, coronary revascularization, resuscitated cardiac arrest, and CV deaths that are confirmed as “cardiovascular” or “sudden”. MACE = Major Adverse Cardiovascular Event.

No meaningful differences were observed in the frequencies of each pre-specified Tier 1 AE between the two treatment groups. The most frequently reported Tier 1 AE was Severe Infections (15 [3.9%] subjects and 11 [3.2%] subjects in the tildrakizumab 100 mg group and the tildrakizumab 200 mg group, respectively), followed by Malignancies (9 [2.4%] subjects and 13 [3.7%] subjects in the tildrakizumab 100 mg group and the tildrakizumab 200 mg group, respectively).

No meaningful differences in the frequencies of Tier 1 AEs were observed between subjects who self-injected and those who did not self-inject at Week 52 during the extension study. A 5-year cumulative exposure-adjusted summary of subjects with Tier 1 AEs during the base study and the extension study found no differences in the adjusted frequencies of Tier 1 AEs between the two tildrakizumab treatment groups.

### Tier 2 Adverse Events

An overall summary of the Tier 2 AEs reported during the extension study is provided by treatment group in Table 38. The frequencies of Tier 2 AEs or drug-related Tier 2 AEs were higher in the tildrakizumab 200 mg group (307 [88.0%] subjects and 99 [28.4%] subjects, respectively) compared to the tildrakizumab 100 mg group (321 [84.3%] subjects and 91 [23.9%] subjects, respectively). The frequency of Tier 2 AEs among subjects with treatment-emergent ADA was higher in the tildrakizumab 100 mg group (32 [8.4%] subjects) compared to the tildrakizumab 200 mg group (18 [5.2%] subjects). No meaningful differences in the frequencies of each pre-specified Tier 2 AE were observed between the two treatment groups.

Across the two treatment groups, 84 (11.5%) subjects with ADA reported any AEs (50 [6.8%] subjects and 34 [4.7%] subjects with treatment-emergent or non-treatment emergent ADA, respectively) while 420 (57.5%) subjects with negative for ADA and 124 (17.0%) subjects with inconclusive for ADA reported any AEs. No correlation of ADA incidence with frequencies of AEs was observed.

A 5-year cumulative exposure-adjusted summary of subjects with Tier 2 AEs during the base study and the extension study found no notable differences in the 5-year cumulative exposure-adjusted frequencies of Tier 2 AEs between the two tildrakizumab treatment group.

TABLE 38
Subjects with Tier 2 Adverse Events (All Subjects as Treated -Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more adverse events | 321 | (84.3) | 307 | (88.0) | 628 | (86.0) |
| With drug-related adverse event | 91 | (23.9) | 99 | (28.4) | 190 | (26.0) |
| With serious adverse event | 87 | (22.8) | 79 | (22.6) | 166 | (22.7) |
| Discontinued^{‡} due to an adverse event | 16 | (4.2) | 13 | (3.7) | 29 | (4.0) |
| Discontinued^{‡} due to drug-related adverse event | 4 | (1.0) | 3 | (0.9) | 7 | (1.0) |
| Who died | 4 | (1.0) | 2 | (0.6) | 6 | (0.8) |
| With adverse events and treatment emergent | 32 | (8.4) | 18 | (5.2) | 50 | (6.8) |
| ADA | | | | | | |
| With adverse events and non-treatment emergent | 13 | (3.4) | 21 | (6.0) | 34 | (4.7) |
| ADA | | | | | | |
| With adverse events and negative for ADA | 220 | (57.7) | 200 | (57.3) | 420 | (57.5) |
| With adverse events and inconclusive for ADA | 56 | (14.7) | 68 | (19.5) | 124 | (17.0) |
| With adverse events and anti-drug antibodies | 45 | (11.8) | 39 | (11.2) | 84 | (11.5) |
| With adverse event not including worsening of | 320 | (84.0) | 306 | (87.7) | 626 | (85.8) |
| psoriasis^{§} | | | | | | |
| With confirmed MACE | 7 | (1.8) | 9 | (2.6) | 16 | (2.2) |
| With confirmed cardiovascular adverse events^{\|\|} | 9 | (2.4) | 10 | (2.9) | 19 | (2.6) |

^{†}Subjects who took at least one dose of extension study medication based on the treatment actually received.
^{‡}Discontinued study medication.
^{§}Unless it meets criteria for serious adverse event.
^{||}Thrombotic/Embolic/Ischemic cardiovascular events (including MACE and Extended MACE). MACE = Major Adverse Cardiovascular Event.

Adverse Events in Subjects with Anti-Drug Antibodies

A summary of AEs reported in subjects with treatment-emergent ADA is provided by treatment group in Table 39. Of 56 subjects with treatment-emergent ADA in the extension study, 50 (89.3%) subjects reported one or more AEs (32 [91.4%] subjects and 18 [85.7%] subjects in the tildrakizumab 100 mg group and the tildrakizumab 200 mg group, respectively).

TABLE 39
Specific Adverse Events for Subjects with Treatment Emergent Anti-
Drug Antibodies (All Subjects as Treated - Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 35 | | 21 | | 56 | |
| With one or more specific adverse events and anti-drug | 32 | (91.4) | 18 | (85.7) | 50 | (89.3) |
| antibodies | | | | | | |
| With no specific adverse events and anti-drug antibodies | 3 | (8.6) | 3 | (14.3) | 6 | (10.7) |
| Blood and lymphatic system disorders | 2 | (5.7) | 0 | | 2 | (3.6) |
| Iron deficiency anaemia | 1 | (2.9) | 0 | | 1 | (1.8) |
| Polycythaemia | 1 | (2.9) | 0 | | 1 | (1.8) |
| Cardiac disorders | 3 | (8.6) | 0 | | 3 | (5.4) |
| Atrial fibrillation | 1 | (2.9) | 0 | | 1 | (1.8) |
| Atrioventricular block first degree | 1 | (2.9) | 0 | | 1 | (1.8) |
| Coronary artery disease | 1 | (2.9) | 0 | | 1 | (1.8) |
| Ear and labyrinth disorders | 1 | (2.9) | 1 | (4.8) | 2 | (3.6) |
| Ear pain | 1 | (2.9) | 0 | | 1 | (1.8) |
| Vertigo | 0 | | 1 | (4.8) | 1 | (1.8) |
| Endocrine disorders | 2 | (5.7) | 0 | | 2 | (3.6) |
| Hypothyroidism | 2 | (5.7) | 0 | | 2 | (3.6) |
| Eye disorders | 1 | (2.9) | 2 | (9.5) | 3 | (5.4) |
| Dry eye | 0 | | 1 | (4.8) | 1 | (1.8) |
| Epiretinal membrane | 1 | (2.9) | 0 | | 1 | (1.8) |
| Retinal detachment | 1 | (2.9) | 0 | | 1 | (1.8) |
| Vitreous degeneration | 0 | | 1 | (4.8) | 1 | (1.8) |
| Gastrointestinal disorders | 9 | (25.7) | 6 | (28.6) | 15 | (26.8) |
| Abdominal pain | 0 | | 1 | (4.8) | 1 | (1.8) |
| Abdominal pain lower | 0 | | 1 | (4.8) | 1 | (1.8) |
| Abdominal pain upper | 1 | (2.9) | 1 | (4.8) | 2 | (3.6) |
| Colitis | 0 | | 1 | (4.8) | 1 | (1.8) |
| Constipation | 1 | (2.9) | 0 | | 1 | (1.8) |
| Dental caries | 0 | | 1 | (4.8) | 1 | (1.8) |
| Diarrhoea | 4 | (11.4) | 4 | (19.0) | 8 | (14.3) |
| Duodenal ulcer | 1 | (2.9) | 0 | | 1 | (1.8) |
| Duodenal ulcer haemorrhage | 1 | (2.9) | 0 | | 1 | (1.8) |
| Dyspepsia | 1 | (2.9) | 0 | | 1 | (1.8) |
| Dysphagia | 1 | (2.9) | 0 | | 1 | (1.8) |
| Gastritis | 1 | (2.9) | 0 | | 1 | (1.8) |
| Gastrooesophageal reflux disease | 0 | | 1 | (4.8) | 1 | (1.8) |
| Oesophagitis | 1 | (2.9) | 0 | | 1 | (1.8) |
| Reflux gastritis | 1 | (2.9) | 0 | | 1 | (1.8) |
| Vomiting | 0 | | 1 | (4.8) | 1 | (1.8) |
| General disorders and administration site conditions | 2 | (5.7) | 3 | (14.3) | 5 | (8.9) |
| Asthenia | 1 | (2.9) | 0 | | 1 | (1.8) |
| Chest pain | 0 | | 1 | (4.8) | 1 | (1.8) |
| Cyst | 1 | (2.9) | 0 | | 1 | (1.8) |
| Injection site erythema | 0 | | 1 | (4.8) | 1 | (1.8) |
| Injection site haematoma | 0 | | 1 | (4.8) | 1 | (1.8) |
| Injection site pain | 0 | | 2 | (9.5) | 2 | (3.6) |
| Hepatobiliary disorders | 0 | | 1 | (4.8) | 1 | (1.8) |
| Hepatomegaly | 0 | | 1 | (4.8) | 1 | (1.8) |
| Immune system disorders | 1 | (2.9) | 0 | | 1 | (1.8) |
| Mite allergy | 1 | (2.9) | 0 | | 1 | (1.8) |
| Infections and infestations | 21 | (60.0) | 12 | (57.1) | 33 | (58.9) |
| Abdominal wall abscess | 1 | (2.9) | 0 | | 1 | (1.8) |
| Acute sinusitis | 0 | | 1 | (4.8) | 1 | (1.8) |
| Appendicitis | 1 | (2.9) | 0 | | 1 | (1.8) |
| Bronchitis | 3 | (8.6) | 4 | (19.0) | 7 | (12.5) |
| Conjunctivitis | 0 | (2.9) | 1 | (4.8) | 1 | (1.8) |
| Cystitis | 1 | (2.9) | 0 | | 1 | (1.8) |
| Ear infection | 1 | (2.9) | 0 | | 1 | (1.8) |
| Fungal infection | 0 | | 1 | (4.8) | 1 | (1.8) |
| Furuncle | 1 | (2.9) | 1 | (4.8) | 2 | (3.6) |
| Gangrene | 1 | (2.9) | 0 | | 1 | (1.8) |
| Gastroenteritis | 2 | (5.7) | 2 | (9.5) | 4 | (7.1) |
| Gastrointestinal infection | 2 | (5.7) | 0 | | 2 | (3.6) |
| Genital candidiasis | 0 | | 1 | (4.8) | 1 | (1.8) |
| Genital herpes | 0 | | 1 | (4.8) | 1 | (1.8) |
| Genitourinary chlamydia infection | 0 | | 1 | (4.8) | 1 | (1.8) |
| Influenza | 0 | | 2 | (9.5) | 2 | (3.6) |
| Laryngitis | 0 | | 1 | (4.8) | 1 | (1.8) |
| Localised infection | 0 | | 1 | (4.8) | 1 | (1.8) |
| Nasopharyngitis | 12 | (34.3) | 5 | (23.8) | 17 | (30.4) |
| Onychomycosis | 0 | | 1 | (4.8) | 1 | (1.8) |
| Oral herpes | 0 | | 2 | (9.5) | 2 | (3.6) |
| Peritonitis | 1 | (2.9) | 0 | | 1 | (1.8) |
| Pneumonia | 1 | (2.9) | 0 | | 1 | (1.8) |
| Pulpitis dental | 1 | (2.9) | 0 | | 1 | (1.8) |
| Respiratory tract infection | 0 | | 1 | (4.8) | 1 | (1.8) |
| Rhinitis | 0 | | 2 | (9.5) | 2 | (3.6) |
| Sinusitis | 2 | (5.7) | 3 | (14.3) | 5 | (8.9) |
| Skin candida | 0 | | 1 | (4.8) | 1 | (1.8) |
| Staphylococcal infection | 1 | (2.9) | 0 | | 1 | (1.8) |
| Tinea pedis | 0 | | 1 | (4.8) | 1 | (1.8) |
| Tooth abscess | 0 | | 1 | (4.8) | 1 | (1.8) |
| Tooth infection | 1 | (2.9) | 1 | (4.8) | 2 | (3.6) |
| Upper respiratory tract infection | 5 | (14.3) | 4 | (19.0) | 9 | (16.1) |
| Upper respiratory tract infection bacterial | 0 | | 1 | (4.8) | 1 | (1.8) |
| Urinary tract infection | 2 | (5.7) | 0 | | 2 | (3.6) |
| Vaginal infection | 0 | | 1 | (4.8) | 1 | (1.8) |
| Vulvovaginal mycotic infection | 0 | | 1 | (4.8) | 1 | (1.8) |
| Wound infection | 1 | (2.9) | 0 | | 1 | (1.8) |
| Wound infection staphylococcal | 1 | (2.9) | 0 | | 1 | (1.8) |
| Injury, poisoning and procedural complications | 7 | (20.0) | 4 | (19.0) | 11 | (19.6) |
| Arthropod bite | 1 | (2.9) | 1 | (4.8) | 2 | (3.6) |
| Chest injury | 1 | (2.9) | 0 | | 1 | (1.8) |
| Clavicle fracture | 1 | (2.9) | 0 | | 1 | (1.8) |
| Humerus fracture | 1 | (2.9) | 0 | | 1 | (1.8) |
| Incisional hernia | 1 | (2.9) | 0 | | 1 | (1.8) |
| Ligament sprain | 0 | | 1 | (4.8) | 1 | (1.8) |
| Limb injury | 0 | | 1 | (4.8) | 1 | (1.8) |
| Procedural pain | 1 | (2.9) | 0 | | 1 | (1.8) |
| Rib fracture | 0 | | 1 | (4.8) | 1 | (1.8) |
| Skin laceration | 1 | (2.9) | 0 | | 1 | (1.8) |
| Tongue injury | 0 | | 1 | (4.8) | 1 | (1.8) |
| Wrist fracture | 1 | (2.9) | 0 | | 1 | (1.8) |
| Investigations | 5 | (14.3) | 4 | (19.0) | 9 | (16.1) |
| Alanine aminotransferase increased | 1 | (2.9) | 0 | | 1 | (1.8) |
| Aspartate aminotransferase increased | 1 | (2.9) | 0 | | 1 | (1.8) |
| Blood creatine phosphokinase increased | 1 | (2.9) | 0 | | 1 | (1.8) |
| Blood thyroid stimulating hormone increased | 0 | | 1 | (4.8) | 1 | (1.8) |
| Bone density decreased | 0 | | 1 | (4.8) | 1 | (1.8) |
| Gamma-glutamyltransferase increased | 1 | (2.9) | 0 | | 1 | (1.8) |
| Heart rate irregular | 0 | | 1 | (4.8) | 1 | (1.8) |
| Intraocular pressure increased | 1 | (2.9) | 0 | | 1 | (1.8) |
| Liver function test increased | 1 | (2.9) | 1 | (4.8) | 2 | (3.6) |
| Protein urine present | 1 | (2.9) | 0 | | 1 | (1.8) |
| Urinary sediment present | 1 | (2.9) | 0 | | 1 | (1.8) |
| Metabolism and nutrition disorders | 5 | (14.3) | 1 | (4.8) | 6 | (10.7) |
| Diabetes mellitus | 1 | (2.9) | 1 | (4.8) | 2 | (3.6) |
| Dyslipidaemia | 1 | (2.9) | 0 | | 1 | (1.8) |
| Gout | 1 | (2.9) | 0 | | 1 | (1.8) |
| Hyperlipidaemia | 1 | (2.9) | 0 | | 1 | (1.8) |
| Iron deficiency | 1 | (2.9) | 0 | | 1 | (1.8) |
| Musculoskeletal and connective tissue disorders | 10 | (28.6) | 12 | (57.1) | 22 | (39.3) |
| Arthralgia | 2 | (5.7) | 6 | (28.6) | 8 | (14.3) |
| Articular calcification | 0 | | 1 | (4.8) | 1 | (1.8) |
| Back pain | 3 | (8.6) | 1 | (4.8) | 4 | (7.1) |
| Chondrodynia | 0 | | 1 | (4.8) | 1 | (1.8) |
| Intervertebral disc protrusion | 0 | | 1 | (4.8) | 1 | (1.8) |
| Joint swelling | 0 | | 1 | (4.8) | 1 | (1.8) |
| Kyphosis | 0 | | 1 | (4.8) | 1 | (1.8) |
| Lumbar spinal stenosis | 1 | (2.9) | 0 | | 1 | (1.8) |
| Muscle contracture | 1 | (2.9) | 0 | | 1 | (1.8) |
| Muscle tightness | 1 | (2.9) | 0 | | 1 | (1.8) |
| Muscular weakness | 0 | | 1 | (4.8) | 1 | (1.8) |
| Myalgia | 0 | | 1 | (4.8) | 1 | (1.8) |
| Neck pain | 0 | | 1 | (4.8) | 1 | (1.8) |
| Osteoarthritis | 1 | (2.9) | 2 | (9.5) | 3 | (5.4) |
| Osteochondrosis | 0 | | 1 | (4.8) | 1 | (1.8) |
| Pain in extremity | 1 | (2.9) | 0 | | 1 | (1.8) |
| Psoriatic arthropathy | 1 | (2.9) | 2 | (9.5) | 3 | (5.4) |
| Spinal disorder | 0 | | 1 | (4.8) | 1 | (1.8) |
| Spinal pain | 0 | | 1 | (4.8) | 1 | (1.8) |
| Synovial cyst | 0 | | 1 | (4.8) | 1 | (1.8) |
| Tenosynovitis stenosans | 0 | | 1 | (4.8) | 1 | (1.8) |
| Neoplasms benign, malignant and unspecified (incl | 4 | (11.4) | 3 | (14.3) | 7 | (12.5) |
| cysts and polyps) | | | | | | |
| Basal cell carcinoma | 1 | (2.9) | 0 | | 1 | (1.8) |
| Dysplastic naevus | 0 | | 1 | (4.8) | 1 | (1.8) |
| Fibrous histiocytoma | 1 | (2.9) | 0 | | 1 | (1.8) |
| Haemangioma | 0 | | 1 | (4.8) | 1 | (1.8) |
| Haemangioma of skin | 0 | | 1 | (4.8) | 1 | (1.8) |
| Rectal adenocarcinoma | 1 | (2.9) | 0 | | 1 | (1.8) |
| Uterine leiomyoma | 1 | (2.9) | 0 | | 1 | (1.8) |
| Nervous system disorders | 5 | (14.3) | 8 | (38.1) | 13 | (23.2) |
| Cerebrovascular accident | 0 | | 1 | (4.8) | 1 | (1.8) |
| Cervical radiculopathy | 0 | | 1 | (4.8) | 1 | (1.8) |
| Cervicobrachial syndrome | 1 | (2.9) | 1 | (4.8) | 2 | (3.6) |
| Complex regional pain syndrome | 0 | | 1 | (4.8) | 1 | (1.8) |
| Dizziness | 0 | | 1 | (4.8) | 1 | (1.8) |
| Epilepsy | 1 | (2.9) | 0 | | 1 | (1.8) |
| Headache | 3 | (8.6) | 5 | (23.8) | 8 | (14.3) |
| Ischaemic stroke | 1 | (2.9) | 0 | | 1 | (1.8) |
| Monoparesis | 0 | | 1 | (4.8) | 1 | (1.8) |
| Paraesthesia | 1 | (2.9) | 0 | | 1 | (1.8) |
| Restless legs syndrome | 1 | (2.9) | 0 | | 1 | (1.8) |
| Sciatica | 1 | (2.9) | 0 | | 1 | (1.8) |
| Psychiatric disorders | 3 | (8.6) | 3 | (14.3) | 6 | (10.7) |
| Anxiety | 1 | (2.9) | 0 | | 1 | (1.8) |
| Depression | 2 | (5.7) | 0 | | 2 | (3.6) |
| Insomnia | 0 | | 3 | (14.3) | 3 | (5.4) |
| Renal and urinary disorders | 4 | (11.4) | 1 | (4.8) | 5 | (8.9) |
| Acute kidney injury | 1 | (2.9) | 0 | | 1 | (1.8) |
| Calculus urinary | 1 | (2.9) | 0 | | 1 | (1.8) |
| Chronic kidney disease | 1 | (2.9) | 0 | | 1 | (1.8) |
| Haematuria | 1 | (2.9) | 0 | | 1 | (1.8) |
| Nephrolithiasis | 0 | | 1 | (4.8) | 1 | (1.8) |
| Urine abnormality | 1 | (2.9) | 0 | | 1 | (1.8) |
| Reproductive system and breast disorders | 0 | | 3 | (14.3) | 3 | (5.4) |
| Cervical dysplasia | 0 | | 1 | (4.8) | 1 | (1.8) |
| Prostatitis | 0 | | 2 | (9.5) | 2 | (3.6) |
| Respiratory, thoracic and mediastinal disorders | 10 | (28.6) | 4 | (19.0) | 14 | (25.0) |
| Asthma | 1 | (2.9) | 1 | (4.8) | 2 | (3.6) |
| Cough | 3 | (8.6) | 0 | | 3 | (5.4) |
| Dysphonia | 1 | (2.9) | 0 | | 1 | (1.8) |
| Dyspnoea | 1 | (2.9) | 0 | | 1 | (1.8) |
| Granulomatous pneumonitis | 0 | | 1 | (4.8) | 1 | (1.8) |
| Haemothorax | 1 | (2.9) | 0 | | 1 | (1.8) |
| Nasal congestion | 0 | | 1 | (4.8) | 1 | (1.8) |
| Oropharyngeal pain | 2 | (5.7) | 1 | (4.8) | 3 | (5.4) |
| Pulmonary embolism | 1 | (2.9) | 0 | | 1 | (1.8) |
| Sleep apnoea syndrome | 1 | (2.9) | 0 | | 1 | (1.8) |
| Skin and subcutaneous tissue disorders | 5 | (14.3) | 3 | (14.3) | 8 | (14.3) |
| Dermatitis allergic | 0 | | 1 | (4.8) | 1 | (1.8) |
| Dermatitis contact | 1 | (2.9) | 0 | | 1 | (1.8) |
| Eczema | 1 | (2.9) | 0 | | 1 | (1.8) |
| Lentigo | 1 | (2.9) | 0 | | 1 | (1.8) |
| Onychoclasis | 1 | (2.9) | 0 | | 1 | (1.8) |
| Pruritus | 1 | (2.9) | 0 | | 1 | (1.8) |
| Psoriasis | 2 | (5.7) | 0 | | 2 | (3.6) |
| Skin mass | 0 | | 1 | (4.8) | 1 | (1.8) |
| Skin ulcer | 0 | | 1 | (4.8) | 1 | (1.8) |
| Vascular disorders | 8 | (22.9) | 3 | (14.3) | 11 | (19.6) |
| Deep vein thrombosis | 1 | (2.9) | 0 | | 1 | (1.8) |
| Essential hypertension | 0 | | 1 | (4.8) | 1 | (1.8) |
| Haematoma | 1 | (2.9) | 0 | | 1 | (1.8) |
| Hypertension | 5 | (14.3) | 1 | (4.8) | 6 | (10.7) |
| Hypotension | 0 | | 1 | (4.8) | 1 | (1.8) |
| Peripheral arterial occlusive disease | 1 | (2.9) | 0 | | 1 | (1.8) |
| Phlebitis | 1 | (2.9) | 0 | | 1 | (1.8) |
| Thrombophlebitis | 1 | (2.9) | 0 | | 1 | (1.8) |

^{†}Subjects with anti-drug antibodies and who took at least one dose of extension study medication based on the treatment actually received. Subjects were counted only once in the overall category. A subject may appear in different categories. A system organ class or specific adverse event appears on this report only if its incidence in one or more of the columns is greater than or equal to the percent incidence specified in the report title, after rounding. MedDRA Version: Merck T2-MedDRA.0 and MedDRA24.0

The most frequently reported AEs by SOC in subjects with treatment-emergent ADA was Infections and infestations SOC (21 [60.0%] subjects and 12 [57.1%] subjects in the tildrakizumab 100 mg group and the tildrakizumab 200 mg group, respectively). The most frequently reported AE by PT was nasopharyngitis (12 [34.3%] subjects and 5 [23.8%] subjects in the tildrakizumab 100 mg group and the tildrakizumab 200 mg group, respectively). AEs by SOC with ≥5 percentage-point higher frequency in subjects with treatment-emergent ADA than subjects in ASaT were Respiratory, thoracic and mediastinal disorders SOC (25.0% vs.17.3%) and Nervous system disorders SOC (23.2% vs.16.8%).

The most frequently reported AEs (≥10%) by PT in subjects with treatment-emergent ADA were nasopharyngitis (30.4%), followed by upper respiratory tract infection (16.1%), diarrhoea (14.3%), arthralgia (14.3%), headache (14.3%), bronchitis (12.5%), and hypertension (10.7%).

AEs by PT reported with ≥5 percentage point higher frequency in subjects with treatment-emergent ADA than subjects in ASaT were headache (14.3% vs. 4.8%), diarrhoea (14.3% vs. 6.3%), upper respiratory tract infection (16.1% vs. 9.5%), and bronchitis (12.5% vs. 7.0%).

Five-year cumulative exposure-adjusted summaries of AEs reported in subjects with treatment-emergent ADA and in subjects with non-treatment-emergent ADA found no notable differences in the 5-year exposure-adjusted frequencies of AEs reported in subjects with treatment-emergent ADA between the two tildrakizumab treatment groups.

Adverse Events in Subjects Who were Negative/Inconclusive for Anti-Drug Antibodies

AEs by SOC with ≥5 percentage-point higher frequency in subjects with treatment-emergent ADA positive (Table 41) than in subjects with ADA negative are as follows: Respiratory, thoracic and mediastinal disorders (25% vs. 16.3%); Gastrointestinal disorders (26.8% vs. 20.8%); Nervous system disorders (23.2% vs. 17.3%); Vascular disorders (19.6% vs. 13.8%); and Investigations (16.1% vs. 10.6%)

AEs by PT reported with ≥5 percentage point higher frequency in subjects with treatment-emergent ADA (Table 41) than subjects with ADA negative were headache (14.3% vs. 4.5%), diarrhoea (14.3% vs. 4.9%), upper respiratory tract infection (16.1% vs. 8.8%), and bronchitis (12.5% vs. 6.5%).

No notable differences in AEs reported by SOC and PT were observed among subjects who were negative, subjects who were inconclusive for ADA, and subjects in ASaT.

Five-year cumulative exposure-adjusted summaries of AEs reported in subjects who were negative or inconclusive for ADA are provided by treatment group in the base study and the extension study. No notable differences were observes in the 5-year cumulative exposure-adjusted frequencies of AEs by SOC in the subjects with ADA negative between the two tildrakizumab groups. No notable differences were observed in the 5-year cumulative exposure-adjusted frequencies of AEs by SOC in the subjects with ADA inconclusive between the two tildrakizumab groups.

### Confirmed Composite Adjudicated Cardiovascular Events

An overall summary of confirmed composite adjudicated CV events during the extension study is provided in Table 40. The overall frequency of confirmed composite adjudicated CV events was low (19 [2.60%] subjects) and was similar between the two treatment groups (9 [2.40%] subjects in the tildrakizumab 100 mg group and 10 [2.90%] subjects in the tildrakizumab 200 mg group).

No meaningful differences were observed in the frequencies of confirmed composite adjudicated CV events between the two treatment groups.

TABLE 40
Subjects with Confirmed Composite Adjudicated Cardiovascular
Events (All Subjects as Treated - Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more confirmed composite | 9 | (2.4) | 10 | (2.9) | 19 | (2.6) |
| adjudicated CV events^{‡} | | | | | | |
| With no confirmed composite | 372 | (97.6) | 339 | (97.1) | 711 | (97.4) |
| adjudicated CV events | | | | | | |
| Confirmed CV Events^{§} | | | | | | |
| MACE^{∥} | 7 | (1.8) | 9 | (2.6) | 16 | (2.2) |
| Extended MACE^{††} | 7 | (1.8) | 9 | (2.6) | 16 | (2.2) |
| Fatal or non-fatal thrombotic/embolic/ | 8 | (2.1) | 10 | (2.9) | 18 | (2.5) |
| ischemic CV events^{‡‡} | | | | | | |

^{†}Subjects who took at least one dose of extension study medication based on the treatment actually received.
^{‡}Includes confirmed MACE, Extended MACE, and fatal or non-fatal thrombotic/embolic/ischemic CV events.
^{§}Subjects were counted only once in the overall category. A subject may appear in different categories.
^{∥}Includes non-fatal myocardial infarction, non-fatal stroke, and CV deaths that were confirmed as “cardiovascular” or “sudden”.
^{††}Includes non-fatal myocardial infarction, non-fatal stroke, unstable angina, coronary revascularization, resuscitated cardiac arrest, and CV deaths that were confirmed as “cardiovascular” or “sudden”.
^{‡‡}Includes fatal and non-fatal events - Acute Myocardial infarction; Ischemic stroke; Unstable angina; Coronary revascularization; Transient ischemic attack; Pulmonary embolism; Peripheral arterial Thrombosis/Thromboembolism; Venous thrombosis.
CV = Cardiovascular; MACE = Major Adverse Cardiovascular Events.

A 5-year cumulative exposure-adjusted overall summary of confirmed composite adjudicated CV events during the extension study found no differences between the two tildrakizumab treatment groups due to the small number of subjects with confirmed composite adjudicated CV events.

### Confirmed Adjudicated Cardiovascular Events

An overall summary of confirmed adjudicated CV events during the extension study is provided in Table 41.

The overall frequency of confirmed adjudicated CV events was relatively low (21 [2.9%] subjects) and was similar in the two treatment groups (10 [2.6%] subjects in the tildrakizumab 100 mg group and 11 [3.20%] subjects in the tildrakizumab 200 mg group).

The frequencies of acute myocardial infarction and coronary revascularization were numerically higher in the tildrakizumab 200 mg group (7 [2.0%] subjects and 6 [1.7%] subjects, respectively) compared to the tildrakizumab 100 mg group (4 [1.0%] subjects and 1 [0.3%] subject, respectively). Except for this, the frequencies of confirmed adjudicated CV events were similar between the two treatment groups.

TABLE 41
Subjects with Confirmed Adjudicated Cardiovascular
Events (All Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 381 | | 349 | | 730 | |
| With one or more confirmed adjudicated CV | 10 | (2.6) | 11 | (3.2) | 21 | (2.9) |
| events^{‡,§} | | | | | | |
| With no confirmed adjudicated CV events | 371 | (97.4) | 338 | (96.8) | 709 | (97.1) |
| Cardiac Events | 5 | (1.3) | 8 | (2.3) | 13 | (1.8) |
| Acute myocardial infarction | 4 | (1.0) | 7 | (2.0) | 11 | (1.5) |
| Cardiac arrhythmias, no evidence of | 2 | (0.5) | 1 | (0.3) | 3 | (0.4) |
| ischemia | | | | | | |
| Coronary revascularization | 1 | (0.3) | 6 | (1.7) | 7 | (1.0) |
| Cerebrovascular Events | 2 | (0.5) | 2 | (0.6) | 4 | (0.5) |
| Cerebrovascular Revascularization | 1 | (0.3) | 0 | | 1 | (0.1) |
| Ischemic stroke | 2 | (0.5) | 2 | (0.6) | 4 | (0.5) |
| Peripheral Vascular Events | 2 | (0.5) | 1 | (0.3) | 3 | (0.4) |
| Peripheral arterial | 1 | (0.3) | 1 | (0.3) | 2 | (0.3) |
| thrombosis/thromboembolism | | | | | | |
| Peripheral venous thrombosis | 1 | (0.3) | 0 | | 1 | (0.1) |
| Cardiovascular Deaths^{∥} | 1 | (0.3) | 1 | (0.3) | 2 | (0.3) |

^{†}Subjects who took at least one dose of extension study medication based on the treatment actually received.
^{‡}Subjects were counted only once in the overall category. A subject may appear in different categories.
^{§}Include subjects with one or more confirmed CV events.
^{∥}Includes deaths that were confirmed as “cardiovascular” and “Sudden” deaths.
CV = Cardiovascular.

A 5-year cumulative exposure-adjusted overall summary of confirmed adjudicated CV events during the base and the extension study found no meaningful differences between the two tildrakizumab treatment groups.

### Laboratory Values Over Time

Overall, the mean changes from baseline in each laboratory parameter were small, and there were no changes of clinical importance in mean laboratory values over time. No meaningful differences in mean changes from baseline of laboratory values were observed between the two treatment groups.

### Electrocardiogram

Overall, the mean changes from baseline in ECG were small, and there were no changes of clinical importance in mean ECG parameters over time. No meaningful differences in mean changes from baseline of any ECG parameters were observed between the two treatment groups.

### Safety Conclusions

No meaningful differences were observed in the overall frequency of AEs between the two treatment groups (321 [84.3%] subjects and 307 [88.0%] subjects in the tildrakizumab 100 mg group and 200 mg group, respectively).

The frequencies of SAEs by SOC were low (<5%) and similar between the two treatment groups. The overall frequency of serious drug-related AEs during the extension study was low (1.9%) and similar between the two treatment groups. The frequencies of AEs that led to discontinuation of study medication were similar in the two treatment groups (16 [4.2%] subjects and 13 [3.7%] subjects in the tildrakizumab 100 mg group and 200 mg group, respectively).

The 5-year cumulative exposure adjusted frequencies of overall AEs and drug-related AEs were slightly higher in the tildrakizumab 200 mg than in the tildrakizumab 100 mg while those of SAEs, serious drug-related AEs, and AEs leading to discontinuation were similar between the two tildrakizumab treatment groups in the base study and the extension study.

No meaningful differences were observed in the frequencies of each prespecified Tier 1 or Tier 2 AE between the two treatment groups. The most frequently reported Tier 1 AEs were Severe Infections (15 [3.9%] subjects and 11 [3.2%] subjects in the tildrakizumab 100 mg group and the tildrakizumab 200 mg group, respectively), followed by Malignancies (9 [2.4%] subjects and 13 [3.7%] subjects in the tildrakizumab 100 mg group and the tildrakizumab 200 mg group, respectively).

The overall frequencies of confirmed composite adjudicated CV events and confirmed adjudicated CV events were relatively low (19 [2.6%] subjects and 21 [2.9%] subjects, respectively) and were similar between the two treatment groups.

No specific correlation of ADA incidence with the overall frequency of AEs were observed. No notable differences in AEs by SOC and PT were observed between the subjects with treatment-emergent ADA and the subjects with ADA negative due to the small number of subjects with treatment-emergent ADA and AEs.

The mean changes from baseline in each laboratory parameter, vital sign, and ECG parameter were small, and there were no mean changes of clinical importance over time. No meaningful differences in mean changes from baseline of these parameters were observed between the two treatment groups.

### Overall Conclusions

### Safety

The most frequently reported AE was nasopharyngitis. AEs by SOC and PT reported during the extension study were similar to those reported in the subjects who treated with tildrakizumab in the base study. The overall frequencies of AEs or drug-related AEs leading to discontinuation of study medication were low (4.0% and 1.0%, respectively).

There was no notable increase in AEs, drug-related AEs, SAEs, Tier 1 AEs, and Tier 2 AEs compared to those who treated with tildrakizumab in the base study. There were no meaningful differences in the frequencies of AEs by SOC and PT were observed between the tildrakizumab 100 mg and the tildrakizumab 200 mg groups, and tildrakizumab up to 200 mg dose is considered well tolerated in long-term administration. No increased risks with a long-term administration of tildrakizumab 100 mg and 200 mg were observed during the extension study.

### Efficacy

Treatment with tildrakizumab 100 mg and 200 mg demonstrated positive changes in proportions of subjects with a PASI 50, 75, PASI 90, or PASI 100 response, and the proportion of subjects with a PGA score of “clear” or “minimal” with at least a 2-grade reduction during the extension study. PASI 50, PASI 75, PASI 90, and PASI 100 responses irrespective of subjects who were responders at Week 52 in both tildrakizumab treatment groups were maintained throughout the extension study.

### Pharmacokinetics

The mean concentrations of tildrakizumab from week 76 to week 244 in extension 1 are generally comparable for each dose group. Additionally, PK concentrations were also estimated and reported from available subjects beyond week 244 in extension 2.

The inter-subject variability was higher in extension 2 possibly due to much lower number of subjects which further reduced with time compared to extension 1. The mean concentrations of tildrakizumab in 200 mg dose group are higher than at 100 mg dose group suggesting a dose dependent increase in PK.

### Immunogenicity

The relatively high proportion of overall conclusive subjects (586 [80.8%] subjects) suggests that tildrakizumab concentration in the last post-baseline sample from the majority of the subjects was low enough for the ADA assay to provide meaningful results. Overall, NAb incidence was low (<3%) in the base study and the extension study. No specific correlation of ADA and NAb incidence with the efficacy (PASI scores and PGA) or the overall frequency of AEs were observed.

### 64-Week Study

This long-term extension study is a follow-up of the 64-Week, phase 3, randomized, active comparator and placebo-controlled, parallel design study to evaluate the efficacy and safety/tolerability of subcutaneous tildrakizumab, in subjects with moderate-to-severe chronic plaque psoriasis. Eligible subjects received one of two active doses of tildrakizumab (MK-3222), 100 mg or 200 mg, for a minimum of 4 years. Dose assignment (100 mg or 200 mg) was the same dose the subject received at the end of the base study. During the open-label long-term extension phase of the study, subjects received tildrakizumab (MK-3222) in either the PFS or AI format.

Subjects: Of the 638 subjects who completed the base study, 506 (79.3%) subjects entered the extension study. Among the 506 subjects who entered the extension study, 239 (47.2%) subjects received tildrakizumab 100 mg and 267 (52.8%) subjects received tildrakizumab 200 mg. Of the 506 subjects, 379 (74.9%) subjects completed the extension 1 (171 [71.5%] subjects in tildrakizumab 100 mg group and 208 [77.9%] subjects in tildrakizumab 200 mg group). A total of 127 (25.1%) subjects discontinued from the study (68 [28.5%] subjects in the tildrakizumab 100 mg group and 59 [22.1%] subjects in the tildrakizumab 200 mg group). The most common reason for discontinuation in each treatment group was withdrawal by subjects (47 [9.3%] subjects overall; and 22 [9.2%] subjects in tildrakizumab 100 mg group and 25 [9.4%] subjects in tildrakizumab 200 mg group). This was followed by AEs (27 [5.3%] subjects overall and 19 [7.9%] subjects in tildrakizumab 100 mg and 8 [3.0%] subjects in tildrakizumab 200 mg groups) and lost to follow-up (24 [4.7%] subjects overall; and 15 [6.3%] subjects in tildrakizumab 100 mg and 9 [3.4%] subjects in tildrakizumab 200 mg group).

Out of 197 subjects who entered the extension 2, a total of 160 (31.6%) subjects completed the extension 2; (70 [29.3%] subjects in tildrakizumab 100 mg group and 90 [33.7%] subjects in tildrakizumab 200 mg group). Of 160 subjects in extension 2, overall, 30 (5.9%) subjects discontinued from the study (12 [5.0%] subjects in the tildrakizumab 100 mg group and 18 [6.7%] subjects in the tildrakizumab 200 mg).

The most common reason for discontinuation in each treatment group was withdrawal by subjects (14 [2.8%] subjects overall; and 4 [1.7%] subjects in tildrakizumab 100 mg group and 10 [3.7%] subjects in tildrakizumab 200 mg group). This was followed by other protocol specified criteria (9 [1.8%] subjects overall; and 4 [1.7%] subjects in tildrakizumab 100 mg and 5 [1.9%] subjects in tildrakizumab 200 mg groups). All 78 (15.4%) subjects who entered completed the extension 3, 33 [13.8%] subjects were in tildrakizumab 100 mg group and 45 [16.9%] subjects in tildrakizumab 200 mg group.

Of the 638 subjects who completed the base study, 132 subjects were excluded from the extension study. The primary reason for exclusion was due to other protocol-specified criteria (121 [91.7%] subjects).

Demography and Baseline Disease Characteristics. Table 42 summarizes subject baseline characteristics by treatment group. Demographic characteristics were generally similar between the two treatment groups. A majority of the subjects in the extension study were males (342 [67.60%] subjects overall) compared to females (164 [32.40%] subjects). Similarly, subjects in the <65 years age category were predominant (461 [91.1%] subjects overall) compared to those in the ≥65 years category (45 [8.9%] subjects overall). The median age for subjects was 47.5 years (range: 18 to 82 years). Most enrolled subjects in the extension study were white (336 [66.4%]) followed by Asians (140 [27.70%] subjects). The Not Hispanic or Latino (449 [88.7%] subjects) was the most common ethnic group.

TABLE 42
Subject Baseline Characteristics (All Subjects
Entered Extension Study) Extension Study

| | MK-3222 100 mg | MK-3222 200 mg | Total |
|---|---|---|---|
| | n (%) | n (%) | n (%) |
| Subjects in population | 239 | 267 | 506 |

Gender

| | | | | | | |
|---|---|---|---|---|---|---|
| Male | 159 | (66.5) | 183 | (68.5) | 342 | (67.6) |
| Female | 80 | (33.5) | 84 | (31.5) | 164 | (32.4) |

Age (years)

| | | | | | | |
|---|---|---|---|---|---|---|
| <65 | 217 | (90.8) | 244 | (91.4) | 461 | (91.1) |
| ≥65 | 22 | (9.2) | 23 | (8.6) | 45 | (8.9) |

| | | | |
|---|---|---|---|
| Mean | 46.9 | 47.1 | 47.0 |
| SD | 13.02 | 13.02 | 13.00 |
| Median | 47.0 | 48.0 | 47.5 |
| Range | 18 to 82 | 18 to 76 | 18 to 82 |

Race

| American Indian or Alaska Native | 0 | | 0 | | 0 | |
|---|---|---|---|---|---|---|
| Black | 7 | (2.9) | 7 | (2.6) | 14 | (2.8) |
| Native Hawaiian or Other Pacific | 1 | (0.4) | 2 | (0.7) | 3 | (0.6) |
| Island | | | | | | |
| White | 163 | (68.2) | 173 | (64.8) | 336 | (66.4) |
| Asian | 60 | (25.1) | 80 | (30.0) | 140 | (27.7) |
| Multi-Racial | 8 | (3.3) | 5 | (1.9) | 13 | (2.6) |

| | | | |
|---|---|---|---|
| Missing | 0 | 0 | 0 |

Ethnicity†

| | | | | | | |
|---|---|---|---|---|---|---|
| Hispanic or Latino | 24 | (10.0) | 33 | (12.4) | 57 | (11.3) |
| Not Hispanic or Latino | 215 | (90.0) | 234 | (87.6) | 449 | (88.7) |

| | | | |
|---|---|---|---|
| Not Reported | 0 | 0 | 0 |
| Unknown | 0 | 0 | 0 |

Weight (kg)

| | | | |
|---|---|---|---|
| Subjects with data | 239 | 267 | 506 |
| Mean | 87.09 | 87.76 | 87.45 |
| SD | 24.376 | 24.230 | 24.277 |
| Median | 84.40 | 84.50 | 84.40 |
| Range | 40.9 to 192.3 | 45.0 to 222.2 | 40.9 to 222.2 |

Height (cm)

| | | | |
|---|---|---|---|
| Subjects with data | 239 | 267 | 506 |
| Mean | 170.01 | 170.26 | 170.14 |
| SD | 9.272 | 9.932 | 9.617 |
| Median | 170.20 | 171.50 | 170.50 |
| Range | 144.8 to 190.5 | 128.4 to 193.0 | 128.4 to 193.0 |

Psoriatic Arthritis

| | | | | | | |
|---|---|---|---|---|---|---|
| Yes | 42 | (17.6) | 44 | (16.5) | 86 | (17.0) |
| No | 197 | (82.4) | 223 | (83.5) | 420 | (83.0) |

Body Surface Area (%)

| | | | |
|---|---|---|---|
| Subjects with data | 239 | 267 | 506 |
| Mean | 30.2 | 31.7 | 31.0 |
| SD | 17.48 | 19.58 | 18.61 |
| Median | 25.0 | 26.0 | 25.5 |
| Range | 10 to 86 | 10 to 100 | 10 to 100 |

^{†}Not reported: if ethnicity is not provided or available, Unknown: if ethnicity is not known, not observed, not recorded or refused.
Baseline is defined as the last measurement taken prior to the first study medication in the base study.
SD = Standard Deviation.

Baseline Disease Characteristics. Table 43 summarizes baseline efficacy endpoints for each treatment group for subjects who entered the extension study. Baseline efficacy parameters were generally similar between the two treatment groups. The mean PASI scores were 20.0 and 21.3 in the tildrakizumab 100 mg and 200 mg groups, respectively. A majority of the subjects had a PGA score of 3 (336 [66.4%] subjects overall; 159 (66.5%) subjects in tildrakizumab 100 mg group and 177 [66.3%] subjects in tildrakizumab 200 mg group).

TABLE 43
Summary of Baseline Efficacy Endpoints (All Subjects
Entered Extension Study) Extension Study

| | MK-3222 100 mg | MK-3222 200 mg | Total |
|---|---|---|---|
| | n (%) | n (%) | n (%) |
| Subjects in population | 239 | 267 | 506 |

PASI Score

| | | | |
|---|---|---|---|
| Subjects with data | 239 | 267 | 506 |
| Mean | 20.0 | 21.3 | 20.7 |
| SD | 7.61 | 9.57 | 8.71 |
| Median | 17.4 | 18.2 | 17.9 |
| Range | 12.0 to 55.0 | 12.0 to 66.0 | 12.0 to 66.0 |

PGA Score

| Subjects with data | 239 | | 267 | | 506 | |
|---|---|---|---|---|---|---|
| <3 | 1 | (0.4) | 0 | | 1 | (0.2) |
| 3 | 159 | (66.5) | 177 | (66.3) | 336 | (66.4) |
| 4 | 73 | (30.5) | 83 | (31.1) | 156 | (30.8) |
| 5 | 6 | (2.5) | 7 | (2.6) | 13 | (2.6) |

PASI = Psoriasis Area Severity Index; PGA = Physician's Global Assessment; SD = Standard deviation.
Baseline is defined as the last measurement taken prior to the first study medication in the base study.

Extent of Exposure. The extent of exposure for subjects receiving tildrakizumab 100 mg and subjects receiving tildrakizumab 200 mg during the extension study is shown in Table 44. The median number of Weeks on study medication in the extension study was 204 Weeks in both the treatment arms. The median number of weeks on tildrakizumab (base+extension study) was 270 weeks and 280 weeks in the tildrakizumab 100 mg group and 200 mg group, respectively.

TABLE 44
Extent of Exposure to MK-3222 (All Subjects
Randomized - Extension) Extension Study

| | | MK-3222 100 mg | MK-3222 200 mg |
|---|---|---|---|
| ≤24 | Weeks | 10 | 5 |
| 25-48 | Weeks | 13 | 4 |
| 49-72 | Weeks | 7 | 3 |
| 73-96 | Weeks | 12 | 8 |
| 97-120 | Weeks | 6 | 9 |
| 121-144 | Weeks | 8 | 10 |
| 145-168 | Weeks | 8 | 10 |
| 169-192 | Weeks | 9 | 22 |
| >192 | Weeks | 166 | 196 |

| | | |
|---|---|---|
| Total Subjects | 239 | 267 |
| Duration Range | 12 to 356 | 12 to 356 |
| Mean Duration | 207.1 | 226.2 |
| Median Duration | 204.0 | 204.0 |

Each subject is counted once on each applicable dosage category row.
This table reflects transient cross-treatments.
Only subjects treated during extension were included in the table.

PASI 50 Response Over Time Among PASI 50 Responders at Week 64. The PASI 50 response among those who were PASI 50 responders at Week 64 was maintained in a majority of subjects who remained in the study at specified time points from extension Week 0 to extension Week 332 (≥95.200 subjects in both the treatment groups). See Table 45.

TABLE 45
Proportion of Subjects with PASI 50 Response
Over Time - PASI 50 Responders at Week 64 (Full
Analysis Set - Extension) Extension Study

| | | |
|---|---|---|
| | MK-3222 100 mg | MK-3222 200 mg |
| Statistics | N = 239 | N = 267 |

PASI 50 (Ext Week 0)

| | | |
|---|---|---|
| Subjects with data | 232 | 263 |
| Responders (%)^{†} | 227 (97.8) | 258 (98.1) |

PASI 50 (Ext Week 12)

| | | |
|---|---|---|
| Subjects with data | 228 | 261 |
| Responders (%)^{†} | 226 (99.1) | 251 (96.2) |

PASI 50 (Ext Week 24)

| | | |
|---|---|---|
| Subjects with data | 227 | 261 |
| Responders (%)^{†} | 224 (98.7) | 253 (96.9) |

PASI 50 (Ext Week 36)

| | | |
|---|---|---|
| Subjects with data | 221 | 249 |
| Responders (%)^{†} | 215 (97.3) | 237 (95.2) |

PASI 50 (Ext Week 48)

| | | |
|---|---|---|
| Subjects with data | 217 | 255 |
| Responders (%)^{†} | 212 (97.7) | 247 (96.9) |

PASI 50 (Ext Week 60)

| | | |
|---|---|---|
| Subjects with data | 210 | 252 |
| Responders (%)^{†} | 206 (98.1) | 246 (97.6) |

PASI 50 (Ext Week 72)

| | | |
|---|---|---|
| Subjects with data | 205 | 251 |
| Responders (%)^{†} | 201 (98.0) | 241 (96.0) |

PASI 50 (Ext Week 84)

| | | |
|---|---|---|
| Subjects with data | 205 | 247 |
| Responders (%)^{†} | 198 (96.6) | 237 (96.0) |

PASI 50 (Ext Week 96)

| | | |
|---|---|---|
| Subjects with data | 200 | 240 |
| Responders (%)^{†} | 194 (97.0) | 231 (96.3) |

PASI 50 (Ext Week 120)

| | | |
|---|---|---|
| Subjects with data | 189 | 235 |
| Responders (%)^{†} | 183 (96.8) | 224 (95.3) |

PASI 50 (Ext Week 144)

| | | |
|---|---|---|
| Subjects with data | 182 | 229 |
| Responders (%)^{†} | 174 (95.6) | 223 (97.4) |

PASI 50 (Ext Week 168)

| | | |
|---|---|---|
| Subjects with data | 176 | 218 |
| Responders (%)^{†} | 171 (97.2) | 214 (98.2) |

PASI 50 (Ext Week 192)

| | | |
|---|---|---|
| Subjects with data | 168 | 206 |
| Responders (%)^{†} | 163 (97.0) | 201 (97.6) |

PASI 50 (Ext Week 216)

| | | |
|---|---|---|
| Subjects with data | 84 | 105 |
| Responders (%)^{†} | 80 (95.2) | 101 (96.2) |

PASI 50 (Ext Week 240)

| | | |
|---|---|---|
| Subjects with data | 83 | 102 |
| Responders (%)^{†} | 81 (97.6) | 98 (96.1) |

PASI 50 (Ext Week 264)

| | | |
|---|---|---|
| Subjects with data | 75 | 93 |
| Responders (%)^{†} | 73 (97.3) | 90 (96.8) |

PASI 50 (Ext Week 288)

| | | |
|---|---|---|
| Subjects with data | 65 | 73 |
| Responders (%)^{†} | 63 (96.9) | 71 (97.3) |

PASI 50 (Ext Week 312)

| | | |
|---|---|---|
| Subjects with data | 19 | 23 |
| Responders (%)^{†} | 19 (100.0) | 22 (95.7) |

PASI 50 (Ext Week 332)

| | | |
|---|---|---|
| Subjects with data | 19 | 22 |
| Responders (%)^{†} | 19 (100.0) | 22 (100.0) |

PASI 50 (Ext Week 344)

| | | |
|---|---|---|
| Subjects with data | 2 | 3 |
| Responders (%)^{†} | 2 (100.0) | 2 (66.7) |

PASI 50 (Ext Week 368)

| | | |
|---|---|---|
| Subjects with data | 1 | 0 |
| Responders (%)^{†} | 1 (100.0) | 0 |

N = Number of randomized subjects who received at least one dose of study medication in the extension study.
PASI = Psoriasis Area and Severity Index.
^{†}Percentages were based on subjects with data.

PASI 75 Response Over Time Among PASI 75 Responders at Week 64. The proportion of subjects with PASI 75 response among those who were PASI 75 responders at Week 64 was maintained in a majority of the subjects who remained in the study at specified time points from extension Week 0 to extension Week 332 (≥85.6%0 in the tildrakizumab 100 mg group and ≥86.600 in the tildrakizumab 200 mg group) (Table 46).

TABLE 46
Proportion of Subjects with PASI 75 Response
Over Time - PASI 75 Responders at Week 64 (Full
Analysis Set - Extension) Extension Study

| | | |
|---|---|---|
| | MK-3222 100 mg | MK-3222 200 mg |
| Statistics | N = 239 | N = 267 |

PASI 75 (Ext Week 0)

| | | |
|---|---|---|
| Subjects with data | 203 | 215 |
| Responders (%)^{†} | 191 (94.1) | 198 (92.1) |

PASI 75 (Ext Week 12)

| | | |
|---|---|---|
| Subjects with data | 200 | 213 |
| Responders (%)^{†} | 187 (93.5) | 195 (91.5) |

PASI 75 (Ext Week 24)

| | | |
|---|---|---|
| Subjects with data | 200 | 213 |
| Responders (%)^{†} | 178 (89.0) | 196 (92.0) |

PASI 75 (Ext Week 36)

| | | |
|---|---|---|
| Subjects with data | 195 | 202 |
| Responders (%)^{†} | 177 (90.8) | 175 (86.6) |

PASI 75 (Ext Week 48)

| | | |
|---|---|---|
| Subjects with data | 193 | 208 |
| Responders (%)^{†} | 174 (90.2) | 189 (90.9) |

PASI 75 (Ext Week 60)

| | | |
|---|---|---|
| Subjects with data | 188 | 206 |
| Responders (%)^{†} | 173 (92.0) | 189 (91.7) |

PASI 75 (Ext Week 72)

| | | |
|---|---|---|
| Subjects with data | 184 | 205 |
| Responders (%)^{†} | 165 (89.7) | 184 (89.8) |

PASI 75 (Ext Week 84)

| | | |
|---|---|---|
| Subjects with data | 184 | 202 |
| Responders (%)^{†} | 163 (88.6) | 175 (86.6) |

PASI 75 (Ext Week 96)

| | | |
|---|---|---|
| Subjects with data | 180 | 195 |
| Responders (%)^{†} | 154 (85.6) | 170 (87.2) |

PASI 75 (Ext Week 120)

| | | |
|---|---|---|
| Subjects with data | 169 | 192 |
| Responders (%)^{†} | 145 (85.8) | 173 (90.1) |

PASI 75 (Ext Week 144)

| | | |
|---|---|---|
| Subjects with data | 164 | 189 |
| Responders (%)^{†} | 143 (87.2) | 171 (90.5) |

PASI 75 (Ext Week 168)

| | | |
|---|---|---|
| Subjects with data | 158 | 180 |
| Responders (%)^{†} | 143 (90.5) | 164 (91.1) |

PASI 75 (Ext Week 192)

| | | |
|---|---|---|
| Subjects with data | 151 | 171 |
| Responders (%)^{†} | 138 (91.4) | 158 (92.4) |

PASI 75 (Ext Week 216)

| | | |
|---|---|---|
| Subjects with data | 79 | 84 |
| Responders (%)^{†} | 68 (86.1) | 76 (90.5) |

PASI 75 (Ext Week 240)

| | | |
|---|---|---|
| Subjects with data | 78 | 82 |
| Responders (%)^{†} | 71 (91.0) | 76 (92.7) |

PASI 75 (Ext Week 264)

| | | |
|---|---|---|
| Subjects with data | 71 | 75 |
| Responders (%)^{†} | 62 (87.3) | 72 (96.0) |

PASI 75 (Ext Week 288)

| | | |
|---|---|---|
| Subjects with data | 62 | 60 |
| Responders (%)^{†} | 56 (90.3) | 56 (93.3) |

PASI 75 (Ext Week 312)

| | | |
|---|---|---|
| Subjects with data | 19 | 22 |
| Responders (%)^{†} | 17 (89.5) | 21 (95.5) |

PASI 75 (Ext Week 332)

| | | |
|---|---|---|
| Subjects with data | 19 | 21 |
| Responders (%)^{†} | 18 (94.7) | 21 (100.0) |

PASI 75 (Ext Week 344)

| | | |
|---|---|---|
| Subjects with data | 2 | 3 |
| Responders (%)^{†} | 2 (100.0) | 2 (66.7) |

PASI 75 (Ext Week 368)

| | | |
|---|---|---|
| Subjects with data | 1 | 0 |
| Responders (%)^{†} | 1 (100.0) | 0 |

^{†}Percentages were based on subjects with data.
N = Number of randomized subjects who received at least one dose of study medication in the extension study.
PASI = Psoriasis Area and Severity Index.

PASI 90 Response Over Time Among PASI 90 Responders at Week 64. The proportion of subjects with PASI 90 response among those who were PASI 90 responders at Week 64 was maintained in a majority of the subjects who remained in the study at specified time points from extension Week 0 to extension Week 332 (≥69.1% in the tildrakizumab 100 mg group and ≥75.2%0 in the tildrakizumab 200 mg group) (Table 47). The 200 mg dose had a better response until week 48; from week 60 to week 96 the responses were comparable between the two treatment arms, and for the remaining weeks tildrakizumab 200 mg treatment group displayed a better response.

TABLE 47
Proportion of Subjects with PASI 90 Response
Over Time - PASI 90 Responders at Week 64 (Full
Analysis Set - Extension) Extension Study

| | | |
|---|---|---|
| | MK-3222 100 mg | MK-3222 200 mg |
| Statistics | N = 239 | N = 267 |

PASI 90 (Ext Week 0)

| | | |
|---|---|---|
| Subjects with data | 125 | 137 |
| Responders (%)^{†} | 111 (88.8) | 120 (87.6) |

PASI 90 (Ext Week 12)

| | | |
|---|---|---|
| Subjects with data | 125 | 136 |
| Responders (%)^{†} | 99 (79.2) | 116 (85.3) |

PASI 90 (Ext Week 24)

| | | |
|---|---|---|
| Subjects with data | 124 | 136 |
| Responders (%)^{†} | 95 (76.6) | 119 (87.5) |

PASI 90 (Ext Week 36)

| | | |
|---|---|---|
| Subjects with data | 122 | 129 |
| Responders (%)^{†} | 96 (78.7) | 110 (85.3) |

PASI 90 (Ext Week 48)

| | | |
|---|---|---|
| Subjects with data | 120 | 134 |
| Responders (%)^{†} | 89 (74.2) | 109 (81.3) |

PASI 90 (Ext Week 60)

| | | |
|---|---|---|
| Subjects with data | 120 | 133 |
| Responders (%)^{†} | 91 (75.8) | 104 (78.2) |

PASI 90 (Ext Week 72)

| | | |
|---|---|---|
| Subjects with data | 118 | 133 |
| Responders (%)^{†} | 89 (75.4) | 100 (75.2) |

PASI 90 (Ext Week 84)

| | | |
|---|---|---|
| Subjects with data | 118 | 131 |
| Responders (%)^{†} | 91 (77.1) | 101 (77.1) |

PASI 90 (Ext Week 96)

| | | |
|---|---|---|
| Subjects with data | 115 | 130 |
| Responders (%)^{†} | 91 (79.1) | 101 (77.7) |

PASI 90 (Ext Week 120)

| | | |
|---|---|---|
| Subjects with data | 110 | 125 |
| Responders (%)^{†} | 76 (69.1) | 104 (83.2) |

PASI 90 (Ext Week 144)

| | | |
|---|---|---|
| Subjects with data | 105 | 124 |
| Responders (%)^{†} | 81 (77.1) | 100 (80.6) |

PASI 90 (Ext Week 168)

| | | |
|---|---|---|
| Subjects with data | 102 | 118 |
| Responders (%)^{†} | 76 (74.5) | 106 (89.8) |

PASI 90 (Ext Week 192)

| | | |
|---|---|---|
| Subjects with data | 97 | 113 |
| Responders (%)^{†} | 74 (76.3) | 99 (87.6) |

PASI 90 (Ext Week 216)

| | | |
|---|---|---|
| Subjects with data | 53 | 55 |
| Responders (%)^{†} | 42 (79.2) | 50 (90.9) |

PASI 90 (Ext Week 240)

| | | |
|---|---|---|
| Subjects with data | 53 | 55 |
| Responders (%)^{†} | 44 (83.0) | 50 (90.9) |

PASI 90 (Ext Week 264)

| | | |
|---|---|---|
| Subjects with data | 49 | 49 |
| Responders (%)^{†} | 42 (85.7) | 42 (85.7) |

PASI 90 (Ext Week 288)

| | | |
|---|---|---|
| Subjects with data | 42 | 40 |
| Responders (%)^{†} | 35 (83.3) | 37 (92.5) |

PASI 90 (Ext Week 312)

| | | |
|---|---|---|
| Subjects with data | 10 | 17 |
| Responders (%)^{†} | 8 (80.0) | 16 (94.1) |

PASI 90 (Ext Week 332)

| | | |
|---|---|---|
| Subjects with data | 12 | 15 |
| Responders (%)^{†} | 11 (91.7) | 15 (100.0) |

PASI 90 (Ext Week 344)

| | | |
|---|---|---|
| Subjects with data | 0 | 1 |
| Responders (%)^{†} | 0 | 1 (100.0) |

PASI 90 (Ext Week 368)

| | | |
|---|---|---|
| Subjects with data | 1 | 0 |
| Responders (%)^{†} | 1 (100.0) | 0 |

N = Number of randomized subjects who received at least one dose of study medication in the extension study.
PASI = Psoriasis Area and Severity Index.
^{†}Percentages were based on subjects with data.

PASI 100 Response Over Time Among PASI 100 Responders at Week 64. The analysis of the proportion of subjects with a PASI 100 response among those who were PASI 100 responders at Week 64 of the base study is presented in Table 48. The proportion of subjects with PASI100 response among PASI 100 responders at week 64 was consistently better in the tildrakizumab 200 mg treatment group throughout the extension phase of study.

TABLE 48
Proportion of Subjects with PASI 100 Response
Over Time - PASI 100 Responders at Week 64 (Full
Analysis Set - Extension) Extension Study

| | | |
|---|---|---|
| | MK-3222 100 mg | MK-3222 200 mg |
| Statistics | N = 239 | N = 267 |

PASI 100 (Ext Week 0)

| | | |
|---|---|---|
| Subjects with data | 70 | 70 |
| Responders (%)^{†} | 48 (68.6) | 55 (78.6) |

PASI 100 (Ext Week 12)

| | | |
|---|---|---|
| Subjects with data | 71 | 70 |
| Responders (%)^{†} | 47 (66.2) | 54 (77.1) |

PASI 100 (Ext Week 24)

| | | |
|---|---|---|
| Subjects with data | 70 | 70 |
| Responders (%)^{†} | 43 (61.4) | 55 (78.6) |

PASI 100 (Ext Week 36)

| | | |
|---|---|---|
| Subjects with data | 68 | 66 |
| Responders (%)^{†} | 41 (60.3) | 49 (74.2) |

PASI 100 (Ext Week 48)

| | | |
|---|---|---|
| Subjects with data | 69 | 69 |
| Responders (%)^{†} | 37 (53.6) | 44 (63.8) |

PASI 100 (Ext Week 60)

| | | |
|---|---|---|
| Subjects with data | 69 | 68 |
| Responders (%)^{†} | 41 (59.4) | 42 (61.8) |

PASI 100 (Ext Week 72)

| | | |
|---|---|---|
| Subjects with data | 67 | 68 |
| Responders (%)^{†} | 40 (59.7) | 42 (61.8) |

PASI 100 (Ext Week 84)

| | | |
|---|---|---|
| Subjects with data | 67 | 66 |
| Responders (%)^{†} | 36 (53.7) | 43 (65.2) |

PASI 100 (Ext Week 96)

| | | |
|---|---|---|
| Subjects with data | 64 | 66 |
| Responders (%)^{†} | 34 (53.1) | 42 (63.6) |

PASI 100 (Ext Week 120)

| | | |
|---|---|---|
| Subjects with data | 62 | 63 |
| Responders (%)^{†} | 36 (58.1) | 41 (65.1) |

PASI 100 (Ext Week 144)

| | | |
|---|---|---|
| Subjects with data | 62 | 64 |
| Responders (%)^{†} | 36 (58.1) | 40 (62.5) |

PASI 100 (Ext Week 168)

| | | |
|---|---|---|
| Subjects with data | 61 | 61 |
| Responders (%)^{†} | 35 (57.4) | 40 (65.6) |

PASI 100 (Ext Week 192)

| | | |
|---|---|---|
| Subjects with data | 57 | 60 |
| Responders (%)^{†} | 30 (52.6) | 35 (58.3) |

PASI 100 (Ext Week 216)

| | | |
|---|---|---|
| Subjects with data | 29 | 28 |
| Responders (%)^{†} | 15 (51.7) | 18 (64.3) |

PASI 100 (Ext Week 240)

| | | |
|---|---|---|
| Subjects with data | 29 | 28 |
| Responders (%)^{†} | 20 (69.0) | 19 (67.9) |

PASI 100 (Ext Week 264)

| | | |
|---|---|---|
| Subjects with data | 29 | 27 |
| Responders (%)^{†} | 19 (65.5) | 19 (70.4) |

PASI 100 (Ext Week 288)

| | | |
|---|---|---|
| Subjects with data | 25 | 20 |
| Responders (%)^{†} | 15 (60.0) | 14 (70.0) |

PASI 100 (Ext Week 312)

| | | |
|---|---|---|
| Subjects with data | 6 | 12 |
| Responders (%)⁼ | 2 (33.3) | 9 (75.0) |

PASI 100 (Ext Week 332)

| | | |
|---|---|---|
| Subjects with data | 8 | 9 |
| Responders (%)⁼ | 6 (75.0) | 7 (77.8) |

N = Number of randomized subjects who received at least one dose of study medication in the extension study.
PASI = Psoriasis Area and Severity Index.
^{†}Percentages were based on subjects with data.

PASI 75, PASI90, and PASI 100 Responses Over Time. Table 49 summarizes the proportion of subjects with PASI 75, PASI 90, and PASI 100 responses irrespective of the response status at week 64 over time for the FAS population for the two treatment groups. Within each PASI response measured (PASI 75, PASI 90, and PASI 100) the proportion of subjects with PASI response was maintained throughout the study among subjects who remained in the study at the specified time points. Overall, although the 100 mg treatment group displayed slightly better response than the 200 mg treatment group at several time points, the response rates were comparable between the two treatment groups.

TABLE 49
Proportion of Subjects with a PASI 75, PASI 90, and PASI 100 Response
Over Time (Full Analysis Set − Extension) Extension Study

| | | PASI75 | | PASI90 | | PASI100 | |
|---|---|---|---|---|---|---|---|
| Treatment | N | n | (%) | n | (%) | n | (%) |

Week 64

| | | | | | | | |
|---|---|---|---|---|---|---|---|
| MK-3222 100 mg | 239 | 209 | (87.4) | 128 | (53.6) | 73 | (30.5) |
| MK-3222 200 mg | 267 | 218 | (81.6) | 140 | (52.4) | 72 | (27.0) |

Ext Week 0

| | | | | | | | |
|---|---|---|---|---|---|---|---|
| MK-3222 100 mg | 233 | 199 | (85.4) | 133 | (57.1) | 60 | (25.8) |
| MK-3222 200 mg | 263 | 210 | (79.8) | 139 | (52.9) | 69 | (26.2) |

Ext Week 12

| | | | | | | | |
|---|---|---|---|---|---|---|---|
| MK-3222 100 mg | 229 | 196 | (85.6) | 124 | (54.1) | 62 | (27.1) |
| MK-3222 200 mg | 261 | 207 | (79.3) | 140 | (53.6) | 74 | (28.4) |

Ext Week 24

| | | | | | | | |
|---|---|---|---|---|---|---|---|
| MK-3222 100 mg | 228 | 186 | (81.6) | 122 | (53.5) | 61 | (26.8) |
| MK-3222 200 mg | 261 | 210 | (80.5) | 145 | (55.6) | 78 | (29.9) |

Ext Week 36

| | | | | | | | |
|---|---|---|---|---|---|---|---|
| MK-3222 100 mg | 221 | 187 | (84.6) | 119 | (53.8) | 58 | (26.2) |
| MK-3222 200 mg | 249 | 188 | (75.5) | 131 | (52.6) | 62 | (24.9) |

Ext Week 48

| | | | | | | | |
|---|---|---|---|---|---|---|---|
| MK-3222 100 mg | 217 | 182 | (83.9) | 113 | (52.1) | 49 | (22.6) |
| MK-3222 200 mg | 255 | 205 | (80.4) | 136 | (53.3) | 59 | (23.1) |

Ext Week 60

| | | | | | | | |
|---|---|---|---|---|---|---|---|
| MK-3222 100 mg | 210 | 182 | (86.7) | 116 | (55.2) | 59 | (28.1) |
| MK-3222 200 mg | 252 | 204 | (81.0) | 132 | (52.4) | 66 | (26.2) |

Ext Week 72

| | | | | | | | |
|---|---|---|---|---|---|---|---|
| MK-3222 100 mg | 205 | 175 | (85.4) | 115 | (56.1) | 60 | (29.3) |
| MK-3222 200 mg | 251 | 197 | (78.5) | 131 | (52.2) | 66 | (26.3) |

Ext Week 84

| | | | | | | | |
|---|---|---|---|---|---|---|---|
| MK-3222 100 mg | 205 | 173 | (84.4) | 119 | (58.0) | 51 | (24.9) |
| MK-3222 200 mg | 247 | 187 | (75.7) | 126 | (51.0) | 64 | (25.9) |

Ext Week 96

| | | | | | | | |
|---|---|---|---|---|---|---|---|
| MK-3222 100 mg | 200 | 165 | (82.5) | 112 | (56.0) | 50 | (25.0) |
| MK-3222 200 mg | 240 | 186 | (77.5) | 127 | (52.9) | 63 | (26.3) |

Ext Week 120

| | | | | | | | |
|---|---|---|---|---|---|---|---|
| MK-3222 100 mg | 189 | 154 | (81.5) | 97 | (51.3) | 51 | (27.0) |
| MK-3222 200 mg | 235 | 190 | (80.9) | 130 | (55.3) | 63 | (26.8) |

Ext Week 144

| | | | | | | | |
|---|---|---|---|---|---|---|---|
| MK-3222 100 mg | 182 | 150 | (82.4) | 103 | (56.6) | 51 | (28.0) |
| MK-3222 200 mg | 229 | 186 | (81.2) | 125 | (54.6) | 63 | (27.5) |

Ext Week 168

| | | | | | | | |
|---|---|---|---|---|---|---|---|
| MK-3222 100 mg | 176 | 152 | (86.4) | 101 | (57.4) | 52 | (29.5) |
| MK-3222 200 mg | 218 | 181 | (83.0) | 135 | (61.9) | 71 | (32.6) |

Ext Week 192

| | | | | | | | |
|---|---|---|---|---|---|---|---|
| MK-3222 100 mg | 168 | 147 | (87.5) | 98 | (58.3) | 49 | (29.2) |
| MK-3222 200 mg | 206 | 173 | (84.0) | 124 | (60.2) | 61 | (29.6) |

Ext Week 216

| | | | | | | | |
|---|---|---|---|---|---|---|---|
| MK-3222 100 mg | 84 | 70 | (83.3) | 49 | (58.3) | 25 | (29.8) |
| MK-3222 200 mg | 105 | 85 | (81.0) | 58 | (55.2) | 26 | (24.8) |

Ext Week 240

| | | | | | | | |
|---|---|---|---|---|---|---|---|
| MK-3222 100 mg | 83 | 72 | (86.7) | 55 | (66.3) | 29 | (34.9) |
| MK-3222 200 mg | 102 | 86 | (84.3) | 60 | (58.8) | 30 | (29.4) |

Ext Week 264

| | | | | | | | |
|---|---|---|---|---|---|---|---|
| MK-3222 100 mg | 75 | 63 | (84.0) | 48 | (64.0) | 29 | (38.7) |
| MK-3222 200 mg | 93 | 79 | (84.9) | 54 | (58.1) | 28 | (30.1) |

Ext Week 288

| | | | | | | | |
|---|---|---|---|---|---|---|---|
| MK-3222 100 mg | 65 | 57 | (87.7) | 42 | (64.6) | 22 | (33.8) |
| MK-3222 200 mg | 73 | 62 | (84.9) | 47 | (64.4) | 21 | (28.8) |

Ext Week 312

| | | | | | | | |
|---|---|---|---|---|---|---|---|
| MK-3222 100 mg | 19 | 17 | (89.5) | 11 | (57.9) | 6 | (31.6) |
| MK-3222 200 mg | 23 | 21 | (91.3) | 18 | (78.3) | 13 | (56.5) |

Ext Week 332

| | | | | | | | |
|---|---|---|---|---|---|---|---|
| MK-3222 100 mg | 19 | 18 | (94.7) | 15 | (78.9) | 12 | (63.2) |
| MK-3222 200 mg | 22 | 22 | (100.0) | 20 | (90.9) | 13 | (59.1) |

Ext Week 344

| | | | | | | | |
|---|---|---|---|---|---|---|---|
| MK-3222 100 mg | 2 | 2 | (100.0) | 2 | (100.0) | 1 | (50.0) |
| MK-3222 200 mg | 3 | 2 | (66.7) | 1 | (33.3) | 0 | |

Ext Week 368

| | | | | | | |
|---|---|---|---|---|---|---|
| MK-3222 100 mg | 1 | 1 | (100.0) | 1 | (100.0) | 0 |
| MK-3222 200 mg | 0 | 0 | | 0 | | 0 |

N = Number of randomized subjects who received at least one dose of study medication in extension and with valid value at baseline and at the time point for endpoint.
n = the number of responders at the visit.
No imputation of missing data.
PASI = Psoriasis Area and Severity Index.

Table 50 summarizes the proportion of subjects with PASI 75, PASI 90, and PASI 100 responses over time for the subjects treated with PFS for the two treatment groups. Within each PASI response measured (PASI 75, PASI 90, and PASI 100) the proportion of subjects with PASI response was maintained throughout the study among subjects who remained in the study at the specified time points. From the extension Week 84 onwards subjects started transitioning to AI with the number of evaluable subjects receiving PFS dropping to single digits from extension Week 120 onwards.

TABLE 50
Proportion of Subjects with a PASI 75, PASI 90, and PASI
100 Response Over Time: Subjects Treated with Pre-Filled
Syringe (Full Analysis Set - Extension) Extension Study

| | | PASI 75 | | PASI 90 | | PASI 100 | |
|---|---|---|---|---|---|---|---|
| Treatment | N | n | (%) | n | (%) | n | (%) |

Week 64

| | | | | | | | |
|---|---|---|---|---|---|---|---|
| MK-3222 100 mg | 161 | 145 | (90.1) | 94 | (58.4) | 52 | (32.3) |
| MK-3222 200 mg | 186 | 159 | (85.5) | 110 | (59.1) | 59 | (31.7) |

Ext Week 0

| | | | | | | | |
|---|---|---|---|---|---|---|---|
| MK-3222 100 mg | 156 | 139 | (89.1) | 93 | (59.6) | 41 | (26.3) |
| MK-3222 200 mg | 182 | 155 | (85.2) | 106 | (58.2) | 54 | (29.7) |

Ext Week 12

| | | | | | | | |
|---|---|---|---|---|---|---|---|
| MK-3222 100 mg | 156 | 142 | (91.0) | 94 | (60.3) | 44 | (28.2) |
| MK-3222 200 mg | 180 | 147 | (81.7) | 108 | (60.0) | 62 | (34.4) |

Ext Week 24

| | | | | | | | |
|---|---|---|---|---|---|---|---|
| MK-3222 100 mg | 158 | 136 | (86.1) | 87 | (55.1) | 43 | (27.2) |
| MK-3222 200 mg | 181 | 150 | (82.9) | 109 | (60.2) | 63 | (34.8) |

Ext Week 36

| | | | | | | | |
|---|---|---|---|---|---|---|---|
| MK-3222 100 mg | 155 | 138 | (89.0) | 85 | (54.8) | 42 | (27.1) |
| MK-3222 200 mg | 175 | 138 | (78.9) | 102 | (58.3) | 53 | (30.3) |

Ext Week 48

| | | | | | | | |
|---|---|---|---|---|---|---|---|
| MK-3222 100 mg | 157 | 136 | (86.6) | 85 | (54.1) | 35 | (22.3) |
| MK-3222 200 mg | 179 | 153 | (85.5) | 104 | (58.1) | 51 | (28.5) |

Ext Week 60

| | | | | | | | |
|---|---|---|---|---|---|---|---|
| MK-3222 100 mg | 156 | 138 | (88.5) | 87 | (55.8) | 44 | (28.2) |
| MK-3222 200 mg | 180 | 151 | (83.9) | 98 | (54.4) | 56 | (31.1) |

Ext Week 72

| | | | | | | | |
|---|---|---|---|---|---|---|---|
| MK-3222 100 mg | 144 | 125 | (86.8) | 80 | (55.6) | 42 | (29.2) |
| MK-3222 200 mg | 171 | 139 | (81.3) | 94 | (55.0) | 52 | (30.4) |

Ext Week 84

| | | | | | | | |
|---|---|---|---|---|---|---|---|
| MK-3222 100 mg | 99 | 85 | (85.9) | 59 | (59.6) | 25 | (25.3) |
| MK-3222 200 mg | 105 | 78 | (74.3) | 51 | (48.6) | 27 | (25.7) |

Ext Week 96

| | | | | | | | |
|---|---|---|---|---|---|---|---|
| MK-3222 100 mg | 47 | 38 | (80.9) | 25 | (53.2) | 12 | (25.5) |
| MK-3222 200 mg | 56 | 45 | (80.4) | 29 | (51.8) | 14 | (25.0) |

Ext Week 120

| | | | | | | | |
|---|---|---|---|---|---|---|---|
| MK-3222 100 mg | 13 | 10 | (76.9) | 6 | (46.2) | 2 | (15.4) |
| MK-3222 200 mg | 10 | 9 | (90.0) | 4 | (40.0) | 2 | (20.0) |

Ext Week 144

| | | | | | | | |
|---|---|---|---|---|---|---|---|
| MK-3222 100 mg | 8 | 5 | (62.5) | 4 | (50.0) | 1 | (12.5) |
| MK-3222 200 mg | 4 | 3 | (75.0) | 1 | (25.0) | 0 | |

Ext Week 168

| | | | | | | | |
|---|---|---|---|---|---|---|---|
| MK-3222 100 mg | 5 | 3 | (60.0) | 3 | (60.0) | 1 | (20.0) |
| MK-3222 200 mg | 3 | 3 | (100.0) | 2 | (66.7) | 1 | (33.3) |

Ext Week 192

| | | | | | | |
|---|---|---|---|---|---|---|
| MK-3222 100 mg | 3 | 1 | (33.3) | 1 | (33.3) | 0 |
| MK-3222 200 mg | 0 | 0 | | 0 | | 0 |

Ext Week 216

| | | | | | |
|---|---|---|---|---|---|
| MK-3222 100 mg | 2 | 1 | (50.0) | 0 | 0 |
| MK-3222 200 mg | 0 | 0 | | 0 | 0 |

Ext Week 240

| | | | | |
|---|---|---|---|---|
| MK-3222 100 mg | 2 | 0 | 0 | 0 |
| MK-3222 200 mg | 0 | 0 | 0 | 0 |

Ext Week 264

| | | | | |
|---|---|---|---|---|
| MK-3222 100 mg | 2 | 0 | 0 | 0 |
| MK-3222 200 mg | 0 | 0 | 0 | 0 |

Ext Week 288

| | | | | |
|---|---|---|---|---|
| MK-3222 100 mg | 1 | 0 | 0 | 0 |
| MK-3222 200 mg | 0 | 0 | 0 | 0 |

N = Number of randomized subjects who received at least one dose of study medication in extension and with valid value at baseline and at the time point for endpoint.
n = the number of responders at the visit.
No imputation of missing data.
PASI = Psoriasis Area and Severity Index.

Table 51 summarizes the proportion of subjects with PASI 75, PASI 90, and PASI 100 responses over time for the subjects treated with AI for the two treatment groups. Within each PASI response measured (PASI 75, PASI 90, and PASI 100) the proportion of subjects with PASI response was maintained among subjects who remained in the study at the specified time points.

TABLE 51
Proportion of Subjects with a PASI 75, PASI 90, and PASI
100 Response Over Time: Subjects Treated with Auto-Injector
(Full Analysis Set - Extension) Extension Study

| | | PASI75 | | PASI90 | | PASI100 | |
|---|---|---|---|---|---|---|---|
| Treatment | N | n | (%) | n | (%) | n | (%) |

Ext Week 48

| | | | | | | | |
|---|---|---|---|---|---|---|---|
| MK-3222 100 mg | 1 | 1 | (100.0) | 1 | (100.0) | 1 | (100.0) |
| MK-3222 200 mg | 1 | 1 | (100.0) | 1 | (100.0) | 0 | |

Ext Week 60

| | | | | | | | |
|---|---|---|---|---|---|---|---|
| MK-3222 100 mg | 2 | 2 | (100.0) | 2 | (100.0) | 1 | (50.0) |
| MK-3222 200 mg | 1 | 1 | (100.0) | 1 | (100.0) | 1 | (100.0) |

Ext Week 72

| | | | | | | | |
|---|---|---|---|---|---|---|---|
| MK-3222 100 mg | 12 | 10 | (83.3) | 9 | (75.0) | 5 | (41.7) |
| MK-3222 200 mg | 10 | 9 | (90.0) | 7 | (70.0) | 4 | (40.0) |

Ext Week 84

| | | | | | | | |
|---|---|---|---|---|---|---|---|
| MK-3222 100 mg | 59 | 51 | (86.4) | 36 | (61.0) | 15 | (25.4) |
| MK-3222 200 mg | 73 | 62 | (84.9) | 47 | (64.4) | 25 | (34.2) |

Ext Week 96

| | | | | | | | |
|---|---|---|---|---|---|---|---|
| MK-3222 100 mg | 107 | 91 | (85.0) | 66 | (61.7) | 29 | (27.1) |
| MK-3222 200 mg | 119 | 95 | (79.8) | 72 | (60.5) | 40 | (33.6) |

Ext Week 120

| | | | | | | | |
|---|---|---|---|---|---|---|---|
| MK-3222 100 mg | 132 | 109 | (82.6) | 71 | (53.8) | 37 | (28.0) |
| MK-3222 200 mg | 161 | 133 | (82.6) | 95 | (59.0) | 51 | (31.7) |

Ext Week 144

| | | | | | | | |
|---|---|---|---|---|---|---|---|
| MK-3222 100 mg | 132 | 113 | (85.6) | 80 | (60.6) | 39 | (29.5) |
| MK-3222 200 mg | 165 | 138 | (83.6) | 96 | (58.2) | 54 | (32.7) |

Ext Week 168

| | | | | | | | |
|---|---|---|---|---|---|---|---|
| MK-3222 100 mg | 130 | 116 | (89.2) | 80 | (61.5) | 41 | (31.5) |
| MK-3222 200 mg | 155 | 133 | (85.8) | 104 | (67.1) | 59 | (38.1) |

Ext Week 192

| | | | | | | | |
|---|---|---|---|---|---|---|---|
| MK-3222 100 mg | 125 | 114 | (91.2) | 78 | (62.4) | 39 | (31.2) |
| MK-3222 200 mg | 146 | 127 | (87.0) | 97 | (66.4) | 51 | (34.9) |

Ext Week 216

| | | | | | | | |
|---|---|---|---|---|---|---|---|
| MK-3222 100 mg | 46 | 42 | (91.3) | 31 | (67.4) | 15 | (32.6) |
| MK-3222 200 mg | 49 | 45 | (91.8) | 36 | (73.5) | 19 | (38.8) |

Ext Week 240

| | | | | | | | |
|---|---|---|---|---|---|---|---|
| MK-3222 100 mg | 45 | 45 | (100.0) | 35 | (77.8) | 19 | (42.2) |
| MK-3222 200 mg | 46 | 44 | (95.7) | 37 | (80.4) | 22 | (47.8) |

Ext Week 264

| | | | | | | | |
|---|---|---|---|---|---|---|---|
| MK-3222 100 mg | 38 | 34 | (89.5) | 28 | (73.7) | 21 | (55.3) |
| MK-3222 200 mg | 42 | 39 | (92.9) | 31 | (73.8) | 20 | (47.6) |

Ext Week 288

| | | | | | | | |
|---|---|---|---|---|---|---|---|
| MK-3222 100 mg | 36 | 33 | (91.7) | 24 | (66.7) | 15 | (41.7) |
| MK-3222 200 mg | 37 | 35 | (94.6) | 31 | (83.8) | 16 | (43.2) |

Ext Week 312

| | | | | | | | |
|---|---|---|---|---|---|---|---|
| MK-3222 100 mg | 19 | 17 | (89.5) | 11 | (57.9) | 6 | (31.6) |
| MK-3222 200 mg | 23 | 21 | (91.3) | 18 | (78.3) | 13 | (56.5) |

Ext Week 332

| | | | | | | | |
|---|---|---|---|---|---|---|---|
| MK-3222 100 mg | 19 | 18 | (94.7) | 15 | (78.9) | 12 | (63.2) |
| MK-3222 200 mg | 22 | 22 | (100.0) | 20 | (90.9) | 13 | (59.1) |

Ext Week 344

| | | | | | | | |
|---|---|---|---|---|---|---|---|
| MK-3222 100 mg | 2 | 2 | (100.0) | 2 | (100.0) | 1 | (50.0) |
| MK-3222 200 mg | 3 | 2 | (66.7) | 1 | (33.3) | 0 | |

Ext Week 368

| | | | | | | |
|---|---|---|---|---|---|---|
| MK-3222 100 mg | 1 | 1 | (100.0) | 1 | (100.0) | 0 |
| MK-3222 200 mg | 0 | 0 | | 0 | | 0 |

N = Number of randomized subjects who received at least one dose of study medication in extension and with valid value at baseline and at the time point for endpoint.
n = the number of responders at the visit.
PASI = Psoriasis Area and Severity Index.
No imputation of missing data.

Change and percent change in PASI score over time. A summary of change and percent change from baseline PASI scores for the two tildrakizumab treatment groups over time in the FAS population is presented in Tables 52 and 53, respectively. Baseline is defined as the last measurement taken prior to the first dose of study medication in the base study. Week 64 is the last scheduled assessment recorded in the base study. Mean changes in PASI scores were maintained throughout the extension study in both treatment groups. Similarly mean percent changes in PASI scores were maintained over time in the two treatment groups (Table 53).

No notable differences in mean changes from baseline in PASI scores over time were observed between subjects who were treated with PFS and those who were treated with autoinjector (AI). Summary of change from baseline in PASI score over time in subjects who self-injected versus ones who did not self-inject is presented. There was no notable difference in change or percent change from baseline in PASI over time observed between subjects who self-injected versus ones who did not self-inject.

TABLE 52
Summary of Change from Baseline in Psoriasis Area and Severity Index
Score Over Time (Full Analysis Set - Extension) Extension Study

| | | Baseline | Time Point | Change from Baseline | | | | |
|---|---|---|---|---|---|---|---|---|
| Treatment | N | Mean (SD) | Mean (SD) | Mean (SD) | Q1 | Median | Q3 | 95% CI |

Week 64

| | | | | | | | | |
|---|---|---|---|---|---|---|---|---|
| MK-3222 100 mg | 239 | 20.0 (7.61) | 2.3 (2.48) | −17.8 (7.33) | −20.8 | −15.9 | −12.6 | (−18.7, −16.8) |
| MK-3222 200 mg | 267 | 21.3 (9.57) | 2.8 (3.50) | −18.5 (8.43) | −21.6 | −16.4 | −12.6 | (−19.5, −17.5) |

Ext Week 0

| | | | | | | | | |
|---|---|---|---|---|---|---|---|---|
| MK-3222 100 mg | 233 | 20.0 (7.66) | 2.3 (2.75) | −17.6 (7.54) | −20.3 | −15.4 | −12.6 | (−18.6, −16.7) |
| MK-3222 200 mg | 263 | 21.4 (9.60) | 3.1 (4.05) | −18.3 (8.39) | −21.7 | −16.2 | −12.5 | (−19.3, −17.3) |

Ext Week 12

| | | | | | | | | |
|---|---|---|---|---|---|---|---|---|
| MK-3222 100 mg | 229 | 20.0 (7.73) | 2.4 (2.65) | −17.6 (7.43) | −20.6 | −15.6 | −12.5 | (−18.6, −16.6) |
| MK-3222 200 mg | 261 | 21.4 (9.62) | 3.0 (4.12) | −18.4 (8.54) | −21.7 | −16.4 | −12.6 | (−19.4, −17.3) |

Ext Week 24

| | | | | | | | | |
|---|---|---|---|---|---|---|---|---|
| MK-3222 100 mg | 228 | 20.0 (7.75) | 2.5 (2.95) | −17.5 (7.36) | −20.1 | −15.8 | −12.3 | (−18.5, −16.5) |
| MK-3222 200 mg | 261 | 21.3 (9.62) | 2.9 (4.01) | −18.4 (8.60) | −21.7 | −16.2 | −12.9 | (−19.5, −17.4) |

Ext Week 36

| | | | | | | | | |
|---|---|---|---|---|---|---|---|---|
| MK-3222 100 mg | 221 | 20.1 (7.76) | 2.5 (3.03) | −17.5 (7.47) | −20.2 | −15.9 | −12.2 | (−18.5, −16.6) |
| MK-3222 200 mg | 249 | 21.5 (9.74) | 3.3 (4.28) | −18.2 (8.66) | −21.0 | −16.2 | −12.3 | (−19.3, −17.1) |

Ext Week 48

| | | | | | | | | |
|---|---|---|---|---|---|---|---|---|
| MK-3222 100 mg | 217 | 20.1 (7.75) | 2.6 (2.88) | −17.5 (7.48) | −20.2 | −15.6 | −12.1 | (−18.5, −16.5) |
| MK-3222 200 mg | 255 | 21.5 (9.69) | 3.2 (4.09) | −18.3 (8.56) | −22.1 | −16.2 | −12.6 | (−19.4, −17.3) |

Ext Week 60

| | | | | | | | | |
|---|---|---|---|---|---|---|---|---|
| MK-3222 100 mg | 210 | 19.9 (7.41) | 2.4 (2.90) | −17.5 (7.13) | −19.7 | −16.1 | −12.6 | (−18.5, −16.6) |
| MK-3222 200 mg | 252 | 21.4 (9.75) | 3.1 (3.94) | −18.3 (8.61) | −21.6 | −16.2 | −12.7 | (−19.4, −17.3) |

Ext Week 72

| | | | | | | | | |
|---|---|---|---|---|---|---|---|---|
| MK-3222 100 mg | 205 | 19.9 (7.43) | 2.4 (3.07) | −17.5 (7.10) | −19.6 | −15.8 | −12.4 | (−18.5, −16.6) |
| MK-3222 200 mg | 251 | 21.5 (9.77) | 3.3 (4.54) | −18.1 (8.50) | −22.3 | −16.2 | −12.5 | (−19.2, −17.1) |

Ext Week 84

| | | | | | | | | |
|---|---|---|---|---|---|---|---|---|
| MK-3222 100 mg | 205 | 19.9 (7.44) | 2.5 (3.37) | −17.5 (7.22) | −20.0 | −15.8 | −12.5 | (−18.5, −16.5) |
| MK-3222 200 mg | 247 | 21.5 (9.74) | 3.4 (4.44) | −18.1 (8.51) | −21.6 | −16.2 | −12.5 | (−19.2, −17.0) |

Ext Week 96

| | | | | | | | | |
|---|---|---|---|---|---|---|---|---|
| MK-3222 100 mg | 200 | 20.0 (7.46) | 2.6 (3.43) | −17.4 (7.22) | −20.2 | −15.7 | −12.5 | (−18.4, −16.4) |
| MK-3222 200 mg | 240 | 21.4 (9.77) | 3.2 (4.56) | −18.2 (8.40) | −21.6 | −16.2 | −12.6 | (−19.2, −17.1) |

Ext Week 120

| | | | | | | | | |
|---|---|---|---|---|---|---|---|---|
| MK-3222 100 mg | 189 | 19.9 (7.31) | 2.8 (3.74) | −17.1 (7.23) | −19.9 | −15.4 | −12.3 | (−18.1, −16.1) |
| MK-3222 200 mg | 235 | 21.4 (9.86) | 3.1 (4.46) | −18.4 (8.49) | −21.6 | −16.2 | −12.6 | (−19.4, −17.3) |

Ext Week 144

| | | | | | | | | |
|---|---|---|---|---|---|---|---|---|
| MK-3222 100 mg | 182 | 20.0 (7.45) | 2.7 (3.94) | −17.3 (7.44) | −19.9 | −15.8 | −12.5 | (−18.4, −16.2) |
| MK-3222 200 mg | 229 | 21.3 (9.72) | 3.1 (4.62) | −18.2 (8.39) | −21.3 | −16.0 | −12.7 | (−19.3, −17.1) |

Ext Week 168

| | | | | | | | | |
|---|---|---|---|---|---|---|---|---|
| MK-3222 100 mg | 176 | 19.7 (7.23) | 2.5 (3.55) | −17.2 (7.01) | −19.8 | −15.6 | −12.5 | (−18.3, −16.2) |
| MK-3222 200 mg | 218 | 21.4 (9.86) | 2.6 (3.79) | −18.8 (8.74) | −21.6 | −16.7 | −12.7 | (−20.0, −17.7) |

Ext Week 192

| | | | | | | | | |
|---|---|---|---|---|---|---|---|---|
| MK-3222 100 mg | 168 | 20.0 (7.34) | 2.3 (2.77) | −17.7 (7.16) | −20.2 | −16.1 | −12.8 | (−18.8, −16.6) |
| MK-3222 200 mg | 206 | 21.6 (10.03) | 2.8 (4.27) | −18.8 (8.62) | −21.6 | −16.8 | −13.0 | (−20.0, −17.6) |

Ext Week 216

| | | | | | | | | |
|---|---|---|---|---|---|---|---|---|
| MK-3222 100 mg | 84 | 20.4 (7.65) | 2.6 (3.50) | −17.8 (7.84) | −20.3 | −15.7 | −13.0 | (−19.5, −16.1) |
| MK-3222 200 mg | 105 | 22.9 (11.66) | 3.4 (5.03) | −19.5 (9.57) | −23.0 | −17.4 | −12.8 | (−21.4, −17.7) |

Ext Week 240

| | | | | | | | | |
|---|---|---|---|---|---|---|---|---|
| MK-3222 100 mg | 83 | 20.5 (7.64) | 2.1 (2.73) | −18.4 (7.71) | −21.3 | −15.9 | −13.3 | (−20.1, −16.8) |
| MK-3222 200 mg | 102 | 23.0 (11.65) | 3.4 (5.77) | −19.7 (9.52) | −23.0 | −17.7 | −13.0 | (−21.5, −17.8) |

Ext Week 264

| | | | | | | | | |
|---|---|---|---|---|---|---|---|---|
| MK-3222 100 mg | 75 | 20.7 (7.84) | 2.3 (3.10) | −18.4 (7.97) | −21.3 | −15.9 | −13.3 | (−20.3, −16.6) |
| MK-3222 200 mg | 93 | 23.3 (12.00) | 3.2 (5.15) | −20.0 (10.03) | −24.0 | −17.7 | −13.6 | (−22.1, −18.0) |

Ext Week 288

| | | | | | | | | |
|---|---|---|---|---|---|---|---|---|
| MK-3222 100 mg | 65 | 20.8 (7.44) | 2.0 (2.56) | −18.8 (7.69) | −22.2 | −16.8 | −13.4 | (−20.7, −16.9) |
| MK-3222 200 mg | 73 | 22.9 (12.39) | 3.1 (6.23) | −19.8 (9.73) | −23.0 | −17.7 | −13.0 | (−22.1, −17.6) |

Ext Week 312

| | | | | | | | | |
|---|---|---|---|---|---|---|---|---|
| MK-3222 100 mg | 19 | 15.8 (3.25) | 1.7 (2.41) | −14.1 (2.59) | −15.6 | −14.8 | −12.5 | (−15.4, −12.9) |
| MK-3222 200 mg | 23 | 17.2 (5.52) | 1.2 (2.20) | −16.0 (6.34) | −19.7 | −14.9 | −12.2 | (−18.7, −13.3) |

Ext Week 332

| | | | | | | | | |
|---|---|---|---|---|---|---|---|---|
| MK-3222 100 mg | 19 | 18.3 (5.57) | 0.9 (1.60) | −17.4 (5.49) | −19.7 | −16.2 | −14.3 | (−20.0, −14.7) |
| MK-3222 200 mg | 22 | 16.4 (2.85) | 0.6 (0.85) | −15.9 (2.82) | −17.7 | −16.4 | −12.9 | (−17.1, −14.6) |

Ext Week 344

| | | | | | | | | |
|---|---|---|---|---|---|---|---|---|
| MK-3222 100 mg | 2 | 15.8 (0.42) | 0.5 (0.64) | −15.4 (0.21) | −15.5 | −15.4 | −15.2 | (−17.3, −13.4) |
| MK-3222 200 mg | 3 | 14.8 (3.30) | 5.5 (6.12) | −9.3 (7.61) | −15.4 | −11.8 | −0.8 | (−28.2, 9.6) |

Ext Week 368

| | | | | | | | | |
|---|---|---|---|---|---|---|---|---|
| MK-3222 100 mg | 1 | 34.5 (N/A) | 0.6 (N/A) | −33.9 (N/A) | −33.9 | −33.9 | −33.9 | N/A |
| MK-3222 200 mg | 0 | | | | | | | |

CI = Confidence Interval; Q1 = 25^{th} percentile; Q3 = 75^{th} percentile; SD = Standard Deviation.
N = Number of randomized subjects who received at least one dose of study medication in extension and with valid value at baseline and at the time point for endpoint.
Baseline is defined as the last measurement taken prior to the first study medication in the base study.
Week 64: Last scheduled assessment recorded prior to the last dose in the base study.

TABLE 53
Summary of Percent Change from Baseline in Psoriasis Area and Severity
Index Score Over Time (Full Analysis Set - Extension) Extension Study

| | | Baseline | Time Point | Percent Change from Baseline | | | | |
|---|---|---|---|---|---|---|---|---|
| Treatment | N | Mean (SD) | Mean (SD) | Mean (SD) | Q1 | Median | Q3 | 95% CI |

Week 64

| | | | | | | | | |
|---|---|---|---|---|---|---|---|---|
| MK-3222 100 mg | 239 | 20.0 (7.61) | 2.3 (2.48) | −88.5 (12.01) | −100.0 | −90.6 | −82.5 | (−90.1, −87.0) |
| MK-3222 200 mg | 267 | 21.3 (9.57) | 2.8 (3.50) | −87.3 (12.85) | −100.0 | −90.6 | −79.0 | (−88.9, −85.8) |

Ext Week 0

| | | | | | | | | |
|---|---|---|---|---|---|---|---|---|
| MK-3222 100 mg | 233 | 20.0 (7.66) | 2.3 (2.75) | −88.0 (13.62) | −100.0 | −92.2 | −82.5 | (−89.7, −86.2) |
| MK-3222 200 mg | 263 | 21.4 (9.60) | 3.1 (4.05) | −86.3 (14.86) | −100.0 | −90.6 | −78.5 | (−88.1, −84.5) |

Ext Week 12

| | | | | | | | | |
|---|---|---|---|---|---|---|---|---|
| MK-3222 100 mg | 229 | 20.0 (7.73) | 2.4 (2.65) | −87.7 (12.83) | −100.0 | −91.1 | −80.6 | (−89.4, −86.1) |
| MK-3222 200 mg | 261 | 21.4 (9.62) | 3.0 (4.12) | −86.3 (15.89) | −100.0 | −92.5 | −78.4 | (−88.2, −84.3) |

Ext Week 24

| | | | | | | | | |
|---|---|---|---|---|---|---|---|---|
| MK-3222 100 mg | 228 | 20.0 (7.75) | 2.5 (2.95) | −87.3 (13.59) | −100.0 | −90.8 | −80.7 | (−89.1, −85.5) |
| MK-3222 200 mg | 261 | 21.3 (9.62) | 2.9 (4.01) | −86.8 (15.15) | −100.0 | −92.4 | −78.8 | (−88.6, −84.9) |

Ext Week 36

| | | | | | | | | |
|---|---|---|---|---|---|---|---|---|
| MK-3222 100 mg | 221 | 20.1 (7.76) | 2.5 (3.03) | −87.3 (13.65) | −100.0 | −90.8 | −79.9 | (−89.1, −85.5) |
| MK-3222 200 mg | 249 | 21.5 (9.74) | 3.3 (4.28) | −85.3 (16.05) | −99.3 | −91.3 | −75.3 | (−87.3, −83.3) |

Ext Week 48

| | | | | | | | | |
|---|---|---|---|---|---|---|---|---|
| MK-3222 100 mg | 217 | 20.1 (7.75) | 2.6 (2.88) | −87.0 (13.29) | −98.5 | −90.6 | −81.1 | (−88.7, −85.2) |
| MK-3222 200 mg | 255 | 21.5 (9.69) | 3.2 (4.09) | −85.9 (15.46) | −99.2 | −91.2 | −77.9 | (−87.8, −84.0) |

Ext Week 60

| | | | | | | | | |
|---|---|---|---|---|---|---|---|---|
| MK-3222 100 mg | 210 | 19.9 (7.41) | 2.4 (2.90) | −88.1 (13.20) | −100.0 | −91.6 | −81.7 | (−89.9, −86.3) |
| MK-3222 200 mg | 252 | 21.4 (9.75) | 3.1 (3.94) | −86.2 (15.00) | −100.0 | −90.5 | −78.3 | (−88.1, −84.3) |

Ext Week 72

| | | | | | | | | |
|---|---|---|---|---|---|---|---|---|
| MK-3222 100 mg | 205 | 19.9 (7.43) | 2.4 (3.07) | −88.2 (13.28) | −100.0 | −91.2 | −81. | (−90.0, −86.3) |
| MK-3222 200 mg | 251 | 21.5 (9.77) | 3.3 (4.54) | −85.3 (16.81) | −100.0 | −90.3 | −78.3 | (−87.4, −83.2) |

Ext Week 84

| | | | | | | | | |
|---|---|---|---|---|---|---|---|---|
| MK-3222 100 mg | 205 | 19.9 (7.44) | 2.5 (3.37) | −87.9 (14.56) | −99.7 | −92.3 | −82.3 | (−89.9, −85.9) |
| MK-3222 200 mg | 247 | 21.5 (9.74) | 3.4 (4.44) | −85.1 (16.35) | −100.0 | −90.6 | −75.9 | (−87.1,−83.0) |

Ext Week 96

| | | | | | | | | |
|---|---|---|---|---|---|---|---|---|
| MK-3222 100 mg | 200 | 20.0 (7.46) | 2.6 (3.43) | −87.0 (15.08) | −99.8 | −91.7 | −80.9 | (−89.1, −84.9) |
| MK-3222 200 mg | 240 | 21.4 (9.77) | 3.2 (4.56) | −85.8 (15.87) | −100.0 | −91.6 | −77.7 | (−87.8, −83.7) |

Ext Week 120

| | | | | | | | | |
|---|---|---|---|---|---|---|---|---|
| MK-3222 100 mg | 189 | 19.9 (7.31) | 2.8 (3.74) | −86.2 (16.79) | −100.0 | −90.8 | −81.4 | (−88.6, −83.8) |
| MK-3222 200 mg | 235 | 21.4 (9.86) | 3.1 (4.46) | −86.6 (15.57) | −100.0 | −92.5 | −79.5 | (−88.6, −84.6) |

Ext Week 144

| | | | | | | | | |
|---|---|---|---|---|---|---|---|---|
| MK-3222 100 mg | 182 | 20.0 (7.45) | 2.7 (3.94) | −86.7 (17.88) | −100.0 | −92.2 | −81.6 | (−89.3, −84.1) |
| MK-3222 200 mg | 229 | 21.3 (9.72) | 3.1 (4.62) | −86.3 (16.74) | −100.0 | −91.8 | −78.9 | (−88.4, −84.1) |

Ext Week 168

| | | | | | | | | |
|---|---|---|---|---|---|---|---|---|
| MK-3222 100 mg | 176 | 19.7 (7.23) | 2.5 (3.55) | −87.7 (16.25) | −100.0 | −92.2 | −82.7 | (−90.1, −85.3) |
| MK-3222 200 mg | 218 | 21.4 (9.86) | 2.6 (3.79) | −88.5 (14.16) | −100.0 | −94.0 | −81.0 | (−90.4, −86.7) |

Ext Week 192

| | | | | | | | | |
|---|---|---|---|---|---|---|---|---|
| MK-3222 100 mg | 168 | 20.0 (7.34) | 2.3 (2.77) | −88.5 (13.89) | −100.0 | −92.6 | −82.0 | (−90.7, −86.4) |
| MK-3222 200 mg | 206 | 21.6 (10.03) | 2.8 (4.27) | −88.1 (14.51) | −100.0 | −93.5 | −81.5 | (−90.1,−86.1) |

Ext Week 216

| | | | | | | | | |
|---|---|---|---|---|---|---|---|---|
| MK-3222 100 mg | 84 | 20.4 (7.65) | 2.6 (3.50) | −86.6 (18.64) | −100.0 | −93.6 | −79.7 | (−90.7, −82.6) |
| MK-3222 200 mg | 105 | 22.9 (11.66) | 3.4 (5.03) | −86.8 (14.65) | −99.3 | −91.4 | −79.7 | (−89.7, −84.0) |

Ext Week 240

| | | | | | | | | |
|---|---|---|---|---|---|---|---|---|
| MK-3222 100 mg | 83 | 20.5 (7.64) | 2.1 (2.73) | −89.5 (14.21) | −100.0 | −93.6 | −84.6 | (−92.6, −86.4) |
| MK-3222 200 mg | 102 | 23.0 (11.65) | 3.4 (5.77) | −87.4 (15.40) | −100.0 | −92.5 | −81.8 | (−90.4, −84.3) |

Ext Week 264

| | | | | | | | | |
|---|---|---|---|---|---|---|---|---|
| MK-3222 100 mg | 75 | 20.7 (7.84) | 2.3 (3.10) | −88.8 (15.40) | −100.0 | −95.7 | −82.1 | (−92.4, −85.3) |
| MK-3222 200 mg | 93 | 23.3 (12.00) | 3.2 (5.15) | −87.4 (18.32) | −100.0 | −92.7 | −81.9 | (−91.1,−83.6) |

Ext Week 288

| | | | | | | | | |
|---|---|---|---|---|---|---|---|---|
| MK-3222 100 mg | 65 | 20.8 (7.44) | 2.0 (2.56) | −89.9 (13.50) | −100.0 | −94.5 | −85.4 | (−93.3, −86.6) |
| MK-3222 200 mg | 73 | 22.9 (12.39) | 3.1 (6.23) | −88.8 (13.61) | −100.0 | −93.4 | −82.5 | (−92.0, −85.6) |

Ext Week 312

| | | | | | | | | |
|---|---|---|---|---|---|---|---|---|
| MK-3222 100 mg | 19 | 15.8 (3.25) | 1.7 (2.41) | −90.0 (11.93) | −100.0 | −92.9 | −85.6 | (−95.8, −84.3) |
| MK-3222 200 mg | 23 | 17.2 (5.52) | 1.2 (2.20) | −92.1 (16.05) | −100.0 | −100.0 | −92.0 | (−99.0, −85.1) |

Ext Week 332

| | | | | | | | | |
|---|---|---|---|---|---|---|---|---|
| MK-3222 100 mg | 19 | 18.3 (5.57) | 0.9 (1.60) | −94.9 (8.47) | −100.0 | −100.0 | −93.1 | (−99.0, −90.8) |
| MK-3222 200 mg | 22 | 16.4 (2.85) | 0.6 (0.85) | −96.7 (4.93) | −100.0 | −100.0 | −93.1 | (−98.9, −94.5) |

Ext Week 344

| | | | | | | | | |
|---|---|---|---|---|---|---|---|---|
| MK-3222 100 mg | 2 | 15.8 (0.42) | 0.5 (0.64) | −97.2 (3.95) | −100.0 | −97.2 | −94.4 | (−132.7, −61.7) |
| MK-3222 200 mg | 3 | 14.8 (3.30) | 5.5 (6.12) | −60.8 (47.75) | −93.7 | −82.8 | −6.1 | (−179.4, 57.8) |

Ext Week 368

| | | | | | | | | |
|---|---|---|---|---|---|---|---|---|
| MK-3222 100 mg | 1 | 34.5 (N/A) | 0.6 (N/A) | −98.3 (N/A) | −98.3 | −98.3 | −98.3 | N/A |
| MK-3222 200 mg | 0 | | | | | | | |

CI = Confidence Interval; Q1 = 25th percentile; Q3 = 75th percentile; SD = Standard Deviation.
N = Number of randomized subjects who received at least one dose of study medication in extension and with valid value at baseline and at the time point for endpoint.
Baseline is defined as the last measurement taken prior to the first study medication in the base study.
Week 64: Last scheduled assessment recorded prior to the last dose in the base study.

Physician's Global Assessment Score of “Clear” or “Minimal” with at Least a 2 Grade Reduction from Baseline Over Time. The proportion of subjects with a PGA score of clear or minimal with at least 2 Grade point reduction from baseline was maintained throughout the extension phase in both treatment groups and there were no notable differences in the two treatment groups Table 54.

The proportion of subjects with PGA score of “clear” or “minimal” with at least 2 Grade point reductions from baseline was higher in subjects who self-injected than in subjects who did not self-inject in both treatment groups, at almost all timepoints. A comparison between the proportions of subjects with PGA score of “clear” or “minimal” with at least 2 Grade point reductions from baseline between subjects who were treated with PFS and those who were treated with AI during the extension study could not be derived due to the differences in timepoints when they were used since subjects treated with PFS gradually started shifting to AI from week Week 84/96 onwards. However, responses were seen to be maintained when the subjects were shifted from PFS to AI.

TABLE 54
Proportion of Subjects with PGA Score of Clear or Minimal,
With at Least 2 Grade Reduction From Baseline Over Time
(Full Analysis Set - Extension) Extension Study

| | | PGA Score of Clear or | | PGA Score of | | PGA Score of | |
|---|---|---|---|---|---|---|---|
| | | Minimal | | Minimal | | Clear | |
| Treatment | N | n | (%) | n | (%) | n | (%) |

Week 64

| | | | | | | | |
|---|---|---|---|---|---|---|---|
| MK-3222 100 mg | 239 | 155 | (64.9) | 78 | (32.6) | 77 | (32.2) |
| MK-3222 200 mg | 267 | 167 | (62.5) | 84 | (31.5) | 83 | (31.1) |

Ext Week 0

| | | | | | | | |
|---|---|---|---|---|---|---|---|
| MK-3222 100 mg | 232 | 150 | (64.7) | 81 | (34.9) | 69 | (29.7) |
| MK-3222 200 mg | 261 | 155 | (59.4) | 79 | (30.3) | 76 | (29.1) |

Ext Week 12

| | | | | | | | |
|---|---|---|---|---|---|---|---|
| MK-3222 100 mg | 227 | 139 | (61.2) | 76 | (33.5) | 63 | (27.8) |
| MK-3222 200 mg | 260 | 153 | (58.8) | 71 | (27.3) | 82 | (31.5) |

Ext Week 24

| | | | | | | | |
|---|---|---|---|---|---|---|---|
| MK-3222 100 mg | 225 | 139 | (61.8) | 72 | (32.0) | 67 | (29.8) |
| MK-3222 200 mg | 258 | 147 | (57.0) | 66 | (25.6) | 81 | (31.4) |

Ext Week 36

| | | | | | | | |
|---|---|---|---|---|---|---|---|
| MK-3222 100 mg | 221 | 123 | (55.7) | 59 | (26.7) | 64 | (29.0) |
| MK-3222 200 mg | 244 | 134 | (54.9) | 73 | (29.9) | 61 | (25.0) |

Ext Week 48

| | | | | | | | |
|---|---|---|---|---|---|---|---|
| MK-3222 100 mg | 217 | 126 | (58.1) | 69 | (31.8) | 57 | (26.3) |
| MK-3222 200 mg | 253 | 139 | (54.9) | 75 | (29.6) | 64 | (25.3) |

Ext Week 60

| | | | | | | | |
|---|---|---|---|---|---|---|---|
| MK-3222 100 mg | 210 | 125 | (59.5) | 59 | (28.1) | 66 | (31.4) |
| MK-3222 200 mg | 249 | 139 | (55.8) | 67 | (26.9) | 72 | (28.9) |

Ext Week 72

| | | | | | | | |
|---|---|---|---|---|---|---|---|
| MK-3222 100 mg | 205 | 120 | (58.5) | 54 | (26.3) | 66 | (32.2) |
| MK-3222 200 mg | 249 | 144 | (57.8) | 70 | (28.1) | 74 | (29.7) |

Ext Week 84

| | | | | | | | |
|---|---|---|---|---|---|---|---|
| MK-3222 100 mg | 205 | 128 | (62.4) | 71 | (34.6) | 57 | (27.8) |
| MK-3222 200 mg | 246 | 141 | (57.3) | 72 | (29.3) | 69 | (28.0) |

Ext Week 96

| | | | | | | | |
|---|---|---|---|---|---|---|---|
| MK-3222 100 mg | 200 | 114 | (57.0) | 58 | (29.0) | 56 | (28.0) |
| MK-3222 200 mg | 239 | 137 | (57.3) | 68 | (28.5) | 69 | (28.9) |

Ext Week 120

| | | | | | | | |
|---|---|---|---|---|---|---|---|
| MK-3222 100 mg | 189 | 105 | (55.6) | 53 | (28.0) | 52 | (27.5) |
| MK-3222 200 mg | 235 | 142 | (60.4) | 74 | (31.5) | 68 | (28.9) |

Ext Week 144

| | | | | | | | |
|---|---|---|---|---|---|---|---|
| MK-3222 100 mg | 182 | 107 | (58.8) | 53 | (29.1) | 54 | (29.7) |
| MK-3222 200 mg | 229 | 137 | (59.8) | 71 | (31.0) | 66 | (28.8) |

Ext Week 168

| | | | | | | | |
|---|---|---|---|---|---|---|---|
| MK-3222 100 mg | 176 | 108 | (61.4) | 51 | (29.0) | 57 | (32.4) |
| MK-3222 200 mg | 218 | 135 | (61.9) | 58 | (26.6) | 77 | (35.3) |

Ext Week 192

| | | | | | | | |
|---|---|---|---|---|---|---|---|
| MK-3222 100 mg | 168 | 98 | (58.3) | 45 | (26.8) | 53 | (31.5) |
| MK-3222 200 mg | 206 | 125 | (60.7) | 59 | (28.6) | 66 | (32.0) |

Ext Week 216

| | | | | | | | |
|---|---|---|---|---|---|---|---|
| MK-3222 100 mg | 84 | 55 | (65.5) | 26 | (31.0) | 29 | (34.5) |
| MK-3222 200 mg | 103 | 60 | (58.3) | 30 | (29.1) | 30 | (29.1) |

Ext Week 240

| | | | | | | | |
|---|---|---|---|---|---|---|---|
| MK-3222 100 mg | 83 | 57 | (68.7) | 24 | (28.9) | 33 | (39.8) |
| MK-3222 200 mg | 101 | 62 | (61.4) | 29 | (28.7) | 33 | (32.7) |

Ext Week 264

| | | | | | | | |
|---|---|---|---|---|---|---|---|
| MK-3222 100 mg | 75 | 50 | (66.7) | 19 | (25.3) | 31 | (41.3) |
| MK-3222 200 mg | 93 | 53 | (57.0) | 23 | (24.7) | 30 | (32.3) |

Ext Week 288

| | | | | | | | |
|---|---|---|---|---|---|---|---|
| MK-3222 100 mg | 65 | 39 | (60.0) | 15 | (23.1) | 24 | (36.9) |
| MK-3222 200 mg | 73 | 40 | (54.8) | 20 | (27.4) | 20 | (27.4) |

Ext Week 312

| | | | | | | | |
|---|---|---|---|---|---|---|---|
| MK-3222 100 mg | 19 | 8 | (42.1) | 2 | (10.5) | 6 | (31.6) |
| MK-3222 200 mg | 23 | 14 | (60.9) | 1 | (4.3) | 13 | (56.5) |

Ext Week 332

| | | | | | | | |
|---|---|---|---|---|---|---|---|
| MK-3222 100 mg | 19 | 14 | (73.7) | 2 | (10.5) | 12 | (63.2) |
| MK-3222 200 mg | 22 | 20 | (90.9) | 7 | (31.8) | 13 | (59.1) |

Ext Week 344

| | | | | | | | |
|---|---|---|---|---|---|---|---|
| MK-3222 100 mg | 2 | 2 | (100.0) | 1 | (50.0) | 1 | (50.0) |
| MK-3222 200 mg | 3 | 0 | | 0 | | 0 | |

Ext Week 368

| | | | | | | |
|---|---|---|---|---|---|---|
| MK-3222 100 mg | 1 | 1 | (100.0) | 1 | (100.0) | 0 |
| MK-3222 200 mg | 0 | 0 | | 0 | | 0 |

N = Number of randomized subjects who received at least one dose of study medication in extension and with valid value at baseline and at the time point for endpoint.
n = the number of responders at the visit.
PGA = Physician's Global Assessment.
No imputation of missing data.

Incidence of Anti-Drug Antibody Summary of anti-tildrakizumab immunogenicity status at the end of the extension study are provided by treatment group for ADA evaluable subjects in the extension study in Table 55. Across the two treatment groups at the end of the extension study, the number of evaluable subjects stood at 503, out of which 402 (79.9%) subjects were categorized as negative, 32 (6.4%) subjects as inconclusive, and 471 [93.6%] subjects as conclusive (evaluable minus inconclusive).

Across the two treatment groups, 69 (13.7%) subjects were ADA positive. Of these, 46 (9.1%) subjects were categorized as treatment emergent ADA positive and 23 (4.6%) subjects were categorized as non-treatment emergent ADA positive. The proportion of treatment emergent ADA positive subjects was numerically higher in the tildrakizumab 200 mg group (30 [11.2%] subjects) compared with the tildrakizumab 100 mg group (16 [6.8%] subjects). The maximum post-dose titer of these subjects ranged from <1 (represented as 0.5) to 4096.

Across the two treatment groups, 23 (4.6%) treatment-emergent ADA positive subjects and 6 (1.2%) non-treatment-emergent ADA positive subjects had at least one sample test positive for neutralizing antibodies.

TABLE 55
Summary of Subject Anti-MK-3222 Immunogenicity Status by Dose Level: End
of Extension Study (ADA Evaluable - Extension) Base and Extension Study

| | MK-3222 Treatment in Extension | | | | | |
|---|---|---|---|---|---|---|
| | MK-3222 | | MK-3222 | | | |
| | 100 mg | | 200 mg | | Total | |
| Evaluable subjects^{†} | 236 | | 267 | | 503 | |
| Conclusive subjects^{†} | 224 | (94.9%) | 247 | (92.5%) | 471 | (93.6%) |
| Inconclusive^{†} | 12 | (5.1%) | 20 | (7.5%) | 32 | (6.4%) |

Immunogenicity status^{‡}

| | | | | | | |
|---|---|---|---|---|---|---|
| Negative | 198 | (83.9%) | 204 | (76.4%) | 402 | (79.9%) |
| Non-treatment emergent positive | 10 | (4.2%) | 13 | (4.9%) | 23 | (4.6%) |
| Treatment emergent positive | 16 | (6.8%) | 30 | (11.2%) | 46 | (9.1%) |

All ADA positive subjects^{‡}

| | | | | | | |
|---|---|---|---|---|---|---|
| Total Positive Subjects | 26 | (11.0%) | 43 | (16.1%) | 69 | (13.7%) |

Maximum Post-dose Titer^{§}

| | | | |
|---|---|---|---|
| 0.5 (<1) | 1 | 6 | 7 |
| 1 | 7 | 11 | 18 |
| 2 | 1 | 2 | 3 |
| 4 | 2 | 2 | 4 |
| 8 | 3 | 1 | 4 |
| 16 | 4 | 2 | 6 |
| 32 | 0 | 2 | 2 |
| 64 | 1 | 4 | 5 |
| 128 | 0 | 3 | 3 |
| 4096 | 0 | 1 | 1 |

Neutralizing antibody results
Non-treatment emergent subjects

| | | | | | | |
|---|---|---|---|---|---|---|
| Neutralizing negative^{†,∥} | 7 | (3.0%) | 10 | (3.7%) | 17 | (3.4%) |
| Neutralizing positive^{†,∥} | 3 | (1.3%) | 3 | (1.1%) | 6 | (1.2%) |

Treatment emergent subjects

| | | | | | | |
|---|---|---|---|---|---|---|
| Neutralizing negative^{†,∥} | 7 | (3.0%) | 14 | (5.2%) | 21 | (4.2%) |
| Neutralizing positive^{†,∥} | 9 | (3.8%) | 14 | (5.2%) | 23 | (4.6%) |

^{†}Included were subjects with at least one ADA sample available after treatment with MK-3222.
^{‡}Denominator was total number of evaluable subjects.
^{§}Represented the number of subjects who showed a post-dose maximum titer in each titer category. Includes subjects who had titers <1, these subjects were counted in the 0.5 category.
^{∥}Only samples which were positive in the anti-MK-3222 antibody assay were tested in the neutralizing anti-MK-3222 assay. Subjects considered positive for neutralizing antibodies had at least one sample that tested positive in the neutralizing antibody assay.

Efficacy: The PASI 50, PASI 75, PASI 90, and PASI 100, and PGA responses were maintained in a majority of subjects during the extension study in both the treatment arms. The response rates were generally comparable between the two treatment arms. Very few subjects experienced any adverse experience or device failure with the use of AI over time. The response rates were numerically lower in subjects who had neutralizing antibodies than in the ADA negative subjects, however, since the number of subjects in TE-POS-NAb-POS category is much less than the overall number of ADA evaluable subjects, a definitive conclusion could not be drawn on the effect of NAb on the efficacy of tildrakizumab.

### Efficacy Results Summary

The proportion of subjects with a PASI 50 response among those who were responders at Week 64, was maintained throughout the extension study and was comparable between the tildrakizumab 100 mg and tildrakizumab 200 mg treatment groups.
The proportion of subjects with a PASI 75 response among those who were responders at Week 64 was maintained during the extension study in a majority of the subjects and was comparable between the tildrakizumab 100 mg and tildrakizumab 200 mg treatment groups.
The proportion of subjects with a PASI 90 response among those who were responders at Week 64, was maintained throughout the extension study in a majority of the subjects; the 200 mg dose had a better response till week 48; from week 60 to week 96 the responses were comparable between the two treatment arms, and for the remaining weeks tildrakizumab 200 mg treatment group displayed a better response.
In the proportion of subjects with PASI 100 response among those who were responders at Week 64, the proportion of responders in 200 mg treatment arm was consistently higher throughout the study. The tildrakizumab 200 mg treatment group displayed a better response at most of the time points through the extension study.
The proportion of subjects with a PASI 75, PASI 90, and PASI 100 response over time irrespective of the response status at week 64, was maintained throughout the study among subjects who remained in the study at the specified time points and was comparable between the tildrakizumab 100 mg and tildrakizumab 200 mg treatment groups.
Mean and percent changes in PASI scores were maintained throughout the extension study in both treatment groups.
No notable difference in change or percent change from baseline in PASI over time observed between subjects who self-injected and who did not self-inject AND between subjects who were treated with PFS and those who were treated with AI.
The proportion of subjects with a PGA score of clear or minimal with at least 2 Grade point reduction from baseline was maintained throughout the extension phase in both treatment groups and there were no notable differences in the two treatment groups. No meaningful differences in the proportions of subjects within each PASI responses measured (PASI 75, 90, and 100) AND PGA score of “clear” or “minimal” with at least 2 Grade point reductions from baseline could be derived between subjects who were treated with PFS and those who were treated with AI during the extension study.
The proportion of subjects who self-injected with the PFS was not meaningfully different between the treatment groups until Week 72 and it decreased from Week 84 onwards across the two treatment groups since the subjects started shifting to treatment with AI.
The proportion of subjects with any adverse experience or with any device failure was very low over time (0.8% and ≤3.4%, respectively).
Out of 503 ADA evaluable subjects, overall, 471 (93.6%) subjects had conclusive results. Of the 503 ADA evaluable subjects, 402 (79.9%) subjects were ADA negative, and 23 (4.6%) subjects were non treatment emergent ADA positive while 46 (9.1%) subjects were treatment emergent ADA positive. The proportion of ADA positive and treatment emergent ADA positive subjects were numerically higher in the tildrakizumab 200 mg group (43 [16.1%] subjects and 30 [11.2%] subjects) compared with the tildrakizumab 100 mg group (26 [11.0%] subjects and 16 [6.8%] subjects).
The proportion of subjects with PASI 75, PASI 90, PASI 100 responses, and PGA score of “minimal” or “clear” was numerically lower in TE-POS-NAb-POS subjects than the ADA negative subjects however, since the number of subjects in TE-POS-NAb-POS category is much less than the overall number of ADA evaluable subjects, drawing a conclusion on the effect of NAb on PASI 75, PASI 90, PASI 100 responses, and PGA score of “minimal” or “clear” is difficult.
No notable differences were observed in the 5 year exposure adjusted frequencies of AEs reported in subjects with treatment emergent ADA, non-treatment-emergent ADA and ADA negative and or inconclusive between the two tildrakizumab treatment groups due to the small number of ADA positive subjects.
The overall number of subjects with treatment-emergent ADA was small compared to the overall subjects who were negative for ADA hence, no meaningful conclusions could be drawn on differences in AEs by SOC and PT in subjects between treatment-emergent ADA and subjects who were negative for ADA.

### Safety Evaluation

The proportion of subjects with one or more AEs was comparable between the two treatment groups (216 [90.4%] in tildrakizumab 100 mg group and 231 [86.5%] in tildrakizumab 200 mg group). The proportions of subjects with SAEs (60 [25.1%] in tildrakizumab 100 mg group, and 58 [21.7%] in tildrakizumab 200 mg group) and drug-related AEs (44 [18.4%] in the tildrakizumab 100 mg group and 50 [18.7%] in the tildrakizumab 200 mg group) were not meaningfully different in the two treatment groups. The frequency of discontinuation due to AEs was numerically higher in the tildrakizumab 100 mg group, 26 (10.9%), compared to the tildrakizumab 200 mg group, 13 (4.9%). The proportion of subjects with serious drug-related AEs was numerically higher in the tildrakizumab 100 mg group, 12 (5.0%), compared to the tildrakizumab 200 mg group, 7 (2.6%).

TABLE 56
Adverse Events Summary (All Subjects as Treated - Extension) Extension Study

| | MK-3222 | | MK-3222 | | | |
|---|---|---|---|---|---|---|
| | 100 mg | | 200 mg | | Total | |
| | n | (%) | n | (%) | n | (%) |
| Subjects in population^{†} | 239 | | 267 | | 506 | |
| with one or more adverse events | 216 | (90.4) | 231 | (86.5) | 447 | (88.3) |
| with no adverse events | 23 | (9.6) | 36 | (13.5) | 59 | (11.7) |
| with drug-related^{‡} adverse events | 44 | (18.4) | 50 | (18.7) | 94 | (18.6) |
| with serious adverse events | 60 | (25.1) | 58 | (21.7) | 118 | (23.3) |
| with serious drug-related^{‡} adverse | 12 | (5.0) | 7 | (2.6) | 19 | (3.8) |
| events | | | | | | |
| who died | 3 | (1.3) | 2 | (0.7) | 5 | (1.0) |
| discontinued^{§} due to an adverse event | 26 | (10.9) | 13 | (4.9) | 39 | (7.7) |
| discontinued^{§} due to a drug-related^{‡} | 9 | (3.8) | 2 | (0.7) | 11 | (2.2) |
| adverse event | | | | | | |
| discontinued due to a serious adverse | 23 | (9.6) | 7 | (2.6) | 30 | (5.9) |
| event | | | | | | |
| discontinued^{§} due to a serious drug- | 7 | (2.9) | 1 | (0.4) | 8 | (1.6) |
| related^{‡} adverse event | | | | | | |

^{†}Subjects who took at least one dose of extension study medication based on the treatment actually received.
^{‡}Determined by the investigator to be related to the drug.
^{§}Study medication withdrawn.

The frequencies of overall AEs and drug-related AEs gradually decreased with longer duration of treatment (overall AEs: Year 1: 55.7%; Year 2: 58.1%; Year 3: 51.2%; Year 4: 40.3%; drug-related AEs: Year 1: 8.7%; Year 2: 7.1%; Year 3: 4.5%; Year 4: 2.8%). The overall year-to-year AEs are higher in tildrakizumab 200 mg group, while there were no notable differences between the two treatment groups for drug-related AEs.

The overall frequencies of AEs, drug-related AEs and SAEs were higher in subjects treated with PFS compared to those treated with AI. This difference could be a reflection of the fact that all subjects started on PFS and some of them shifted to AI.

The cumulative exposure-adjusted frequencies of overall AEs, drug-related AEs and SAEs were comparable between the two treatment groups. Similarly, the 5-year cumulative exposure adjusted frequencies of overall AEs, drug-related AEs and SAEs in the base and extension study were similar between two treatment groups

### Safety Conclusions

No meaningful differences were observed in the overall frequency of AEs between the two treatment groups (216 [90.4%] subjects and 231 [86.5%] subjects in the tildrakizumab 100 mg group and 200 mg group, respectively).
Incidence rates of SAEs were comparable in the two treatment groups. Incidence of SAEs by SOC was similar in the tildrakizumab 100 mg, and tildrakizumab 200 mg groups except for SAE of Neoplasms benign, malignant and unspecified (8.8% in tildrakizumab 100 mg group and 4.1% tildrakizumab 200 mg group).
The incidence rate of serious drug-related AEs was low overall in the study (19 [3.8%]) and numerically higher (12 [5.0%]) in the tildrakizumab 100 mg group compared to (7 [2.6%]) in the tildrakizumab 200 mg group.
The frequency of AEs that led to discontinuation of study medication was numerically higher in the tildrakizumab 100 mg group (26 [10.9%]) compared to the tildrakizumab 200 mg group (13 [4.9%]).
A total of five deaths were reported due to six SAEs during this extension study, and out of these six SAEs, the events cardiac failure chronic and metastatic carcinoma of bladder deaths were drug-related.
The incidence rates of drug-related AEs showed no notable difference between the two treatment groups. The most common SOC for drug-related AEs in the extension study was the infections and infestations SOC (12.1% overall; 10% in the tildrakizumab 100 mg group and 13.9% in tildrakizumab 200 mg group) and the most common drug-related AE was upper respiratory tract infection (3.4% overall; 2.9% in the tildrakizumab 100 mg and 3.7% in the tildrakizumab 200 mg group).
The 5-year cumulative exposure adjusted frequencies of overall AEs, drug-related AEs and SAEs were similar between two treatment groups while no notable differences were observed between the two treatment groups in frequencies of drug-related AEs (by SOC and PT) and serious drug-related AEs. In the categories of AEs resulting in discontinuation of study medication, and drug-related AEs resulting in discontinuation of study medication, exposure adjusted incidence of Neoplasms benign, malignant and unspecified (incl. cysts and polyps) SOC was slightly higher in 100 mg treatment group and compared to 200 mg treatment group.
No notable differences in the incidence of Tier 1 AEs were observed between the two treatment groups during the extension study except for malignancies which were reported in numerically higher numbers in 100 mg treatment group.
The frequencies of Tier 2 SAEs, discontinued due to an AE, and discontinued due to drug-related adverse event were numerically higher in the tildrakizumab 100 mg group compared to the tildrakizumab 200 mg group while the Tier 2 AEs among subjects with treatment emergent ADAs were numerically higher in the tildrakizumab 200 mg group compared to the tildrakizumab 100 mg group.
The overall incidence rate of confirmed composite adjudicated CV events during the extension study was relatively low (17 [3.4%] subjects overall) and was comparable in the two treatment groups (7 [2.9%] subjects in tildrakizumab 100 mg group and 10 [3.7%] subjects in tildrakizumab 200 mg group). The incidence rate of confirmed MACE and confirmed extended MACE was similar between the two treatment groups. The incidence rate for fatal or non-fatal thrombotic/embolic/ischemic events was numerically higher in the tildrakizumab 200 mg group (9 [3.4%] subjects) compared to the tildrakizumab 100 mg group (5 [2.1%] subjects).
The overall incidence rate of confirmed adjudicated CV events was low (19 [3.8%] subjects) and was not notably different in the two treatment groups (8 [3.3%] subjects in the tildrakizumab 100 mg group and 11[4.1%] subjects in tildrakizumab 200 mg group).
No notable differences were observed between the treatment groups for the performed laboratory tests and vital signs during the extension study.

### Example 14. Low Cardiotoxicity in Long-Term Treatment of Psorirasis with Tildrakizumab

The FDA Adverse Event Reporting System (FAERS) is a publicly available database that collects information on adverse events (AEs) reported for approved pharmaceutical and biologic products. The database provides information on the pharmacological agent, indications for use, and reported adverse effects.

Cardiovascular side effects reported in FAERS were investigated by using the following key medical terms according to the Medical Dictionary for Regulatory Activities: “atrial fibrillation,” “pericarditis,” “coronary artery disease,” “coronary artery occlusion,” “angina unstable,” acute coronary syndrome,” and “heart failure”.

Over 25 million adverse effects from all drugs have been reported in the FAERS database, with 334,399 adverse effects recorded for therapeutic biologics for psoriasis. Among all therapeutic biologics for psoriasis in the FAERS database, tildrakizumab had the lowest number of AEs with 723. Of the adverse effects reported for psoriasis biologics, 3,852 AEs were related to cardiac related complications. Cardiac AEs included pericarditis, atrial fibrillation, coronary artery disease, and heart failure, with the most common reported AEs being coronary artery disease (1567, 40.68%), pericarditis (801, 20.79%), and atrial fibrillation (579, 15.03%). The death rate of cardiac AEs was 5-9% for the psoriasis biologics investigated. However, tildrakizumab had no reported deaths from cardiac associated AEs. Further, tildrakizumab has been demonstrated post phase 3 studies to have no significant changes in cardiometabolic risk, regardless of whether treated individuals have metabolic syndrome. See Table 57.

TABLE 57
Reported Cardiac Adverse Events With Biologics for Psoriasis in FAERS Databasea

| Adverse | | | | | | |
|---|---|---|---|---|---|---|
| Event | Secukinumab | Ixekizumab | Brodalumab | Guselkumab | Risankizumab | Tildrakizumab |
| Pericarditis | 539 | 13 | 6 | 5 | 9 | 0 |
| Atrial | 249 | 27 | 4 | 16 | 84 | 4 |
| Fibrillation | | | | | | |
| Coronary | 937 | 111 | 27 | 68 | 288 | 7 |
| Artery | | | | | | |
| Disease | | | | | | |

^{a}Among the AEs recorded in the FAERS database, which contains >25 million adverse events, a total of 334,399 events were associated with newly approved therapeutic agents for psoriasis.
Cardiac adverse events accounted for 3,852 cases, including pericarditis, atrial fibrillation, and coronary artery disease. FAERS, FDA Adverse Event Reporting System.

Conclusion: Tildrakizumab has a lower reported incidence of cardiac-related adverse effects and no significant changes in cardiometabolic risk in treating plaque psoriasis.

### Example 15. Efficacy and Safety Through Week 28 in Patients with Early Vs Late Onset Psoriasis

Psoriasis characteristics and response to treatment can vary by age of onset. A post-hoc analysis of Phase 3 reSURFACE 1 (NCT01722331) and reSURFACE 2 (NCT01729754) investigated efficacy and safety over time through week 28 based on early (<40 years) or late (≥40 years) onset of psoriasis. Efficacy was assessed through Week 28 using absolute Psoriasis Area and Severity Index (PASI), a Physician's Global Assessment (PGA) score of clear or almost clear (PGA 0/1), and a Dermatology Life Quality Index (DLQI). Estimates and P-values were obtained from a logistic regression model. Missing data were handled using non-responder imputation for absolute PASI, relative PASI, PGA, and DLQI responses. Mean DLQI was analysed as observed. Safety was assessed from treatment-emergent adverse events (TEAEs) through Week 28.

Among patients treated with tildrakizumab 100 mg, 225/248 (90.7%) with early-onset psoriasis and 141/153 (92.2%) with late-onset psoriasis completed Week 28. Mean±standard deviation age of patients randomized to tildrakizumab 100 mg vs placebo was 50.0±8.2 years vs 50.6±7.8 in patients with psoriasis onset at <40 years of age and 58.4±8.7 vs 58.3±9.0 in patients with psoriasis onset at ≥40 years of age. At Week 28, significantly more late-onset vs early-onset patients achieved PASI 90 responses (81 [52.9%] vs 111 [44.8%]; P=0.0040), PASI 100 responses (36 [23.5%] vs 42 [16.9%]; P=0.0015), and PASI<1 (61 [39.9%] vs 80 [32.3%]; P=0.0016), whereas significantly more early-onset vs late-onset patients achieved DLQI 0/1 (125 [50.4%]) vs 71 [46.4%]; P=0.0270). There were no significant differences by age of onset in PASI 75 response, PGA 0/1, or PASI<3 rates. Age at enrollment was a significant factor for achievement of PASI 90 (P=0.0040), PASI 100 (P=0.0002), and PASI<1 (P=0.0030). In patients treated with tildrakizumab 100 mg, TEAEs were recorded in 160 (64.5%) early-onset and 97 (63.4%) late-onset patients. Of these, 7 (2.8%) patients in the early-onset group and 10 (6.5%) in the late-onset group had TEAEs considered severe. See Table 58.

TABLE 58
Efficacy at Week 28 in patients originally
randomized to tildrakizumab onset

| | Age of psoriasis onset | | |
|---|---|---|---|
| | <40 years | ≥40 years | |
| Responders, | TIL 100 mg | TIL 100 mg | |
| n (%) | (n = 248) | (n = 153) | P-value |
| PASI 75 | 171 (69.0) | 114 (74.5) | 0.5989 |
| PASI 90 | 111 (44.8) | 81 (52.9) | 0.0040 |
| PASI 100 | 42 (16.9) | 36 (23.5) | 0.0015 |
| PGA 0/1 | 144 (58.1) | 96 (62.7) | 0.7276 |
| DLQI 0/1 | 125 (50.4) | 71 (46.4) | 0.0270 |
| PASI <3 | 140 (56.5) | 96 (62.7) | 0.6383 |
| PASI <1 | 80 (32.3) | 61 (39.9) | 0.0016 |

Estimates and P-values were obtained from a logistic regression model with the following: treatment group, psoriasis onset group, prior biologic use (yes/no), baseline weight group (≤90 kg vs >90 kg), and week as fixed effects; age at enrollment and baseline score as covariates; and subject as a random effect.
DLQI, Dermatology Life Quality Index, DLQI 0/1, DLQI of 0 or 1;
PASI, Psoriasis Area Severity Index;
PASI 70/90/100, ≥75%/90%/100% reduction in PASI from baseline;
PGA, Physician Global Assessment;
PGA 0/1, PGA score of 0 or 1;
TIL, tildrakizumab

Conclusion: Tildrakizumab was equally or more effective in patients with late-onset vs early-onset psoriasis, whereas early-onset patients were more likely to report minimal impact of psoriasis on quality of life. The safety profile of tildrakizumab was comparable in patients with early versus late-onset psoriasis.

Disclosed herein are the results of two multicenter, randomized, double-blind, placebo-controlled studies to assess the long-term safety and tolerability of tildrakizumab in subjects with moderate-to-severe chronic plaque psoriasis. Of the at least 1,000 subjects from the combination of both studies, there were no notable differences in the overall frequency of adverse events observed between the two treatment groups (100 mg or 200 mg). Furthermore, PASI scores were maintained throughout both studies in both treatment groups (100 mg or 200 mg). Thus, the safety and efficacy data from both Phase 3 extension studies presented herein support the assessment of long-term safety and tolerability of tildrakizumab.

Having described the subject matter of the disclosure in detail and by reference to specific embodiments thereof, it will be apparent that modifications and variations are possible without departing from the scope of the claimed subject matter.

What is claimed is:

1: A method of treating plaque psoriasis comprising administering an anti-IL-23p19 antibody hum13B8-b to a patient in need thereof, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2; and wherein hum13B8-b is administered to the patient for at least up to about 60 weeks.

2: The method according to claim 1, wherein the plaque psoriasis is moderate to severe plaque psoriasis.

3: The method according to claim 1, wherein hum13B8-b is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

4: The method according to claim 1, wherein hum13B8-b is administered to the patient about every 12 weeks.

5: The method according to claim 1, wherein hum13B8-b is administered to the patient subcutaneously.

6: The method according to claim 5, wherein hum13B8-b is administered to the patient by subcutaneous injection.

7: The method according to claim 6, wherein hum13B8-b is administered to the patient using an auto-injector or prefilled syringe.

8: The method according to claim 1, wherein a therapeutically effective amount of hum13B8-b is administered to the patient.

9: The method according to claim 1, wherein 100 mg or 200 mg of hum13B8-b is administered to the patient.

10: The method according to claim 1, wherein a first dose of hum13B8-b is administered to the patient on week 0 and a subsequent dose of hum13B8-b is administered to the patient about every 12 weeks thereafter.

11: The method according to claim 10, wherein:
(a) the first dose and the subsequent dose are 100 mg; or
(b) the first dose and the subsequent dose are 200 mg.

12: The method according to claim 10, wherein:
(a) the first dose is 100 mg and the subsequent dose is 200 mg; or
(b) the first dose is 200 mg and the subsequent dose is 100 mg.

13: The method according to claim 10, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

14: The method according to claim 1, wherein a first dose of hum13B8-b is administered to the patient on week 0, a second dose of hum13B8-b is administered to the patient at about 4 weeks, and a subsequent dose of hum13B8-b is administered to the patient about every 4 to 12 weeks thereafter.

15: The method according to claim 14, wherein the subsequent dose is administered about every 12 weeks for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

16: The method according to claim 1, wherein administration of hum13B8-b results in the patient maintaining a Physician's Global Assessment (PGA) score of “clear” or “almost clear” with at least a 2-point reduction from Baseline for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

17: The method according to claim 1, wherein administration of hum13B8-b results in the patient maintaining at least a 50% reduction in the Psoriasis Area and Severity Index (PASI 50) for at least up to about 60 weeks, for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

18: The method according to claim 1, wherein administration of hum13B8-b results in the patient experiencing no increase in adverse events (AEs), drug-related AEs, severe adverse events (SAEs), Tier 1 AEs, or Tier 2 AEs for at least up to about 60 weeks as compared to treatment for up to about 52 weeks.

19: The method according to claim 18, wherein administration of hum13B8-b results in the patient experiencing no increase in adverse events (AEs), drug-related AEs, severe adverse events (SAEs), Tier 1 AEs, or Tier 2 AEs for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.

20: A method for maintaining a Physician's Global Assessment (PGA) score of “clear” or “almost clear” with at least a 2-point reduction from Baseline in a patient with plaque psoriasis comprising administering an anti-IL-23p19 antibody hum13B8-b to a patient in need thereof, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2; and wherein hum13B8-b is administered to the patient for at least up to about 60 weeks.

21: The method according to claim 20, wherein the plaque psoriasis is moderate to severe plaque psoriasis.

22: The method according to claim 20, wherein hum13B8-b is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

23: A method for maintaining at least a 50% reduction in the Psoriasis Area and Severity Index (PASI 50) in a patient with plaque psoriasis comprising administering an anti-IL-23p19 antibody hum13B8-b to a patient in need thereof, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2; and wherein hum13B8-b is administered to the patient for at least up to about 60 weeks.

24: The method according to claim 23, wherein the plaque psoriasis is moderate to severe plaque psoriasis.

25: The method according to claim 23, wherein hum13B8-b is administered to the patient for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks.

26: A method of treating plaque psoriasis comprising administering an anti-IL-23p19 antibody hum13B8-b to a patient in need thereof, wherein hum13B8-b comprises:
(i) a light chain polypeptide comprising the amino acid sequence of SEQ ID NO: 1, and
(ii) a heavy chain polypeptide comprising the amino acid sequence of SEQ ID NO: 2; wherein hum13B8-b is administered to the patient for at least up to about 60 weeks; and wherein the patient has a reduced risk of a cardiac adverse event as compared to the risk of a cardiac adverse event for treatment with an anti-IL-23p19 antibody other than hum13B8-b or treatment with an anti-IL-17 antibody for at least up to about 60 weeks.

27: The method according to claim 26, wherein the cardiac adverse event is pericarditis, atrial fibrillation, or coronary artery disease.

28: The method according to claim 26, wherein the plaque psoriasis is moderate to severe plaque psoriasis.

29: The method according to claim 26, wherein administration of hum13B8-b results in a reduced risk of a cardiac adverse event as compared to the risk of a cardiac adverse event for treatment with an anti-IL-23p19 antibody other than hum13B8-b or treatment with an anti-IL-17 antibody for at least up to about 64 weeks, for at least up to about 76 weeks, for at least up to about 88 weeks, for at least up to about 100 weeks, for at least up to about 112 weeks, for at least up to about 124 weeks, for at least up to about 136 weeks, for at least up to about 148 weeks, for at least up to about 160 weeks, for at least up to about 172 weeks, for at least up to about 184 weeks, for at least up to about 196 weeks, for at least up to about 208 weeks, for at least up to about 220 weeks, for at least up to about 232 weeks, for at least up to about 244 weeks, for at least up to about 256 weeks, for at least up to about 268 weeks, for at least up to about 280 weeks, for at least up to about 292 weeks, for at least up to about 304 weeks, for at least up to about 316 weeks, for at least up to about 328 weeks, for at least up to about 340 weeks, for at least up to about 352 weeks, for at least up to about 364 weeks, for at least up to about 384 weeks, for at least up to about 396 weeks, for at least up to about 408 weeks, or for at least up to about 432 weeks as compared to treatment for up to about 52 weeks.
